SEQUENCE LISTING (51 sequences)

SEQ ID NO: 1 - DNA, 449 bp, Psammomys Obesus
  misc_feature (1)..(425): “n” is unknown nucleotide
tgagaattaagattattgtatgattgaaacatgaaacagctcatgtctctgttagtaacatcaaaggcagtcacgtttacactgcagttagaactcttagggcacgttgcttttcatcaggcttcccctgcttttgatttgtggctgttgctgatttttcgtatgtggacatacacctacctcttctgttggaaagaacatttaaaatgaaacaaattttacttaaaaaaaaatcaaggagtcctttaatgtaaattttaactttcaaattactttctttatcttactttattacaaatagcacccactagcatttctgtgatgtacccacctctttcnatgtgctatttgactgatgcttgctccctgggatgacgttgcaaaaaatcgactagtagaaatgaagtgnaaatgcattttggtgnataccttgactccttggtgctaat

SEQ ID NO: 2 - DNA, 1702 bp, Psammomys Obesus
  misc_feature (1)..(1518): “n” is unknown nucleotide
cccacgcgtccggtgaagaattagtctgccttggcatttataaatgattcacaaccagtttccagttggaccccaaaaaactatttaattatcaatgagataatatgtttcctttctgaataatacataaaagaaatcagaggctgggaaaaaaagttatggataagaagttccagtccaataaaattatatttatctgtaggagtcatttttcctagatttctgtttctaacatcctggaaaaaaaattgagtttctggtaaatagtccagaaattatttagctaatttaatttctttacatttgaagactttagatttattttcagtatttagttaatataaaattaaaggaaaatcaacttaaacttagatcaacatttaatataactttatttttttgtgtacataataaatttatccagaagttaaatgcatggaaacatgattttataaaacaaaactaaccatgcttcccctccattgccatatctgctcttgtaggatgctattttctgtatgttaaaatcgggtgaatgagatagcatttgtggaatgagggtgtgcagtgctttatgtacaggtagttagagtaaggtgccttgttcttcactcgaatatatgaacatgccagttaaacctctttaaatttttttcttttactattcacctccttaactgaatcacaccagcttgcttagaatctcatggaagcaatgctcaggcaatttcacttttttgttgntgntattgncttcttttgaaaagctaatcatggcacatgtaagggacnggcgcgttcactgcccaggccattgcatggaatcactgcagtcttgtcctgcacatgggatctacactggctatctatagtaactagtgtaattgaaggcagacaagcttgacgcagataagctttgggcagctgaataatctacagggttagttttgaggatcaaatactggctgataattatgatattgccacatggtttgagtctatgtaagcttgaataagtgcatcttgtagcatctatgggaagtgtggggggaattaaagtgttagtgaagagaatatctaccaagtggctttttgtaagataggaaacaatagagtccaaagggaacttttaagagattgaaaagggggaataatatttcagcataaaatcctaaatttgaataaaagaaagaagaggagattggngctgttgctggtgtgtgcagattttctgggttcttccttttaatgttccactgtttttcctgtcagtattgtagatgtgtgatgacttgcctgtcaaatttatcaactgttgagaaagtaatagtgtaaatctttgtgccaacgaaacctctgccagtgttgtgtctctttacctcattcttgtatttggtcctgtcaagattgtagcttgtctgacagaagccacagcgnagnctgatgaaacttgccaggncagttttttgagcagctcccaaagccggaggcccagcttctcagccttggacagcttcngcctacccgtaaacctcagtggcatactctctaacagtgtgtgtagactgggatgctgttcacatgtggctttggatcatttctaatgtgacggaattttatttgtctcttgacagctttgtagaaatgcaatttttatagtaaagacattgttcatttggaagtcaatacgttataaaattgg

SEQ ID NO: 3 - DNA, 429 bp, Psammomys Obesus
  misc_feature (1)..(429): “n” is unknown nucleotide
agcgtggtcgcggccgaggtacataaaatggctaacttatagagatcaaaggttattttggtttgttgtttcaactgtttaactgttgctttacagtctgtggcaaagcagtgcatggtttagaatgtggcagaacaaagcagttctcacaaccactggatgcaaagagaagggaagaaaaaaaaagagaatagggttgaatatcccattcaagggcatgcctccatgtatgggttcctctnattagccaccctccctccccgccacaagtaaatcctaatagcaccagggtctggggtcaatcctttaccatacagccatttgtgagaaacttgttaagataacatatatcacactgcctgtgtatgctgaaataaattcataaattaataacatttcaaaaaaaacctgcccgggcggccgctcgaa

SEQ ID NO: 4 - DNA, 332 bp, Psammomys Obesus
  misc_feature (1)..(332): “n” is unknown nucleotide
tagcgtggtcgcggccgaggtactgacnttccattctgcttcacatgaaatcaacggaagctctttattcaagctttgggctctcagacacngaaatgagaccgtgaccatggcagtnggacaggaaaattcttgtttgaccctgaatgagagcacanaggcatgaatgtggaggtgccagcttgcctaatgaatgagggaacagccctttatgtaggtcagccgctttccttcccctgggctgccgtgctttcttcccaaagtcatccatttctgataatgatgtgaattgtctgttagctatcaagtacctgcccgggcggccgctcgaa

SEQ ID NO: 5 - DNA, 156 bp, Psammomys Obesus
agcgtggtcgcggccgaggtcagctttatacagaaaacaaggcttagcctctggacaacagagaagcgctcagtcccaaagccacaggaaggccttttacctgctccagccctcggccatggagtacctgcccgggcggccgcctcgaaagccgaa

SEQ ID NO: 6 - DNA, 447 bp, Psammomys Obesus
agactgcccgtgatccatgagcttaggatcaggcataaactgtgttttatctgaggtggattctgatttcatctttgtcacagaagcaaattcttttggcaaagacactgttttggcatgtatttcaccagaccttcctgccttagtttcttcttttggttcagggagtgattgtttttcactgctttttgtccacaatgacttaatggcttgataaatttgttggcttgtatcatatgtgtttctcccagttatctttgcaattattactaactgagctgggtagcaaacagctgctcctaatgtggccatccctagaggataagtaatcttctgaaacctagaacctcttcttgctgacagcaaacctgccagtcctgaagctgtgatcactcctattttgggcagaaaatcttgaggagggttcttcagatagatatatgtatc

SEQ ID NO: 7 - DNA, 389 bp, Psammomys Obesus
ccggaattccgggatatcgtcgacccacgcgtccggtggccaggccggccttgttggggctgctgactccaggtctattctcagcaatgggtcacagagactgaactgccaccatgattggaggcttattcatctataatcacaagggggaggtgctcatctcccgagtctacagagatgacatcgggaggaacgctgtggatgccttccgggtcaatgtgattcatgcgcggcagcaggtgcgaagccctgtcacaaacatcgctcgcaccagcttcttccatgttaagcggtccaacatctggctagcagcagtcaccaagcagaatgtcaacgccgccatggtcttcgaattcctctataagatgtgtgatgtaatggctgcttac

SEQ ID NO: 8 - DNA, 781 bp, Psammomys Obesus
gtcatcacacctgtgggaaataccttacacattactgttcgtgagtagctctgagacatcacagtggcctggtctgcagttgttgctctaaataattcgtttagagtgtttttctcgtttatgtctgcgtaatgactaaaatagtgagctcaggcccagtggcacagctgtactcccagctcttcagggggctgtggccggaggactgctagtttgaggccagccctgcagtaaagtagttaccaacagtgagactaaataaaaaaagttcagagcacaaacgaatacttgaagcaaaagagtctttcattttctttctctgactgaaaaaaacaagcactttccggaagccaagcacttttcattgtagcaaggatgagttatttacgttttaggaaattttatcatggattagtctgataatttaaattctggccagtctgttttcatctttttctaggttttacagtatttccatcatacctatgtaaaaggagaattatatacttgtatcatattggtgttgataatgtatattcagataataatattttgttagtgtctttattatctcaaaaggagttctaaaagtaaatactggaagcatatactttttagacattaattattttataatgaaacaactggcagtaagacattagtggttaatatagtctatttgttccagtgactgtaaagcttgtctcatagttctgtctgctcattagagaacgtattgtgaaattaccttctgtgcatgatatatctgtgcaattgcagt

SEQ ID NO: 9 - DNA, 1357 bp, Psammomys Obesus
tttttttttttttttgttagaaatgggaaaatttttcatcatctccagaactgtttattaatgatgctaataactttcttatagaccatttagaccatacagttccagaccacaacttcctgtttccagaatttgaaattgtaacagaaacagaagatgagattttgcctgaggctgtggaaatggaagattattctgacattgtaggaagcatgggtaaggagtccgtccatcaatctgacagttcagggaagacaggcaccatcagtatgtgtctgtggtgaggtagagaatatcatggtagaagactgcagatcaaaacagttaactgctgaatatatgacaagggtcctttgtaatttagtcttttagttttacttttagatacatgctagcttattttctcgggagctcatgctttagggctgtgtctgtggtgaccaacacgggtgctgatcataaatccagcattcaccctgccataggttgcttatgcatgtgctcactcaaacactctttgcatcgtgcttccagtaaagaaactgaagttctaggtatcctgtgtttgaacaaagaacttaggttcagatctgacactacagtgtgaactataatcaggtttgtaaaatcgtaatcagattctcaagtttagtcgtttgtttgccacaagcattaaacaacgcgtgtgagaattgtttcgtggctcagctcatgcttgctaacagaaatacacaacaggaagaatggtaccacatagtcaggggtctgttcgaaggagggagtgcagagagcgctcagctatgtaaagtgtcttgtgactggtaaaagttaaggagaaaggctggagagatggtcagaagtcacaagcacttgctgttcttgtagacgacccaggtttggtccctaaaacctgcacggcaatttgtaactctagttccaggtgatccagcgccctcctctggccttgaagtacacatacatataaacaaaacattcaaaaacaaacaaagaaacaaacaaataggaatacatacagcgtaggagctacctggatagaacttaaatgtgtctattcatgtgtgtactgtaaaaaaaactgaaaaaatcagtgagcatgttttttctttcattttgagagaggttctaatgaaggccaagctggctttgaacttgtcctcctgcctccacttcctgaatacagatattacatcattttacatctgtctgctgttatcttaatgagtaaacttatatataggtctaggaaaatagtacagagttacaaaacaatataggtctgaacttttattggaagatgccagcagctcatgagtacatttatctacaacaaagattt

SEQ ID NO: 10 - DNA, 1314 bp, Psammomys Obesus
ggctagaaaactacaagagaaaaattcacatgaaaaaccaaaatgaagaaaaggctgctgagcagtttcggatgcgactgaaaaacaagcaagatgaaatgaggcttgaaggagacctgagaagaagccagcgtgcttgccagcagcttgatgcccagaagaatattcaggttcccagagaggtatggtactggataaggcctgaagaagacactgaagaagaggaagtggaggaggaacaggatgaagatgaatacacgagtgaagatttgagtgtgctggaaaaactgcagatcctcacaggctacttacgagaagaacatctgtattgcatttggtgtgggacagcctatgaagataaagaagacttatcttccaattgcccaggaccaacctctgctactacagtgctcagttagaatgaagccagaactggggtagctacagccgtctaactatgaggacatcaggaagcacttcctgagtggaggacgttctacagaaggatgactagttcctcaagcaaatgttgagagaacagaaaacacagggagtatgccacagagtaaaagtcaaccaaatgaaaccttggatcctcctgtttataaacagctatatacaaaagacagttttgagaatataaaattgaatccagactaattatatgataattatatttctgtttgtctgagtagcagtgatgtgtgattttgttaatatttcaaaatacttgtccatataagtcaacttttaagtatttgcagctgaaacactacaatgctaagagtaactttaaaattttccagccaaagaaacaaaaaggaaggggtaggtgtcacagcaaaggtaaaatattctctgttaaagctgcacaatggcacatacaatctcattattcttgtatacgtttgaagacaaaaaaatatatgtaataattaatcttgaatgaaaatattttgtcttaaaaaaatgttaatgtgccagagaacctcaagttatggtgaacattcaagtggggatgcatgttgtaagtctttgcctattgagcctgcagatgtgtttttaatagaaatgatatatttggtctgtgataaaaatttcttaggtctgtttctattgattcaactagttgtttgtttgtttgtttgtttgtttgtttgtttcaaaactgtacctgaaattagcaattgaggtcctgctctgccccactgttactctgattaagaagatacttgttatttcctggtttttggagacagacgatatatcctcagaagtaaataaaagatgctaagagataaaaaaaaaaaaaaa

SEQ ID NO: 11 - DNA, 319 bp, Psammomys Obesus
caccgcccgccgtcacgtcggcggagggctgcggggcgcgtgcgattgagctgctggagttctcaagatgaagttttcattggcgatctccttttttattttaatgtccttgtggtttgaagaagcttgttctaaagaaaagtcttccaagaaagggaaggggaaaaagaagcagtatttgtgcccatctcagcagtcaccagaggacctggcgcgtgtgccccccaactccaccagtaatatcctgaacaggctgctggtcagttatgaccccaggatcagaccaaatttcaaaggtattcctgtcgatgtagtagtc

SEQ ID NO: 12 - DNA, 1262 bp, Psammomys Obesus
  misc_feature (1)..(1262): “n” is unknown nucleotide
aaactctgtctcaaaacaaaacaaagtaacaaagggataggccaagtcttctgagaagttagaggcaaagtgcttgcttgagcctaatgctcttcccacagccgtcctcagcccaggtcctctcctctcctgcaatcaagaggatattgctttagttgacatgggcctttccacgcatctgctgaggtgattttccaggtaactacaggggtgaaggctaactcaacacaagccaagaaatacagaatctagatatacctggtcttacctagatgggagaccagcaccgtccaatagcagggcagccagccgcccagatccttataggtctaggaacactggatgacagctggtgaacatgggctggagtgactgtgaccccttcaagggaaagcctcagctctttttacctgttaaggaatgatggagatgcagacagtaggagagtggtgtgggagaaatccaaagtggcaatttcccaaacactttttaaaataaatcttgtgtaggatgacatagggctttgattgtggctcagtggtaccatgcttgtcttggacactttaatgtgacaagtctgagggtccggtctatcactggaactgcaaaaaccaagaaacaactgaaatgataaagcagttcagagctgggaagtttataatttctttcttcttataccttttcccccaagtggaaaatgcttaattagttgaagggccccggaacagtcctaagagcctgggaaacacttatttgcaagatgggtgcattctggtttccctaaaaccacaactcaaaaggaaggttgtggggacagatgagtcatgctgcctcagactagtgggtgaaagaggaagacagtgtcctgcctgtggcctcatgccagcagaacacctgagagtctgagggaggttgtgtgaccttgccaccaaatggtatacagccgagcaggcgaaganagggtgacagagtgggcagccttctcttcagcaagatgtaaggaacaatgccagttgctcatactaacgagaagcagcccctttgccagaaggttctcagtacgccctccctccttacattcactttgtccctttcagagagccaggtcaccacaaaggccacctggcctgcccactcacttctgccaaaatgttgcatgccagcgtggaagacactgcacaaatcccagtgtgtatttacttagtggacacagatacctttaaaataaaataaattcattaatgaaaaaaaaaaaaaaaaaa

SEQ ID NO: 13 - DNA, 364 bp, Psammomys Obesus
gctgctttagccaaagccatcgaaaagaacgtgttattttcacaccttgatgacaatgagagaagtgacatttttgatgctatgtttccagtctcctttattgctggagagacagttattcagcaaggtgatgaaggggataacttctatgtgattgatcaaggagaaatggatgtctatgtcaataatgaatgggcaaccagcgttggggaaggagggagcttcggagagttggctttgatttatggaacacctagagcagccactgtcaaagcaaagacaaatgtgaaactgtggggcatcgaccgagacagctaccgaagaatcctcatgggaagcactctgagaaagaggaagatgtatg

SEQ ID NO: 14 - DNA, 390 bp, Psammomys Obesus
cccacgcgtccgcccacgcgtccgcggacgcgtgggctgtcctagccttgctttatagaccaggctggctttgacctcacagagatctgcttgttggaccctcccccaagtgctggaattacaggcgtgcgccaccatacccagctctgatatctcttacatgacaaaaatcaagtcactattaatgaaaatgatctgctcattatagatgggaggcttaacaaataagtgatacatgctaatttctgccagtgtctattgcttaattgttaattgtgagcagattactgaatgcctcgtctattttctacattttattttacaataactctttgagtaagttgaagtttaattgtgtagcaaatttctatcagagaacaatttaaagtg

SEQ ID NO: 15 - DNA, 582 bp, Psammomys Obesus
ctatgacatgatgccagattacaggcagatcccatgtaaccagaaggacaatgtgtcttctaaaaagggaaggtgttactggagcttttctttcctctgcacagcatagtatcaaagtatgccattaatgaagatttattccattactgctataaatttttagtataggaagaaaactttatcacgatcatgggccagttagcctgatgtattcaagaacaaacaagagaccctgtctcaagttaggaatgaggacaagtgcctgaaggtgccctccaacctccacagttgtccgaagcacacatccgtgatcatgcacacatcagcacgcgtacaaaattacacttgaggctttagtatcatgtgttgatcatttcaaaccatcagaggcaaacactgaagtggtattttctgtctcctgcttgccagtatctacattttccctcacattctaggttaaaaaatggttcttttataacatgagcaatttgtgatgtttattataagtaaatgttgatgtcagtgttagaattaaaattacttgtagtgataaaaaaaaaaaaaaaaaaaaaaaaaaaaaaa

SEQ ID NO: 16 - DNA, 341 bp, Psammomys Obesus
agagaaataaagatggaactcggatgtttgaaaatctttacattcaaataaggccaaatgtcacggggagaaaaaaaaaaatgggaaggtgagctgctatgactgagacccctgaacttcccactcacactctctgcagaaccccacaaaacgacacagaacactatgtggttgttctatggcttttgttttttcgaggttctttcaaatatcctttggcgattgaagcttaaattccatttgattggcttcaccgtctgtaaatacttagctattagctgtaagcactacacgtagatgacttaacgacgggcaggtcccagcgatcatagctttatgac

SEQ ID NO: 17 - DNA, 22 bp, Psammomys Obesus
gcaaagacctgtatgccaacac

SEQ ID NO: 18 - DNA, 23 bp, Psammomys Obesus
gccagagcagtgatctctttctg

SEQ ID NO: 19 - DNA, 25 bp, Psammomys Obesus
tccggtccacaatgcctgggtacat

SEQ ID NO: 20 - DNA, 23 bp, Psammomys Obesus
cacccactagcatttctgtgatg

SEQ ID NO: 21 - DNA, 23 bp, Psammomys Obesus
cattagcaccaaggagtcaaggt

SEQ ID NO: 22 - DNA, 21 bp, Psammomys Obesus
ctaaccatgcttcccctccat

SEQ ID NO: 23 - DNA, 23 bp, Psammomys Obesus
caccctcattccacaaatgctat

SEQ ID NO: 24 - DNA, 23 bp, Psammomys Obesus
gttgctttacagtctgtggcaaa

SEQ ID NO: 25 - DNA, 21 bp, Psammomys Obesus
tgcatccagtggttgtgagaa

SEQ ID NO: 26 - DNA, 23 bp, Psammomys Obesus
gaggaacagccctttatgtaggt

SEQ ID NO: 27 - DNA, 23 bp, Psammomys Obesus
gaaatggatgactttgggaagaa

SEQ ID NO: 28 - DNA, 22 bp, Psammomys Obesus
aggcttacggtctggacaacag

SEQ ID NO: 29 - DNA, 22 bp, Psammomys Obesus
cgctttgccgaatacctctaaa

SEQ ID NO: 30 - DNA, 30 bp, Psammomys Obesus
aagtaatcttctgaaacctagaacctcttc

SEQ ID NO: 31 - DNA, 23 bp, Psammomys Obesus
ctgcccaaaataggagtgatcac

SEQ ID NO: 32 - DNA, 20 bp, Psammomys Obesus
ccgggtcaatgtgattcatg

SEQ ID NO: 33 - DNA, 19 bp, Psammomys Obesus
ggtgcgagcgatgtttgtg

SEQ ID NO: 34 - DNA, 25 bp, Psammomys Obesus
tctcgtttatgtctgcgtaatgact

SEQ ID NO: 35 - DNA, 21 bp, Psammomys Obesus
ccctgaagagctgggagtaca

SEQ ID NO: 36 - DNA, 20 bp, Psammomys Obesus
aggctggctttgacctcaca

SEQ ID NO: 37 - DNA, 18 bp, Psammomys Obesus
gcgcacgcctgtaattcc

SEQ ID NO: 38 - DNA, 22 bp, Psammomys Obesus
gaggcaggaggacaagttcaaa

SEQ ID NO: 39 - DNA, 26 bp, Psammomys Obesus
tcagtgagcatgttttttctttcatt

SEQ ID NO: 40 - DNA, 24 bp, Psammomys Obesus
cctcaagcaaatgttgagagaaca

SEQ ID NO: 41 - DNA, 23 bp, Psammomys Obesus
aacaggaggatccaaggtttcat

SEQ ID NO: 42 - DNA, 25 bp, Psammomys Obesus
gagaccctgtctcaagttaggaatg

SEQ ID NO: 43 - DNA, 21 bp, Psammomys Obesus
gtgtgcatgatcacggatgtg

SEQ ID NO: 44 - DNA, 22 bp, Psammomys Obesus
agcagtatttgtgcccatctca

SEQ ID NO: 45 - DNA, 21 bp, Psammomys Obesus
aactgaccagcagcctgttca

SEQ ID NO: 46 - DNA, 20 bp, Psammomys Obesus
agttgacatgggcctttcca

SEQ ID NO: 47 - DNA, 23 bp, Psammomys Obesus
ggcttgtgttgagttagccttca

SEQ ID NO: 48 - DNA, 23 bp, Psammomys Obesus
cctgaacttcccactcacactct

SEQ ID NO: 49 - DNA, 26 bp, Psammomys Obesus
caaaagccatagaacaaccacatagt

SEQ ID NO: 50 - DNA, 20 bp, Psammomys Obesus
gcttcggagagttggctttg

SEQ ID NO: 51 - DNA, 21 bp, Psammomys Obesus
gccccacagtttcacatttgt

(19) United States
(12) Patent Application Publication
(10) Pub. No.: US 2006/0228775 A1
(43) Pub. Date: Oct. 12, 2006

(54) Differential expression of nucleic acid molecules

(75) Inventors: Greg Royce Collier, Victoria (AU); Ken Russell Walder, Victoria (AU)

Correspondence Address: DUANE MORRIS LLP;PATENT DEPARTMENT, 380 LEXINGTON AVENUE, NEW YORK, NY, 10168-0002, US

(73) Assignee: CHEMGENEX PHARMACEUTICALS LLIMITED

(21) Appl. No.: 10/564,077

(22) PCT Filed: Jul. 8, 2004
(86) PCT No.: PCT/AU04/00919

§ 371 Date: May 18, 2006

Related U.S. Application Data
(60) Provisional application No. 60/485,790, filed on Jul. 8, 2003.

(51) Int. Cl. A61K 38/16; C07H 21/04; C12P 21/06; C07K 14/705

(52) U.S. Cl. 435/69.1; 435/320.1; 435/325; 514/2; 530/350; 536/23.5

(57) ABSTRACT

The present invention relates generally to nucleic acid molecules expressed at least in the hypothalamus, liver, mesenteric adipose tissue, or red gastrocnemius muscle conveniently identified using differential display techniques under differing physiological conditions. The nucleic acid molecules are associated with or act as markers for conditions of inter alia a healthy state, myopathy, obesity, anorexia, weight maintenance, diabetes, disorders associated with mitochondrial dysfunction, genetic disorders and/or metabolic energy levels. More particularly, the present invention is directed to a nucleic acid molecule and/or its expression product for use in therapeutic and diagnostic protocols for conditions such as inter alia a myopathy, obesity, anorexia, weight maintenance, diabetes, disorders associated with mitochondrial dysfunction, genetic disorders and/or metabolic energy levels. The subject nucleic acid molecule and expression product and their derivatives, homologs, analogs and mimetics are proposed to be useful, therefore, as therapeutic and diagnostic agents for inter alia a myopathy, obesity, anorexia, weight maintenance, diabetes, disorders associated with mitochondrial dysfunction, genetic disorders and/or metabolic energy levels or as targets for the design and/or identification of modulators of their activity and/or function.

### BACKGROUND OF THE INVENTION

1. Field of the Invention

The present invention relates generally to nucleic acid molecules identified by a pattern of their expression in at least the hypothalamus, liver, mesenteric adipose tissue or red gastrocnemius muscle. More particularly, the present invention provides nucleic acid molecules which are associated with or act as markers for conditions of inter alia a healthy state, myopathy, obesity, anorexia, weight maintenance, diabetes, disorders associated with mitochondrial dysfunction, genetic disorders and/or metabolic energy levels. The present invention is also directed to a nucleic acid molecule and/or its expression product for use in therapeutic and diagnostic protocols for conditions such as inter alia a myopathy, obesity, anorexia, weight maintenance, diabetes, disorders associated with mitochondrial dysfunction, genetic disorders and energy imbalance.

2. Description of the Prior Art

Reference to any prior art in this specification is not, and should not be taken as, an acknowledgment or any form of suggestion that this prior art forms part of the common general knowledge in any country.

Bibliographic details of the publications referred to by author in this specification are collected at the end of the description.

The increasing sophistication of recombinant DNA technology is greatly facilitating research and development in the medical, veterinary and allied human and animal health fields. This is particularly the case in the investigation of the genetic bases involved in the etiology of certain disease conditions. One particularly significant condition from the stand point of morbidity and mortality is obesity and its association with type 2 diabetes (formerly non-insulin-dependent diabetes mellitus or NIDDM) and cardiovascular disease.

Obesity is defined as a pathological excess of body fat and is the result of an imbalance between energy intake and energy expenditure for a sustained period of time. Obesity is the most common metabolic disease found in affluent nations. The prevalence of obesity in these nations is alarmingly high, ranging from 10% to upwards of 50% in some subpopulations (Bouchard, The genetics of obesity. Boca Raton: CRC Press, 1994). Of particular concern is the fact that the prevalence of obesity appears to be rising consistently in affluent societies and is now increasing rapidly in less prosperous nations as they become more affluent and/or adopt cultural practices from the more affluent countries (Zimmet, Diabetes Care 15(2): 232-247, 1992).

In 1995 in Australia, for example, 19% of the adult population were obese (BMI>30). On average, women in 1995 weighed 4.8 kg more than their counterparts in 1980 while men weighed 3.6 kg more (Australian Institute of Health and Welfare (AIHW), Heart, Stroke and Vascular diseases, Australian facts. AIHW Cat. No. CVD 7 Canberra: AIHW and the Heart Foundation of Australia, 1999.). More recently, the AusDiab Study conducted between the years 1999 and 2000 showed that 65% of males and 45% of females aged 25-64 years were considered overweight (de Looper and Bhatia, Australia's Health Trends 2001. Australian Institute of Health and Welfare (AIHW) Cat. No. PHE 24. Canberra: AIHW, 2001). The prevalence of obesity in the U.S. also increased substantially between 1991 and 1998, rising from 12% to 18% in Americans during this period (Mokdad et al., JAMA. 282(16): 1519-22, 1999).

The high and increasing prevalence of obesity has serious health implications for both individuals and society as a whole. Obesity is a complex and heterogeneous disorder and has been identified as a key risk indicator of preventable morbidity and mortality since obesity increases the risk of a number of other metabolic conditions including type 2 diabetes mellitus and cardiovascular disease (Must et al., JAMA. 282(16): 1523-1529, 1999; Kopelman, Nature 404: 635-643, 2000). Alongside obesity, the prevalence of diabetes continues to increase rapidly. It has been estimated that there were about 700,000 persons with diabetes in Australia in 1995 while in the US, diabetes prevalence increased from 4.9% in 1990 to 6.9% in 1999 (Mokdad, Diabetes Care 24(2): 412, 2001). In Australia, the annual costs of obesity associated with diabetes and other disease conditions has been conservatively estimated to be AU$810 million for 1992-3 (National Health and Medical Research Council, Acting on Australia's weight: A strategy for the prevention of overweight and obesity. Canberra: National Health and Medical Research Council, 1996). In the US, the National Health Interview Survey (NHIS) estimated the economic cost of obesity in 1995 as approximately US$99 billion, thereby representing 5.7% of total health costs in the U.S. at that time (Wolf and Colditz, Obes Res. 6: 97-106, 1998).

A genetic basis for the etiology of obesity is indicated inter alia from studies in twins, adoption studies and population-based analyses which suggest that genetic effects account for 25-80% of the variation in body weight in the general population (Bouchard [1994; supra]; Kopelman et al., Int J Obesity 18: 188-191, 1994; Ravussin, Metabolism 44(Suppl 3): 12-14, 1995). It is considered that genes determine the possible range of body weight in an individual and then the environment influences the point within this range where the individual is located at any given time (Bouchard [1994; supra]). However, despite numerous studies into genes thought to be involved in the pathogenesis of obesity, there have been surprisingly few significant findings in this area. In addition, genome-wide scans in various population groups have not produced definitive evidence of the chromosomal regions having a major effect on obesity.

A number or organs/tissues have been implicated in the pathophysiology of obesity and type 2 diabetes, including the hypothalamus and liver. One organ of particular interest is the hypothalamus. Early studies led to the dual-center hypothesis which proposed that two opposing centers in the hypothalamus were responsible for the initiation and termination of eating, the lateral hypothalamus (LHA; “hunger center”) and ventromedial hypothalamus (VMH; “satiety center”; Stellar, Psychol. Rev. 61: 5-22, 1954). The dual-center hypothesis has been repeatedly modified to accommodate the increasing information about the roles played by various other brain regions, neurotransmitter systems and hormonal and neural signals originating in the gut on the regulation of food intake. In addition to the LHA and VMH, the paraventricular nucleus (PVN) is now considered to have an important integrative function in the control of energy intake.

A large number of neurotransmitters has been investigated as possible hypothalamic regulators of feeding behavior including neuropeptide Y (NPY), glucagon-like peptide 1 (GLP-1), melanin-concentrating hormone (MCH), serotonin, cholecystokinin and galanin. Some of these neurotransmitters stimulate food intake, some act in an anorexigenic manner and some have diverse effects on energy intake depending on the site of administration. For example, γ-aminobutyric acid (GABA) inhibits food intake when injected into the LHA, but stimulates eating when injected into the VMH or PVN (Leibowitz, 1985). Feeding behavior is thought to be greatly influenced by the interaction of stimulatory and inhibitory signals in the hypothalamus.

The liver also plays a significant role in a number of important physiological pathways. It has a major role in the regulation of metabolism of glucose, amino acids and fat. In addition the liver is the only organ (other than the gut) that comes into direct contact with a large volume of ingested food and therefore the liver is able to “sense” or monitor the level of nutrients entering the body, particularly the amounts of protein and carbohydrate. It has been proposed that the liver may also have a role in the regulation of food intake through the transmission of unidentified signals relaying information to the brain about nutrient absorption from the gut and metabolic changes throughout the body (Russek, Nature 200: 176, 1963; Koopmans, 1998, supra). The liver also plays a crucial role in maintaining circulating glucose concentrations by regulating pathways such as gluconeogenesis and glycogenolysis. Alterations in glucose homeostasis are important factors in the pathophysiology of impaired glucose tolerance and the development of type 2 diabetes mellitus.

In accordance with the present invention, genetic sequences were sought which are differentially expressed in lean and obese animals or in fed compared to unfed animals. Nucleic acid moles are identified which are proposed to be associated with or act as markers for energy balance as well as inter alia a healthy state, myopathy, obesity, anorexia, weight maintenance, disorders associated with mitochondrial dysfunction, genetic disorders and diabetes.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identified numbers is provided in Table 2. A sequence listing is provided after the claims.

Differential display analysis of genetic material isolated from either hypothalamus or liver, mesenteric adipose tissue or red gastrocnemius muscle tissue was used to identify candidate genetic sequences associated with inter alia a healthy state or with physiological conditions such as inter alia a myopathy, obesity, anorexia, weight maintenance, diabetes, disorders associated with mitochondrial dysfunction, genetic disorders and/or metabolic energy levels. An animal model was employed comprising the Israeli Sand Rat (Psammomys obesus). Three groups of anrimals were used designated Groups A, B and C based on metabolic phenotype as follows:

Group A: lean animals (normoglycemic; normoinsulinemic);

Group B: obese, non-diabetic animals (normoglycemic; hyperinsulinemic); and

Group C: obese, diabetic animals (hyperglycemic; hyperinsulinemic).

Animals were maintained under two study conditions: (1) they were either fed ad libitum (“fed”) or fasted for 24 hours (“fasted”) prior to analysis; or (2) maintained by being fed ad libitum (“control”) or placed on an energy restricted diet (“restricted”), and genetic sequences analyzed by differential display analysis. In a preferred embodiment using these techniques, sixteen differentially expressed sequences were identified from cells of either the hypothalamus, liver, mesenteric adipose tissue and/or red gastrocnemius muscle and designated herein AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 with sequence identifiers SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, respectively.

Differential expression means an elevation in levels of expression of a genetic sequence under one set of conditions compared to another.

In one particular embodiment, AGT-711 gene expression was elevated in the liver of Group B control animals and Group C energy restricted animals compared to Group C control animals. Additionally, when AGT-711 gene expression was examined in the liver, it was shown to be elevated in Group C fed animals compared to Group A fed animals. When comparisons were made across all animals, gene expression in hepatic tissue was found to be positively correlated with body weight, glucose levels and insulin levels. Cell culture experiments show AGT-711 gene expression in liver cells was regulated by glucose levels, and AGT-711 gene expression in skeletal muscle cells was regulated by insulin levels.

AGT-712 gene expression was significantly elevated in the hypothalamus in Group B control and Group C control animals when compared to Group A control animals. Following dietary energy restrictions, AGT-712 gene expression was found to be positively correlated with glucose and insulin levels in fed animals. AGT-712 gene expression, when examined across all animals, was also positively correlated insulin levels.

AGT-713 gene expression was reduced in the liver under fasting conditions compared to animals that were fed. AGT-713 gene expression in liver was found to be negatively correlated with percent body fat when measured in fasted animals.

AGT-714 gene was elevated in the liver under fasting conditions in Group A, B and C animals. AGT-714 gene expression, when examined across all animals, was negatively correlated with body weight and insulin levels. When AGT-714 gene expression in the liver was examined in animals maintained under fasting conditions, it was found to be negatively correlated with body weight and positively correlated with the change in blood glucose levels after fasting. In animals that were fed ad libitum, AGT-714 gene expression was found to be positively correlated with percent body fat.

AGT-715 gene expression was significantly elevated in liver under fasting conditions in Group B animals, compared with the level of expression in the liver under fasting conditions of Group A animals. AGT-715, when examined in fed animals, was shown to be positively correlated with percent body fat.

AGT-716 gene expression in the red gastrocnemius muscle was significantly lower in Group C obese/diabetic control animals compared to Group A and Group B animals. Further, AGT-716 gene expression was significantly lower in Group A fasted animals compared to group A fed animals. AGT-716 gene expression was significantly lower in mesenteric adipose tissue from Group A fasted animals when compared to Group C fasted animals, with gene expression being shown to be lower in all fasted animals when compared to all fed animals. AGT-716 gene expression, when examined in the gastrocnemius muscle of all animals, was shown to be negatively correlated with blood insulin and percentage body fat. AGT-716 gene expression in adipose tissue was shown to be positively correlated with body weight in fasted animals.

AGT-717 gene expression was significantly lower in hypothalamus tissue from Group A control animals compared to both Group B and C control animals. Additionally, AGT-717 gene expression was significantly lower after dietary energy restriction in Group C animals, but not in Group A and Group B animals. Further, when AGT-717 gene was examined in the hypothalamus of P. obesus, it was found to be positively correlated with body weight, blood glucose levels, blood insulin levels and fat oxidation.

AGT-718 gene expression was found to be significantly higher in the hypothalamus of Group C animals when compared to Group A animals. Additionally, AGT-718 gene expression in mesenteric fat was found to be significantly lower in Group A fed animals compared to both Group B and C fed animals. In addition, AGT-718 levels were higher in the mesenteric fat of fed animals, compared to those kept under fasting conditions. When AGT-718 gene expression was examined in the red gastronnemius muscle, it was found to be significantly elevated in Group B fed animals compared to Group C fed animals. When AGT-718 gene expression was examined across all groups, with fed animals being compared to fasted animals, it was shown to be elevated in fed animals versus fasted animals in both red gastronnemius muscle and liver. When AGT-718 gene expression was examined in all animals, post-restriction, there was a positive correlation with body weight. When the hypothalamuses of all control animals were examined, AGT-718 gene expression was shown to be positively correlated with blood insulin, blood glucose, total activity (total beam interruption) and fat oxidation. AGT-718 gene expression, when examined in the mesenteric fat of fed/fasted animals, was shown to be positively correlated with body weight, blood insulin levels and percent body fat.

AGT-720 gene expression was shown to be elevated in the hypothalamus of Group B and C animals when compared to Group A animals. Additionally, hypothalamic gene expression of AGT-720 was positively correlated with body weight in dietary energy restricted animals and ad libitum fed animals. When AGT-720 gene expression in the hypothalamus was examined across all control groups, it was shown to be positively correlated with body weight, plasma glucose levels and total energy expenditure.

AGT-721 gene expression was shown to be elevated in the hypothalamus of Group C animals when compared to Group A animals. AGT-721 gene expression, when examined across all control groups, was shown to be positively correlated with blood glucose levels and blood insulin levels.

AGT-723 gene expression was shown to be elevated in the hypothalamus of Group C animals when compared to both Group A and B animals. When AGT-723 gene expression was measured in mesenteric fat, it was shown to be significantly elevated in Group B fed animals, when compared to Group A fed animals, Group B fasted animals and Group C fed animals, and was significantly reduced after fasting in these animals. When gene expression in mesenteric fat was compared across all control animals, it was found to be positively correlated with body weight and plasma insulin levels. In the hypothalamus, when AGT-723 gene expression was examined in fasted animals, it was shown to be negatively correlated with blood glucose levels. When AGT-723 gene expression was examined in all animals from the fed/fasted study, it was shown to be positively correlated with body weight and blood insulin levels. Finally, AGT-723 gene expression levels were examined in the hypothalamus of fed and fasted Sprague Dawley rats. Gene expression was found to be significantly elevated in Sprague Dawley rats that were under fasting conditions for 48 hours when compared to animals that were kept under fasting conditions for 24 hours.

AGT-724 gene expression was shown to be significantly lower in Group A animals compared to Group B and Group C animals. When AGT-724 gene expression in the hypothalamus was examined across all animals, it was shown to be positively correlated with body weight and blood glucose levels.

AGT-726 gene expression was shown to be elevated in the hypothalamus of Group B and Group C animals compared to Group A animals. In control animals hypothalamic gene expression was shown to be positively correlated with body weight, blood glucose, total activity and insulin levels, as well as being positively correlated with body weight in dietary restricted animals. When AGT-726 gene expression was examined in the hypothalamus after 24 hours of fasting, its expression was reduced in Group C compared to Group A and Group B animals. When AGT-726 gene levels were examined in mesenteric fat, it was shown to be elevated in Group C fed animals compared to Group A and Group B fed animals. When AGT-726 expression was examined in animals after 24 hours of fasting, levels were significantly reduced in Group C, but not in Group A or B. Mesenteric fat AGT-726 gene expression was positively correlated with body weight, blood glucose and plasma insulin concentration. AGT-726 gene expression was also analyzed in the red gastrocnemius muscle after fasting or feeding. In the fed state, AGT-726 gene expression was reduced in group C compared with Group A and B. AGT-726 gene expression in the red gastrocnemius muscle was negatively correlated with blood glucose levels.

AGT-719 gene expression in the hypothalamus was found to be significantly elevated in Group C control animals compared both Group A and B control animals. When AGT-719 gene expression was examined in the hypothalamus, there was a positive correlation between body weight and blood glucose levels. Hypothalamic gene expression was shown to be positively correlated with body weight, blood glucose, total activity, insulin levels and percent body fat in dietary restricted animals.

AGT-722 gene expression in the hypothalamus was significantly increased in Group B control and Group C control animals versus Group A control animals. Expression of AGT-722 was also significantly increased in Group A energy restricted animals as compared to Group A control animals. Significant positive correlations were observed between AGT-722 gene expression levels and blood glucose, insulin levels, activity and percent body fat. AGT-725 gene expression in the hypothalamus AGT-725 gene expression is significantly lower in A controls when compared to C controls. Gene expression significantly lower in C controls when compared to C energy restricted and gene expression positively correlated with blood glucose in control animals, and positively correlated with total activity in energy restricted animals.

A summary of the AGT sequences is provided in Table 1.

TABLE 1
Summary of Differentially Expressed Genes

| GENE | SEQ ID NO: | TISSUE |
|---|---|---|
| AGT-711 | 1 | Liver |
| AGT-712 | 2 | Hypothalamus |
| AGT-713 | 3 | Liver |
| AGT-714 | 4 | Liver |
| AGT-715 | 5 | Liver |
| AGT-716 | 6 | Red gastrocnemius muscle, mesenteric adipose |
| | | tissue |
| AGT-717 | 7 | Hypothalamus |
| AGT-718 | 8 | Hypothalamus, Red gastrocnemius muscle, |
| | | liver |
| AGT-720 | 9 | Hypothalamus |
| AGT-721 | 10 | Hypothalamus |
| AGT-723 | 11 | Hypothalamus, mesenteric adipose tissue |
| AGT-724 | 12 | Hypothalamus |
| AGT-726 | 13 | Hypothalamus, Red gastrocnemius muscle |
| AGT-719 | 14 | Hypothalamus |
| AGT-722 | 15 | Hypothalamus |
| AGT-725 | 16 | Hypothalamus |

The identification of these variably expressed sequences permits the rationale design and/or selection of molecules capable of antagonizing or agonizing the expression products and/or permits the development of screening assays. The screening assays, for example, include assessing the physiological status of a particular subject.

Accordingly, one aspect of the present invention provides a nucleic acid molecule comprising a sequence of nucleotides encoding or complementary to a sequence encoding a protein or mRNA or a derivative, homolog, analog or mimetic thereof wherein the nucleic acid molecule is expressed in larger amounts in hypothalamus of fasted animals compared to fed animals.

In a preferred embodiment, the nucleic acid molecule comprises a nucleotide sequence substantially as set forth in SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 or a nucleotide sequence having at least about 40% similarity to all or part of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 or their complementary forms under low stringency conditions at 42° C.

Another aspect of the present invention provides an isolated molecule or a derivative, homologue, analog or mimetic thereof which is produced in a larger amount in hypothalamus tissue of obese animals compared to lean animals and/or which is produced in a larger amount in hypothalamus tissue of fasted animals compared to fed animals.

The molecule is generally a protein but may also be an mRNA, intron or exon. In this respect, the molecule may be considered an expression product of the subject nucleotide sequences.

In a preferred embodiment, the nucleic acid molecule comprises a nucleotide sequence substantially set forth in SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 or a nucleotide sequence having at least about 40% similarity to all or part of SEQ ID NO:1 or SEQ ID NO:2 or SEQ ID NO:3 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 or their complementary forms under low stringency conditions.

The preferred genetic sequence of the present invention are referred to herein AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 . The expression products encoded by AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 are referred to herein as AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725, respectively. The expression product may be an RNA (e.g. mRNA) or a protein. Where the expression product is an RNA, the present invention extends to RNA-related molecules associated thereto such as RNAi.

A further aspect of the present invention relates to a composition AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or its derivatives, homologs, analogs or mimetics or agonists or antagonists of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 together with one or more pharmaceutically acceptable carriers and/or diluents.

Yet a further aspect of the present invention contemplates a method for treating a subject comprising administering to said subject a treatment effective amount AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or a derivative, homolog, analog or mimetic thereof or a genetic sequence encoding same or an agonist or antagonist AGT-711, AGT-712, AGT-713, AGT-714, AGT-715, AGT-716,,AGT-717, AGT-718, AGT-720, AGT-721, AGT-723, AGT-724, AGT-726, AGT-719, AGT-722 and/or AGT-725 activity AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 gene expression for a time and under conditions sufficient to effect treatment.

In accordance with this and other aspects of the present invention, treatments contemplated herein include but are not limited for inter alia a myopathy, obesity, anorexia, weight maintenance, diabetes, disorders associated with mitochondrial dysfunction, genetic disorders and/or metabolic energy levels. Treatment may be by the administration of a pharmaceutical composition or genetic sequences via gene therapy. Treatment is contemplated for human subjects as well as animals such as animals important to livestock industry.

Still yet another aspect of the present invention is directed to a diagnostic agent for use in monitoring or diagnosing conditions such as but not limited inter alia a myopathy, obesity, anorexia, weight maintenance, diabetes, disorders associated with mitochondrial dysfunction, genetic disorders and/or metabolic energy levels, said diagnostic agent selected from an antibody to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 or AGT-725 or its derivatives, homologs, analogs or mimetics and a genetic sequence comprising or capable of annealing to a nucleotide strand associated with AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 or AGT-725 useful inter alia in PCR, hybridization and/or RFLP.

A summary of sequence identifiers used throughout the subject specification is provided in Table 2.

TABLE 2
Summary of Sequence Identifiers

| SEQUENCE ID NO: | DESCRIPTION |
|---|---|
| SEQ ID NO: 1 | Nucleotide sequence of AGT-711 |
| SEQ ID NO: 2 | Nucleotide sequence of AGT-712 |
| SEQ ID NO: 3 | Nucleotide sequence of AGT-713 |
| SEQ ID NO: 4 | Nucleotide sequence of AGT-714 |
| SEQ ID NO: 5 | Nucleotide sequence of AGT-715 |
| SEQ ID NO: 6 | Nucleotide sequence of AGT-716 |
| SEQ ID NO: 7 | Nucleotide sequence of AGT-717 |
| SEQ ID NO: 8 | Nucleotide sequence of AGT-718 |
| SEQ ID NO: 9 | Nucleotide sequence of AGT-720 |
| SEQ ID NO: 10 | Nucleotide sequence of AGT-721 |
| SEQ ID NO: 11 | Nucleotide sequence of AGT-723 |
| SEQ ID NO: 12 | Nucleotide sequence of AGT-724 |
| SEQ ID NO: 13 | Nucleotide sequence of AGT-726 |
| SEQE ID NO: 14 | Nucleotide sequence of AGT-719 |
| SEQE ID NO: 15 | Nucleotide sequence of AGT-722 |
| SEQE ID NO: 16 | Nucleotide sequence of AGT-725 |
| SEQE ID NO: 17 | β-Actin forward primer |
| SEQE ID NO: 18 | β-Actin reverse primer |
| SEQE ID NO: 19 | β-Actin probe |

### BRIEF DESCRIPTION OF THE DRAWINGS

FIG. 1 is a graphical representation of AGT-711 gene expression from liver in Groups A, B and C in the energy restricted study.

FIG. 2 is a graphical representation of the tissue distribution of AGT-711.

FIG. 3 is a graphical representation of AGT-711 gene expression from liver in Groups A, B and C in the fed/fasted study.

FIG. 4 is a graphical representation of AGT-711 gene versus body weight in all animals.

FIG. 5 is a graphical representation of AGT-711 gene versus blood glucose in all animals.

FIG. 6 is a graphical representation of AGT-711 gene versus serum insulin in all animals.

FIG. 7 is a graphical representation of AGT-711 gene in H4IIE cells treated for 24 hours with varying concentrations of Glucose.

FIG. 8 is a graphical representation of AGT-711 gene in H4IIE cells treated for 6 hours with varying concentrations of insulin.

FIG. 9 is a graphical representation of AGT-711 gene in L6 cells treated for 6 hours with varying concentrations of insulin.

FIG. 10 is a graphical representation of AGT-712 gene expression from hypothalamus in Groups A, B and C in the energy restricted study.

FIG. 11 is a graphical representation of AGT-712 gene expression versus blood glucose in ad libitum fed animals.

FIG. 12 is a graphical representation of AGT-712 gene expression versus serum insulin in ad libitum fed animals.

FIG. 13 is a graphical representation of AGT-712 gene expression versus serum insulin in all animals.

FIG. 14 is a graphical representation of AGT-713 gene expression in the liver of fed animals versus fasted animals.

FIG. 15 is a graphical representation of AGT-713 gene expression versus percent body fat in fasted animals.

FIG. 16 is a graphical representation of AGT-714 gene expression in the liver of fed animals versus fasted animals.

FIG. 17 is a graphical representation of AGT-714 gene expression from liver in Groups A, B and C in the fed/fasted study.

FIG. 18 is a graphical representation of AGT-714 gene expression versus body weight in all animals.

FIG. 19 is a graphical representation of AGT-714 gene expression versus serum insulin in all animals.

FIG. 20 is a graphical representation of AGT-714 gene expression versus body weight in fasted animals.

FIG. 21 is a graphical representation of AGT-714 gene expression versus change in blood glucose in fasted animals.

FIG. 22 is a graphical representation of AGT-714 gene expression versus percent body fat in fed animals.

FIG. 23 is a graphical representation of AGT-715 gene expression from liver in Groups A, B and C in the fed/fasted study

FIG. 24 is a graphical representation of AGT-715 gene expression versus percent body fat in fed animals.

FIG. 25 is a graphical representation of AGT-716 gene expression from red gastrocnemius muscle in Groups A, B and C in the fed/fasted study.

FIG. 26 is a graphical representation of AGT-716 gene expression in red gastrocnemius muscle versus percent body fat.

FIG. 27 is a graphical representation of AGT-716 gene expression in red gastrocnemius muscle versus serum insulin.

FIG. 28 is a graphical representation of AGT-716 gene expression from mesenteric adipose tissue in Groups A, B and C in the fed/fasted study.

FIG. 29 is a graphical representation of AGT-716 gene expression from mesenteric adipose tissue in fed animals versus fasted animals.

FIG. 30 is a graphical representation of AGT-716 gene expression from mesenteric adipose tissue versus body weight.

FIG. 31 is a graphical representation of AGT-716 gene expression in 3T3-L1 cells treated for 24 hours with varying concentrations of insulin.

FIG. 32 is a graphical representation of AGT-716 gene expression in 3T3-L1 cells treated for 24 hours with varying concentrations of glucose.

FIG. 33 is a graphical representation of AGT-717 gene expression from hypothalamus in Groups A, B and C in the energy restricted study.

FIG. 34 is a graphical representation of AGT-717 gene expression from hypothalamus versus post restriction body weight in control animals.

FIG. 35 is a graphical representation of AGT-717 gene expression from hypothalamus versus post restriction glucose levels in control animals.

FIG. 36 is a graphical representation of AGT-717 gene expression from hypothalamus versus post restriction body insulin levels in control animals.

FIG. 37 is a graphical representation of AGT-717 gene expression from hypothalamus versus fat oxidation in control animals.

FIG. 38 is a graphical representation of AGT-718 gene expression from hypothalamus in Groups A, B and C in the energy restricted study.

FIG. 39 is a graphical representation of AGT-718 gene expression from hypothalamus versus post restriction body weight in all animals.

FIG. 40 is a graphical representation of AGT-718 gene expression from hypothalamus versus post restriction insulin levels in control animals.

FIG. 41 is a graphical representation of AGT-718 gene expression from hypothalamus versus post restriction blood glucose in control animals.

FIG. 42 is a graphical representation of AGT-718 gene expression from hypothalamus versus total activity in control animals.

FIG. 43 is a graphical representation of AGT-718 gene expression from hypothalamus versus fat oxidation in control animals.

FIG. 44 is a graphical representation of the distribution of AGT-718 gene expression.

FIG. 45 is a graphical representation of AGT-718 gene expression from mesenteric fat in Groups A, B and C in the fed/fast study.

FIG. 46 is a graphical representation of AGT-718 gene expression in mesenteric fat from fed animals versus fasted animals.

FIG. 47 is a graphical representation of AGT-718 gene expression in mesenteric fat versus body weight in fed/fasted animals.

FIG. 48 is a graphical representation of AGT-718 gene expression in mesenteric fat versus insulin levels in fed/fasted animals.

FIG. 49 is a graphical representation of AGT-718 gene expression in mesenteric fat versus percent body fat in fed/fasted animals.

FIG. 50 is a graphical representation of AGT-718 gene expression from red gastrocnemius muscle in Groups A, B and C in the fed/fast study.

FIG. 51 is a graphical representation of AGT-718 gene expression from red gastrocnemius muscle in fed animals versus fasted animals.

FIG. 52 is a graphical representation of AGT-718 gene expression from liver in fed animals versus fasted animals.

FIG. 53 is a graphical representation of AGT-720 gene expression from hypothalamus in Groups A, B and C in the energy restricted study.

FIG. 54 is a graphical representation of AGT-720 gene expression from hypothalamus versus post experimental body weight in all animals.

FIG. 55 is a graphical representation of AGT-720 gene expression from hypothalamus versus post experimental body weight control animals.

FIG. 56 is a graphical representation of AGT-720 gene expression in hypothalamus versus post experimental plasma glucose levels in control animals.

FIG. 57 is a graphical representation of AGT-720 gene expression in hypothalamus versus total energy expenditure.

FIG. 58 is a graphical representation of AGT-721 gene expression from hypothalamus in Groups A, B and C in the energy restricted study.

FIG. 59 is a graphical representation of AGT-721 gene expression versus post restriction glucose in control animals.

FIG. 60 is a graphical representation of AGT-721 gene expression versus post restriction insulin in control animals.

FIG. 61 is a graphical representation of AGT-723 gene expression from hypothalamus in Groups A, B and C in the energy restricted study.

FIG. 62 is a graphical representation of AGT-723 gene expression versus post restriction glucose in control animals.

FIG. 63 is a graphical representation of AGT-723 gene expression versus post restriction body weight in control animals.

FIG. 64 is a graphical representation of AGT-723 gene expression versus post restriction insulin in control animals.

FIG. 65 is a graphical representation of AGT-723 gene expression versus percentage body fat in control animals.

FIG. 66 is a graphical representation of the tissue distribution of AGT-723 gene expression in the hypothalamus.

FIG. 67 is a graphical representation of AGT-723 gene expression from hypothalamus in Groups A, B and C in the fed/fast study.

FIG. 68 is a graphical representation of AGT-723 gene expression versus glucose levels in fasted animals.

FIG. 69 is a graphical representation of AGT-723 gene expression in fed and fasted Sprague Dawley rats.

FIG. 70 is a graphical representation of AGT-723 gene expression from mesenteric fat in Groups A, B and C in the fed/fasted study.

FIG. 71 is a graphical representation of AGT-723 gene expression versus body weight in all animals.

FIG. 72 is a graphical representation of AGT-723 gene expression versus insulin in all animals.

FIG. 73 is a graphical representation of AGT-724 gene expression in Groups A, B and C in the energy restricted group.

FIG. 74 is a graphical representation of AGT-724 gene expression versus body weight in all animals.

FIG. 75 is a graphical representation of AGT-724 gene expression versus blood glucose in control animals.

FIG. 76 is a graphical representation of AGT-726 gene expression from hypothalamus in Groups A, B and C in the energy restricted study.

FIG. 77 is a graphical representation of AGT-726 gene expression versus body weight in control animals.

FIG. 78 is a graphical representation of AGT-726 gene expression versus blood glucose in control animals.

FIG. 79 is a graphical representation of AGT-726 gene expression versus total activity in control animals.

FIG. 80 is a graphical representation of AGT-726 gene expression versus insulin levels in control animals.

FIG. 81 is a graphical representation of AGT-726 gene expression versus body weight in all animals.

FIG. 82 is a graphical representation of AGT-726 gene expression tissue distribution.

FIG. 83 is a graphical representation of AGT-726 gene expression from the hypothalamus is Groups A, B and C from the fed/fasted study.

FIG. 84 is a graphical representation of AGT-726 gene expression from mesenteric fat in Groups A, B and C in the fed/fasted study.

FIG. 85 is a graphical representation of AGT-726 gene expression versus body weight in all animals.

FIG. 86 is a graphical representation of AGT-726 gene expression versus log glucose in all animals.

FIG. 87 is a graphical representation of AGT-726 gene expression versus log insulin in all animals.

FIG. 88 is a graphical representation of AGT-726 gene expression in muscle from Groups A, B and C in the fed/fasted study.

FIG. 89 is a graphical representation of AGT-726 gene expression versus log glucose in all animals.

FIG. 90 is a graphical representation of AGT-719 gene expression from hypothalamus in Groups A, B and C in the energy restricted study.

FIG. 91 is a graphical representation of AGT-719 gene expression versus weight in all animals.

FIG. 92 is a graphical representation of AGT-719 gene expression versus glucose in all animals.

FIG. 93 is a graphical representation of AGT-719 gene expression versus post restriction glucose in control animals.

FIG. 94 is a graphical representation of AGT-719 gene expression versus post restriction insulin in control animals.

FIG. 95 is a graphical representation of AGT-719 gene expression versus post restriction body weight in control animals.

FIG. 96 is graphical representation of AGT-719 gene expression versus total fat with epididymal tissue in control animals.

FIG. 97 is a graphical representation of AGT-719 gene expression versus total physical activity in control animals.

FIG. 98 is a graphical representation of AGT-722 gene expression from hypothalamus in Groups A, B and C in the energy restricted study.

FIG. 99 is a graphical representation of AGT-722 gene expression versus post restriction glucose in control animals.

FIG. 100 is a graphical representation of AGT-722 gene expression versus post restriction insulin in control animals.

FIG. 101 is a graphical representation of AGT-722 gene expression versus total physical activity in control animals.

FIG. 102 is a graphical representation of AGT-722 gene expression versus total body fat in control animals.

FIG. 103 is a graphical representation of AGT-725 gene expression in the hypothalamus of control animals versus dietary restricted animals.

FIG. 104 is a graphical representation of AGT-725 gene expression from hypothalamus in Groups A, B and C in the energy restricted study.

FIG. 105 is a graphical representation of AGT-725 gene expression versus blood glucose in control animals.

FIG. 106 is a graphical representation of AGT-725 gene expression versus total physical activity in the energy restricted animals.

FIG. 107: AGT-717 gene expression in all fed versus all fasted animals compared to fed animals.

FIG. 108: Linear association between AGT-717 and glucose in red gastrocnemius muscle of P. obesus fasted for 24 hr.

FIG. 109: AGT-717 gene expression in mesenteric fat of fed and 24 hr fasted P. obesus compared to group A fed.

FIG. 110: Linear associations of AGT-717 gene expression in mesenteric fat with body weight and insulin values in all animals.

FIG. 111: AGT-717 gene expression in 3T3 cells treated with insulin for 24 hrs compared to 0 nM, 0.1 nM and 1 nM groups.

FIG. 112: AGT-717 gene expression in 3T3 cells treated with glucose for 24 hr compared to 0 mM compared to 12.5 mM.

### DETAILED DESCRIPTION OF THE PREFERRED EMBODIMENTS

The present invention is predicated in part on the identification of novel genes associated inter alia with regulation of myopathy, obesity, anorexia, weight maintenance, diabetes, disorders associated with mitochondrial dysfunction, genetic disorders and/or metabolic energy levels. The genes were identified following differential screening of either hypothalamus, liver, mesenteric adipose tissue or red gastrocnemius muscle mRNA between lean and obese animals and/or between fed animals and fasted animals.

In describing or claiming the present invention, the following terminology are used in accordance with the definition set forth below.

The term “differential” array is used in its broadest sense to include the expression of nucleic acid sequences in one type of tissue relative to another type of tissue in the same or different animals. Reference to “different” animals include the same animals but in different gastronomical states such as in a fed or non-fed state.

Accordingly, one aspect of the present invention provides a nucleic acid molecule comprising a sequence of nucleotides encoding or complementary to a sequence encoding an expression product or a derivative, homolog, analog or mimetic thereof wherein said nucleic acid molecule is expressed in larger amounts in hypothalamus tissue of fasted animals compared to fed animals.

In a related embodiment, there is provided a nucleic acid molecule comprising a sequence of nucleotides encoding or complementary to a sequence encoding an expression product or a derivative, homolog, analog or mimetic thereof wherein said nucleic acid molecule is expressed in larger amounts in hypothalamus tissue of fasted animals compared to fed animals.

It must be noted that, as used in the subject specification, the singular forms “a”, “an” and “the” include plural aspects unless the context clearly dictates otherwise. Thus, for example, reference to a “compound” includes a single compound, as well as two or more compounds; reference to “an active agent” includes a single active agent, as well as two or more active agents; “a holocyclotoxin” includes a single holocyclotoxin or two or more holocyclotoxins and so forth.

The elevated expression levels may be in healthy animals or in obese, diabetic or non-diabetic animals.

The terms “lean” and “obese” are used in their most general sense but should be considered relative to the standard criteria for determining obesity. Generally, for human subjects, the definition of obesity is BMI>30 (Risk Factor Prevalence Study Management Committee. Risk Factor Prevalence Study: Survey No. 3 1989. Canberra: National Heart Foundation of Australia and Australian Institute of Health, 1990; Waters and Bennett, Risk Factors for cardiovascular disease: A summary of Australian data. Canberra: Australian Institute of Health and Welfare, 1995).

Conveniently, an animal model may be employed to study the differences in gene expression between obese and lean animals and fasted and fed animals. In particular, the present invention is exemplified using the Psammomys obesus (the Israeli sand rat) animal model of dietary-induced obesity and NIDDM. In its natural desert habitat, an active lifestyle and saltbush diet ensure that they remain lean and normoglycemic (Shafrir and Gutman, J Basic Clin Physiol Pharm 4: 83-99, 1993). However, in a laboratory setting on a diet of ad libitum chow (on which many other animal species remain healthy), a range of pathophysiological responses are seen (Barnett et al., Diabetologia 37: 671-676, 1994a; Barnett et al., Int. J. Obesity 18: 789-794, 1994b, Barnett et al., Diabete Nutr Metab 8: 42-47, 1995). By the age of 16 weeks, more than half of the animals become obese and approximately one-third develop NIDDM. Only hyperphagic animals go on to develop hyperglycemia, highlighting the importance of excessive energy intake in the pathophysiology of obesity and NIDDM in Psammomys obesus (Collier et al., Ann New York Acad Sci 827: 50-63, 1997a; Walder et al., Obesity Res 5: 193-200, 1997a). Other phenotypes found include hyperinsulinemia, dyslipidemia and impaired glucose tolerance (Collier et al., [1997a; supra]; Collier et al., Exp Clin Endocrinol Diabetes 105: 36-37, 1997b). Psammomys obesus exhibit a range of bodyweight and blood glucose and insulin levels which forms a continuous curve that closely resembles the patterns found in human populations, including the inverted U-shaped relationship between blood glucose and insulin levels known as “Starling's curve of the pancreas” (Barnett et al., [1994a; supra]). It is the heterogeneity of the phenotypic response of Psammomys obesus which make it an ideal model to study the etiology and pathophysiology of obesity and NIDDM.

Psammomys obesus animals are conveniently divided into three groups viz Group A animals which are lean, normoglycemic and normoinsulinemic, Group B animals which are obese, normoglycemic and hyperinuslinemic and Group C animals which are obese, hyperglycemic and h yperinsulinemic.

Another aspect of the present invention provides a nucleic acid molecule comprising a nucleotide sequence encoding or complementary to a sequence encoding an expression product or a derivative, homolog, analog or mimetic thereof wherein said nucleotide sequence is as substantially set forth in SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 a nucleotide sequence having at least about 40% similarity to all or part of SEQ ID NO:1 or SEQ ID NO:2 or SEQ ID NO:3 SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 and/or is capable of hybridizing to one or more of SEQ ID NO:1 or SEQ ID NO:2 or SEQ ID NO:3 SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 or their complementary forms under low stringency conditions and wherein said nucleic acid molecule is expressed in a larger amount in hypothalamus tissue of obese animals compared to lean animals and/or in fed animals compared to fasted animals.

Higher similarities are also contemplated by the present invention such as greater than 40% or 50% or 60% or 70% or 80% or 90% or 95% or 96% or 97% or 98% or 99% or above. Examples include 41, 42, 43, 44, 45, 46, 47, 48, 49, 50, 51, 52, 53, 54, 55, 56, 57, 58, 59; 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

An expression product includes an RNA molecule such as a mRNA transcript as well as a protein. Some genes are non-protein encoding genes and produce mRNA or other RNA type molecules and are involved in regulation by RNA:DNA, RNA:RNA or RNA:protein interaction. The RNA (e.g. mRNA) may act directly or via the induction of other molecules such as RNAi or via products mediated from splicing events (e.g. exons or introns). Other genes encode mRNA transcripts which are then translated into proteins. A protein includes a polypeptide. The differentially expressed nucleic acid molecules, therefore, may encode mRNAs only or, in addition, proteins. Both mRNAs and proteins are forms of “expression products”.

Reference herein to similarity is generally at a level of comparison of at least 15 consecutive or substantially consecutive nucleotides.

The term “similarity” as used herein includes exact identity between compared sequences at the nucleotide level. Where there is non-identity at the nucleotide level, “similarity” includes differences between sequences which result in different amino acids that are nevertheless related to each other at the structural, functional, biochemical and/or conformational levels. In a particularly preferred embodiment, nucleotide sequence comparisons are made at the level of identity rather than similarity.

Terms used to describe sequence relationships between two or more polynucleotides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units in length, examples include 12, 13, 14, 15, 16, 17, 18, 19, 20, 21, 22, 23, 24, 25, 26, 27, 28, 29 and 30. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best aligmnent (i.e. resulting in the highest. percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refers to the extent that sequences are identical or functionally or structurally similar on a nucleotide-by-nucleotide basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C., such as 25, 26, 27, 28, 29, 30, 31, 32, 33, 34, 35, 36, 37, 38, 39, 40, 41 and 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide, such as 16, 17, 18, 19, 20, 21, 22, 23, 24, 25, 26, 27, 28, 29 and 30% and from at least about 0.5 M to at least about 0.9 M salt, such as 0.5, 0.6, 0.7, 0.8 or 0.9 M for hybridization, and at least about 0.5 M to at least about 0.9 M salt, such as 0.5, 0.6, 0.7, 0.8 or 0.9 M for washing conditions, or high stringency, which includes and encompasses from at least about 31% v/v to at least about 50% v/v formamide, such as 31, 32, 33, 34, 35, 36, 37, 38, 39, 40, 41, 42, 43, 44, 45, 46, 47, 48, 49 and 50% and from at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for hybridization, and at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for washing conditions. In general, washing is carried out Tₘ=69.3+0.41 (G+C)% (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974. Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The nucleotide sequence or amino acid sequence of the present invention may correspond to exactly the same sequence of the naturally occurring gene (or corresponding cDNA) or protein or other expression product or may carry one or more nucleotide or amino acid substitutions, additions and/or deletions. The nucleotide sequences set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and/or SEQ ID NO:16 correspond to novel genes referred to herein as AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725, respectively. The corresponding expression products AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725, respectively. Reference herein AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 includes, where appropriate, reference to the genomic gene or cDNA as well as any naturally occurring or induced derivatives. Apart from the substitutions, deletions and/or additions to the nucleotide sequence, the present invention further encompasses mutants, fragments, parts and portions of the nucleotide sequence corresponding to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:1 or a derivative, homolog or mimetic thereof or having at least about 40% identity to all or part of SEQ ID NO:1.

Yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:2 or a derivative, homolog or mimetic thereof or having at least about 40% identity to all or part of SEQ ID NO:2.

Still yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:3 or a derivative, homolog or mimetic thereof or having at least about 40% identity to all or part of SEQ ID NO:3.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:4 or a derivative, homolog or mimetic thereof or having at least about 40% identity to all or part of SEQ ID NO:4.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:5 or a derivative, homolog or mimetic thereof or having at least about 40% identity to all or part of SEQ ID NO:5.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:6 or a derivative, homolog or mimetic thereof or having at least about 40% identity to all or part of SEQ ID NO:6.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:7 or a derivative, homolog or mimetic thereof or naving at least about 40% identity to all or part of SEQ ID NO:7.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:8 or a derivative, homolog or mimetic thereof or having at least about 40% identity to all or part of SEQ ID NO:8.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:9 or a derivative, homolog or mimetic thereof or having at least about 40% identity to all or part of SEQ ID NO:9.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:10 or a derivative, homolog or mimetic thereof or having at least about 40% identity to all or part of SEQ ID NO:10.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:11 or a derivative, homolog or mimetic thereof or having at least about 40% identity to all or part of SEQ ID NO:11.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:12 or a derivative, homolog or mimetic thereof or having at least about 40% identity to all or part of SEQ ID NO:12.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:13 or a derivative, homolog or mimetic thereof or having at least about 40% identity to all or part of SEQ ID NO:13.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:14 or a derivative, homolog or mimetic thereof or having at least about 40% identity to all or part of SEQ ID NO:14.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:15 or a derivative, homolog or mimetic thereof or having at least about 40% identity to all or part of SEQ ID NO:15.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:16 or a derivative, homolog or mimetic thereof or having at least about 40% identity to all or part of SEQ ID NO:16.

The expression pattern of AGT-711, AGT-712, AGT-713, AGT-714, AGT-715, AGT-716; AGT-717, AGT-718, AGT-720, AGT-721, AGT-723, AGT-724, AGT-726, AGT-719, AGT-722 and AGT-725 has been determined, inter alia, to indicate an involvement in the regulation of one or more of inter alia a myopathy, obesity, anorexia, weight maintenance, diabetes, disorders associated with mitochondrial dysfunction, genetic disorders and/or metabolic energy levels. In addition to the differential expression of AGT-711, AGT-712, AGT-713, AGT-714, AGT-711, AGT-716, AGT-717, AGT-718, AGT-720, AGT-721, AGT-723, AGT-724, AGT-726, AGT-719, AGT-722 and AGT-725 in the hypothalamus, liver, mesenteric adipose tissue and/or red gastrocnemius muscle tissues of lean versus obese animals and fed/or versus fasted animals, these genes may also be expressed in other tissues including but in no way limited to brain stem, cerebellum, cortex, hippocampus and mid-brain. The nucleic acid molecule encoding each of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 or AGT-725 is preferably a sequence of deoxyribonucleic acids such as a cDNA sequence or a genomic sequence. A genomic sequence may also comprise exons and introns. A genomic sequence may also include a promoter region or other regulatory regions.

A homolog is considered to be a gene from another animal species which has the same or greater than 40% similarity to one of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 or AGT-725 and/or which has a similar function. The AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 or AGT-725 genes are exemplified herein from Psammomys obesus hypothalamus. The present invention extends, however, to the homologous gene, as determined by nucleotide sequence and/or amino acid sequences and/or function, from primates, including humans, marmosets, orangutans and gorillas, livestock animals (e.g. cows, sheep, pigs, horses, donkeys), laboratory test animals (e.g. mice, rats, guinea pigs, hamsters, rabbits), companion animals (e.g. cats, dogs) and captured wild animals (e.g. rodents, foxes, deer, kangaroos). The present invention also contemplates deimmunized forms of the expression products from one species relative to another species. In a part preferred embodiment, the deimmunized form of the expression product is a mamalianized form relative to a particular target animal. In a most preferred embodiment where the target mammal is a human, the present invention contemplates use of a humanized form of a non-human expression product.

The nucleic acids of the present invention and in particular AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 and their derivatives and homologs may be in isolated or purified form and/or may be ligated to a vector such as an expression vector. Expression may be in a eukaryotic cell line (e.g. mammalian, insect or yeast cells) or in microbial cells (e.g. E. coli) or both. By “isolated” is meant a nucleic acid molecule having undergone at least one purification step and this is conveniently defined, for example, by a composition comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure. The nucleic acid molecule may be ligated to an expression vector capable of expression in a prokaryotic cell (e.g. E. coli) or a eukaryotic cell (e.g. yeast cells, flngal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector.

The derivatives of the nucleic acid molecule of the present invention include oligonucleotides, PCR primers, antisense molecules, molecules suitable for use in co-suppression and fusion nucleic acid molecules. Ribozymes and DNAzymes are also contemplated by the present invention directed to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or their mRNAs. Derivatives and homologs AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 are conveniently encompassed by those nucleotide sequences capable of hybridizing to one or more of SEQ ID NO:1, SEQ ID NO:2 or SEQ ID NO:3 SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 or their complementary forms under low stringency conditions.

Derivatives include fragments, parts, portions, mutants, variants and mimetics from natural, synthetic or recombinant sources including fusion nucleic acid molecules. Derivatives may be derived from insertion, deletion or substitution of nucleotides.

Another aspect of the present invention provides an isolated expression product or a derivative, homolog, analog or mimetic thereof which is produced in larger amounts in hypothalamus tissue in obese animals compared to lean animals.

Amino acid insertional derivatives include amino and/or carboxylic terminal fusions as well as intrasequence insertions of single or multiple amino acids. Insertional amino acid sequence variants are those in which one or more amino acid residues are introduced into a predetermined site in a protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of protein forms of the expression AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Reference herein to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 includes reference to isolated or purified naturally AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 as well as any derivatives, homologs, analogs and mimetics thereof. Derivatives include parts, fragments and portions AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 as well as single and multiple amino acid substitutions, deletions and/or additions to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 when the expression products are proteins. A derivative AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 is conveniently encompassed by molecules encoded by a nucleotide sequence capable of hybridizing SEQ ID NO:1, SEQ ID NO:2 or SEQ ID NO:3 SEQ ID NO:4 or SEQ ID NO:5 or SEQ ID NO:6 or SEQ ID NO:7 or SEQ ID NO:8 or SEQ ID NO:9 or SEQ ID NO:10 or SEQ ID NO:11 or SEQ ID NO:12 or SEQ ID NO:13 or SEQ ID NO:14, SEQ ID NO:15 or SEQ ID NO:16 under low stringency conditions.

Other derivatives of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 include chemical analogs. Analogs of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 contemplated herein include, but are not limited to, modifications to side chains, incorporation of unnatural amino acids and/or their derivatives during peptide, polypeptide or protein synthesis and the use of crosslinkers and other methods which impose confirmational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 3.

TABLE 3
Codes for non-convention amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

All such modifications may also be useful in stabilizing the AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 molecule for use in in vivo administration protocols or for diagnostic purposes.

As stated above, the expression product may be an RNA or protein.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particularly preferred embodiment, the expression product is encoded by a sequence of nucleotides as set forth in SEQ ID NO:1 or a derivative, homolog or analog thereof including a nucleotide sequence having at least about 40% identity to SEQ ID NO:1.

In another particularly preferred embodiment, the expression product is encoded by a sequence of nucleotides as set forth in SEQ ID NO:2 or a derivative, homolog or analog thereof including a nucleotide sequence having at least about 40% identity to SEQ ID NO:2.

In still another particularly preferred embodiment, the expression product is encoded by a sequence of nucleotides as set forth in SEQ ID NO:3 or a derivative homolog or analog thereof including a nucleotide sequence having at least about 40% identity to SEQ ID NO:3.

In still another particularly preferred embodiment, the expression product is encoded by a sequence of nucleotides as set forth in SEQ ID NO:4 or a derivative homolog or analog thereof including a nucleotide sequence having at least about 40% identity to SEQ ID NO:4.

In still another particularly preferred embodiment, the expression product is encoded by a sequence of nucleotides as set forth in SEQ ID NO:5 or a derivative homolog or analog thereof including a nucleotide sequence having at least about 40% identity to SEQ ID NO:5.

In still another particularly preferred embodiment, the expression product is encoded by a sequence of nucleotides as set forth in SEQ ID NO:6 or a derivative homolog or analog thereof including a nucleotide sequence having at least about 40% identity to SEQ ID NO:6.

In still another particularly preferred embodiment, the expression product is encoded by a sequence of nucleotides as set forth in SEQ ID NO:7 or a derivative homolog or analog thereof including a nucleotide sequence having at least about 40% identity to SEQ ID NO:7.

In still another particularly preferred embodiment, the expression product is encoded by a sequence of nucleotides as set forth in SEQ ID NO:8 or a derivative homolog or analog thereof including a nucleotide sequence having at least about 40% identity to SEQ ID NO:8.

In still another particularly preferred embodiment, the expression product is encoded by a sequence of nucleotides as set forth in SEQ ID NO:9 or a derivative homolog or analog thereof including a nucleotide sequence having at least about 40% identity to SEQ ID NO:9.

In still another particularly preferred embodiment, the expression product is encoded by a sequence of nucleotides as set forth in SEQ ID NO:10 or a derivative homolog or analog thereof including a nucleotide sequence having at least about 40% identity to SEQ ID NO:10.

In still another particularly preferred embodiment, the expression product is encoded by a sequence of nucleotides as set forth in SEQ ID NO:11 or a derivative homolog or analog thereof including a nucleotide sequence having at least about 40% identity to SEQ ID NO:11.

In still another particularly preferred embodiment, the expression product is encoded by a sequence of nucleotides as set forth in SEQ ID NO:12 or a derivative homolog or analog thereof including a nucleotide sequence having at least about 40% identity to SEQ ID NO:12.

In still another particularly preferred embodiment, the expression product is encoded by a sequence of nucleotides as set forth in SEQ ID NO:13 or a derivative homolog or analog thereof including a nucleotide sequence having at least about 40% identity to SEQ ID NO:13.

In still another particularly preferred embodiment, the expression product is encoded by a sequence of nucleotides as set forth in SEQ ID NO:14 or a derivative homolog or analog thereof including a nucleotide sequence having at least about 40% identity to SEQ ID NO:14.

In still another particularly preferred embodiment, the expression product is encoded by a sequence of nucleotides as set forth in SEQ ID NO:15 or a derivative homolog or analog thereof including a nucleotide sequence having at least about 40% identity to SEQ ID NO:15.

In still another particularly preferred embodiment, the expression product is encoded by a sequence of nucleotides as set forth in SEQ ID NO:16 or a derivative homolog or analog thereof including a nucleotide sequence having at least about 40% identity to SEQ ID NO:16.

Higher similarities are also contemplated by the present invention such as greater than 40% or 50% or 60% or 70% or 80% or 90% or 95% or 96% or 97% or 98% or 99% or above. Further examples include 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

Another aspect of the present invention is directed to an isolated expression product selected from the list consisting of:
(i) an mRNA or protein encoded by a novel nucleic acid molecule which molecule is differentially expressed in hypothalamus tissue of obese animals compared to lean Psammomys obesus animals or a derivative, homolog, analog, chemical equivalent or mimetic thereof;
(ii) an mRNA or protein encoded by a novel nucleic acid molecule which molecule is differentially expressed in hypothalamus tissue of fasted animals compared to fed Psammomys obesus animals or a derivative, homolog, analog, chemical equivalent or mimetic thereof;
(iii) AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or a derivative, homolog, analog, chemical equivalent or mimetic thereof;
(iv) a protein encoded by a nucleotide sequence substantially as set forth in SEQ ID NO:1 or a derivative, homolog or analog thereof or a sequence encoding an amino acid sequence having at least about 40% similarity to this sequence or a derivative, homolog, analog, chemical equivalent or mimetic of said protein;
(v) a protein encoded by a nucleotide sequence substantially as set forth in SEQ ID NO:2 or a derivative, homolog or analog thereof or a sequence encoding an amino acid sequence having at least about 40% similarity to this sequence or a derivative, homolog, analog, chemical equivalent or mimetic of said protein;
(vi) a protein encoded by a nucleotide sequence substantially as set forth in SEQ ID NO:3 or a derivative, homolog or analog thereof or a sequence encoding an amino acid sequence having at least about 40% similarity to this sequence or a derivative, homolog, analog, chemical equivalent or mimetic of said protein;
(vii) a protein encoded by a nucleotide sequence substantially as set forth in SEQ ID NO:4 or a derivative, homolog or analog thereof or a sequence encoding an amino acid sequence having at least about 40% similarity to this sequence or a derivative, homolog, analog, chemical equivalent or mimetic of said protein;
(viii) a protein encoded by a nucleotide sequence substantially as set forth in SEQ ID NO:5 or a derivative, homolog or analog thereof or a sequence encoding an amino acid sequence having at least about 40% similarity to this sequence or a derivative, homolog. analog, chemical equivalent or mimetic of said protein;
(ix) a protein encoded by a nucleotide sequence substantially as set forth in SEQ ID NO:6 or a derivative, homolog or analog thereof or a sequence encoding an amino acid sequence having at least about 40% similarity to this sequence or a derivative, homolog, analog, chemical equivalent or mimetic of said protein;
(x) a protein encoded by a nucleotide sequence substantially as set forth in SEQ ID NO:7 or a derivative, homolog or analog thereof or a sequence encoding an amino acid sequence having at least about 40% similarity to this sequence or a derivative, homolog, analog, chemical equivalent or mimetic of said protein;
(xi) a protein encoded by a nucleotide sequence substantially as set forth in SEQ ID NO:8 or a derivative, homolog or analog thereof or a sequence encoding an amino acid sequence having at least about 40% similarity to this sequence or a derivative, homolog, analog, chemical equivalent or mimetic of said protein;
(xii) a protein encoded by a nucleotide sequence substantially as set forth in SEQ ID NO:9 or a derivative, homolog or analog thereof or a sequence encoding an amino acid sequence having at least about 40% similarity to this sequence or a derivative, homolog, analog, chemical equivalent or mimetic of said protein;
(xiii) a protein encoded by a nucleotide sequence substantially as set forth in SEQ ID NO:10 or a derivative, homolog or analog thereof or a sequence encoding an amino acid sequence having at least about 40% similarity to this sequence or a derivative, homolog, analog, chemical equivalent or mimetic of said protein;
(xiv) a protein encoded by a nucleotide sequence substantially as set forth in SEQ ID NO:11 or a derivative, homolog or analog thereof or a sequence encoding an amino acid sequence having at least about 40% similarity to this sequence or a derivative, homolog, analog, chemical equivalent or mimetic of said protein;
(xv) a protein encoded by a nucleotide sequence substantially as set forth in SEQ ID NO:12 or a derivative, homolog or analog thereof or a sequence encoding an amino acid sequence having at least about 40% similarity to this sequence or a derivative, homolog, analog, chemical equivalent or mimetic of said protein;
(xvi) a protein encoded by a nucleotide sequence substantially as set forth in SEQ ID NO:13 or a derivative, homolog or analog thereof or a sequence encoding an amino acid sequence having at least about 40% similarity to this sequence or a derivative, homolog, analog, chemical equivalent or mimetic of said protein;
(xvii) a protein encoded by a nucleotide sequence substantially as set forth in SEQ ID NO:14 or a derivative, homolog or analog thereof or a sequence encoding an amino acid sequence having at least about 40% similarity to this sequence or a derivative, homolog, analog, chemical equivalent or mimetic of said protein;
(xviii) a protein encoded by a nucleotide sequence substantially as set forth in SEQ ID NO:15 or a derivative, homolog or analog thereof or a sequence encoding an amino acid sequence having at least about 40% similarity to this sequence or a derivative, homolog, analog, chemical equivalent or mimetic of said protein;
(xix) a protein encoded by a nucleotide sequence substantially as set forth in SEQ ID NO:16 or a derivative, homolog or analog thereof or a sequence encoding an amino acid sequence having at least about 40% similarity to this sequence or a derivative, homolog, analog, chemical equivalent or mimetic of said protein;
(xx) a protein encoded by a nucleic acid molecule capable of hybridizing to the nucleotide sequence as set forth in SEQ ID NO:1 or its complementary form or a derivative, homolog or analog thereof under low stringency conditions;
(xxi) a protein encoded by a nucleic acid molecule capable of hybridizing to the nucleotide sequence as set forth in SEQ ID NO:2 or its complementary form or a derivative, homolog or analog thereof under low stringency conditions;
(xxii) a protein encoded by a nucleic acid molecule capable of hybridizing to the nucleotide sequence as set forth in SEQ ID NO:3 or its complementary form or a derivative, homolog or analog thereof under low stringency conditions;
(xxiii) a protein encoded by a nucleic acid molecule capable of hybridizing to the nucleotide sequence as set forth in SEQ ID NO:4 or its complementary form or a derivative, homolog or analog thereof under low stringency conditions;
(xxiv) a protein encoded by a nucleic acid molecule capable of hybridizing to the nucleotide sequence as set forth in SEQ ID NO:5 or its complementary form or a derivative, homolog or analog thereof under low stringency conditions;
(xxv) a protein encoded by a nucleic acid molecule capable of hybridizing to the nucleotide sequence as set forth in SEQ ID NO:6 or its complementary form or a derivative, homolog or analog thereof under low stringency conditions;
(xxvi) a protein encoded by a nucleic acid molecule capable of hybridizing to the nucleotide sequence as set forth in SEQ ID NO:7 or its complementary form or a derivative, homolog or analog thereof under low stringency conditions;
(xxvii) a protein encoded by a nucleic acid molecule capable of hybridizing to the nucleotide sequence as set forth in SEQ ID NO:8 or its complementary form or a derivative, homolog or analog thereof under low stringency conditions;
(xxviii) a protein encoded by a nucleic acid molecule capable of hybridizing to the nucleotide sequence as set forth in SEQ ID NO:9 or its complementary form or a derivative, homolog or analog thereof under low stringency conditions;
(xxix) a protein encoded by a nucleic acid molecule capable of hybridizing to the nucleotide sequence as set forth in SEQ ID NO:10 or its complementary form or a derivative, homolog or analog thereof under low stringency conditions;
(xxx) a protein encoded by a nucleic acid molecule capable of hybridizing to the nucleotide sequence as set forth in SEQ ID NO:11 or its complementary form or a derivative, homolog or analog thereof under low stringency conditions;
(xxxi) a protein encoded by a nucleic acid molecule capable of hybridizing to the nucleotide sequence as set forth in SEQ ID NO:12 or its complementary form or a derivative, homolog or analog thereof under low stringency conditions;
(xxxii) a protein encoded by a nucleic acid molecule capable of hybridizing to the nucleotide sequence as set forth in SEQ ID NO:13 or its complementary form or a derivative, homolog or analog thereof under low stringency conditions;
(xxxiii) a protein encoded by a nucleic acid molecule capable of hybridizing to the nucleotide sequence as set forth in SEQ ID NO:14 or its complementary form or a derivative, homolog or analog thereof under low stringency conditions;
(xxxiv) a protein encoded by a nucleic acid molecule capable of hybridizing to the nucleotide sequence as set forth in SEQ ID NO:15 or its complementary form or a derivative, homolog or analog thereof under low stringency conditions; and
(xxxv) a protein encoded by a nucleic acid molecule capable of hybridizing to the nucleotide sequence as set forth in SEQ ID NO:16 or its complementary form or a derivative, homolog or analog thereof under low stringency conditions.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% of subject protein relative to other components as determined by molecular weight, amino acid sequence or other convenient means. The protein of the present invention may also be considered, in a preferred embodiment, to be biologically pure.

Without limiting the theory or mode of action of the present invention, the expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 is thought to relate to regulation of body weight and glucose homeostasis. Modulation of these genes expression is thought inter alia to regulate energy balance via effects on energy intake and also effects on carbohydrate/fat metabolism. The energy intake effects are likely to be mediated via the central nervous system but peripheral effects on the metabolism of both carbohydrate and fat are possible. The expression of these genes may also be regulated by fasting and feeding. Accordingly, regulating the expression and/or activity of these genes or their expression products provides a mechanism for regulating both body weight and energy metabolism, including carbohydrate and fat metabolism.

The identification of AGT-711, AGT-712, AGT-713, AGT-714, AGT-715, AGT-716, AGT-717, AGT-718, AGT-720, AGT-721, AGT-723, AGT-724, AGT-726, AGT-71, AGT-722 and AGT-725 permits the generation of a range of therapeutic molecules capable of modulating expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or modulating the activity of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 . Modulators contemplated by the present invention include agonists and antagonists of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 expression. Antagonists of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 expression include antisense molecules, ribozymes and co-suppression molecules including RNAi-type molecules. Agonists include molecules which increase promoter activity or which interfere with negative regulatory mechanisms. Antagonists of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 include antibodies and inhibitor peptide fragments. All such molecules may first need to be modified to enable such molecules to penetrate cell membranes. Alternatively, viral agents may be employed to introduce genetic elements to modulate expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725. Insofar as AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 act in association with other genes such as the ob gene which encodes leptin, the therapeutic molecules may target AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 and ob genes or their translation products. The present invention contemplates, therefore, a method for modulating expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 in a mammal, said method comprising contacting the AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 gene with an effective amount of a modulator of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 expression for a time and under conditions sufficient to up-regulate or down-regulate or otherwise modulate expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725. For example, a nucleic acid molecule encoding AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or a derivative or homolog thereof may be introduced into a cell to enhance the ability of that cell to produce AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725, conversely, AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 sense and/or antisense sequences such as oligonucleotides may be introduced to decrease the availability of AGT-711, AGT-712, AGT-713, AGT-714, AGT-715, AGT-716, AGT-717, AGT-718, AGT-720, AGT-721, AGT-723, AGT-724, AGT-726, AGT-71, AGT-722 and AGT-725 molecules.

Another aspect of the present invention contemplates a method of modulating activity of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 in a mammal, said method comprising administering to said mammal a modulating effective amount of a molecule for a time and under conditions sufficient to increase or decrease AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 activity. The molecule may be a proteinaceous molecule or a chemical entity and may also be a derivative AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or its ligand.

Modulating levels of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 expression or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 activity or function is important in the treatment of a range of conditions such as inter alia a myopathy, obesity, anorexia, weight maintenance, diabetes, disorders associated with mitochondrial dysfunction, genetic disorders and/or metabolic energy levels, metabolic syndrome, dyslipidemia, hypertension and insulin resistance. It may also be useful in the agricultural industry to assist in the generation of leaner animals, or where required, more obese animals. Accordingly, mammals contemplated by the present invention include but are not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particularly preferred host is a human, primate or livestock animal.

Accordingly, the present invention contemplates therapeutic and prophylactic use of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 expression products or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 genetic mutants and/or agonists or antagonists agents thereof.

The present invention contemplates, therefore, a method of modulating expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 in a mammal, said method comprising contacting the AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 genes with an effective amount of an agent for a time and under conditions sufficient to up-regulate, down-regulate or otherwise module expression of AGT-711, AGT-712, AGT-713, AGT-714, AGT-715, AGT-716, AGT-717, AGT-718, AGT-720. AGT-721, AGT-723, AGT-724, AGT-726, AGT-719, AGT-722 and AGT-725.

Another aspect of the present invention contemplates a method of modulating activity of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 in a subject, said method comprising administering to said subject a modulating effective amount of an agent for a time and under conditions sufficient to increase or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 activity or function.

Modulation of activity by the administration of an agent to a mammal can be achieved by one of several techniques, including, but in no way limited to, introducing into a mammal a proteinaceous or non-proteinaceous molecule which:
(i) modulates expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725;
(ii) functions as an antagonist of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725; and/or
(iii) functions as an agonist of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725.

The molecules which may be administered to a mammal in accordance with the present invention may also be linked to a targeting means such as a monoclonal antibody, which provides specific delivery of these molecules to the target cells.

A further aspect of the present invention relates to the use of the invention in relation to mammalian disease conditions. For example, the present invention is particularly useful but in no way limited to use in a therapeutic or prophylactic treatment of inter alia a myopathy, obesity, anorexia, weight maintenance, diabetes, disorders associated with mitochondrial dysfunction, genetic disorders and/or metabolic energy levels.

Accordingly, another aspect of the present invention relates to a method of treating a mammal suffering from a condition characterized by one or more symptoms of inter alia a myopathy, obesity, anorexia, weight maintenance, diabetes, disorders associated with mitochondrial dysfunction, genetic disorders and/or metabolic energy levels, said method comprising administering to said mammal an effective amount of an agent for a time and under conditions sufficient to modulate the expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or sufficient to modulate the activity of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725.

In another aspect, the present invention relates to a method of treating a mammal suffering from a disease condition characterized by one or more symptoms of inter alia a myopathy, obesity, anorexia, weight maintenance, diabetes, disorders associated with mitochondrial dysfunction, genetic disorders and/or metabolic energy levels, said method comprising administering to said mammal an effective amount of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725.

As used herein “myopathy” refers to any abnormal conditions or disease of the muscle tissues, which include the muscles over our bones (skeletal muscle) and the heart (cardiac muscle).

Obesity, inter alia a myopathy, anorexia, diabetes and disorders associated with imbalances in metabolic energy levels are disease and disorders associated with mitochondrial dysfunction, genetic disorders. Mitochondria are part of the cell (organelle) that is responsible for energy production. The organelle consists of two sets of membranes, a smooth continuous outer coat and an inner membrane arranged in tubules or in folds that form plate-like double membranes (cristae). Mitochondria are the principal energy source of the cell, containing the cytochrome enzymes of terminal electron transport and the enzymes of the citric acid cycle, fatty acid oxidation, and oxidative phosphorylation. They are responsible for converting nutrients into energy as well as many other specialized tasks. Mitochondria are complex organelles located in virtually all cells of the body. A large degree of their complexity is due to the fact that over 1000 proteins are located in the mitochondria. Thirteen of these proteins are encoded by the mitochondrial DNA (mtDNA), while the remainder are nuclear-encoded, and imported into the mitochondria.

As used herein a “mitochondrial disease or disorder” refers to any illness resulting from a deficiency of any mitochondrial-located protein which is involved in energy metabolism. Therefore, deficiencies of the respiratory (electron transport) chain, either resulting from a deficiency in none or more of the mitochondrial or nuclear-encoded proteins, are mitochondrial disorders. Also, by definition, disorders of the fatty acid (beta) oxidation, Krebs cycle and pyruvate dehydrogenase complex deficiency are mitochondrial disorders. Although theses disorders may be genetically dissimilar, all disorders contemplated by the present invention are similar in that they result in an energy deficient state.

There is no one identifying feature of mitochondrial disease. Subjects can have combinations of problems whose onset may occur from before birth to late adult life. Mitochondrial diseases should be considered in the differential diagnosis when there are these unexplained features, especially when these occur in combination. Mitochondria disease and disorders can affect multiple organs, resulting in a vast array of symptoms. Symptoms which may affect the brain include, developmental delays, mental retardation, dementia, seizures, neuro-psychiatric disturbances, atypical cerebral palsy, migraines, strokes.

Symptoms which affect the nervous system may include, weakness (which may be intermittent), neuropathic pain, absent reflexes, gastrointestinal problem (gastroesophogeal reflux, delayed gastric emptying, constipation, pseudo-obstruction), fainting, absent or excessive sweating resulting in temperature regulation problems.

Symptoms which affect muscle may include, weakness, hypotonia, cramping and muscle pain.

Symptoms which affect the kidneys include proximal renal tubular wasting resulting in loss of protein, magnesium, phosphorous, calcium and other electrolytes.

Symptoms which affect the heart include cardiac conduction defects (heart blocks) and cardiomyopathy.

Symptoms which affect the liver include hypoglycemia (low blood sugar) and liver failure.

Symptoms which affect the eyes include visual loss and blindness.

Symptoms which affect the ears include hearing loss and deafness.

Symptoms which affect the pancreas include diabetes and exocrine pancreatic failure (inability to make digestive enzymes).

There may also be systemic problems associated with mitochondrial dysfunction, including failure to gain weight, short statue, fatigue, respiratory problems.

Mitochondrial defects have been linked to Alzheimer's, Parkinson's, diabetes, autism, and the aging process. Other disease associated with mitochondrial dysfunction include, LIC (Lethal Infantile Cardiomyopathy), Beta-oxidation Defects, COX Deficiency, Mitochondrial Cytopathy, Alpers Disease, Barth syndrome, Carnitine-Acyl-Carnitine Deficiency, Carnitine Deficiency, Co-Enzyme Q10 Deficiency, Complex I Deficiency, Complex II Deficiency, Complex III Deficiency, Complex IV Deficiency, Complex V Deficiency, CPEO, CPT I Deficiency, Glutaric Aciduria Type II, KSS, lactic acidosis, LCAD, LCHAD, Leigh Disease, LHON, Luft Disease, MAD, MCA, MELAS, MERRF, mitochondrial DNA depletion, Mitochondrial Encephalopath, MNGIE, NARP, Pearson Syndrome, Pyruvate Carboxylase Deficiency, Pyruvate Dehydrogenase Deficiency, SCAD, SCHAD and VLCAD.

Alpers Disease, or Progressive Infantile Poliodystrophy, includes symptoms such as seizures, dementia, spasticity, blindness, liver dysfunction, and cerebral degeneration. (Luft; The development of mitochondrial medicine. Proceedings of the National Academy of sciences of the United States of America; 1994 ; 91(19); 8731-8).

Barth syndrome or LIC (Lethal Infantile Cardiomyopathy) is an X-linked recessive disorder the symptoms of which include skeletal myopathy, cardiomyopathy, short stature, and neutropenia. (Christodoulou; Barth syndrome: clinical observations and genetic linkage studies. American Journal of Medical Genetics; 1994; 50(3); 255-64).

Carnitine-Acyl-Carnitine Deficiency is an autosomal recessive disorder, the symptoms of which are seizures, apnea, bradycardia, vomiting, lethargy, coma, enlarged liver, limb weakness, myoglobin in the urine, Reye-like symptoms triggered by fasting.

Carnitine Deficiency is an autosomal recessive disease, the symptoms of which include Cardiomyopathy, failure to thrive, and altered consciousness or coma, sometimes hypotonia.

Co-Enzyme Q10 Deficiency is most likely an autosomal recessive disease, the symptoms of which include Encephalomyopathy, mental retardation, exercise intolerance, ragged-red fibers, and recurrent myoglobin in the urine.

Complex I Deficiency or NADH dehydrogenase (NADH—CoQ reductase) deficiency is an autosomal disease, the symptoms of which are classified by three major forms: (1) fatal infantile multisystem disorder, characterized by developmental delay, muscle weakness, heart disease, congenital lactic acidosis, and respiratory failure; (2) myopathy beginning in childhood or in adult life, manifesting as exercise intolerance or weakness. Elevated lactic acid common; and (3) mitochondrial encephalomyopathy (including MELAS), which may begin in childhood or adult life and consists of variable combinations of symptoms and signs, including ophthalmoplegia, seizures, dementia, ataxia, hearing loss, pigmentary retinopathy, sensory neuropathy, and uncontrollable movements. In addition, this disorder may cause Leigh Syndrome.

Complex II Deficiency or Succinate dehydrogenase deficiency, the symptoms of which include encephalomyopathy and various manifestations, including failure to thrive, developmental delay, hyoptonia, lethargy, respiratory failure, ataxia, myoclonus and lactic acidosis. May also cause Leigh Syndrome.

Complex III Deficiency or Ubiquinone-cytochrome c oxidoreductase deficiency, symptoms of which are categorized in four major forms: (1) fatal infantile encephalomyopathy, congenital lactic acidosis, hypotonia, dystrophic posturing, seizures, and coma. Ragged-red fibers common; (2) encephalomyopathies of later onset (childhood to adult life): various combinations of weakness, short stature, ataxia, dementia, hearing loss, sensory neuropathy, pigmentary retinopathy, and pyramidal signs. Ragged-red fibers common. Possible lactic acidosis; (3) myopathy, with exercise intolerance evolving into fixed weakness. Ragged-red fibers common. Possible lactic acidosis; and (4) infantile histiocytoid cardiomyopathy.

Complex IV Deficiency or Cytochrorre c oxidase deficiency is caused by a defect in Complex IV of the respiratory chain, the symptoms of which can be categorized in two major forms: (1) encephalomyopathy, which is typically normal for the first 6 to 12 months of life and then show developmental regression, ataxia, lactic acidosis, optic atrophy, ophthalmoplegia, nystagmus, dystonia, pyramidal signs, respiratory problems and frequent seizures; and (2) myopathy: Two main variants: (a) Fatal infantile myopathy: may begin soon after birth and accompanied by hypotonia, weakness, lactic acidosis, ragged-red fibers, respiratory failure, and kidney problems: and b) Benign infantile myopathy: may begin soon after birth and accompanied by hypotonia, weakness, lactic acidosis, ragged-red fibers, respiratory problems, but (if the child survives) followed by spontaneous improvement.

Complex V Deficiency or ATP synthase deficiency includes symptoms such as slow, progressive myopathy.

CPEO or Chronic Progressive External Ophthalmoplegia Syndrome includes symptoms such as visual myopathy, retinitis pigmentosa, dysfunction of the central nervous system. It is caused by single mitochondrial DNA deletions, with Mitochondrial DNA point mutation, A3243G being the most common (Luft; The development of mitochondrial medicine. [Review]; Proceedings of the National Academy of Sciences of the United States of America; 1994 ; 91(19); 8731-8).

CPT I Deficiency is an autosomal recessive disease and includes symptoms such as enlarged liver and recurrent Reye-like episodes triggered by fasting or illnesses.

CPT II Deficiency is an autosomal recessive disease, the symptoms of which include exercise intolerance, fasting intolerance, muscle pain, muscle stiffness, and myoglobin in the urine and in infants, Reye-like syndrome, enlarged liver, hypoglycemia, enlarged heart and cardiac arrhythmia.

KSS or Kearns-Sayre Syndrome, in most cases is caused by large mitochondria DNA deletions. Symptoms associated with KSS include progressive external ophthalmoplegia, pigmentary retinopathy, heart block, and high cerebrospinal protein.

Lactic Acidosis is associated with the accumulation of lactic acid due to its production exceeding its use. Chronic lactic acidosis is a common symptom of mitochondrial disease.

LCAD or Long-Chain Acyl-CoA Dehydrongenase Deficiency, is an autosomal recessive disorder, which causes a fatal syndrome, in infants, typified by failure to thrive, enlarged liver, enlarged heart, metabolic encephalopathy and hypotonia.

LCHAD is an autosomal recessive disorder, characterized by symptoms such as encephalopathy, liver dysfunction, cardiomyopathy, and myopathy. Also pigmentary retinopathy and peripheral neuropathy.

Leigh Disease or Syndrome or Subacute Necrotizing Encephalomyelopathy is characterized by symptoms such as Seizures, hypotonia, fatigue, nystagmus, poor reflexes, eating and swallowing difficulties, breathing problem and poor motor function.

LHON or Leber Hereditary Optic Neuropathy is caused by mitochondrial DNA point mutations, including G14459A, among others. Symptoms associated with LHON include primarily blindness in young men. Less common symptoms include mild dementia, ataxia, spasticity, peripheral neuropathy and heart conduction defects.

Luft Disease is characterized by symptoms such as hypermetabolism, with fever, heat intolerance, profuse perspiration, polyphagia, polydipsia, ragged-red fibers, and resting tachycardia. In addition to exercise intolerance with mild weakness.

MAD or Glutaric Aciduria Type II or multiple Acyl-CoA Dehydrogenase Deficiency is caused by defects of the flavoproteins responsible for transferring electrons (ETF or ETF-dehydrogenase) therefor affecting the function of all six ETF-fimneling acyl-CoA dehydrogenases

MCAD or Medium-Chain Acyl-CoA Dehydrongen.ase Deficiency is ans autosomal recessive disorder, which afflicts infants or young children with episodes of encephalopathy, enlarged and fatty degeneration of the liver, and low carnitine in the blood.

MELAS or Mitochondrial Encephalomyopathy Lactic Acidosis and Strokelike Episodes is caused by mitochondrial DNA point mutations, the most common of which is A3243G. It is characterized by symptoms: Short statue, seizures, stroke-like episodes with focused neurological deficits, recurrent headaches, cognitive regression, disease progression ragged-red fibers (Koo, et. al.; Mitochondrial encephalomyopathy, lactic acidosis, stroke-like episodes (MELAS): clinical, radiological, pathological, and genetic observations; Annals of Neurology; 1993; 34(1); (25-32).

MERRF or Myoclonic Epilepsy and Ragged-Red Fiber Disease is caused by the mitochondrial DNA point mutations A8344G and T8356C. Its symptoms include myoclonus, epilepsy, progressive ataxia, muscle weakness and degeneration, deafness and dementia (Luft; The development of mitochondrial medicine; Proceedings of the National Academy of Sciences of the United States of America; 1994; 91(19); (8731-8).

There are three forms of mitochondrial DNA Depletion. These include: (1) congenital myopathy: Neonatal weakness, hypotonia requiring assisted ventilation, possible renal dysfunction. Severe lactic acidosis. Prominent ragged-red fibers. Death due to respiratory failure usually occurs prior to one year of age; (2) infantile myopathy: Following normal early development until one year old, weakness appears and worsens rapidly, causing respiratory failure and death typically within a few years; and (3) hepatopathy, enlarged liver and intractable liver failure, myopathy. Severe lactic acidosis. Death is typical within the first year.

Mitochondrial Encephalopathy, also includes Encephalomyopathy and Encephalomyelopathy.

MNGIE or Myoneurogastointestinal Disorder and Encephalopathy, include symptoms such as progressive external ophthalmoplegia, limb weakness, peripheral neuropathy, digestive tract disorders, leukodystrophy, lactic acidosis and ragged red fibers.

NARP or Neuropathy, Ataxia, and Retinitis Pigmentosa is caused by mitochondrial DNA point mutations in genes associated with Complex V, including T8993G, (also T8993C by some researchers). Leigh Syndrome may result if the percentage of mutation is high enough.

Pearson Syndrome is characterized by symptoms associated with bone marrow and pancreas dysfunction. It is caused by single mitochondrial DNA deletions. Inheritance is usually sporadic. Those who survive infancy usually develop Kearns-Sayre Syndrome.

Pyruvate Carboxylase Deficiency is an autosomal recessive disorder, the symptoms of which include lactic acidosis, hypoglycemia, severe retardation, failure to thrive, in addition to seizures and spasticity.

Pyruvate Dehydrogenase Deficiency is characterized by symptoms such as lactic acidosis, ataxia, pyruvic acidosis, spinal and cerebellar degeneration. Less common symptoms include agenesis of the corpus callosum and lesions in the basal ganglia, cerebelum, and brain stem, growth delay, hypotonia, seizures and polyneuropathy.

SCAD or Short-Chain Acyl-CoA Dehydrogenase Deficiency, is an autosomal recessive disorder characterized by symptoms such as failure to thrive, developmental delay and hypoglycemia.

SCHAD is an autosomal recessive disorder, characterized by encephalopathy and possibly liver disease or cardiomyopathy.

VLCAD or Very Long-Chain Acyl-CoA Dehydrongenase Deficiency is an autosomal recessive disorder, characterized by various manifestations, ranging from fatal infantile encephalopathy to recurrent myoglobin in the urine, similar to the myopathic form of CPT II deficiency.

In addition, other diseases and disorders which can be treated using the methods of the present invention include, without being limited to, A-Beta-Lipoproteinemia, A-V, A Beta-2-Microglobulin Amyloidosis, A-T, A1AD, A1AT, Aagenaes, Aarskog syndrome, Aarskog-Scott Syndrome, Aase-smith syndrome, Aase Syndrome, AAT, Abderhalden-Kaufmann-Lignac Syndrome, Abdominal Muscle Deficiency Syndrome, Abdominal Wall Defect, Abdominal Epilepsy, Abdominal Migraine, Abductor Spasmodic Dysphonia, Abductor Spastic Dysphonia, Abercrombie Syndrome, blepharon-Macrostomia Syndrome, ABS, Absence of HPRT, Absence of Corpus Callosum Schinzel Typ, Absence Defect of Limbs Scalp and Skull, Absence of Menstruation Primar, Absence of HGPRT, Absorptive Hyperoxaluriaor Enteric, Abt-Letterer-Siwe Disease, ACADL, ACADM Deficiency, ACADM, ACADS, Acanthocytosis-Neurologic Disorder, Acanthocytosis, Acantholysis Bullosa, Acanthosis Nigricans, Acanthosis Bullosa, Acanthosis Nigricans With Insulin Resistance Type A, Acanthosis Nigricans With Insulin Resistance Type B, Acanthotic Nevus, Acatalasemia, Acatalasia, ACC, Accessory Atrioventricular Pathways, Accessory Atrioventricular Pathways, Acephaly, ACF with Cardiac Defects, Achalasia, Achard-Thiers Syndrome, ACHARD (Marfan variant), Achard's syndrome, Acholuric Jaundice, Achondrogenesis, Achondrogenesis Type IV, Achondrogenesis Type III, Achondroplasia, Achondroplasia Tarda, Achondroplastic Dwarfism, Achoo Syndrome, Achromat, Achromatope, Achromatopic, Achromatopsia, Achromic Nevi, Acid Ceramidase Deficiency, Acid Maltase Deficiency, Acid Maltase Deficiency, Acid Beta-glucosidase Deficiency, Acidemia Methylmalonic, Acidemia Propionic, Acidemia with Episodic Ataxia and Weakness, Acidosis, Aclasis Tarsoepiphyseal, ACM, Acoustic Neurilemoma, Acoustic Neuroma, ACPS with Leg Hypoplasia, ACPS II, ACPS IV, ACPS III, Acquired Aphasia with Convulsive Disorder, Acquired Brown Syndrome, Acquired Epileptic Aphasia, Acquired Factor XIII Deficiency, Acquired Formn of ACC (caused by infection while still in womb), Acquired Hyperoxaluria, Acquired Hypogammaglobulinemia, Acquired Immunodeficiency Syndrome (AIDS), Acquired Iron Overload, Acquired Lipodystrophy, Acquired Partial Lipodystrophy, Acquired Wandering Spleen, ACR, Acral Dysostosis with Facial and Genital Abnormalities, Acro Renal, Acrocallo sal Syndrome Schinzel Type, Acrocephalosyndactyly, Acrocephalosyndactyly Type I, Acrocephalosyndactyly Type I Subtype I, Acrocephalopolysyndactyly Type II, Acrocephalosyndactyly type II, Acrocephalopolysyndactyly Type III, Acrocephalosyndactyly Type III, Acrocephalopolysyndactyly Type IV, Acrocephalosyndactyly V (ACS5 or ACS V) Subtype I, Acrocephaly Skull Asymmetry and Mild Syndactyly, Acrocephaly, Acrochondrohyperplasia, Acrodermatitis Enteropathica, Acrodysostosis, Acrodystrophic Neuropathy, Acrodystrophic Neuropathy, Acrofacial Dysostosis Nager Type, Acrofacial Dysostosis Nager Type, Acrofacial Dysostosis Postaxial Type, Acrofacial Dysostosis Type Genee-Wiedep, Acrogeria Familial, Acromegaly, Acromelalgia Hereditary, Acromesomelic Dysplasia, Acromesomelic Dwarfism, Acromicric Skeletal Dysplasia, Acromicric Dysplasia, Acroosteolysis with Osteoporosis and Changes in Skull and Mandible, Acroosteolysis, Acroparesthesia, ACS I, ACS Type III, ACS Type III, ACS, ACS3, ACS3, ACTH Deficiency, Action Myoclonus, Acute Brachial Neuritis Syndrome, Acute Brachial Radiculitis Syndrome, Acute Cerebral Gaucher Disease, Acute Cholangitis, Acute Disseminated Encephalomyeloradiculopathy, Acute Disseminated Histiocytosis-X, Acute Hemorrhagic Polioencephalitis, Acute Idiopathic Polyneuritis, Acute Immune-Mediation Polyneuritis, Acute Infantile Pelizaeus-Merzbacher Brain Sclerosis, Acute Intermittant Porphyria, Acute Porphyrias, Acute Sarcoidosis, Acute Shoulder Neuritis, Acute Toxic Epidermolysis, Acyl-CoA Dehydrogenase Deficiency Long-Chain, Acyl-CoA Dehydrogenase Deficiency Short-Chain, Acyl-CoA Dihydroxyacetone Acyltransferase, Acyl-coenzyme A Oxidase Deficiency, ADA, ADA Deficiency, Adam Complex, Adamantiades-Behcet's Syndrome, Adamantinoma, Adams Oliver Syndrome, Adaptive Colitis, ADD combined type, ADD, ADD, Addison Disease with Cerebral Sclerosis, Addison's Anemia, Addison's Anemia, Addison's Disease, Addison's Disease, Addison's Disease, Addison-Biermer Anemia, Addison-Biermer Anemia, Addison-Schilder Disease, Addisonian Pernicious Anemia, Addisonian Pernicious Anemia, Adducted Thumbs-Mental Retardation, Adductor Spasmodic Dysphonia, Adductor Spastic Dysphonia, Adenoma Associated Virilism of Older Women, Adenomatosis of the Colon and Rectumn, Adenomatous polyposis of the Colon, Adenomatous Polyposis Familial, Adenosine Deaminase Deficienc-y, Adenosine Deaminase Deficiency, Adenylosuccinase deficiency, ADHD predominantly hyperactive-impulsive type, ADHD predominantly inattentive type, ADHD, Adhesive Arachnoiditis, Adie Syndrome, Adie's Syndrome, Adie's Tonic Pupil, Adie's Pupil, Adipogenital Retinitis Pigmentosa Polydactyly, Adipogenital-Retinitis Pigmentosa Syndrome, Adiposa Dolorosa, Adiposis Dolorosa, Adiposogenital Dystrophy, Adolescent Cystinosis, ADPKD, Adrenal Cortex Adenoma, Adrenal Disease, Adrenal Hyperfunction resulting from Pituitary ACTH Excess, Adrenal Hypoplasia, Adrenal Insufficiency, Adrenal Neoplasm, Adrenal Virilism, Adrenal Virilism, Adreno-Retinitis Pigmentosa-Polydactyly Syndrome, Adrenocortical Insufficiency, Adrenocortical Hypofunction, Adrenocorticotropic Hormone Deficiency Isolated, Adrenogenital Syndrome, Adrenogenital Syndrome, Adrenoleukodystrophy, Adrenoleukodystrophy, Adrenomyeloneuropathy, Adrenomyeloneuropathy, Adreno-Retinitis Pigmentosa-Polydactyly Syndrome, Adult Cystinosis, Adult Dermatomyositis, Adult Hypophosphatasia, Adult Macula Lutea Retinae Degeneration, Adult Onset ALD, Adult-Onset Ceroidosis, Adult Onset Medullary Cystic Disease, Adult Onset Pernicious Anemia, Adult Onset Pernicious Anemia, Adult Onset Schindler Disease, Adult-Onset Subacute Necrotizing Encephalomyelopathy, Adult Onset Pernicious Anemia, Adult Polycystic Kidney Disease, Adult Onset Medullary Cystic Disease, Adynlosuccinate Lyase Deficiency, AE, AEC Syndrome, AFD, AFD, Afibrinogenemia, African Siderosis, AGA, Aganglionic Megacolon, Age Related Macular Degeneration, Agenesis of Commissura Magna Cerebri, Agenesis of Corpus Callosum, Agenesis of Corpus Callosum-Infantile Spasms-Ocular Anomalies, Agenesis of Corpus Callosum and Chorioretinal Abnormality, Agenesis of Corpus Callosum-Chorioretinitis Abnormality, Aggressive mastocytosis, Agnosis Primary, AGR Triad, AGU, Agyria, Agyria-pachygria-band spectrum, AHC, AHD, AHDS, AHF Deficiency, AHG Deficiency, AHO, Ahumada Del Castillo, Aicardi Syndrome, Aicardi Syndrome, AIED, AIMP, AIP, AIS, AIS, Akinetic Seizure, ALA-D Porphyria, Alactasia, Alactasia, Alagille Syndrome, Aland Island Eye Disease (X-Linked), Alaninuria, Albers-Schonberg Disease, Albinism, Albinism, Albinismus, Albinoidism, Albright Hereditary Osteodystrophy, Alcaptonuria, Alcaptonuria, Alcohol-Related Birth Defects, Alcoholic Embryopathy, Ald, ALD, ALD, Aldosterone, Aldosteronism With Normal Blood Pressure, Aldrich Syndrome, Alexander's Disease, Alexanders Disease, Algodystrophy, Algoneurodystrophy, Alkaptonuria, Alkaptonuric Ochronosis, Alkyl DHAP synthase deficiency, Allan-Herndon-Dudley Syndrome, Allan-Herndon Syndrome, Allan-Herndon-Dudley Mental Retardation, Allergic Granulomatous Antitis, Allergic Granulomatous Angiitis of Cronkhite-Canada, Alobar Holoprosencephaly, Alopecia Areata, Alopecia Areata, Alopecia Celsi, Alopecia Cicatrisata, Alopecia Circumscripta, Alopecia-Poliosis-Uveitis-Vitiligo-Deafness-Cutaneous-Uveo-O, Alopecia Seminuniversalis, Alopecia Totalis, Alopecia Universalis, Alpers Disease, Alpers Disease, Alpers Diffuse Degeneration of Cerebral Gray Matter with Hepatic Cirrhosis, Alpers Progressive Infantile Poliodystrophy, Alpha-1-Antitrypsin Deficiency, Alpha-1 4 Glucosidase Deficiency, Alpha-1 4 Glucosidase Deficiency, Alpha-Galactosidase A Deficiency, Alpha-Galactosidase B Deficiency, Alpha-1 4 Glucosidase Deficiency, Alpha High-Density Lipoprotein Deficieny, Alpha-L-Fucosidase Deficiency Fucosidosis Type 3, Alpha-GalNAc Deficiency Schindler Type, Alpha-1 4 Glucosidase Deficiency, Alpha-L-Fucosidase Deficiency Fucosidosis Type 3, Alphalipoproteinemia, Alpha Mannosidosis, Alpha-N-Acetylgalactosaminidase Deficiency Schindler Type, Alpha-NAGA Deficiency Schindler Type, Alpha-Neuraminidase Deficiency, Alpha-Thalassemia/mental retardation syndorme non-deletion type, Alphalipoproteinemia, Alport Syndrome, ALS, Alstroem's Syndrome, Alstroem, Alstrom Syndrome, Alternating Hemiplegia Syndrome, Alternating Hemiplegia of Childhood, Alzheimer's Disease, Amaurotic Familial Idiocy, Amaurotic Familial Idiocy, Amaurotic Familial Idiocy Adult, Amaurotic Familial Infantile Idiocy, Amaurotic Familial Infantile Idiocy, Ambiguous Genitalia, AMC, AMD, Ameloblastoma, Amelogenesis Imperfecta, Amenorrhea-Galactorrhea Nonpuerperal, Amenorrhea-Galactorrhea-FSH Decrease Syndrome, Amenorrhea, Amino Acid Disorders, Aminoaciduria-Osteomalacia-Hyperphosphaturia Syndrome, AMN, AMN, Amniocentesis, Amniocentesis, Amniotic Bands, Amniotic Band Syndrome, Amniotic Band Disruption Complex, Amniotic Band Sequence, Amniotic Rupture Sequence, Amputation Congenital, AMS, Amsterdam Dwarf Syndrome de Lange, Amylo-1 6-Glucosidase Deficiency, Amyloid Arthropathy of Chronic Hemodialysis, Amyloid Corneal Dystrophy, Amyloid Polyneuropathy, Amyloidosis, Amyloidosis of Familial Mediterranean Fever, Amylopectinosis, Amyoplasia Congenita, Amyotrophic Lateral Sclerosis, Amyotrophic Lateral Sclerosis, Amyotrophic Lateral Sclerosis-Polyglucosan Bodies, AN, AN 1, AN 2, Anal Atresia, Anal Membrane, Anal Rectal Malformations, Anal Rectal Malformations, Anal Stenosis, Analine 60 Amyloidosis, Analphalipoproteinemia, Analrectal, Analrectal, Analrectal, Anaplastic Astrocytoma, Andersen Disease, Anderson-Fabry Disease, Andersen Glycogenosis, Anderson-Warburg Syndrome, Andre Syndrome, Andre Syndrome Type II, Androgen Insensitivity, Androgen Insensitivity Syndrome Partial, Androgen Insensitivity Syndrome, Androgen Insensitivity Syndrome Partial, Androgenic Steroids, Anemia Autoimmune Hemolytic, Anemia Blackfan Diamond, Anemia, Congenital, Triphalangeal Thumb Syndrome, Anemia Hemolytic Cold Antibody, Anemia Hemolytic Cold Antibody, Anemia Hemolytic with PGK Deficiency, Anemia Pernicious, Anencephaly, Angelman Syndrome, Angio-Osteohypertrophy Syndrome, Angiofollicular Lymph Node Hyperplasia, Angiohemophilia, Angiokeratoma Corporis, Angiokeratoma Corporis Difflusum, Angiokeratoma Difflise, Angiomatosis Retina, Angiomatous Lymphoid, Angioneurotic Edema Hereditary, Anhidrotic Ectodermal Dysplasia, Anhidrotic X-Linked Ectodermal Dysplasias, Aniridia, Aniridia-Ambiguous Genitalia-Mental Retardation, Aniridia Associated with Mental Retardation, Aniridia-Cerebellar Ataxia-Mental Deficiency, Aniridia Partial-Cerebellar Ataxia-Mental Retardation, Aniridia Partial-Cerebellar Ataxia-Oligophrenia, Aniridia Type I, Aniridia Type II, Aniridia-Wilms' Tumor Association, Aniridia-Wilms' Tumor-Gonadoblastoma, Ankyloblepharon-Ectodermal Defects-Cleft Lip/Palate, Ankylosing Spondylitis, Ankylosing Spondylitis, Annular groves, Anodontia, Anodontia, Anodontia Vera, Anomalous Trichromasy, Anomalous Dysplasia of Dentin,Coronal Dentin Dysplasia, Anomic Aphasia, Anophthalmia, Anorectal, Anorectal Malformations, Anosmia, Anterior Bowing of the Legs with Dwarfism, Anterior Membrane Corneal Dystrophy, Anti-Convulsant Syndrome, Anti-Epstein-Barr Virus Nuclear Antigen (EBNA) Antibody Deficiency, Antibody Deficiency, Antibody Deficiency with near normal Immunoglobulins, Antihemophilic Factor Deficiency, Antihemophilic Globulin Deficiency, Antiphospholipid Syndrome, Antiphospholipid Syndrome, Antiphospholipid Antibody Syndrome, Antithrombin III Deficiency, Antithrombin III Deficiency Classical (Type I), Antitrypsin Deficiency, Antley-Bixler Syndrome, Antoni's Palsy, Anxietas Tibialis, Aorta Arch Syndrome, Aortic and Mitral Atresia with Hypoplasic Left Heart Syndrome, Aortic Stenosis, Aortic Stenosis, Aparoschisis, APC, APECED Syndrome, Apert Syndrome, Aperts, Aphasia, Aplasia Axialis Extracorticales Congenital, Aplasia Cutis Congenita, Aplasia Cutis Congenita with Terminal Transverse Limb Defects, Aplastic Anemia, Aplastic Anemia with Congenital Anomalies, APLS, Apnea, Appalachian Type Amyloidosis, Apple Peel Syndrome, Apraxia, Apraxia, Apraxia Buccofacial, Apraxia Constructional, Apraxia Ideational, Apraxia Ideokinetic, Apraxia Ideomotor, Apraxia Motor, Apraxia Oculomotor, APS, Arachnitis, Arachnodactyly Contractural Beals Type, Arachnodactyly, Arachnoid Cysts, Arachnoiditis Ossificans, Arachnoiditis, Aran-Duchenne, Aran-Duchenne Muscular Atrophy, Aregenerative Anemia, Arginase Deficiency, Argininemia, Arginino Succinase Deficiency, Argininosuccinase Deficiency, Argininosuccinate Lyase Deficiency, Argininosuccinic Acid Lyase-ASL, Argininosuccinic Acid Synthetase Deficiency, Argininosuccinic Aciduria, Argonz-Del Castillo Syndrome, Arhinencephaly, Armenian Syndrome, Amold-Chiari Malformation, Arnold-Chiari Syndrome, ARPKD, Arrhythmic Myoclonus, Arrhythmogenic Right Ventricular Dysplasia, Arteriohepatic Dysplasia, Arteriovenous Malformation, Arteriovenous Malformation, Arteriovenous Malformation of the Brain, Arteritis Giant Cell, Arthritis, Arthritis Urethritica, Arthro-Dento-Osteodysplasia, Arthro-Ophthalmopathy, Arthrochalasis Multiplex Congenita, Arthrogryposis Multiplex Congenita, Arthrogryposis Multiplex Congenita, Distal, Type IIA, ARVD, Arylsulfatase-B Deficiency, AS, AS, AS, AS, ASA Deficiency, Ascending Paralysis, ASD,Atrioseptal Defects, ASH, Ashermans Syndrome, Ashkenazi Type Amyloidosis, ASL Deficiency, Aspartylglucosaminuria, Aspartylglycosaminuria, Asperger's Syndrome, Asperger's Type Autism, Asphyxiating Thoracic Dysplasia, Asplenia Syndrome, ASS Deficiency, Asthma, Astrocytoma Grade I (Benign), Astrocytoma Grade II (Benign), Asymmetric Crying Facies with Cardiac Defects, Asymmetrical septal hypertrophy, Asymptomatic Callosal Agenesis, AT, AT III Deficiency, AT III Variant IA, AT III Variant Ib, AT 3, Ataxia, Ataxia Telangiectasia, Ataxia Telangiectasia, Ataxia with Lactic Acidosis Type II, Ataxia Cerebral Palsy, Ataxiadynamia, Ataxiophemia, ATD, Athetoid Cerebral Palsy, Atopic Eczema, Atresia of Esophagus with or without Tracheoesophageal Fistula, Atrial Septal Defects, Atrial Septal Defect Primum, Atrial and Septal and Small Ventricular Septal Defect, Atrial Flutter, Atrial Fibrillation, Atriodigital Dysplasia, Atrioseptal Defects, Atrioventricular Block, Atrioventricular Canal Defect, Atrioventricular Septal Defect, Atrioventricular Septal Defect, Atrophia Bulborum Hereditaria, Atrophic Beriberi, Atrophy Olivoporntocerebellar, Attention Deficit Disorder, Attention Deficit Hyperactivity Disorder, Attentuated Adenomatous Polyposis Coli, Atypical Amyloidosis, Atypical Hyperphenylalaninemia, Atypical Hyperphenylalaninemia, Auditory Canal Atresia, Auriculotemporal Syndrome, Autism, Autism Asperger's Type, Autism Dementia Ataxia and Loss of Purposeful Hand Use, Autism Infantile Autism, Autoimmune Addison's Disease, Autoimmune Hemolytic Anemia, Autoimmune Hemolytic Anemia, Autoimmune Hemolytic Anemia, Autoimmune Hemolytic Anemia, Autoimmune Hepatitis, Autoimmune-Polyendocrinopathy-Candidias, Autoimmune Polyglandular Disease Type I, Autosomal Dominant Albinism, Autosomal Dominant Compelling Helioophthalmic Outburst Syndrome, Autosomal Dominant Desmin Distal Myopathy with Late Onset, Autosomal Dominant EDS, Autosomal Dominant Emery-Dreifuss Muscular Dystrophy, Autosomal Dominant Keratoconus, Autosomal Dominant Pelizaeus-Merzbacher Brain Sclerosis, Autosomal Dominant Polycystic Kidney Disease, Autosomal Dominant Spinocerebellar Degeneration, Autosomal Recessive Agammaglobulinemia, Autosomal Recessive Centronuclear Myopathy, Autosomal Recessive Conradi-Hunermann Syndrome, Autosomal Recessive EDS, Autosomal Recessive Emery-Dreifuss Muscular Dystrophy, Autosomal Recessive Forms of Ocular Albinism, Autosomal Recessive Inheritance Agenesis of Corpus Callosum, Autosomal Recessive Keratoconus, Autosomal Recessive Polycystic Kidney Disease, Autosomal Recessive Severe Combined Immunodeficiency, AV, AV, AVM, AVSD, AWTA, Axilla Abscess, Axonal Neuropathy Giant, Azorean Neurologic Disease, B-K Mole Syndrome, Babinski-Froelich Syndrome, BADS, Baillarger's Syndrome, Balkan Disease, Baller-Gerold Syndrome, Ballooning Mitral Valve, Balo Disease Concentric Sclerosis, Baltic Myoclonus Epilepsy, Bannayan-Zonana syndrome (BZS), Bannayan-Riley-Ruvalcaba syndrome, Banti's Disease, Bardet-Biedl Syndrome, Bare Lymphocyte Syndrome, Barlow's syndrome, Barraquer-Simons Disease, Barrett Esophagus, Barrett Ulcer, Barth Syndrome, Barth syndrome, Bartter's Syndrome, Basal Cell Nevus Syndrome, Basedow Disease, Bassen-Komzweig Syndrome, Batten Disease, Batten-Mayou Syndrome, Batten-Spielmeyer-Vogt's Disease, Batten Turner Syndrome, Batten Turner Type Congenital Myopathy, Batten-Vogt Syndrome, BBB Syndrome, BBB Syndrome (Opitz), BBB Syndrome, BBBG Syndrome, BCKD Deficiency, BD, BDLS, BE, Beals Syndrome, Beals Syndrome, Beals-Hecht Syndrome, Bean Syndrome, BEB, BEB, Bechterew Syndrome, Becker Disease, Becker Muscular Dystrophy, Becker Muscular Dystrophy, Becker Nevus, Beckwith Wiedemann Syndrome, Beckwith-Syndrome, Begnez-Cesar's Syndrome, Behcet's syndrome, Behcet's Disease, Behcet's Disease, Behr 1, Behr 2, Bell's Palsy, Benign Acanthosis Nigricans, Benign Astrocytoma, Benign Cranial Nerve Tumors, Benign Cystinosis, Benign Essential Blepharospasm, Benign Essential Tremor, Benign Familial Hematuria, Benign Focal Amyotrophy, Benign Focal Amyotrophy of ALS, Benign Hydrocephalus, Benign Hypermobility Syndrome, Benign Keratosis Nigricans, Benign Paroxysmal Peritonitis, Benign Recurrent Hematuria, Benign Recurrent Intrahepatic Cholestasis, Benign Spinal Muscular Atrophy with Hypertrophy of the Calves, Benign Symmetrical Lipomatosis, Benign Tumors of the Central Nervous System, Berardinelli-Seip Syndrome, Berger's Disease, Beriberi, Berman Syndrome, Bernard-Homer Syndrome, Bernard-Soulier Syndrome, Besnier Prurigo, Best Disease, Beta-Alanine-Pyruvate Aminotransferase, Beta-Galactosidase Deficiency Morquio Syndrome, Beta-Glucuronidase Deficiency, Beta Oxidation Defects, Beta-oxidation Defects, Beta Thalassemia Major, Beta Thalassemia Minor, Betalipoprotein Deficiency, Bethlem myopathy, Beuren Syndrome, BH4 Deficiency, BH4 Deficiency, Biber-Haab-Dimmer Corneal Dystrophy, Bicuspid Aortic Valve, Bicuspid Aortic Valve, Biedl-Bardet, Bifid Cranium, Bifunctional Enzyme Deficiency, Bilateral Acoustic Neurofibromatosis, Bilateral Acoustic Neuroma, Bilateral Right-Sidedness Sequence, Bilateral Renal Agenesis, Bilateral Temporal Lobe Disorder, Bilious Attacks, Bilirubin Glucuronosyltransferase Deficiency Type I, Binder Syndrome, Binswanger's Disease, Binswanger's Encephalopathy, Biotinidase deficiency, Bird-Headed Dwarfism Seckel Type, Birth Defects, Birthmark, Bitemporal Forceps Marks Syndrome, Biventricular Fibrosis, Bjomstad Syndrome, B-K Mole Syndrome, Black Locks-Albinism-Deafness of Sensoneural Type (BADS), Blackfan-Diamond Anemia, Blennorrheal Idiopathic Arthritis, Blepharophimosis, Ptosis, Epicanthus Inversus Syndrome, Blepharospasm, Blepharospasm, Blepharospasm Benign Essential, Blepharospasm Oromandibular Dystonia, Blessig Cysts, BLFS, Blindness, Bloch-Siemens Incontinentia Pigmenti Melanoblastosis Cutis Linearis, Bloch-Siemens-Sulzberger Syndrome, Bloch-Sulzberger Syndrome, Blood types, Blood type A, Blood type B, Blood type AB, Blood type O, Bloom Syndrome, Bloom-Torre-Mackacek Syndrome, Blue Rubber Bleb Nevus, Blue Baby, Blue Diaper Syndrome, BMD, BOD, BOFS, Bone Tumor-Epidermoid Cyst-Polyposis, Bonnet-Dechaume-Blanc Syndrome, Bonnevie-Ulrich Syndrome, Book Syndrome, BOR Syndrome, BORJ, Borjeson Syndrome, Borjeson-Forssman-Lehmann Syndrome, Bowen Syndrome, Bowen-Conradi Syndrome, Bowen-Conradi Hutterite, Bowen-Conradi Type Hutterite Syndrome, Bowman's Layer, BPEI, BPES, Brachial Neuritis, Brachial Neuritis Syndrome, Brachial Plexus Neuritis, Brachial-Plexus-Neuropathy, Brachiocephalic Ischemia, Brachmann-de Lange Syndrome, Brachycephaly, Brachycephaly, Brachymorphic Type Congenital, Bradycardia, Brain Tumors, Brain Tumors Benign, Brain Tumors Malignant, Branched Chain Alpha-Ketoacid Dehydrogenase Deficiency, Branched Chain Ketonuria I, Brancher Deficiency, Branchio-Oculo-Facial Syndrome, Branchio-Oto-Renal Dysplasia, Branchio-Oto-Renal Syndrome, Branchiooculofacial Syndrome, Branchiootic Syndrome, Brandt Syndrome, Brandywine Type Dentinogenesis Imperfecta, Brandywine type Dentinogenesis Imperfecta, Breast Cancer, BRIC Syndrome, Brittle Bone Disease, Broad Beta Disease, Broad Thumb Syndrome, Broad Thumbs and Great Toes Characteristic Facies and Mental Retardation, Broad Thumb-Hallux, Broca's Aphasia, Brocq-Duhring Disease, Bronze Diabetes, Bronze Schilder's Disease, Brown Albinism, Brown Enamel Hereditary, Brown-Sequard Syndrome, Brown Syndrome, BRRS, Brueghel Syndrome, Bruton's Agammaglobulinemia Common, BS, BSS, Buchanan's Syndrome, Budd's Syndrome, Budd-Chiari Syndrome, Buerger-Gruetz Syndrome, Bulbospinal Muscular Atrophy-X-linked, Bulldog Syndrome, Bullosa Hereditaria, Bullous CIE, Bullous CIE, Bullous Congenital Ichthyosiform Erythroderma, Bullous Ichthyosis, Bullous Pemphigoid, Burkitt's Lymphoma, Burkitt's Lymphoma African type, Burkitt's Lymphoma Non-african type, BWS, Byler's Disease, C Syndrome, C1 Esterase Inhibitor Dysfunction Type II Angioedema, C1-INH, C1 Esterase Inhibitor Deficiency Type I Angioedema, C1NH, Cacchi-Ricci Disease, CAD, CADASIL, CAH, CAH, Calcaneal Valgus, Calcaneovalgus, Calcium Pyrophosphate Dihydrate Deposits, Callosal Agenesis and Ocular Abnormalities, Calves-Hypertrophy of Spinal Muscular Atrophy, Campomelic Dysplasia, Campomelic Dwarfism, Campomelic Syndrome, Camptodactyly-Cleft Palate-Clubfoot, Camptodactyly-Limited Jaw Excursion, Camptomelic Dwarfism, Camptomelic Syndrome, Camptomelic Syndrome Long-Limb Type, Camurati-Engelmann Disease, Camurati-Engelmann Disease, Canada-Cronkhite Disease, Canavan disease, Canavan's Disease Included, Canavan's Leukodystrophy, Cancer, Cancer Family Syndrome Lynch Type, Cantrell Syndrome, Cantrell-Haller-Ravich Syndrome, Cantrell Pentalogy, Carbamyl Phosphate Synthetase Deficiency, Carbohydrate Deficient Glycoprotein Syndrome, Carbohydrate-Deficient Glycoprotein Syndrome Type Ia, Carbohydrate-Induced Hyperlipemia, Carbohydrate Intolerance of Glucose Galactose, Carbon Dioxide Acidosis, Carboxylase Deficiency Multiple, Cardiac-Limb Syndrome, Cardio-auditory Syndrome, Cardioauditory Syndrome of Jervell and and Lange-Nielsen, Cardiocutaneous Syndrome, Cardio-facial-cutaneous syndrome, Cardiofacial Syndrome Cayler Type, Cardiomegalia Glycogenica Diffusa, Cardiomegalia Glycogenica Diffusa, Cardiomyopathic Lentiginosis, Cardiomyopathy, Cardiomyopathy, Cardiomyopathy Associated with Desmin Storage Myopathy, Cardiomyopathy Due to Desmin Defect, Cardiomyopathy-Neutropenia Syndrome, Cardiomyopathy-Neutropenia Syndrome, Cardiomyopathy-Neutropenia Syndrome Lethal Infantile Cardiomyopathy, Cardiopathic Amyloidosis, Cardiospasm, Cardocardiac Syndrome, Camitine-Acylcarnitine Translocase Deficiency, Camitine Deficiency and Disorders, Camitine Deficiency Primary, Carnitine Deficiency Secondary, Carnitine Deficiency Secondary to MCAD Deficiency, Carnitine Deficiency Syndrome, Carnitine Palmitoyl Transferase I & II (CPT I & II), Camitine Patmitoyltransferase Deficiency, Carnitine Palmitoyltransferase Deficiency Type 1, Carnitine Palmitoyltransferase Deficiency Type 2 benign classical muscular form included severe infantile form included, Carnitine Transport Defect (Primary Camitine Deficiency), Carnosinase Deficiency, Carnosinemia, Caroli Disease, Carpenter syndrome, Carpenter's, Cartilage-Hair Hypoplasia, Cartilage-Hair Hypoplasia, Castleman's Disease, Castleman's Disease Hyaline Vascular Type, Castleman's Disease Plasma Cell Type, Castleman Tumor, Cat Eye Syndrome, Cat's Cry Syndrome, Catalayse deficiency, Cataract-Dental Syndrome, Cataract X-Linked with Hutchinsonian Teeth, Catecholamine hormones, Catel-Manzke Syndrome, Catel-Manzke Type Palatodigital Syndrome, Caudal Dysplasia, Caudal Dysplasia Sequence, Caudal Regression Syndrome, Causalgia Syndrome Major, Cavernomas, Cavernous Angioma, Cavernous Hemangioma, Cavernous Lymphangioma, Cavernous Malformations, Cayler Syndrome, Cazenave's Vitiligo, CBGD, CBGD, CBPS, CBPS, CCA, CCD, CCD, CCHS, CCM Syndrome, CCMS, CCO, CD, CDG1a, CDG1A, CDGS Type Ia, CDGS Type Ia, CDGS, CDI, CdLS, Celiac Disease, Celiac sprue, Celiac Sprue-Dermatitis, Cellelar Immunodeficiency with Purine Nucleoside Phosphorylase Deficiency, Celsus' Vitiligo, Central Apnea, Central Core Disease, Central Core Disease, Central Diabetes Insipidus, Central Form Neurofibromatosis, Central Hypoventilation, Central Sleep Apnea, Centrifugal Lipodystrophy, Centronuclear Myopathy, CEP, Cephalocele, Cephalothoracic Lipodystrophy, Ceramide Trihexosidase Deficiency, Cerebellar Agenesis, Cerebellar Aplasia, Cerebellar Hemiagenesis, Cerebellar Hypoplasia, Cerebellar Vermis Aplasia, Cerebellar Vermis Agenesis-Hypernea-Episodic Eye Moves-Ataxia-Retardation, Cerebellar Syndrome, Cerebellarparenchymal Disorder IV, Cerebellomedullary Malformation Syndrome, Cerebellomedullary Malformation Syndrome, Cerebello-Oculocutaneous Telangiectasia, Cerebelloparenchymal Disorder IV Familial, Cerebellopontine Angle Tumor, Cerebral Arachnoiditis, Cerebral Autosomal Dominant Arteriopathy with Subcortical Infarcts and Leukodystrophy, Cerebral Beriberi, Cerebral Diplegia, Cerebral Gigantism, Cerebral Malformations Vascular, Cerebral Palsy, Cerebro-Oculorenal Dystrophy, Cerebro-Oculo-Facio-Skeletal Syndrome, Cerebrocostomandibular syndrome, Cerebrohepatorenal Syndrome, Cerebromacular Degeneration, Cerebromacular Degeneration, Cerebromuscular Dystrophy Fukuyama Type, Cerebroocular Dysgenesis, Cerebroocular Dysplasia-Muscular Dystrophy Syndrome, Cerebrooculofacioskeletal Syndrome, Cerebroretinal Arteriovenous Aneurysm, Cerebroside Lipidosis, Cerebrosidosis, Cerebrotendinous Xanthomatosis, Cerebrovascular Ferrocalcinosis, Ceroid-Lipofuscinosis Adult form, Cervical Dystonia, Cervical Dystonia, Cervico-Oculo-Acoustic Syndrome, Cervical Spinal Stenosis, Cervical Vertebral Fusion, CES, CF, CFC syndrome, CFIDS, CFND, CGD, CGF, CGF, Chalasodermia Generalized, Chanarin Dorfman Disease, Chanarin Dorfman Syndrome, Chanarin Dorfman Ichthyosis Syndrome, Chandler's Syndrome, Charcot's Disease, Charcot-Marie-Tooth, Charcot-Marie-Tooth Disease, Charcot-Marie-Tooth Disease Variant, Charcot-Marie-Tooth-Roussy-Levy Disease, CHARGE Association, Charge Syndrome, CHARGE Syndrome, Chaund's Ectodermal Dysplasias, Chediak-Higashi Syndrome, Chediak-Higashi Syndrome, Chediak-Steinbrinck-Higashi Syndrome, Cheilitis Granulomatosa, Cheiloschisis, Chemke Syndrome, Cheney Syndrome, Cherry Red Spot and Myoclonus Syndrome, CHF, CHH, CHH, Chiari's Disease, Chiari Malformation I, Chiari Malformation, Chiari Type I (Chiari Malformation I), Chiari Type II (Chiari Malformation II), Chiari I Syndrome, Chiari-Budd Syndrome, Chiari-Frommel Syndrome, Chiari Malformation II, CHILD Syndrome, CHILD Ichthyosis Syndrome, CHILD Syndrome Ichthyosis, Childhood Adrenoleukodystrophy, Childhood Dermatomyositis, Childhood-onset Dystonia, Childhood Cyclic Vomiting, Childhood Giant Axonal Neuropathy, Childhood Hypophasphatasia, Childhood Muscular Dystrophy, CHN, Cholestasis, Cholestasis Hereditary Norwegian Type, Cholestasis Intrahepatic, Cholestasis Neonatal, Cholestasis of Oral Contraceptive Users, Cholestasis with Peripheral Pulmonary Stenosis, Cholestasis of Pregnancy, Cholesterol Desmolase Deficiency, Cholesterol Desmolase Deficiency, Chondrodysplasia Punctata, Chondrodystrophia Calcificans Congenita, Chondrodystrophia Fetalis, Chondrodystrophic Myotonia, Chondrodystrophy, Chondrodystrophy with Clubfeet, Chondrodystrophy Epiphyseal, Chondrodystrophy Hyperplastic Form, Chondroectodermal Dysplasias, Chondrogenesis Imperfecta, Chondrohystrophia, Chondroosteodystrophy, Choreoacanthocytosis, Chorionic Villi Sampling, Chorioretinal Anomalies, Chorioretinal Anomalies with ACC, Chorireninal Coloboma-Joubert Syndrome, Choroidal Sclerosis, Choroideremia, Chotzen Syndrome, Chotzen Syndrome, Christ-Siemens-Touraine Syndrome, Christ-Siemans-Touraine Syndrome, Christmas Disease, Christmas Tree Syndrome, Chromosome 3 Deletion of Distal 3p, Chromosome 3 Distal 3p Monosomy, Chromosome 3-Distal 3q2 Duplication, Chromosome 3-Distal 3q2 Trisomy, Chromosome 3 Monosomy 3p2, Chromosome 3q Partial Duplication Syndrome, Chromosome 3q, Partial Trisomy Syndrome, Chromosome 3-Trisomy 3q2, Chromosome 4 Deletion 4q31-qter Syndrome, Chromosome 4 Deletion 4q32-qter Syndrome, Chromosome 4 Deletion 4q33-qter Syndrome, Chromosome 4 Long Arm Deletion, Chromosome 4 Long Arm Deletion, Chromosome 4 Monosomy 4q, Chromosome 4-Monosomy 4q, Chromosome 4 Monosomy Distal 4q, Chromosome 4 Partial Deletion 4p, Chromosome 4, Partial Deletion of the Short Arm, Chromosome 4 Partial Monosomy of Distal 4q, Chromosome 4 Partial Monosomy 4p, Chromosome 4 Partial Trisomy 4 (q25-qter), Chromosome 4 Partial Trisomy 4 (q26 or q27-qter), Chromosome 4 Partial Trisomy 4 (q31 or 32-qter), Chromosome 4 Partial Trisomy 4p, Chromosome 4 Partial Trisomies 4q2 and 4q3, Chromosome 4 Partial Trisomy Distal 4, Chromosome 4 Ring, Chromosome 4 4q Terminal Deletion Syndrome, Chromosome 4q-Syndrome, Chromosome 4q-Syndrome, Chromosome 4 Trisomy 4, Chromosome 4 Trisomy 4p, Chromosome 4 XY/47 XXY (Mosiac), Chromosome 5 Monosomy 5p, Chromosome 5, Partial Deletion of the Short Arm Syndrome, Chromosome 5 Trisomy 5p, Chromosome 5 Trisomy 5p Complete (5p11-pter), Chromosome 5 Trisomy 5p Partial (5p13 or 14-pter), Chromosome 5p-Syndrome, Chromosome 6 Partial Trisomy 6q, Chromosome 6 Ring, Chromosome 6 Trisomy 6q2, Chromosome 7 Monosomy 7p2, Chromosome 7 Partial Deletion of Short Arm (7p2-), Chromosome 7 Terminal 7p Deletion [del (7) (p21 -p22)], Chromosome 8 Monosomy 8p2, Chromosome 8 Monosomy 8p21-pter, Chromosome 8 Partial Deletion (short arm), Chromosome 8 Partial Monosomy 8p2, Chromosome 9 Complete Trisomy 9P, Chromosome 9 Partial Deletion of Short Arm, Chromosome 9 Partial Monosomy 9p, Chromosome 9 Partial Monosomy. 9p22, Chromosome 9 Partial Monosomy 9p22-pter, Chromosome 9 Partial Trisomy 9P Included, Chromosome 9 Ring, Chromosome 9 Tetrasomy 9p, Chromosome 9 Tetrasomy 9p Mosaicism, Chromosome 9 Trisomy 9p (Multiple Variants), Chromosome 9 Trisomy 9 (pter-p21 to q32) Included, Chromosome 9 Trisomy Mosaic, Chromosome 9 Trisomy Mosaic, Chromosome 10 Distal Trisomy 10q, Chromosome 10 Monosomy, Chromosome 10 Monosomy 10p, Chromosome 10, Partial Deletion (short arm), Choromsome 10, 10p-Partial, Chromosome 10 Partial Trisomy 10q24-qter, Chromosome 10 Trisomy 10q2, Partial Monosomy of Long Arm of Chromosome 11, Chromosome 11 Partial Monosomy 11q, Chromosome 11 Partial Trisomy, Chromosome 11 Partial Trisomy 11q13-qter, Chromosome 11 Partial Trisomy 11q21-qter, Chromosome 11 Partial Trisomy 11q23-qter, Chromosome 11q, Partial Trisomy, Chromosome 12 Isochromosome 12p Mosaic, Chromosome 13 Partial Monosomy 13q, Chromosome 13, Partial Monosomy of the Long Arm, Chromosome 14 Ring, Chromosome 14 Trisomy, Chromosome 15 Distal Trisomy 15q, Chromosome r15, Chromosome 15 Ring, Chromosome 15 Trisomy 15q2, Chromosome 15q, Partial Duplication Syndrome, Chromosome 17 Interstitial Deletion 17p, Chromosome 18 Long Arm Deletion Syndrome, Chromosome 18 Monosomy 18p, Chromosome 18 Monosomy 18Q, Chromosome 18 Ring, Chromosome 18 Tetrasomy 18p, Chromosome 18q-Syndrome, Chromosome 21 Mosaic 21 Syndrome, Chromosome 21 Ring, Chromosome 21 Translocation 21 Syndrome, Chromosome 22 Inverted Duplication (22pter-22q11), Chromosome 22 Partial Trisomy (22pter-22q11), Chromosome 22 Ring, Chromosomc 22 Trisomy Mosaic, Chromosome 48 XXYY, Chromosome 48 XXXY, Chromosome r15, Chromosomal Triplication, Chromosome Triplication, Chromosome Triploidy Syndrome, Chromosome X, Chromosome XXY, Chronic Acholuric Jaundice, Chronic Adhesive Arachnoiditis, Chronic Adrenocortical Insufficiency, Chronic Cavernositis, Chronic Congenital Aregenerative Anemia, Chronic Dysphagocytosis, Chronic Familial Granulomatosis, Chronic Familial Icterus, Chronic Fatigue Immune Dysfunction Syndrome (CFIDS), Chronic Granulomatous Disease, Chronic Guillain-Barre Syndrome, Chronic Idiopathic Jaundice, Chronic Idiopathic Polyneuritis (CIP), Chronic Inflammatory Demyelinating Polyneuropathy, Chronic Inflammatory Demyelinating Polyradiculoneuropathy, Chronic Motor Tic, Chronic Mucocutaneous Candidiasis, Chronic Multiple Tics, Chronic Non-Specific Ulcerative Colitis, Chronic Non-Specific Ulcerative Colitis, Chronic Obliterative Cholangitis, Chronic Peptic Ulcer and Esophagitis Syndrome, Chronic Progressive Chorea, Chronic Progressive External Ophthalmoplegia Syndrome, Chronic Progressive External Ophthalmoplegia and Myopathy, Chronic Progressive External Ophthalmoplegia with Ragged Red Fibers, Chronic Relapsing Polyneuropathy, Chronic Sarcoidosis, Chronic Spasmodic Dysphonia, Chronic Vomiting in Childhood, CHS, Churg-Strauss Syndrome, Cicatricial Pemphigoid, CIP, Cirrhosis Congenital Pigmentary, Cirrhosis, Cistinuria, Citrullinemia, CJD, Classic Schindler Disease, Classic Type Pfeiffer Syndrome, Classical Maple Syrup Urine Disease, Classical Hemophilia, Classical Form Cockayne Syndrome Type I (Type A), Classical Leigh's Disease, Classical Phenylketonuria, Classical X-Linked Pelizaeus-Merzbacher Brain Sclerosis, CLE, Cleft Lip/Palate Mucous Cysts Lower Lip PP Digital and Genital Anomalies, Cleft Lip-Palate Blepharophimosis Lagophthalmos and Hypertelorism, Cleft Lip/Palate with Abnormal Thumbs and Microcephaly, Cleft palate-joint contractures-dandy walker malformations, Cleft Palate and Cleft Lip, Cleidocranial Dysplasia w/Micrognathia, Absent Thumbs, & Distal Aphalangia, Cleidocranial Dysostosis, Cleidocranial Dysplasia, Click murmur syndrome, CLN1, Clonic Spasmodic, Cloustons Syndrome, Clubfoot, CMDI, CMM, CMT, CMTC, CMTX, COA Syndrome, Coarctation of the aorta, Coarctation of the aorta, Coats' Disease, Cobblestone dysplasia, Cochin Jewish Disorder, Cockayne Syndrome, COD-MD Syndrome, COD, Coffin Lowry Syndrome, Coffin Syndrome, Coffin Siris Syndrome, COFS Syndrome, Cogan Corneal Dystrophy, Cogan Reese Syndrome, Cohen Syndrome, Cold Aggiutinin Disease, Cold Antibody Disease, Cold Antibody Disease, Cold Antibody Hemolytic Anemia, Cold Agglutinin Disease, Cold Agglutinin Disease, Colitis Ulcerative, Colitis Gravis, Colitis Gravis, Colitis Ulcerative Chronic Non-Specific Ulcerative Colitis, Collodion Baby, Coloboma Heart Defects Atresia of the Choanae Retardation of Growth and Development Genital and Urinary Anomalies and Ear Anomalies, Coloboma, Coloboma, Colonic Neurosis, Color blindness, Color blindness, Colour blindness, Colour blindness, Colpocephaly, Columnar-Like Esophagus, Combined Cone-Rod Degeneration, Combined Immunodeficiency with Immunoglobulins, Combined Mesoectodermal Dysplasia, Common Variable Hypogammaglobulinemia, Common Variable Immunodeficiency, Common Ventricle, Communicating Hydrocephalus, Complete Absense of Hypoxanthine-Guanine Phosphoribosyltranferase, Complete Atrioventricular Septal Defect, Complement Component 1 Inhibitor Deficiciency, Complement Component Cl Regulatory Component Deficiency, Complete Heart Block, Complex Carbohydrate Intolerance, Complex Regional Pain Syndrome, Complex V ATP Synthase Deficiency, Complex I, Complex I NADH dehydrogenase deficiency, Complex II, Complex II Succinate dehydrogenase deficiency, Complex III, Complex III Ubiquinone-cytochrome c oxidoreductase deficiency, Complex IV, Complex IV Cytochrome c oxidase deficiency, Complex IV Deficiency, Complex V, Cone-Rod Degeneration, Cone-Rod Degeneration Progressive, Cone Dystrophy, Cone-Rod Dystrophy, Confluent Reticular Papillomatosis, Congenital with low PK Kinetics, Congenital Absence of Abdominal Muscles, Congenital Absence of the Thymus and Parathyroids, Congenital Achromia, Congenital Addison's Disease, Congenital Adrenal Hyperplasia, Congenital Adreneal Hyperplasia, Congenital Afibrinogenemia, Congenital Alveolar Hypoventilation, Congenital Anemia of Newborn, Congenital Bilateral Persylvian Syndrome, Congenital Brown Syndrome, Congenital Cardiovascular Defects, Congenital Central Hypoventilation Syndrome, Congenital Cerebral Palsy, Congenital Cervical Synostosis, Congenital Clasped Thumb with Mental Retardation, Congenital Contractural Arachnodactyly, Congenital Contractures Multiple with Arachnodactyly, Congenital Cyanosis, Congenital Defect of the Skull and Scalp, Congenital Dilatation of Intrahepatic Bile Duct, Congenital Dysmyelinating Neuropathy, Congenital Dysphagocytosis, Congenital Dysplastic Angiectasia, Congenital Erythropoietic Porphyria, Congenital Erythropoietic Porphyria, Congenital Factor XIII Deficiency, Congenital Failure of Autonomic Control of Respiration, Congenital Familial Nonhemolytic Jaundice Type I, Congenital Familial Protracted Diarrhea, Congenital Form Cockayne Syndrome Type II (Type B), Congenital Generalized Fibromatosis, Congenital German Measles, Congenital Giant Axonal Neuropathy, Congenital Heart Block, Congenital Heart Defects, Congenital Hemidysplasia with Ichthyosis Erythroderma and Limb Defects, Congenital Hemolytic Jaundice, Congenital Hemolytic Anemia, Congenital Hepatic Fibrosis, Congenital Hereditary Corneal Dystrophy, Congenital Hereditary Lymphedema, Congenital Hyperchondroplasia, Congenital Hypomyelinating Polyneuropathy, Congenital Hypomyelination Neuropathy, Congenital Hypomyelination, Congenital Hypomyelination Neuropathy, Congenital Hypomyelination (Onion Bulb) Polyneuropathy, Congenital Ichthyosiform Erythroderma, Congenital Keratoconus, Congenital Lactic Acidosis, Congenital Lactose Intolerance, Congenital Lipodystrophy, Congenital Liver Cirrhosis, Congenital Lobar Emphysema, Congenital Localized Emphysema, Congenital Macroglossia, Congenital Medullary Stenosis, Congenital Megacolon, Congenital Melanocytic Nevus, Congenital Mesodermal Dysmorphodystrophy, Congenital Mesodermal Dystrophy, Congenital Microvillus Atrophy, Congenital Multiple Arthrogryposis, Congenital Myotonic Dystrophy, Congenital Neuropathy caused by Hypomyelination, Congenital Pancytopenia, Congenital Pernicious Anemia, Congenital Pernicious Anemia due to Defect of Intrinsic Factor, Congenital Pernicious Anemia due to Defect of Intrinsic Factor, Congenital Pigmentary Cirrhosis, Congenital Porphyria, Congenital Proximal Myopathy Associated with Desmin Storage Myopathy, Congenital Pulmonary Emphysema, Congenital Pure Red Cell Anemia, Congenital Pure Red Cell Aplasia, Congenital Retinal Blindness, Congenital Retinal Cyst, Congenital Retinitis Pigmentosa, Congenital Retinoschisis, Congenital Rod Disease, Congenital Rubella Syndrome, Congenital Scalp Defects with Distal Limb Reduction Anomalies, Congenital Sensory Neuropathy, Congenital SMA with arthrogryposis, Congenital Spherocytic Anemia, Congenital Spondyloepiphyseal Dysplasia, Congenital Tethered Cervical Spinal Cord Syndrome, Congenital Tyrosinosis, Congenital Varicella Syndrome, Congenital Vascular Cavernous Malformations, Congenital Vascular Veils in the Retina, Congenital Word Blindness, Congenital Wandering Spleen (Pediatric), Congestive Cardiomyopathy, Conical Cornea, Conjugated Hyperbilirubinemia, Conjunctivitis, Conjunctivitis Ligneous, Conjunctivo-Urethro-Synovial Syndrome, Conn's Syndrome, Connective Tissue Disease, Conradi Disease, Conradi Hunermann Syndrome, Constitutional Aplastic Anemia, Constitutional Erythroid Hypoplasia, Constitutional Eczema, Constitutional Liver Dysfunction, Constitutional Thrombopathy, Constricting Bands Congenital, Constrictive Pericarditis with Dwarfism, Continuous Muscle Fiber Activity Syndrome, Contractural Arachnodactyly, Contractural Arachnodactyly, Contractures of Feet Muscle Atrophy and Oculomotor Apraxia, Convulsions, Cooley's anemia, Copper Transport Disease, Coproporphyria Porphyria Hepatica, Cor Triatriatum, Cor Triatriatum Sinistrum, Cor Triloculare Biatriatum, Cor Biloculare, Cori Disease, Cornea Dystrophy, Corneal Amyloidosis, Corneal Clouding-Cutis Laxa-Mental Retardation, Corneal Dystrophy, Cornelia de Lange Syndrome, Coronal Dentine Dysplasia, Coronary Artery Disease, Coronary Heart Disease, Corpus Callosum Agenesis, Cortical-Basal Ganglionic Degeneration, Corticalis Deformaris, Cortico-Basal Ganglionic Degeneration (CBGD), Corticobasal Degeneration, Corticosterone Methloxidase Deficiency Type I, Corticosterone Methyloxidase Deficiency Type II, Cortisol, Costello Syndrome, Cot Death, COVESDEM Syndrome, COX, COX Deficiency, COX Deficiency French-Canadian Type, COX Deficiency Infantile Mitochondrial Myopathy de Toni-Fanconi-Debre included, COX Deficiency Type Benign Infantile Mitochondrial Mypoathy, CP, CPEO, CPEO with Myopathy, CPEO with Ragged-Red Fibers, CPPD Familial Form, CPT Deficiency, CPTD, Cranial Arteritis, Cranial Meningoencephalocele, Cranio-Oro-Digital Syndrome, Craniocarpotarsal dystrophy, Craniocele, Craniodigital Syndrome-Mental Retardation Scott Type, Craniofacial Dysostosis, Craniofacial Dysostosis-PD Arteriosus-Hypertrichosis-Hypoplasia of Labia, Craniofrontonasal Dysplasia, Craniometaphyseal Dysplasia, Cranioorodigital Syndrome, Cranioorodigital Syndrome Type II, Craniostenosis Crouzon Type, Craniostenosis, Craniostenosis, Craniosynostosis-Choanal Atresia-Radial Humeral Synostosis, Craniosynostosis-Hypertrichosis-Facial and Other Anomalies, Craniosynostosis Midfacial Hypoplasia and Foot Abnormalities, Craniosynostosis Primary, Craniosynostosis-Radial Aplasia Syndrome, Craniosynostosis with Radial Defects, Cranium Bifidum, CREST Syndrome, CREST Syndrome, Creutzfeldt Jakob Disease, Cri du Chat Syndrome, Crib Death, Crigler Najjar Syndrome Type I, Crohn's Diseas, Crohn's Disease, Cronkhite-Canada Syndrome, Cross Syndrome, Cross' Syndrome, Cross-McKusick-Breen Syndrome, Crouzon, Crouzon Syndrome, Crouzon Craniofacial Dysostosis, Cryoglobulinemia Essential Mixed, Cryptophthalmos-Syndactyly Syndrome, Cryptorchidism-Dwarfism-Subnormal Mentality, Crystalline Corneal Dystrophy of Schnyder, CS, CSD, CSID, CSO, CST Syndrome, Curly Hair-Ankyloblephanon-Nail Dysplasia, Curschmann-Batten-Steinert Syndrome, Curth Macklin Type Ichthyosis Hystric, Curth-Macklin Type, Cushing's, Cushing Syndrome, Cushing's III, Cutaneous Malignant Melanoma Hereditary, Cutaneous Porphyrias, Cutis Laxa, Cutis Laxa, Cutis Laxa-Growth Deficiency Syndrome, Cutis Marmorata Telangiectatica Congenita, CVI, CVID, CVS, CVS, Cyclic vomiting syndrome, Cystic Disease of the Renal Medulla, Cystic Disease of the Renal Medulla, Cystic Hygroma, Cystic Fibrosis, Cystic Lymphangioma, Cystine-Lysine-Arginine-Ornithinuria, Cystine Storage Disease, Cystinosis, Cystinuria, Cystinuria with Dibasic Aminoaciduria, Cystinuria Type I, Cystinuria Type II, Cystinuria Type III, Cysts of the Renal Medulla Congenital, Cysts of the Renal Medulla Congenital, Cytochrome C Oxidase Deficiency, D.C., Dacryosialoadenopathy, Dacryosialoadenopathia, Dalpro, Dalton, Daltonism, Danbolt-Cross Syndrome, Dancing Eyes-Dancing Feet Syndrome, Dandy-Walker Syndrome, Dandy-Walker Cyst, Dandy-Walker Deformity, Dandy Walker Malformation, Danish Cardiac Type Amyloidosis (Type III), Darier Disease, Davidson's Disease, Davies' Disease, DBA, DBS, DC, DD, De Barsy Syndrome, De Barsy-Moens-Diercks Syndrome, de Lange Syndrome, De Morsier Syndrome, De Santis Cacchione Syndrome, de Toni-Fanconi Syndrome, Deafness Congenital and Functional Heart Disease, Deafness-Dwarfism-Retinal Atrophy, Deafness-Functional Heart Disease, Deafness Onychodystrophy Osteodystrophyand Mental Retardation, Deafness and Pili Torti Bjornstad Type, Deafness Sensorineural with Imperforate Anus and Hypoplastic Thumbs, Debrancher Deficiency, Deciduous Skin, Defect of Enterocyte Intrinsic Factor Receptor, Defect of Enterocyte Intrinsic Factor Receptor, Defect in Natural Killer Lymphocytes, Defect of Renal Reabsorption of Carnitine, Deficiency of Glycoprotein Neuraminidase, Deficiency of Mitochondrial Respiratory Chain Complex IV, Deficiency of Platelet Glycoprotein Ib, Deficiency of Von Willebrand Factor Receptor, Deficiency of Short-Chain Acyl-CoA Dehydrogenase (ACADS, Deformity with Mesomelic Dwarfism, Degenerative Chorea, Degenerative Lumbar SpinaL Stenosis, Degos Disease, Degos-Kohlmeier Disease, Degos Syndrome, DEH, Dejerine-Roussy Syndrome, Dejerine Sottas Disease, Deletion 9p Syndrome Partial, Deletion 11q Syndrome Partial, Deletion 13q Syndrome Partial, Delleman-Oorthuys Syndrome, Delleman Syndrome, Dementia with Lobar Atrophy and Neuronal Cytoplasmic Inclusions, Demyelinating Disease, DeMyer Syndrome, Dentin Dysplasia Coronal, Dentin Dysplasia Radicular, Dentin Dysplasia Type I, Dentin Dysplasia Type II, Dentinogenesis Imperfecta Brandywine type, Dentinogenesis Imperfecta Shields Type, Dentinogenesis Imperfecta Shields Type, Dentinogenesis Imperfecta Type III, Dentinogenesis Imperfecta Type III, Dento-Oculo-Osseous Dysplasia, Dento-Oculo-Osseous Dysplasia, Dentooculocutaneous Syndrome, Denys-Drash Syndrome, Depakene, DepakeneTM exposure, Depakote, Depakote Sprinkle, Depigmentation-Gingival Fibromatosis-Microphthalmia, Dercum Disease, Dercum Disease, Dermatitis Atopic, Dermatitis Exfoliativa, Dermatitis Herpetiformis, Dermatitis Multiformis, Dermatochalasia Generalized, Dermatolysis Generalized, Dermatomegaly, Dermatomyositis sine myositis, Dermatomyositis, Dermatosparaxis, Dermatostomatitis Stevens Johnson Type, Desbuquois Syndrome, Desmin Storage Myopathy, Desquamation of Newborn, Deuteranomaly, Deuteranomaly, Developmental Reading Disorder, Developmental Gerstmann Syndrome, Devergie Disease, Devic Disease, Devic Syndrome, Dextrocardia-Bronchiectasis and Sinusitis, Dextrocardia with Situs Inversus, DGS, DGSX Golabi-Rosen Syndrome Included, DH, DHAP alkyl transferase deficiency, DHBS Deficiency, DHBS Deficiency, DHOF, DHPR Deficiency, DHPR Deficiency, Diabetes Insipidus, Diabetes Insipidus Diabetes Mellitus Optic Atrophy and Deafness, Diabetes Insipidus Neurohypophyseal, Diabetes Insulin Dependent, Diabetes Mellitus, Diabetes Mellitus Addison's Disease Myxedema, Diabetic Acidosis, Diabetic Bearded Woman Syndrome, Diamond-Blackfan Anemia, Diaphragmatic Apnea, Diaphyseal Aclasis, Diastrophic Dwarfism, Diastrophic Dysplasia, Diastrophic Nanism Syndrome, Dicarboxylic Aminoaciduria, Dicarboxylicaciduria Caused by Defect in Beta-Oxidation of Fatty Acids, Dicarboxylicaciduria due to Defect in Beta-Oxidation of Fatty Acids, Dicarboxylicaciduria due to MCADH Deficiency, Dichromasy, Dicker-Opitz, DIDMOAD, Diencephalic Syndrome, Diencephalic Syndrome of Childhood, Diencephalic Syndrome of Emaciation, Dienoyl-CoA Reductase Deficiency, Diffuse Cerebral Degeneration in Infancy, Diffuse Degenerative Cerebral Disease, Diffuse Idiopathic Skeletal Hyperostosis, Diffusum-Glycopeptiduria, DiGeorge Syndrome, DiGeorge Syndrome, Digital-Oro-Cranio Syndrome, Digito-Oto-Palatal Syndrome, Digito-Oto-Palatal Syndrome Type I, Digito-Oto-Palatal Syndrome Type II, Dihydrobiopterin Synthetase Deficiency, Dihydrobiopterin Synthetase Deficiency, Dihydropteridine Reductase Deficiency, Dihydropteridine Reductase Deficiency, Dihydroxyacetonephosphate synthase, Dilated (Congestive) Cardiomyopathy, Dimitri Disease, Diplegia of Cerebral Palsy, Diplo-Y Syndrome, Disaccharidase Deficiency, Disaccharide Intolerance I, Discoid Lupus, Discoid Lupus Erythematosus, DISH, Disorder of Cornification, Disorder of Comification Type I, Disorder of Cornification 4, Disorder of Comification 6, Disorder of Comification 8, Disorder of Cornification 9 Netherton's Type, Disorder of Comification 11 Phytanic Acid Type, Disorder of Cornification 12 (Neutral Lipid Storage Type), Disorder of Conification 13, Disorder of Comification 14, Disorder of Comification 14 Trichothiodystrophy Type, Disorder of Cornification 15 (Keratitis Deafness Type), Disorder of Cornification 16, Disorder of Cornification 18 Erythrokeratodermia Variabilis Type, Disorder of Cornification 19, Disorder of Cornification 20, Disorder of Cornification 24, Displaced Spleen, Disseminated Lupus Erythematosus, Disseminated Neurodermatitis, Disseminated Sclerosis, Distal 11q Monosomy, Distal 11q-Syndrome, Distal Arthrogryposis Multiplex Congenita Type IIA, Distal Arthrogryposis Multiplex Congenita Type IIA, Distal Arthrogryposis Type IIA, Distal Arthrogryposis Type 2A, Distal Duplication 6q, Distal Duplication 10q, Dup(10q) Syndrome, Distal Duplication 15q, Distal Monosomy 9p, Distal Trisomy 6q, Distal Trisomy 10q Syndrome, Distal Trisomy 11q, Divalproex, DJS, DKC, DLE, DLPIII, DM, DMC Syndrome, DMC Disease, DMD, DNS Hereditary, DOC I, DOC 2, DOC 4, DOC 6 (Harlequin Type), DOC 8 Curth-Macklin Type, DOC 11 Phytanic Acid Type, DOC 12 (Neutral Lipid Storage Type), DOC 13, DOC 14, DOC 14 Trichothiodystrophy Type, DOC 15 (Keratitis Deafness Type), DOC 16, DOC 16 Unilateral Hemidysplasia Type, DOC 18, DOC 19, DOC 20, DOC 24, Dohle's Bodies-Myelopathy, Dolichospondylic Dysplasia, Dolichostenomelia, Dolichostenomelia Syndrome, Dominant Type Kenny-Caffe Syndrome, Dominant Type Myotonia Congenita, Donahue Syndrome, Donath-Landsteiner Hemolytic Anemia, Donath-Landsteiner Syndrome, DOOR Syndrome, DOORS Syndrome, Dopa-responsive Dystonia (DRD), Dorfman Chanarin Syndrome, Dowling-Meara Syndrome, Down Syndrome, DR Syndrome, Drash Syndrome, DRD, Dreifuss-Emery Type Muscular Dystrophy with Contractures, Dressler Syndrome, Drifting Spleen, Drug-induced Acanthosis Nigricans, Drug-induced Lupus Erythematosus, Drug-related Adrenal Insufficiency, Drummond's Syndrome, Dry Beriberi, Dry Eye, DTD, Duane's Retraction Syndrome, Duane Syndrome, Duane Syndrome Type IA 1B and IC, Duane Syndrome Type 2A 2B and 2C, Duane Syndrome Type 3A 3B and 3C, Dubin Johnson Syndrome, Dubowitz Syndrome, Duchenne, Duchenne Muscular Dystrophy, Duchenne's Paralysis, Duhring's Disease, Duncan Disease, Duncan's Disease, Duodenal Atresia, Duodenal Stenosis, Duodenitis, Duplication 4p Syndrome, Duplication 6q Partial, Dupuy's Syndrome, Dupuytren's Contracture, Dutch-Kennedy Syndrome, Dwarfism, Dwarfism Campomelic, Dwarfism Cortical Thickening of the Tubular Bones & Transient Hypocalcemia, Dwarfism Levi's Type, Dwarfism Metatropic, Dwarfism-Onychodysplasia, Dwarfism-Pericarditis, Dwarfism with Renal Atrophy and Deafness, Dwarfism with Rickets, DWM, Dyggve Melchior Clausen Syndrome, Dysautonomia Familial, Dysbetalipoproteinemia Familial, Dyschondrodysplasia with Hemangiomas, Dyschondrosteosis, Dyschromatosis Universalis Hereditaria, Dysencephalia Splanchnocystica, Dyskeratosis Congenita, Dyskeratosis Congenita Autosomal Recessive, Dyskeratosis Congenita Scoggins Type, Dyskeratosis Congenita Syndrome, Dyskeratosis Follicularis Vegetans, Dyslexia, Dysmyelogenic Leukodystrophy, Dysmyelogenic Leukodystrophy-Megalobare, Dysphonia Spastica, Dysplasia Epiphysialis Punctata, Dysplasia Epiphyseal Hemimelica, Dysplasia of Nails With Hypodontia, Dysplasia Cleidocranial, Dysplasia Fibrous, Dysplasia Gigantism SyndromeX-Linked, Dysplasia Osteodental, Dysplastic Nevus Syndrome, Dysplastic Nevus Syndrome, Dysplastic Nevus Type, Dyssynergia Cerebellaris Myoclonica, Dyssynergia Esophagus, Dystonia, Dystonia, Dystopia Canthorum, Dystopia Canthorum, Dystrophia Adiposogenitalis, Dystrophia Endothelialis Cornea, Dystrophia Mesodermalis, Dystrophic Epidermolysis Bullosa, Dystrophic Epidermolysis Bullosa, Dystrophy, Asphyxiating Thoracic, Dystrophy Myotonic, E-D Syndrome, Eagle-Barrett Syndrome, Bales Retinopathy, Eales Disease, Ear Anomalies-Contractures-Dysplasia of Bone with Kyphoscoliosis, Ear Patella Short Stature Syndrome, Early Constraint Defects, Early Hypercalcemia Syndrome with Elfin Facie, Early-onset Dystonia, Eaton Lambert Syndrome, EB, Ebstein's anomaly, EBV Susceptibility (EBVS), EBVS, ECD, ECPSG, Ectodermal Dysplasias, Ectodermal Dysplasia Anhidrotic with Cleft Lip and Cleft Palate, Ectodermal Dysplasia-Exocrine Pancreatic Insufficiency, Ectodermal Dysplasia Rapp-Hodgkin type, Ectodermal and Mesodermal Dysplasia Congenital, Ectodermal and Mesodermal Dysplasia with Osseous Involvement, Ectodermosis Erosiva Pluriorificialis, Ectopia Lentis, Ectopia Vesicae, Ectopic ACTH Syndrome, Ectopic Adrenocorticotropic Hormone Syndrome, Ectopic Anus, Ectrodactilia of the Hand, Ectrodactyly, Ectrodactyly-Ectodermal Dysplasia-Clefting Syndrome, Ectrodactyly Ectodermal Dysplasias Clefting Syndrome, Ectrodactyly Ectodermal Dysplasia Cleft Lip/Cleft Palate, Eczema, Eczema-Thrombocytopenia-Immunodeficiency Syndrome, EDA, EDMD, EDS, EDS Arterial-Ecchymotic Type, EDS Arthrochalasia, EDS Classic Severe Form, EDS Dysfibronectinemic, EDS Gravis Type, EDS Hypermobility, EDS Kyphoscoliotic, EDS Kyphoscoliosis, EDS Mitis Type, EDS Ocular-Scoliotic, EDS Progeroid, EDS Periodontosis, EDS Vascular, EEC Syndrome, EFE, EHBA, EHK, Ehlers Danlos Syndrome, Ehlers-Danlos syndrome, Ehlers Danlos IX, Eisenmenger Complex, Eisenmenger's complex, Eisenmenger Disease, Eisenmenger Reaction, Eisenmenger Syndrome, Eisenmenger Syndrome, Ekbom Syndrome, Ekman-Lobstein Disease, Ektrodactyly of the Hand, Ektrodactyly of the Hand, EKV, Elastin fiber disorders, Elastorrhexis Generalized, Elastosis Dystrophica Syndrome, Elective Mutism (obsolete), Elective Mutism, Electrocardiogram (ECG or EKG), Electron Transfer Flavoprotein (ETF) Dehydrogenase Deficiency: (GAII & MADD), Electrophysiologic study (EPS), Elephant Nails From Birth, Elephantiasis Congenita Angiomatosa,Hemangiectatic Hypertrophy, Elfin Facies with Hypercalcemia, Ellis-van Creveld Syndrome, Ellis Van Creveld Syndrome, Embryoma Kidney, Embryonal Adenomyosarcoma Kidney, Embryonal Carcinosarcoma Kidney, Embryonal Mixed Tumor Kidney, EMC, Emery Dreyfus Muscular Dystrophy, Emery-Dreifuss Muscular Dystrophy, Emery-Dreifuss Syndrome, EMF, EMG Syndrome, Empty Sella Syndrome, Encephalitis Periaxialis Diffusa, Encephalitis Periaxialis Concentrica, Encephalocele, Encephalofacial Angiomatosis, Encephalopathy, Encephalotrigeminal Angiomatosis, Enchondromatosis with Multiple Cavernous Hemangiomas, Endemic Polyneuritis, Endocardial Cushion Defect, Endocardial Cushion Defect, Endocardial Cushion Defects, Endocardial Dysplasia, Endocardial Fibroelastosis (EFE), Endogenous Hypertriglyceridemia, Endolymphatic Hydrops, Endometrial Growths, Endometriosis, Endomyocardial Fibrosis, Endothelial Corneal Dystrophy Congenital, Endothelial Epithelial Corneal Dystrophy, Endothelium, Engelmann Disease, Enlarged Tongue, Enterocolitis, Enterocyte Cobalamin Malabsorption, Eosinophia Syndrome, Eosinophilic Cellulitis, Eosinophilic Fasciitis, Eosinophilic Granuloma, Eosinophilic Syndrome, Epidermal Nevus Syndrome, Epidermolysis bullosa, Epidermolysis Bullosa, Epidermolysis Bullosa Acquisita, Epidermolysis Bullosa Hereditaria, Epidermolysis Bullosa Letalias, Epidermolysis Hereditaria Tarda, Epidermolytic Hyperkeratosis, Epidermolytic Hyperkeratosis (Bullous CIE), Epilepsia Procursiva, Epilepsy, Epinephrine, Epiphyseal Changes and.High Myopia, Epiphyseal Osteochondroma Benign, Epiphysealis Hemimelica Dysplasia, Episodic-Abnormal Eye Movement, Epithelial Basement Membrane Corneal Dystrophy, Epithelial Corneal Dystrophy of Meesmann Juvenile, Epitheliomatosis Multiplex with Nevus, Epithelium, Epival, EPS, Epstein-Barr Virus-Induced Lymphoproliferative Disease in Males, Erb-Goldflam syndrome, Erdheim Chester Disease, Erythema Multiforme Exudativum, Erythema Polymorphe Stevens Johnson Type, Erythroblastophthisis, Erythroblastosis Fetalis, Erythroblastosis Neonatorum, Erythroblastotic Anemia of Childhood, Erythrocyte Phosphoglycerate Kinase Deficiency, Erythrogenesis Imperfecta, Erythrokeratoderrnia Progressiva Symmetrica, Erythrokeratodermia Progressiva Symmetrica Ichthyosis, Erythrokeratodermia Variabilis, Erythrokeratodermia Variabilis, Erythrokeratodermia Variabilis Type, Erythrokeratolysis Hiemalis, Erythrokeratolysis Hiemalis, Erythrokeratolysis Hiemalis, Erythropoietic Porphyrias, Erythropoietic Porphyria, Escobar Syndrome, Esophageal Atresia, Esophageal Aperistalsis, Esophagitis-Peptic Ulcer, Esophagus Atresia and/or Tracheoesophageal Fistula, Essential Familial Hyperlipemia, Essential Fructosuria, Essential Hematuria, Essential Hemorrhagic Thrombocythemia, Essential Hemorrhagic Thrombocythemia, Essential Mixed Cryoglobulinemia, Essential Moschowitz Disease, Essential Thrombocythemia, Essential Thrombocythemia, Essential Thrombocytopenia, Essential Thrombocytosis, Essential Thrombocytosis, Essential Tremor, Esterase Inhibitor Deficiency, Estren-Dameshek variant of Fanconi Anemia, Estrogen-related Cholestasis, ET, ET, ETF, Ethylmalonic Adipicaciduria, Eulenburg Disease, pc, EVCS, Exaggerated Startle Reaction, Exencephaly, Exogenous Hypertriglyceridemia, Exomphaios-Macroglossia-Gigantism Syndrom, Exophthalmic Goiter, Expanded Rubella Syndrome, Exstrophy of the Bladder, EXT, External Chondromatosis Syndrome, Extrahepatic Biliary Atresia, Extramedullary Plasmacytoma, Exudative Retinitis, Eye Retraction Syndrome, FA1, FAA, Fabry Disease, FAC, FACB, FACD, FACE, FACF, FACG, FACH, Facial Nerve Palsy, Facial Paralysis, Facial Ectodermal Dysplasias, Facial Ectodermal Dysplasia, Facio-Scapulo-Humeral Dystrophy, Facio-Auriculo-Vertebral Spectrum, Facio-cardio-cutaneous syndrome, Facio-Fronto-Nasal Dysplasia, Faciocutaneoskeletal Syndrome, Faciodigitogenital syndrome, Faciogenital dysplasia, Faciogenitopopliteal Syndrome, Faciopalatoosseous Syndrome, Faciopalatoosseous Syndrome Type II, Facioscapulohumeral muscular dystrophy, Factitious Hypoglycemia, Factor VIII Deficiency, Factor IX Deficiency, Factor IX Deficiency, Factor XI Deficiency, Factor XII deficiency, Factor XIII Deficiency, Fahr Disease, Fahr's Disease, Failure of Secretion Gastric Intrinsic Factor, Fairbank Disease, Fallot's Tetralogy, Familial Acrogeria, Familial Acrogeria, Familial Acromicria, Familial Acromicria, Familial Adenomatous Colon Polyposis, Familial Adenomatous Polyposis with Extraintestinal Manifestations, Familial Alobar Holoprosencephaly, Familial Alpha-Lipoprotein Deficiency, Familial Amyotrophic Chorea with Acanthocytosis, Familial Arrhythmic Myoclonus, Familial Articular Chondrocalcinosis, Familial Atypical Mole-Malignant Melanoma Syndrome, Familial Broad Beta Disease, Familial Calcium Gout, Familial Calcium Pyrophosphate Arthropathy, Familial Chronic Obstructive Lung Disease, Familial Continuous Skin Peeling, Familial Cutaneous Amyloidosis, Familial Dysproteinemia, Familial Emphysema, Familial Enteropathy Microvillus, Familial Foveal Retinoschisis, Familial Hibernation Syndrome, Familial High Cholesterol, Familial Hemochromatosis, Familial High Blood Cholesterol, Familial High-Density Lipoprotein Deficiency, Familial High Serum Cholesterol, Familial Hyperlipidema, Familial Hypoproteinemia with Lymphangietatic Enteropathy, Familial Jaundice, Familial Juvenile Nephronophtisis-Associated Ocular Anomaly, Familial Lichen Amyloidosis (Type IX), Familial Lumbar Stenosis, Familial Lymphedema Praecox, Familial Mediterranean Fever, Familial Multiple Polyposis, Familial Nuchal Bleb, Familial Paroxysmal Polyserositis, Familial Polyposis Coli, Familial Primary Pulmonary Hypertension, Familial Renal Glycosuria, Familial Splenic Anemia, Familial Startle Disease, Familial Visceral Amyloidosis (Type VIII), FAIMM, FANCA, FANCB, FANCC, FANCD, FANCE, Fanconi Panmyelopathy, Fanconi Pancytopenia, Fanconi II, Fanconi's Anemia, Fanconi's Anemia Type I, Fanconi's Anemia Complementation Group, Fanconi's Anemia Complementation Group A, Fanconi's Anemia Complementation Group B, Fanconi's Anemia Complementation Group C, Fanconi's Anemia Complementation Group D, Fanconi's Anemia Complementation Group E, Fanconi's Anemia Complementation Group G, Fanconi's Anemia Complementation Group H, Fanconi's Anemia Estren-Dameshek Variant, FANF, FANG, FANH, FAP, FAPG, Farber's Disease, Farber's Lipogranulomatosis, FAS, Fasting Hypoglycemia, Fat-Induced Hyperlipemia, Fatal Granulomatous Disease of Childhood, Fatty Oxidation Disorders, Fatty Liver with Encephalopathy, FAV, FCH, FCMD, FCS Syndrome, FD, FDH, Febrile Mucocutaneous Syndrome Stevens Johnson Type, Febrile Neutrophilic Dermatosis Acute, Febrile Seizures, Feinberg's syndrome, Feissinger-Leroy-Reiter Syndrome, Female Pseudo-Turner Syndrome, Femoral Dysgenesis Bilateral-Robin Anomaly, Femoral Dysgenesis Bilateral, Femoral Facial Syndrome, Femoral Hypoplasia-Unusual Facies Syndrome, Fetal Alcohol Syndrome, Fetal Anti-Convulsant Syndrome, Fetal Cystic Hygroma, Fetal Effects of Alcohol, Fetal Effects of Chickenpox, Fetal Effects of Thalidomide, Fetal Effects of Varicella Zoster Virus, Fetal Endomyocardial Fibrosis, Fetal Face Syndrome, Fetal Iritis Syndrome, Fetal Transfusion Syndrome, Fetal Valproate Syndrome, Fetal Valproic Acid Exposure Syndrome, Fetal Varicella Infection, Fetal Varicella Zoster Syndrome, FFDD Type II, FG Syndrome, FGDY, FHS, Fibrin Stabilizing Factor Deficiency, Fibrinase Deficiency, Fibrinoid Degeneration of Astrocytes, Fibrinoid Leukodystrophy, Fibrinoligase Deficiency, Fibroblastoma Perineural, Fibrocystic Disease of Pancreas, Fibrodysplasia Ossificans, Progressiva, Fibroelastic Endocarditis, Fibromyalgia, Fibromyalgia-Fibromyositis, Fibromyositis, Fibrosing Cholangitis, Fibrositis, Fibrous Ankylosis of Multiple Joints, Fibrous Cavemositis, Fibrous Dysplasia, Fibrous Plaques of the Penis, Fibrous Sclerosis of the Penis, Fickler-Winkler Type, Fiedler Disease, Fifth Digit Syndrome, Filippi Syndrome, Finnish Type Amyloidosis (Type V), First Degree Congenital Heart Block, First and Second Branchial Arch Syndrome, Fischer's Syndrome, Fish Odor Syndrome, Fissured Tongue, Flat Adenoma Syndrome, Flatau-Schilder Disease, Flavin Containing Monooxygenase 2, Floating Beta Disease, Floating-Harbor Syndrome, Floating Spleen, Floppy Infant Syndrome, Floppy Valve Syndrome, Fluent aphasia, FMD, FMF, FMO Adult Liver Form, FMO2, FND, Focal Dermal Dysplasia Syndrome, Focal Dermal Hypoplasia, Focal Dermato-Phalangeal Dysplasia, Focal Dystonia, Focal Epilepsy, Focal Facial Dermal Dysplasia Type II, Focal Neuromyotonia, FODH, Folling Syndrome, Fong Disease, FOP, Forbes Disease, Forbes-Albright Syndrome, Forestier's Disease, Forsius-Eriksson Syndrome (X-Linked), Fothergill Disease, Fountain Syndrome, Foveal Dystrophy Progressive, FPO Syndrome Type II, FPO, Fraccaro Type Achondrogenesis (Type IB), Fragile X syndrome, Franceschetti-Zwalen-Klein Syndrome, Francois Dyscephaly Syndrome, Francois-Neetens Speckled Dystrophy, Flecked Corneal Dystrophy, Fraser Syndrome, FRAXA, FRDA, Fredrickson Type I Hyperlipoproteinemia, Freeman-Sheldon Syndrome, Freire-Maia Syndrome, Frey's Syndrome, Friedreich's Ataxia, Friedreich's Ataxia, Friedreich's Disease, Friedreich's Tabes, FRNS, Froelich's Syndrome, Frommel-Chiari Syndrome, Frommel-Chiari Syndrome Lactation-Uterus Atrophy, Frontodigital Syndrome, Frontofacionasal Dysostosis, Frontofacionasal Dysplasia, Frontonasal Dysplasia, Frontonasal Dysplasia with Coronal Craniosynostosis, Fructose- l-Phosphate Aldolase Deficiency, Fructosemia, Fructosuria, Fryns Syndrome, FSH, FSHD, FSS, Fuchs Dystrophy, Fucosidosis Type 1, Fucosidosis Type 2, Fucosidosis Type 3, Fukuhara Syndrome, Fukuyama Disease, Fukuyama Type Muscular Dystrophy, Fukuyama Type Muscular Dystrophy, Fumarylacetoacetase deficiency, Furrowed Tongue, G Syndrome, G6PD Deficiency, G6PD, GA I, GA IIB, GA IIA, GA II, GAII & MADD, Galactorrhea-Amenorrhea Syndrome Nonpuerperal, Galactorrhea-Amenorrhea without Pregnancy, Galactosamine-6-Sulfatase Deficiency, Galactose-1-Phosphate Uridyl Transferase Deficiency, Galactosemia, GALB Deficiency, Galloway-Mowat Syndrome, Galloway Syndrome, GALT Deficiency, Gammaglobulin Deficiency, GAN, Ganglioside Neuraminidase Deficiency, Ganglioside Sialidase Deficiency, Gangliosidosis GMI Type 1, Gangliosidosis GM2 Type 2, Gangliosidosis Beta Hexosaminidase B Defeciency, Gardner Syndrome, Gardner Syndrome, Gargoylism, Garies-Mason Syndrome, Gasser Syndrome, Gastric Intrinsic Factor Failure of Secretion, Enterocyte Cobalamin, Gastrinoma, Gastritis, Gastroesophageal Laceration-Hemorrhage, Gastrointestinal Polyposis and Ectodermal Changes, Gastroschisis, Gaucher Disease, Gaucher-Schlagenhaufer, Gayet-Wemicke Syndrome, GBS, GCA, GCM Syndrome, GCPS, Gee-Herter Disease, Gee-Thaysen Disease, Gehrig's Disease, Gelineau's Syndrome, Genee-Wiedemann Syndrome, Generalized Dystonia, Generalized Familial Neuromyotonia, Generalized Fibromatosis, Generalized Flexion Epilepsy, Generalized Glycogenosis, Generalized Glycogenosis, Generalized Hyperhidrosis, Generalized Lipofuscinosis, Generalized Myasthenia Gravis, Generalized Myotonia, Generalized Sporadic Neuromytonia, Genetic Disorders, Genital Defects, Genital and Urinary Tract Defects, Genital and Urinary Tract Defects, Gerstmann Syndrome, Gerstmann Tetrad, GHBP, GHD, GHR, Giant Axonal Disease, Giant Axonal Neuropathy, Giant Benign Lymphoma, Giant Cell Glioblastoma Astrocytoma, Giant Cell Arteritis, Giant Cell Disease of the Liver, Giant Cell Hepatitis, Giant Cell of Newborns Cirrhosis, Giant Cyst of the Retina, Giant Lymph Node Hyperplasia, Giant Platelet Syndrome Hereditary, Giant Tongue, gic Macular Dystrophy, Gilbert's Disease, Gilbert Syndrome, Gilbert-Dreyfus Syndrome, Gilbert-Dreyfus Syndrome, Gilbert-Lereboullet Syndrome, Gilford Syndrome, Gilles de la Tourette's syndrome, Gillespie Syndrome, Gingival Fibromatosis-Abnormal Fingers Nails Nose Ear Splenomegaly, GLA Deficiency, GLA, GLB1, Glioma Retina, Global aphasia, Globoid Leukodystrophy, Glossoptosis Micrognathia and Cleft Palate, Glucocerebrosidase deficiency, Glucocerebrosidosis, Glucose-6-Phosphate Dehydrogenase Deficiency, Glucose-6-Phosphate Tranport Defect, Glucose-6-Phospate Translocase Deficiency, Glucose-G-Phosphatase Deficiency, Glucose-Galactose Malabsorption, Glucose-Galactose Malabsorption, Glucosyl Ceramide Lipidosis, Glutaric Aciduria I, Glutaric Acidemia I, Glutaric Acidemia II, Glutaric Aciduria II, Glutaric Aciduria Type II, Glutaric Aciduria Type III, Glutaricacidemia I, Glutaricacidemia II, Glutaricaciduria I, Glutaricaciduria II, Glutaricaciduria Type IIA, Glutaricaciduria Type IIB, Glutaryl-CoA Dehydrogenase Deficiency, Glutaurate-Aspartate Transport Defect, Gluten-Sensitive Enteropathy, Glycogen Disease of Muscle Type VII, Glycogen Storage Disease I, Glycogen Storage Disease III, Glycogen Storage Disease IV, Glycogen Storage Disease Type V, Glycogen Storage Disease VI, Glycogen Storage Disease VII, Glycogen Storage Disease VIII, Glycogen Storage Disease Type II, Glycogen Storage Disease-Type II, Glycogenosis, Glycogenosis Type I, Glycogenosis Type IA, Glycogenosis Type IB, Glycogenosis Type II, Glycogenosis Type II, Glycogenosis Type III, Glycogenosis Type IV, Glycogenosis Type V, Glycogenosis Type VI, Glycogenosis Type VII, Glycogenosis Type VIII, Glycolic Aciduria, Glycolic Aciduria, Glycolipid Lipidosis, GM-2 Gangliosidosis Type 1, GM2 Gangliosidosis Type 1, GNPTA, Goitrous Autoimmune Thyroiditis, Goldenhar Syndrome, Goldenhar-Gorlin Syndrome, Goldscheider's Disease, Goltz Syndrome, Goltz-Gorlin Syndrome, Gonadal Dysgenesis 45 X, Gonadal Dysgenesis XO, Goniodysgenesis-Hypodontia, Goodman Syndrome, Goodman, Goodpasture Syndrome, Gordon Syndrome, Gorlin's Syndrome, Gorlin-Chaudhry-Moss Syndrome, Gottron Erythrokeratodermia Congenitalis Progressiva Symmetrica, Gottron's Syndrome, Gougerot-Carteaud Syndrome, Grand Mal Epilepsy, Granular Type Comeal Dystrophy, Granulomatous Arteritis, Granulomatous Colitis, Granulomatous Dermatitis with Eosinophilia, Granulomatous Ileitis, Graves Disease, Graves' Hyperthyroidism, Graves' Disease, Greig Cephalopolysyndactyly Syndrome, Groenouw Type I Comeal Dystrophy, Groenouw Type II Comeal Dystrophy, Gronblad-Strandberg Syndrome, Grotton Syndrome, Growth Hormone Receptor Deficiency, Growth Hormone Binding Protein Deficiency, Growth Hormone Deficiency, Growth-Mental Deficiency Syndrome of Myhre, Growth Retardation-Rieger Anomaly, GRS, Gruber Syndrome, GS, GSD6, GSD8, GTS, Guanosine Triphosphate-Cyclohydrolase Deficiency, Guanosine Triphosphate-Cyclohydrolase Deficiency, Guenther Porphyria, Guerin-Stern Syndrome, Guillain-Barre, Guillain-Barre Syndrome, Gunther Disease, H Disease, H. Gottron's Syndrome, H. Gottron's Syndrome, Habit Spasms, HAE, Hageman Factor Deficiency, Hageman factor, Haim-Munk Syndrome, Hajdu-Cheney Syndrome, Hajdu Cheney, HAL Deficiency, Hall-Pallister Syndrome, Hallermann-Streiff-Francois syndrome, Hallermann-Streiff Syndrome, Hallervorden-Spatz Disease, Hallervorden-Spatz Syndrome, Hallopeau-Siemens Disease, Hallux Duplication Postaxial Polydactyly and Absence of Corpus Callosum, Halushi-Behcet's Syndrome, Hamartoma of the Lymphatics, Hand-Schueller-Christian Syndrome, HANE, Hanhart Syndrome, Happy Puppet Syndrome, Harada Syndrome, HARD ±E Syndrome, HARD Syndrome, Hare Lip, Harlequin Fetus, Harlequin Type DOC 6, Harlequin Type Ichthyosis, Harlequin Type Ichthyosis, Harley Syndrome, Harrington Syndrome, Hart Syndrome, Hartnup Disease, Hartnup Disorder, Hartnup Syndrome, Hashimoto's Disease, Hashimoto-Pritzker Syndrome, Hashimoto's Syndrome, Hashimoto's Thyroiditis, Hashimoto's Thyroiditis, Hashimoto-Pritzker Syndrome, Hay Well's Syndrome, Hay-Wells Syndrome of Ectodermal Dysplasia, HCMM, HCP, HCTD, HD, Heart-Ha&nd Syndrome (Holt-Oram Type), Heart Disease, Hecht Syndrome, HED, Heerferdt-Waldenstrom and Lofgren's Syndromes, Hegglin's Disease, Heinrichsbauer Syndrome, Hemangiomas, Hemangioma Familial, Hemangioma-Thrombocytopenia Syndrome, Hemangiomatosis Chondrodystrophica, Hemangiomatous Branchial Clefts-Lip Pseudocleft Syndrome, Hemifacial Microsomia, Hemimegalencephaly, Hemiparesis of Cerebral Palsy, Hemiplegia of Cerebral Palsy, Hemisection of the Spinal Cord, Hemochromatosis, Hemochromatosis Syndrome, Hemodialysis-Related Amyloidosis, Hemoglobin Lepore Syndromes, Hemolytic Anemia of Newborn, Hemolytic Cold Antibody Anemia, Hemolytic Disease of Newborn, Hemolytic-Uremic Syndrome, Hemolytic-Uremic Syndrome, Hemophilia, Hemophilia A, Hemophilia B, Hemophilia B Factor IX, Hemophilia C, Hemorrhagic Dystrophic Thrombocytopenia, Hemorrhagica Aleukia, Hemosiderosis, Hepatic Fructokinase Deficiency, Hepatic Phosphorylase Kinase Deficiency, Hepatic Porphyria, Hepatic Porphyrias, Hepatic Porphyrias, Hepatic Veno-Occlusive Diseas, Hepato-Renal Syndrome, Hepatolenticular Degeneration, Hepatophosphorylase Deficiency, Hepatorenal Glycogenosis, Hepatorenal Syndrome, Hepatorenal Tyrosinemia, Hereditary Acromeialgia, Hereditary Alkaptonuria, Hereditary Amyloidosis, Hereditary Angioedema, Hereditary Areflexic Dystasia, Heredopathia Atactica Polyneuritiformis, Hereditary Ataxia, Hereditary Ataxia, Hereditary Ataxia Friedrich's Type, Hereditary Benign Acanthosis Nigricans, Hereditary Cerebellar Ataxia, Hereditary Chorea, Hereditary Chronic Progressive Chorea, Hereditary Connective Tissue Disorders, Hereditary Coproporphyria, Hereditary Coproporphyria Porphyria, Hereditary Cutaneous Malignant Melanoma, Hereditary Deafness-Retinitis Pigmentosa, Heritable Disorder of Zinc Deficiency, Hereditary DNS, Hereditary Dystopic Lipidosis, Hereditary Emphysema, Hereditary Fructose Intolerance, Hereditary Hemorrhagic Telangiectasia, Hereditary Hemorrhagic Telangiectasia Type I, Hereditary Hemorrhagic Telangiectasia Type II, Hereditary Hemorrhagic Telangiectasia Type III, Hereditary Hyperuricemia and Choreoathetosis Syndrome, Hereditary Leptocytosis Major, Hereditary Leptocytosis Minor, Hereditary Lymphedema, Hereditary Lymphedema Tarda, Hereditary Lymphedema Type I, Hereditary Lymphedema Type II, Hereditary Motor Sensory Neuropathy, Hereditary Motor Sensory Neuropathy I, Hereditary Motor Sensory Neuropathy Type III, Hereditary Nephritis, Hereditary Nephritis and Nerve Deafness, Hereditary Nephropathic Amyloidosis, Hereditary Nephropathy and Deafness, Hereditary Nonpolyposis Colorectal Cancer, Hereditary Nonpolyposis Colorectat Carcinoma, Hereditary Nonspherocytic Hemolytic Anemia, Hereditary Onychoosteodysplasia, Hereditary Optic Neuroretinopathy, Hereditary Polyposis Coli, Hereditary Sensory and Autonomic Neuropathy Type I, Hereditary Sensory and Autonomic Neuropathy Type II, Hereditary Sensory and Autonomic Neuropathy Type III, Hereditary Sensory Motor Neuropathy, Hereditary Sensory Neuropathy type I, Hereditary Sensory Neuropathy Type I, Hereditary Sensory Neuropathy Type II, Hereditary Sensory Neuropathy Type III, Hereditary Sensory Radicular Neuropathy Type I, Hereditary Sensory Radicular Neuropathy Type I, Hereditary Sensory Radicular Neuropathy Type II, Hereditary Site Specific Cancer, Hereditary Spherocytic Hemolytic Anemia, Hereditary Spherocytosis, Hereditary Tyrosinemia Type 1, Heritable Connective Tissue Disorders, Herlitz Syndrome, Hermans-Herzberg Phakomatosis, Hermansky-Pudlak Syndrome, Hermansky-Pudlak Syndrome, Hermaphroditism, Herpes Zoster, Herpes Iris Stevens-Johnson Type, Hers Disease, Heterozygous Beta Thalassemia, Hexoaminidase Alpha-Subunit Deficiency. (Variant B), Hexoaminidase Alpha-Subunit Deficiency (Variant B), HFA, HFM, HOPS, HH, HHHO, HHRH, HHT, Hiatal Hemia-Microcephaly-Nephrosis Galloway Type, Hidradenitis Suppurativa, Hidrosadenitis Axillaris, Hidrosadenitis Suppurativa, Hidrotic Ectodermal Dysplasias, HIE Syndrome, High Imperforate Anus, High Potassium, High Scapula, HIM, Hirschsprung's Disease, Hirschsprung's Disease Acquired, Hirschsprung Disease Polydactyly of Ulnar & Big Toe and VSD, Hirschsprung Disease with Type D Brachydactyly, Hirsutism, HIS Deficiency, Histidine Ammonia-Lyase (HAL) Deficiency, Histidase Deficiency, Histidinemia, Histidinemia, Histiocytosis, Histiocytosis X, HLHS, HLP Type II, HMG, HMI, HMSN I, HNHA, HOCM, Hodgkin Disease, Hodgkin's Disease, Hodgkin's Lymphoma, Hollaender-Simons Disease, Holmes-Adie Syndrome, Holocarboxylase Synthetase Deficiency, Holoprosencephaly, Holoprosencephaly Malformation Complex, Holoprosencephaly Sequence, Holt-Oram Syndrome, Holt-Oram Type Heart-Hand Syndrome, Homocystinemia, Homocystinuria, Homocystinuria, Homogentisic Acid Oxidase Deficiency, Homogentisic Acidura, Homozygous Alpha-1-Antitrypsin Deficiency, HOOD, Homer Syndrome, Horton's disease, HOS, HOS 1, Houston-Harris Type Achrondrogenesis (Type IA), HPS, HRS, HS, HS, HS, HS, HS, HSAN Type I, HSAN Type II, HSAN-III, HSMN, HSMN Type III, HSN I, HSN-III, Huebner-Herter Disease, Hunner's Patch, Hunner's Ulcer, Hunter Syndrome, Hunter Syndrome, Hunter-Thompson Type Acromesomelic Dysplasia, Huntington's Chorea, Huntington's Disease, Hurler Disease, Hurler Disease, Hurler Syndrome, Hurler-Scheie Syndrome, HUS, HUS, Hutchinson-Gilford Progeria Syndrome, Hutchinson-Gilford Syndrome, Hutchinson-Weber-Peutz Syndrome, Hutchinson-Weber-Peutz Syndrome, Hutterite Syndrome Bowen-Conradi Type, Hyaline Panneuropathy, Hydranencephaly, Hydrocephalus, Hydrocephalus Agyria and Retinal Dysplasia, Hydrocephalus Internal Dandy-Walker Type, Hydrocephalus Noncommunicating Dandy-Walker Type, Hydrocephaly, Hydronephrosis With Peculiar Facial Expression, Hydroxylase Deficiency, Hygroma Colli, Hyper-IgE Syndrome, Hyper-IgM Syndrome, Hyper IgM Syndrome, Hyperaldosteronism, Hyperaldosteronism With Hypokalemic Alkatosis, Hyperaldosteronism Without Hypertension, Hyperammonemia, Hyperammonemia Due to Carbamylphosphate Synthetase Deficiency, Hyperammonemia Due to Ornithine Transcarbamylase Deficiency, Hyperammonemia Type II, Hyper-Beta Carnosinemia, Hyperbilirubinemia I, Hyperbilirubinemia II, Hypercalcemia Familial with Nephrocalcinosis and Indicanuria, Hypercalcemia-Supravalvar Aortic Stenosis, Hypercalciuric Rickets, Hypercapnic acidosis, Hypercatabolic Protein-Losing Enteropathy, Hyperchloremic acidosis, Hypercholesterolemia, Hypercholesterolemia Type IV, Hyperchylomicronemia, Hypercystinuria, Hyperekplexia, Hyperextensible joints, Hyperglobulinemic Purpura, Hyperglycinemia with Ketoacidosis and Lactic Acidosis Propionic Type, Hyperglycinemia Nonketotic, Hypergonadotropic Hypogonadism, Hyperimmunoglobulin E Syndrome, Hyperimmunoglobulin E-Recurrent Infection Syndrome, Hyperimmunoglobulinemia E-Staphylococcal, Hyperkalemia, Hyperkinetic Syndrome, Hyperlipemic Retinitis, Hyperlipidemia I, Hyperlipidemia IV, Hyperlipoproteinemia Type I, Hyperlipoproteinemia Type III, Hyperlipoproteinemia Type IV, Hyperoxaluria, Hyperphalangy-Clinodactyly of Index Finger with Pierre Robin Syndrome, Hyperphenylalanemia, Hyperplastic Epidermolysis Bullosa, Hyperpnea, Hyperpotassemia, Hyperprebeta-Lipoproteinemia, Hyperprolinemia Type I, Hyperprolinemia Type II, Hyperspienism, Hypertelorism with Esophageal Abnormalities and Hypospadias, Hypertelorism-Hypospadias Syndrome, Hypertrophic Cardiomyopathy, Hypertrophic Interstitial Neuropat.y, Hypertrophic Interstitial Neuritis, Hypertrophic Interstitial Radiculoneuropathy, Hypertrophic Neuropathy of Refsum, Hypertrophic Obstructive Cardiomyopathy, Hyperuricemia Choreoathetosis Self-multilation Syndrome, Hyperuricemia-Oligophrenia, Hypervalinemia, Hypocalcified (Hypomineralized) Type, Hypochondrogenesis, Hypochrondroplasia, Hypogammaglobulinemia, Hypogammaglobulinemia Transient of Infancy, Hypogenital Dystrophy with Diabetic Tendency, Hypoglossia-Hypodactylia Syndrome, Hypoglycemia, Hypoglycemia, Exogenous Hypoglycemia, Hypoglycemia with Macroglossia, Hypoglycosylation Syndrome Type la, Hypoglycosylation Syndrome Type la, Hypogonadism with Anosmia, Hypogonadotropic Hypogonadism and Anosmia, Hypohidrotic Ectodermal Dysplasia, Hypohidrotic Ectodermal Dysplasia Autosomal Dominant type, Hypohidrotic Ectodermal Dysplasias Autorecessive, Hypokalemia, Hypokalemic Alkalosis with Hypercalciuria, Hypokalemic Syndrome, Hypolactasia, Hypomaturation Type (Snow-Capped Teeth), Hypomelanosis of Ito, Hypomelia-Hypotrichosis-Facial Hemangioma Syndrome, Hypomyelination Neuropathy, Hypoparathyroidism, Hypophosphatasia, Hypophosphatemic Rickets with Hypercalcemia, Hypopigmentation, Hypopigmentation, Hypopigmented macular lesion, Hypoplasia of the Depressor Anguli Oris Muscle with Cardiac Defects, Hypoplastic Anemia, Hypoplastic Congenital Anemia, Hypoplastic Chondrodystrophy, Hypoplastic Enamel-Onycholysis-Hypohidrosis, Hypoplastic (Hypoplastic-Explastic) Type, Hypoplastic Left Heart Syndrome, Hypoplastic Left Heart Syndrome, Hypoplastic-Triphalangeal Thumbs, Hypopotassemia Syndrome, Hypospadias-Dysphagia Syndrome, Hyposmia, Hypothalamic Hamartoblastoma Hypopituitarism Imperforate Anus Polydactyly, Hypothalamic Infantilism-Obesity, Hypothyroidism, Hypotonia-Hypomentia-Hypogonadism-Obesity Syndrome, Hypoxanthine-Guanine Phosphoribosyltranferase Defect (Complete Absense of), I-Cell Disease, Iatrogenic Hypoglycemia, IBGC, IBIDS Syndrome, IBM, IBS, IC, I-Cell Disease, ICD, ICE Syndrome Cogan-Reese Type, Icelandic Type Amyloidosis (Type VI), I-Cell Disease, Ichthyosiform Erythroderma Corneal Involvement and Deafness, Ichthyosiform Erythroderma Hair Abnormality Growth and Men, Ichthyosiform Erythroderma with Leukocyte Vacuolation, Ichthyosis, Ichthyosis Congenita, Ichthyosis Congenital with Trichothiodystrophy, Ichthyosis Hystrix, Ichthyosis Hystrix Gravior, Ichthyosis Linearis Circumflexa, Ichtlhyosis Simplex, Ichthyosis Tay Syndrome, Ichthyosis Vulgaris, Ichthyosis Vulgaris, Ichthyotic Neutral Lipid Storage Disease, Icteric. Leptospirosis, Icterohemorrhagic Leptospirosis, Icterus (Chronic Familial), Icterus Gravis Neonatorum, Icterus Intermittens Juvenalis, Idiopathic Alveolar Hypoventilation, Idiopathic Amyloidosis, Idiopathic Arteritis of Takayasu, Idiopathic Basal Ganglia Calcification (IBGC), Idiopathic Brachial Plexus Neuropathy, Idiopathic Cervical Dystonia, Idiopathic Dilatation of the Pulmonary Artery, Idiopathic Dilatation of the Pulmonary Artery, Idiopathic Facial Palsy, Idiopathic Familial Hyperlipemia, Idiopathic Hypertrophic Subaortic Stenosis, Idiopathic Hypoproteinemia, Idiopathic Immunoglobulin Deficiency, Idiopathic Neonatal Hepatitis, Idiopathic Non-Specific Ulcerative Colitis, Idiopathic Non-Specific Ulcerative Colitis, Idiopathic Peripheral Periphlebitis, Idiopathic Pulmonary Fibrosis, Idiopathic Refractory Sideroblastic Anemia, Idiopathic Refractory Sideroblastic Anemia, Idiopathic Renal Hematuria, Idiopathic Steatorrhea, Idiopathic Thrombocythemia, Idiopathic Thrombocythemia, Idiopathic Thrombocytopenic Purpura, Idiopathic Thrombocytopenia Purpura (ITP), IDPA, IDPA, IgA Nephropathy, IgA Nephropathy, IHSS, Ileitis, Ileocolitis, Illinois Type Amyloidosis, ILS, IM, IMD2, IMD5, IMD5, Immune Defect due to Absence of Thymus, Immune Hemolytic Anemia Paroxysmal Cold, Immunodeficiency with Ataxia Telangiectasia, Immunodeficiency Cellular with Abnormal Immunoglobulin Synthesis, Immunodeficiency Common Variable Unclassifiable, Immunodeficiency with Hyper-IgM, Immunodeficiency with Leukopenia, Immunodeficiency-2, Immunodeficiency-5 (IMD5), Immunoglobulin Deficiency, Imperforate Anus, Imperforate Anus with Hand Foot and Ear Anomalies, Imperforate Nasolacrimal Duct and Premature Aging Syndrome, Impotent Neutrophil Syndrome, Inability To Open Mouth Completely And Short Finger-Flexor, INAD, INAD, Inborn Error of Urea Synthesis Arginase Type, Inborn Error of Urea Synthesis Arginino Succinic Type, Inborn Errors of Urea Synthesis Carbamyl Phosphate Type, Inborn Error of Urea Synthesis Citrullinemia Type, Inborn Errors of Urea Synthesis Glutamate Synthetase Type, INCL, Inclusion body myositis, Incomplete Atrioventricular Septal Defect, Incomplete Testicular Feminization, Incomplete Testicular Feminization, Incontinentia Pigmenti, Incontinentia Pigmenti, Incontinenti Pigmenti Achromians, Index Finger Anomaly with Pierre Robin Syndrome, Indiana Type Amyloidosis (Type II), Indolent systemic mastocytosis, Infantile Acquired Aphasia, Infantile Autosomal Recessive Polycystic Kidney Disease, Infantile Beriberi, Infantile Cerebral Ganglioside, Infantile Cerebral Ganglioside, Infantile Cerebral Paralysis, Infantile Cystinosis, Infantile Epileptic, Infantile Fanconi Syndrome with Cystinosis, Infantile Finnish Type Neuronal Ceroid Lipofuscinosis, Infantile Gaucher Disease, Infantile Hypoglycemia, Infantile Hypophasphatasia, Infantile Lobar Emphysema, Infantile Myoclonic Encephalopathy, Infantile Myoclonic Encephalopathy and Polymyoclonia, Infantile Myofibromatosis, Infantile Necrotizing Encephalopathy, Infantile Neuronal Ceroid Lipofuscinosis, Infantile Neuroaxonal Dystrophy, Infantile Onset Schindler Disease, Infantile Phytanic Acid Storage Disease, Infantile Refsum Disease (IRD), Infantile Sipoidosis GM-2 Gangliosideosis (Type S), Infantile Sipoidosis GM-2 Gangliosideosis (Type S, Infantile Sleep Apnea, Infantile Spasms, Infantile Spinal Muscular Atrophy (all types), Infantile Spinal Muscular Atrophy ALS, Infantile Spinal Muscular Atrophy Type I, Infantile Type Neuronal Ceroid Lipofuscinosis, Infectious Jaundice, Inflammatory Breast Cancer, Inflammatory Linear Nevus Sebaceous Syndrome, Iniencephaly, Insulin Resistant Acanthosis Nigricans, Insulin Lipodystrophy, Insulin dependent Diabetes, Intention Myoclonus, Intermediate Cystinosis, Intermediate Maple Syrup Urine Disease, Intermittent Ataxia with Pyruvate Dehydrogenase Deficiency, Intermittent Ataxia with Pyruvate Dehydrogenase Deficiency, Intermittent Maple Syrup Urine Disease, Internal Hydrocephalus, Interstitial Cystitis, Interstitial Deletion of 4q Included, Interstitial Deletion of 4q-Included, Intestinal Lipodystrophy, Intestinal Lipophagic Granulomatosis, Intestinal Lymphangiectasia, Intestinal Polyposis I, Intestinal Polyposis II, Intestinal Polyposis II, Intestinal Polyposis III, Intestinal Polyposis-Cutaneous Pigmentation Syndrome, Intestinal Polyposis-Cutaneous Pigmentation Syndrome, Intestinal Pseudoobstruction with External Ophthalmoplegia, Intracranial Neoplasm, Intracranial Tumors, Intracranial Vascular Malformations, Intrauterine Dwarfism, Intrauterine Synechiae, Inverted Smile And Occult Neuropathic Bladder, Iowa Type Amyloidosis (Type IV), IP, IPA, Iridocorneal Endothelial Syndrome, Iridocorneal Endothelial (ICE) Syndrome Cogan-Resse Type, Iridogoniodysgenesis With Somatic Anomalies, Iris Atrophy with Comeal Edema and Glaucoma, Iris Nevus Syndrome, Iron Overload Anemia, Iron Overload Anemia, Iron Overload Disease, Irritable Bowel Syndrome, Irritable Colon Syndrome, Isaacs Syndrome, Isaacs-Merten Syndrome, Ischemic Cardiomyopathy, Isolated Lissencephaly Sequence, Isoleucine 33 Amyloidosis, Isovaleric Acid CoA Dehydrogenase Deficiency, Isovaleric Acidaemia, Isovalericacidemia, Isovaleryl CoA Carboxylase Deficiency, ITO Hypomelanosis, ITO, ITP, ITP, IVA, Ivemark Syndrome, Iwanoff Cysts, Jackknife Convulsion, Jackson-Weiss Craniosynostosis, Jackson-Weiss Syndrome, Jacksonian Epilepsy, Jacobsen Syndrome, Jadassohn-Lewandowsky Syndrome, Jaffe-Lichenstein Disease, Jakob's Disease, Jakob-Creutzfeldt Disease, Janeway I, Janeway Dysgammaglobulinemia, Jansen Metaphyseal Dysostosis, Jansen Type Metaphyseal Chondrodysplasia, Jarcho-Levin Syndrome, Jaw-Winking, JBS, JBS, JDMS, Jegher's Syndrome, Jegher's Syndrome, Jejunal Atresia, Jejunitis, Jejunoileitis, Jervell and Lange-Nielsen Syndrome, Jeune Syndrome, JMS, Job Syndrome, Job-Buckley Syndrome, Johanson-Blizzard Syndrome, John Dalton, Johnson-Stevens Disease, Jonston's Alopecia, Joseph's Disease, Joseph's Disease Type I, Joseph's Disease Type II, Joseph's Disease Type III, Joubert Syndrome, Joubert-Bolthauser Syndrome, JRA, JRA, Juberg Hayward Syndrome, Juberg-Marsidi Syndrome, Juberg-Marsidi Mental Retardation Syndrome, Jumping Frenchmen, Jumping Frenchmen of Maine, Juvenile Arthritis, Juvenile Arthritis, Juvenile Autosomal Recessive Polycystic Kidney Disease, Juvenile Cystinosis, Juvenile (Childhood) Dermatomyositis (JDMS), Juvenile Diabetes, Juvenile Gaucher Disease, Juvenile Gout Choreoathetosis and Mental Retardation Syndrome, Juvenile Intestinal Malabsorption of Vit B12, Juvenile Intestinal Malabsorption of Vitamin B12, Juvenile Macular Degeneration, Juvenile Pernicious Anemia, Juvenile Retinoschisis, Juvenile Rheumatoid Arthritis, Juvenile Rheumatoid Arthritis, Juvenile Spinal Muscular Atrophy Included, Juvenile Spinal Muscular Atrophy ALS Included, Juvenile Spinal Muscular Atrophy Type III, Juxta-Articular Adiposis Dolorosa, Juxta-Articular Adiposis Dolorosa, Juxtaglomerular Hyperplasia, Kabuki Make-Up Syndrome, Kahler Disease, Kallmann Syndrome, Kanner Syndrome, Kanzaki Disease, Kaposi Disease (not Kaposi Sarcoma), Kappa Light Chain Deficiency, Karsch-Neugebauer Syndrome, Karsch-Neugebauer Syndrome, Kartagener Syndrome-Chronic Sinobronchial Disease and Dextrocardia, Kartagener Triad, Kasabach-Merritt Syndrome, Kast Syndrome, Kawasaki Disease, Kawasaki Syndrome, KBG Syndrome, KD, Kearns-Sayre Disease, Kearns-Sayre Syndrome, Kearns-Sayre Syndrome, Kennedy Disease, Kennedy Syndrome, Kennedy Type Spinal and Bulbar Muscular Atrophy, Kennedy-Stefanis Disease, Kenny Disease, Kenny Syndrome, Kenny Type Tubular Stenosis, Kenny-Caffe Syndrome, Kera. Palmoplant. Con. Pes Planus Ony. Periodon. Arach., Keratitis Ichthyosis Deafniess Syndrome, Keratoconus, Keratoconus, Keratoconus Posticus Circumscriptus, Keratolysis, Keratolysis Exfoliativa Congenita, Keratolytic Winter Erythema, Keratomalacia, Keratosis Follicularis, Keratosis Follicularis Spinulosa Decalvans, Keratosis Follicularis Spinulosa Decalvans Ichthyosis, Keratosis Nigricans, Keratosis Palmoplantaris with Periodontopathia and Onychogryposis, Keratosis Palmoplantaris Congenital Pes Planus Onychogryposis Periodontosis Arachnodactyly, Keratosis Palmoplantaris Congenital, Pes Planus, Onychogryphosis, Periodontosis, Arachnodactyly, Acroosteolysis, Keratosis Rubra Figurata, Keratosis Seborrheica, Ketoacid Decarboxylase Deficiency, Ketoaciduria, Ketotic Glycinemia, Ketotic Glycinemia, KFS, KID Syndrome, Kidney Agenesis, Kidneys Cystic-Retinal Aplasia Joubert Syndrome, Killian Syndrome, Killian/Teschler-Nicola Syndrome, Kiloh-Nevin syndrome III, Kinky Hair Disease, Kinsbourne Syndrome, Kleeblattschadel Deformity, Kleine-Levin Syndrome, Kleine-Levin Hibernation Syndrome, Klinefelter, Klippel-Feil Syndrome, Klippel-Feil Syndrome Type I, Klippel-Feil Syndrome Type II, Klippel-Feil Syndrome Type III, Klippel Trenaunay Syndrome, Klippel-Trenaunay-Weber Syndrome, Kluver-Bucy Syndrome, KMS, Kniest Dysplasia, Kniest Syndrome, Kobner's Disease, Koebberling-Dunnigan Syndrome, Kohlmeier-Degos Disease, Kok Disease, Korsakoff Psychosis, Korsakoff s Syndrome, Krabbe's Disease Included, Krabbe's Leukodystrophy, Kramer Syndrome, KSS, KSS, KTS, KTW Syndrome, Kufs Disease, Kugelberg-Welander Disease, Kugelberg-Welander Disease, Kugelberg-Welander Syndrome, Kugelberg-Welander Syndrome, Kugelberg-Welander Syndrome, Kussmaul-Landry Paralysis, KWS, L-3-Hydroxy-Acyl-CoA Dehydrogenase (LCHAD) Deficiency, Laband Syndrome, Labhart-Willi Syndrome, Labyrinthine Syndrome, Labyrinthine Hydrops, Lacrimo-Auriculo-Dento-Digital Syndrome, Lactase Isolated Intolerance, Lactase Deficiency, Lactation-Uterus Atrophy, Lactic Acidosis Leber Hereditary Optic Neuropathy, Lactic and Pyruvate Acidemia with Carbohydrate Sensitivity, Lactic and Pyruvate Acidemia with Episodic Ataxia and Weakness, Lactic and Pyruvate Acidemia with Carbohydrate Sensitivity,Lactic and Pyruvate, Lactic acidosis, Lactose Intolerance of Adulthood, Lactose Intolerance, Lactose Intolerance of Childhood, Lactose Intolerar.ce, LADD Syndrome, LADD, Lafora Disease Included, Lafora Body Disease, Laki-Lorand Factor Deficiency, LAM, Lambert Type Ichthyosis, Lambert-Eaton Syndrome, Lambert-Eaton Myasthenic Syndrome, Lamellar Recessive Ichthyosis, Lamellar Recessive Ichthyosis, Lamellar Ichthyosis, Lamellar Recessive Ichthyosis, Lancereaux-Mathieu-Weil Spirochetosis, Landau-Kleffner Syndrome, Landouzy Dejerine Muscular Dystrophy, Landry Ascending Paralysis, Langer-Salidino Type Achondrogensis (Type II), Langer Giedion Syndrome, Langerhans-Cell Granulomatosis, Langerhans-Cell Histiocytosis (LCH), Large Atrial and Ventricular Defect, Laron Dwarfism, Laron Type Pituitary Dwarfism, Larsen Syndrome, Laryngeal Dystonia, Latah (Observed in Malaysia), Late Infantile Neuroaxonal Dystrophy, Late Infantile Neuroaxonal Dystrophy, Late Onset Cockayne Syndrome Type III (Type C), Late-Onset Dystonia, Late-Onset Immunoglobulin Deficiency, Late-Onset Immunoglobulin Deficiency, Late Onset Pelizaeus-Merzbacher Brain Sclerosis, Lattice Comeal Dystrophy, Lattice Dystrophy, Launois-Bensaude, Launois-Cleret Syndrome, Laurence Syndrome, Laurence-Moon Syndrome, Laurence-Moon/Bardet-Biedl, Lawrence-Seip Syndrome, LCA, LCAD Deficiency, LCAD, LCAD, LCAD, LCADH Deficiency, LCH, LCHAD, LCHAD, LCPD, Le Jeune Syndrome, Leband Syndrome, Leber's Amaurosis, Leber′ s Congenital Amaurosis,Congenital Absence of the Rods and Cones, Leber's Congenital Tapetoretinal Degeneration, Leber's Congenital Tapetoretinal Dysplasia, Leber's Disease, Leber's Optic Atrophy, Leber's Optic Neuropathy, Left Ventricular Fibrosis, Leg Ulcer, Legg-Calve-Perthes Disease, Leigh's Disease, Leigh's Syndrome, Leigh's Syndrome (Subacute Necrotizing Encephalomyelopathy), Leigh Necrotizing Encephalopathy, Lennox-Gastaut Syndrome, Lentigio-Polypose-Digestive Syndrome, Lentigio-Polypose-Digestive Syndrome, Lenz Dysmorphogenetic Syndrome, Lenz Dysplasia, Lenz Microphthalmia Syndrome, Lenz Syndrome, LEOPARD Syndrome, Leprechaunism, Leprechaunism, Leptomeningeal Angiomatosis, Leptospiral Jaundice, Leri-Weill Disease, Leri-Weil Dyschondrosteosis, Leri-Weil Syndrome, Lermoyez Syndrome, Leroy Disease, Lesch Nyhan Syndrome, Lethal Infantile Cardiomyopathy, Lethal Neonatal Dwarfism, Lethal Osteochondrodysplasia, Letterer-Siwe Disease, Leukocytic Anomaly Albinism, Leukocytic Inclusions with Platelet Abnormality, Leukodystrophy, Leukodystrophy with Rosenthal Fibers, Leukoencephalitis Periaxialis Concentric, Levine-Critchley Syndrome, Levulosuria, Levy-Hollister Syndrome, LGMD, LGS, LHON, LHON, LIC, Lichen Ruber Acuminatus, Lichen Acuminatus, Lichen Amyloidosis, Lichen Planus, Lichen Psoriasis, Lignac-Debre-Fanconi Syndrome, Lignac-Fanconi Syndrome, Ligneous Conjunctivitis, Limb-Girdle Muscular Dystrophy, Limb Girdle Muscular Dystrophy, Limb Malformations-Dento-Digital Syndrome, Limit Dextrinosis, Linear Nevoid Hypermelanosis, Linear Nevus Sebacous Syndrome, Linear Scleroderma, Linear Sebaceous Nevus Sequence, Linear Sebaceous Nevus Syndrome, Lingua Fissurata, Lingua Plicata, Lingua Scrotalis, Linguofacial Dyskinesia, Lip Pseudocleft-hemangiomatous Branchial Cyst Syndrome, Lipid Granulomatosis, Lipid Histiocytosis, Lipid Kerasin Type, Lipid Storage Disease, Lipid-Storage Myopathy Associated with SCAD Deficiency, Lipidosis Ganglioside Infantile, Lipidosis Ganglioside Infantile, Lipoatrophic Diabetes Mellitus, Lipodystrophy, Lipoid Corneal Dystrophy, Lipoid Hyperplasia-Male Pseudohermaphroditism, Lipoid Hyperplasia-Male Pseudohermaphroditism, Lipomatosis of Pancreas Congenital, Lipomucopolysaccharidosis Type I, Lipomyelomeningocele, Lipoprotein Lipase Deficiency Familial, LIS, LIS 1, Lissencephaly 1, Lissencephaly Type I, Lissencephaly variants with agenesis of the corpus callosum cerebellar hypoplasia or other anomalies, Little Disease, Liver Phosphorylase Deficiency, LKS, LM Syndrome, Lobar Atrophy, Lobar Atrophy of the Brain, Lobar Holoprosencephaly, Lobar Tension Emphysema in Infancy, Lobstein Disease (Type I), Lobster Claw Deformity, Lobster Claw Deformity, Localized Epidermolysis Bullosa, Localized Lipodystrophy, Localized Neuritis of the Shoulder Girdle, Loeffler's Disease, Loeffler Endomyocardial Fibrosis with Eosinophilia, Loeffler Fibroplastic Parietal Endocarditis, Loken Syndrome, Loken-Senior Syndrome, Long-Chain 3-hydroxyacyl-CoA Dehydrogenase (LCHAD), Long Chain Acyl CoA Dehydrogenase Deficiency, Long-Chain Acyl-CoA Dehydrogenase (ACADL), Long-Chain Acyl-CoA Dehydrogenase Deficiency, Long QT Syndrome without Deafness, Lou Gehrig's Disease, Lou Gehrig's Disease Included, Louis-Bar Syndrome, Low Blood Sugar, Low-Density Beta Lipoprotein Deficiency, Low Imperforate Anus, Low Potassium Syndrome, Lowe syndrome, Lowe's Syndrome, Lowe-Bickel Syndrome, Lowe-Terry-MacLachlan Syndrome, LS, LS, LTD, Lubs Syndrome, Lubs Syndrome, Luft Disease, Lumbar Canal Stenosis, Lumbar Spinal Stenosis, Lumbosacral Spinal Stenosis, Lundborg-Unverricht Disease, Lundborg-Unverricht Disease Included, Lupus, Lupus, Lupus Erythematosus, L-uscika-Magendie Foramrina Atresia, Lyell Syndrome, Lyelles Syndrome, Lymphadenoid Goiter, Lymphangiectatic Protein-Losing Enteropathy, Lymphangioleiomatosis, Lymphangioleimyomatosis, Lymphangiomas, Lymphatic Malformations, Lynch Syndromes, Lynch Syndrome I, Lynch Syndrome II, Lysosomal Alpha-N-Acetylgalactosaminidase Deficiency Schindler Type, Lysosomal Glycoaminoacid Storage Disease-Angiokeratoma Corporis Diffusum, Lysosomal Glucosidase Deficiency, Lysosomal Glucosidase Deficiency, MAA, Machado Disease, Machado-Joseph Disease, Macrencephaly, Macrocephaly, Macrocephaly Hemihypertrophy, Macrocephaly with Multiple Lipomas and Hemangiomata, Macrocephaly with Pseudopapilledema and Multiple Hemangiomata, Macroglobulinemia, Macroglossia, Macroglossia-Omphalocele-Visceromegaly Syndrome, Macrostomia Ablepheron Syndrome, Macrothrombocytopenia Familial Bernard-Soulier Type, Macula Lutea degeneration, Macular Amyloidosis, Macular Degeneration, Macular Degeneration Disciform, Macular Degeneration Senile, Macular Dystrophy, Macular Type Corneal Dystrophy, MAD, MAD, Madelung's Disease, Maffucci Syndrome, Major Epilepsy, Malabsorption, Malabsorption-Ectodermal Dysplasia-Nasal Alar Hypoplasia, Maladie de Roger, Maladie de Tics, Male Malformation of Limbs and Kidneys, Male Turner Syndrome, Malignant Acanthosis, Malignant Acanthosis Nigricans, Malignant Astrocytoma, Malignant Atrophic Papulosis, Malignant Fever, Malignant Hyperphenylalaninemia, Malignant Hyperphenylalaninemia, Malignant Hyperpyrexia, Malignant Hyperthermia, Malignant Melanoma, Malignant Tumors of the Central Nervous System, Mallory-Weiss Laceration, Mallory-Weiss Tear, Mallory-Weiss Syndrome, Mammary Paget's Disease, Mandibular Ameloblastoma, Mandibulofacial Dysostosis, Mannosidosis, Map-Dot-Fingerprint Type Corneal Dystrophy, Maple Syrup Urine Disease, Maple Syrup Urine Disease, Marble Bones, Marchiafava-Micheli Syndrome, Marcus Gunn Jaw-Winking Syndrome, Marcus Gunn Phenomenon, Marcus Gunn Ptosis with jaw-winking, Marcus Gunn Syndrome, Marcus Gunn (Jaw-Winking) Syndrome, Marcus Gunn Ptosis (with jaw-winking), Marden-Walker Syndrome, Marden-Walker Type Connective Tissue Disorder, Marfan's Abiotrophy, Marfan-Achard syndrome, Marfan Syndrome, Marfan Syndrome, Marfan's Syndrome I, Marfan's Variant, Marfan-Achard syndrome, Marfanoid Hypermobility Syndrome, Marginal Corneal Dystrophy, Marie's Ataxia, Marie's Ataxia, Marie Disease, Marie-Sainton Disease, Marie Strumpell Disease, Marie-Strumpell Spondylitis, Marinesco-Sjogren Syndrome, Marinesco-Sjogren-Gorland Syndrome, Marker X Syndrome, Maroteaux Lamy Syndrome, Maroteaux Type Acromesomelic Dysplasia, Marshall's Ectodermal Dysplasias With Ocular and Hearing Defects, Marshall-Smith Syndrome, Marshall Syndrome, Marshall Type Deafness-Myopia-Cataract-Saddle Nose, Martin-Albright Syndrome, Martin-Bell Syndrome, Martorell Syndrome, MASA Syndrome, Massive Myoclonia, Mast Cell Leukemia, Mastocytosis, Mastocytosis With an Associated Hematologic Disorder, Maumenee Corneal Dystrophy, Maxillary Ameloblastoma, Maxillofacial Dysostosis, Maxillonasal Dysplasia, Maxillonasal Dysplasia Binder Type, Maxillopalpebral Synkinesis, May-Hegglin Anomaly, MCAD Deficiency, MCAD, MCAD, MCAD, McArdle Disease, McCune-Albright, MCD, McKusick Type Metaphyseal Chondrodysplasia, McKusick Type Metaphyseal Chondrodysplasia, MCR, MCTD, Meckel Syndrome, Meckel-Gruber Syndrome, Median Cleft Face Syndrome, Mediterranean Anemia, Medium-Chain Acyl-CoA dehydrogenase (AC-ADM), Medium Chain Acyl-CoA Dehydrogenase (MCAD) Deficiency, Medium-Chain Acyl-CoA Dehydrogenase Deficiency, Medium Chain Acyl CoA Dehydrogenase Deficiency, Medullary Cystic Disease, Medullary Cystic Disease, Medullary Sponge Kidney, MEF, Megaesophagus, Megalencephaly, Megalencephaly with Hyaline Inclusion, Megalencephaly with Hyaline Panneuropathy, Megaloblastic Anemia, Megaloblastic Anemia of Pregnancy, Megalocornea-Mental Retardation Syndrome, Meier-Gorlin Syndrome, Meige's Lymphedema, Meige's Syndrome, Melanodermic Leukodystrophy, Melanoplakia-Intestinal Polyposis, Melanoplakia-Intestinal Polyposis, MELAS Syndrome, MELAS, Melkersson Syndrome, Melnick-Fraser Syndrome, Melnick-Needles Osteodysplasty, Melnick-Needles Syndrome, Membranous Lipodystrophy, Mendes Da Costa Syndrome, Meniere Disease, Meniere's Disease, Meningeal Capillary Angiomatosis, Menkes Disease, Menke's Syndrome I, Mental Retardation Aphasia Shuffling Gait Adducted Thumbs (MASA), Mental Retardation-Deafness-Skeletal Abnormalities-Coarse Face with Full Lips, Mental Retardation with Hypoplastic 5th Fingernails and Toenails, Mental Retardation with Osteocartilaginous Abnormalities, Mental Retradation-X-linked with Growth Delay-Deafness-Microgenitalism, Menzel Type OPCA, Mermaid Syndrome, MERRF, MERRF Syndrome, MERRF, Merten-Singleton Syndrome MES. Mesangial IGA Nephropathy, Mesenteric Lipodystrophy, Mesiodens-Cataract Syndrome, Mesodermal Dysmorphodystrophy, Mesomelic Dwarfism-Madelung Deformity, Metabolic Acidosis, Metachromatic Leukodystrophy, Metatarsus Varus, Metatropic Dwarfism Syndrome, Metatropic Dysplasia, Metatropic Dysplasia I, Metatropic Dysplasia II, Methylmalonic Acidemia, Methylmalonic Aciduria, Meulengracht's Disease, MFD1, MG, MH, MHA, Micrencephaly, Microcephalic Primordial Dwarfism I, Microcephaly, Microcephaly-Hiatal Hernia-Nephrosis Galloway Type, Microcephaly-Hiatal Hernia-Nephrotic Syndrome, Microcystic Corneal Dystrophy, Microcythemia, Microlissencephaly, Microphthalmia, Microphthalmia, Microphthalmia or Anophthalmos with Associated Anomalies, Micropolygyria With Muscular Dystrophy, Microtia Absent Patellae Micrognathia Syndrome, Microvillus Inclusion Disease, MID, Midsystolic-click-late systolic murmur syndrome, Miescher's Type I Syndrome, Mikulicz Syndrome, Mikulicz-Radecki Syndrome, Mikulicz-Sjogren Syndrome, Mild Autosomal Recessive, Mild Intermediate Maple Syrup Urine Disease, Mild Maple Syrup Urine Disease, Miller Syndrome, Miller-Dieker Syndrome, Miller-Fisher Syndrome, Milroy Disease, Minkowski-Chauffard Syndrome, Minor Epilepsy, Minot-Von Willebrand Disease, Mirror-Image Dextrocardia, Mitochondrial Beta-Oxidation Disorders, Mitrochondrial and Cytosolic, Mitochondrial Cytopathy, Mitochondrial Cytopathy, Kearn-Sayre Type, Mitochondrial Encephalopathy, Mitochondrial Encephalomyopathy Lactic Acidosis and Strokelike Episodes, Mitochondrial Myopathy, Mitochondrial Myopathy Encephalopathy Lactic Acidosis Stroke-Like Episode, Mitochondrial PEPCK Deficiency, Mitral-valve prolapse, Mixed Apnea, Mixed Connective Tissue Disease, Mixed Connective Tissue Disease, Mixed Hepatic Porphyria, Mixed Non-Fluent Aphasia, Mixed Sleep Apnea, Mixed Tonic and Clonic Torticollis, MJD, MKS, ML I, ML II, ML II, ML III, ML IV, ML Disorder Type I, ML Disorder Type II, ML Disorder Type III, ML Disorder Type IV, MLNS, MMR Syndrome, MND, MNGIE, MNS, Mobitz I, Mobitz II, Mobius Syndrome, Moebius Syndrome, Moersch-Woltmann Syndrome, Mohr Syndrome, Monilethrix, Monomodal Visual Amnesia, Mononeuritis Multiplex, Mononeuritis Peripheral, Mononeuropathym Peripheral, Monosomy 3p2, Monosomy 9p Partial, Monosomy 11q Partial, Monosomy 13q Partial, Monosomy 18q Syndrome, Monosomy X, Monostotic Fibrous Dysplasia, Morgagni-Tumer-Albright Syndrome, Morphea, Morquio Disease, Morquio Syndrome, Morquio Syndrome A, Morquio Syndrome B, Morquio-Brailsford Syndrome, Morvan Disease, Mosaic Tetrasomy 9p, Motor Neuron Disease, Motor Neuron Disease, Motor Neuron Syndrome, Motor Neurone Disease, Motoneuron Disease, Motoneurone Disease, Motor System Disease (Focal and Slow), Moya-moya Disease, Moyamoya Disease, MPS, MPS I, MPS I H, MPS 1 H/S Hurler/Scheie Syndrome, MPS I S Scheie Syndrome, MPS II, MPS IIA, MWS IIB, MPS II-AR Autosomal Recessive Hunter Syndrome, MPS II-XR, MPS II-XR Severe Autosomal Recessive, MPS III, MPS III A B C and D Sanfiloppo A, MPS IV, MPS IV A and B Morquio A, MWS V, MPS VI, MPS VI Severe Intermediate Mild Maroteaux-Lamy, MWS VII, MWS VII Sly Syndrome, MPS VIII, MPS Disorder, MPS Disorder I, MPS Disorder II, MPS Disorder III, MPS Disorder VI, MPS Disorder Type VII, MRS, MS, MSA, MSD, MSL, MSS, MSUD, MSUD, MSUD Type Ib, MSUD Type II, Mucocutaneous Lymph Node Syndrome, Mucolipidosis I, Mucolipidosis II, Mucolipidosis II, Mucolipidosis III, Mucolipidosis IV, Mucopolysaccharidosis, Mucopolysaccharidosis I-H, Mucopolysaccharidosis I-S, Mucopolysaccharidosis II, Mucopolysaccharidosis III, Mucopolysaccharidosis IV, Mucopolysaccharidosis VI, Mucopolysaccharidosis VII, Mucopolysaccharidosis Type I, Mucopolysaccharidosis Type II, Mucopolysaccharidosis Type III, Mucopolysaccharidosis Type VII, Mucosis, Mucosulfatidosis, Mucous Colitis, Mucoviscidosis, Mulibrey Dwarfism, Mulibrey Nanism Syndrome, Mullerian Duct Aplasia-Renal Aplasia-Cervicothoracic Somite Dysplasia, Mullerian Duct-Renal-Cervicothoracic-Upper Limb Defects, Mullerian Duct and Renal Agenesis with Upper Limb and Rib Anomalies, Mullerian-Renal-Cervicothoracic Somite Abnormalities, Multi-Infarct Dementia Binswanger's Type, Multicentric Castleman's Disease, Multifocal Eosinophilic Granuloma, Multiple Acyl-CoA Dehydrogenase Deficiency, Multiple Acyl-CoA Dehydrogenase Deficiency, Multiple Acyl-CoA Dehydrogenase Deficiency/Glutaric Aciduria Type II, Multiple Angiomas and Endochondromas, Multiple Carboxylase Deficiency, Multiple Cartilaginous Enchondroses, Multiple Cartilaginous Exostoses, Multiple Enchondromatosis, Multiple Endocrine Deficiency Syndrome Type II, Multiple Epiphyseal Dysplasia, Multiple Exostoses, Multiple Exostoses Syndrome, Multiple Familial Polyposis, Multiple Lentigines Syndrome, Multiple Myeloma, Multiple Neuritis of the Shoulder Girdle, Multiple Osteochondromatosis, Multiple Peripheral Neuritis, Multiple Polyposis of the Colon, Multiple Pterygium Syndrome, Multiple Sclerosis, Multiple Sclerosis, Multiple Sulfatase Deficiency, Multiple Symmetric Lipomatosis, Multiple System Atrophy, Multisynostotic Osteodysgenesis, Multisynostotic Osteodysgenesis with Long Bone Fractures, Mulvihill-Smith Syndrome, MURCS Association, Murk Jansen Type Metaphyseal Chondrodysplasia, Muscle Camitine Deficiency, Muscle Core Disease, Muscle Phosphofructokinase Deficiency, Muscular Central Core Disease, Muscular Dystrophy, Muscular Dystrophy Classic X-linked Recessive, Muscular Dystrophy Congenital With Central Nervous System Involvement, Muscular Dystrophy Congenital Progressive with Mental Retardation, Muscular Dystrophy Facioscapulohumeral, Muscular Rheumatism, Muscular Rigidity—Progressive Spasm, Musculoskeletal Pain Syndrome, Mutilating Acropathy, Mutilating Acropathy, Mutism, mvp, MVP, MWS, Myasthenia Gravis, Myasthenia Gravis, Myasthenia Gravis Pseudoparalytica, Myasthenic Syndrome of Lambert-Eaton, Myelinoclastic Diff-use Sclerosis, Myelomatosis, Myhre Syndrome, Myoclonic Astatic Petit Mal Epilepsy, Myoclonic Dystonia, Myoclonic Encephalopathy of Infants, Myoclonic Epilepsy, Myoclonic Epilepsy Hartung Type, Myoclonus Epilepsy Associated with Ragged Red Fibers, Myoclonic Epilepsy and Ragged-Red Fiber Disease, Myoclonic Progressive Familial Epilepsy, Myoclonic Progressice Familial Epilepsy, Myoclonic Seizure, Myoclonus, Myoclonus Epilepsy, Myoencephalopathy Ragged-Red Fiber Disease, Myofibromatosis, Myofibromatosis Congenital, Myogenic Facio-Scapulo-Peroneal Syndrome, Myoneurogastointestinal Disorder and Encephalopathy, Myopathic Arthrogryposis Multiplex Congenita, Myopathic Camitine Deficiency, Myopathy Central Fibrillar, Myopathy Congenital Nonprogressive, Myopathy Congenital Nonprogressive with Central Axis, Myopathy with Deficiency of Camitine Palmitoyltransferase, Myopathy-Marinesco-Sjogren Syndrome, Myopathy-Metabolic Carnitine Palmitoyltransderase Deficiency, Myopathy Mitochondrial-Encephalopathy-Lactic Acidosis-Stroke, Myopathy with Sarcoplasmic Bodies and Intermediate Filaments, Myophosphorylase Deficiency, Myositis Ossificans Progressiv, Myotonia Atrophica, Myotonia Congenita, Myotonia Congenita Intermittens, Myotonic Dystrophy, Myotonic Myopathy Dwarfism Chondrodystrophy Ocular and Facial Anomalies, Myotubular Myopathy, Myotubular Myopathy X-linked, Myproic Acid, Myriachit (Observed in Siberia), Myxedema, N-Acetylglucosamine-1-Phosphotransferase Deficiency, N-Acetyl Glutamate Synthetase Deficiency, NADH-CoQ reductasedeficiency, Naegeli Ectodermal Dysplasias, Nager Syndrome, Nager Acrofacial Dysostosis Syndrome, Nager Acrofacial Dysostosis Syndrome, Nager Syndrome, NAGS Deficiency, Nail Dystrophy-Deafness Syndrome, Nail Dysgenesis and Hypodontia, Nail-Patella Syndrome, Nance-Horan Syndrome, Nanocephalic Dwarfism, Nanocephaly, Nanophthalmia, Narcolepsy, Narcoleptic syndrome, NARP, Nasal-fronto-faciodysplasia, Nasal Alar Hypoplasia Hypothyroidism Pancreatic Achylia Congenital Deafness, Nasomaxillary Hypoplasia, Nasu Lipodystrophy, NBIA1, ND, NDI, NDP, Necrotizing Encephalomyelopathy of Leigh's, Necrotizing Respiratory Granulomatosis, Neill-Dingwall Syndrome, Nelson Syndrome, Nemaline Myopathy, Neonatal Adrenoleukodystrophy, Neonatal Adrenoleukodystrophy (NALD), Neonatal Adrenoleukodystrophy (ALD), Neonatal Autosomal Recessive Polycystic Kidney Disease, Neonatal Dwarfism, Neonatal Hepatitis, Neonatal Hypoglycemia, Neonatal Lactose Intolerance, Neonatal Lymphedema due to Exudative Enteropathy, Neonatal Progeroid Syndrome, Neonatal Pseudo-Hydrocephalic Progeroid Syndrome of Wiedemann-Rautenstrauch, Neoplastic Arachnoiditis, Nephroblastom, Nephrogenic Diabetes Insipidus, Nephronophthesis Familial Juvenile, Nephronophthesis Familial Juvenile, Nephropathic Cystinosis, Nephropathy-Pseudohermaphroditism-Wilms Tumor, Nephrosis-Microcephaly Syndrome, Nephrosis-Neuronal Dysmigration Syndrome, Nephrotic-Glycosuric-Dwarfism-Rickets-Hypophosphatemic Syndrome, Netherton Disease, Netherton Syndrome, Netherton Syndrome Ichthyosis, Nettleship Falls Syndrome (X-Linked), Neu-Laxova Syndrome, Neuhauser Syndrome, Neural-tube defects, Neuralgic Amyotrophy, Neuralgic Amyotrophy, Neuraminidase Deficiency, Neuraocutaneous melanosis, Neurinoma of the Acoustic Nerve, Neurinoma, Neuroacanthocytosis, Neuroaxonal Dystrophy Schindler Type, Neurodegeneration with brain iron accumulation type 1 (NBIA1), Neurofibroma of the Acoustic Nerve, Neurogenic Arthrogryposis Multiplex Congenita, Neuromyelitis Optica, Neuromyotonia, Neuromyotonia, Focal, Neuromyotonia, Generalized, Familial, Neuromytonia, Generalized,Sporadic, Neuronal Axonal Dystrophy Schindler Type, Neuronal Ceroid Lipofuscinosis Adult Type, Neuronal Ceroid Lipofuscinosis Juvenile Type, Neuronal Ceroid Lipofuscinosis Type 1, Neuronopathic Acute Gaucher Disease, Neuropathic Amyloidosis, Neuropathic Beriberi, Neuropathy Ataxia and Retinitis Pigmentosa, Neuropathy of Brachialpelxus Syndrome, Neuropathy Hereditary Sensory Type I, Neuropathy Hereditary Sensory Type II, Neutral Lipid Storage Disease, Nevii, Nevoid Basal Cell Carcinoma Syndrome, Nevus, Nevus Cavemosus, Nevus Comedonicus, Nevus Depigmentosus, Nevus Sebaceous of Jadassohn, Nezelof's Syndrome, Nezelof's Thymic Aplasia, Nezelof Type Severe Combined Immunodeficiency, NF, NF1, NF2, NF-1, NF-2, NHS, Niemann Pick Disease, Nieman Pick disease Type A (acute neuronopathic form), Nieman Pick disease Type B, Nieman Pick Disease Type C (chronic neuronopathic form), Nieman Pick disease Type D (Nova Scotia variant), Nieman Pick disease Type E, Nieman Pick disease Type F (sea-blue histiocyte disease), Night Blindness, Nigrospinodentatal Degeneration, Niikawakuroki Syndrome, NLS, NM, Noack Syndrome Type I, Nocturnal Myoclonus Hereditary Essential Myoclonus, Nodular Cornea Degeneration, Non-Bullous CIE, Non-Bullous Congenital Ichthyosiform Erythroderma, Non-Communicating Hydrocephalus, Non-Deletion Type Alpha-Thalassemia/Mental Retardation syndrome, Non-Ketonic Hyperglycinemia Type I (NKHI), Non-Ketotic Hyperglycinemia, Non-Lipid Reticuloendotheliosis, Non-Neuronopathic Chronic Adult Gaucher Disease, Non-Scarring Epidermolysis Bullosa, Nonarteriosclerotic Cerebral Calcifications, Nonarticular Rheumatism, Noncerebral,Juvenile Gaucher Disease, Nondiabetic Glycosuria, Nonischemic Cardiomyopathy, Nonketotic Hypoglycemia and Carnitine Deficiency due to MCAD Deficiency, Nonketotic Hypoglycemia Caused by Deficiency of Acyl-CoA Dehydrogenase, Nonketotic Glycinemia, Nonne's Syndrome, Nonne-Milroy-Meige Syndrome, Nonopalescent Opalescent Dentine, Nonpuerperal Galactorrhea-Amenorrhea, Nonsecretory Myeloma, Nonspherocytic Hemolytic Anemia, Nontropical Sprue, Noonan Syndrome, Norepinephrine, Normal Pressure Hydrocephalus, Norman-Roberts Syndrome, Norrbottnian Gaucher Disease, Norrie Disease, Norwegian Type Hereditary Cholestasis, NPD, NPS, NS, NSA, Nuchal Dystonia Dementia Syndrome, Nutritional Neuropathy, Nyhan Syndrome, OAV Spectrum, Obstructive Apnea, Obstructive Hydrocephalus, Obstructive Sleep Apnea, OCC Syndrome, Occlusive Thromboaortopathy, OCCS, Occult Intracranial Vascular Malformations, Occult Spinal Dysraphism Sequence, Ochoa Syndrome, Ochronosis, Ochronotic Arthritis, OCR, OCRL, Octocephaly, Ocular Albinism, Ocular Herpes, Ocular Myasthenia Gravis, Oculo-Auriculo-Vertebral Dysplasia, Ocuio-Auriculo-Vertebral Spectrum, Oculo-Bucco-Genital Syndrome, Oculocerebral Syndrome with Hypopigmentation, Oculocerebrocutaneous Syndrome, Oculo-Cerebro-Renal, Oculocerebrorenal Dystrophy, Oculocerebrorenal Syndrome, Oculocraniosomatic Syndrome (obsolete), Oculocutaneous Albinism, Oculocutaneous Albinism Chediak-Higashi Type, Oculo-Dento-Digital Dysplasia, Oculo-Dento-Digital Dysplasia, Oculodentodigital Syndrome, Oculo-Dento-Osseous Dysplasia, Oculo-Dento-Osseous Dysplasia, Oculo Gastrointestinal Muscular Dystrophy, Oculo Gastrointestinal Muscular Dystrophy, Oculogastrointestinal Muscular Dystrophy, Oculomandibulodyscephaly with hypotrichosis, Oculomandibulofacial Syndrome, Oculomotor with Congenital Contractures and Muscle Atrophy, Oculosympathetic Palsy, ODD Syndrome, ODD Syndrome, ODOD, ODOD, Odontogenic Tumor, Odontotrichomelic Syndrome, OFD, OFD Syndrome, Ohio Type Amyloidosis (Type VII), OI, OI Congenita, OI Tarda, Oldfield Syndrome, Oligohydramnios Sequence, Oligophrenia Microphthalmos, Oligophrenic Polydystrophy, Olivopontocerebellar Atrophy, Olivopontocerebellar Atrophy, Olivopontocerebellar Atrophy with Dementia and Extrapyramidal Signs, Olivopontocerebellar Atrophy with Retinal Degeneration, Olivopontocerebellar Atrophy I, Olivopontocerebellar Atrophy II, Olivopontocerebellar Atrophy III, Olivopontocerebellar Atrophy IV, Olivopontocerebellar Atrophy V, Ollier Disease, Ollier Osteochondromatosis, Omphalocele-Visceromegaly-Macroglossia Syndrome, Ondine's Curse, Onion-Bulb Neuropathy, Onion Bulb Polyneuropathy, Onychoosteodysplasia, Onychotrichodysplasia with Neutropenia, OPCA, OPCA I, OPCA II, OPCA III, OPCA IV, OPCA V, OPD Syndrome, OPD Syndrome Type I, OPD Syndrome Type II, OPD I Syndrome, OPD II Syndrome, Ophthalmoarthropathy, Ophthalmoplegia-Intestinal Pseudoobstruction, Ophthalmoplegia, Pigmentary Degeneration of the Retina and Cadiomyopathy, Ophthalmoplegia Plus Syndrome, Ophthalmoplegia Syndrome, Opitz BBB Syndrome, Opitz BBB/G Compound Syndrome, Opitz BBBG Syndrome, Opitz-Frias Syndrome, Opitz G Syndrome, Opitz G/BBB Syndrome, Opitz Hypertelorism-Hypospadias Syndrome, Opitz-Kaveggia Syndrome, Opitz Oculogenitolaryngeal Syndrome, Opitz Trigonocephaly Syndrome, Opitz Syndrome, Opsoclonus, Opsoclonus-Myoclonus, Opthalmoneuromyelitis, Optic Atrophy Polyneuropathy and Deafness, Optic Neuroencephalomyelopathy, Optic Neuromyelitis, Opticomyelitis, Optochiasmatic Arachnoiditis, Oral-Facial Clefts, Oral-facial Dyskinesia, Oral Facial Dystonia, Oral-Facial-Digital Syndrome, Oral-Facial-Digital Syndrome Type I, Oral-Facial-Digital Syndrome I, Oral-Facial-Digital Syndrome II, Oral-Facial-Digital Syndrome III, Oral-Facial-Digital Syndrome IV, Orbital Cyst with Cerebral and Focal Dermal Malformations, Omithine Carbamyl Transferase Deficiency, Omithine Transcarbamylase Deficiency, Orocraniodigital Syndrome, Orofaciodigital Syndrome, Oromandibular Dystonia, Orthostatic Hypotension, Osler-Weber-Rendu disease, Osseous-Oculo-Dento Dysplasia, Osseous-Oculo-Dento Dysplasia, Osteitis deformans, Osteochondrodystrophy Deformans, Osteochondroplasia, Osteodysplasty of Melnick and Needles, Osteogenesis Imperfect, Osteogenesis Imperfecta, Osteogenesis Imperfecta Congenita, Osteogenesis Imperfecta Tarda, Osteohypertrophic Nevus Flammeus, Osteopathia Hyperostotica Scleroticans Multiplex Infantalis, Osteopathia Hyperostotica Scleroticans Multiplex Infantalis, Osteopathyrosis, Osteopetrosis, Osteopetrosis Autosomal Dominant Adult Type, Osteopetrosis Autosomal Recessive Malignant Infantile Type, Osteopetrosis Mild Autosomal Recessive Intermediate Typ, Osteosclerosis Fragilis Generalisata, Osteosclerotic Myeloma, Ostium Primum Defect (endocardial cushion defects included), Ostium Secundum Defect, OTC Deficiency, Oto Palato Digital Syndrome, Oto-Palato-Digital Syndrome Type I, Oto-Palatal-Digital Syndrome Type II, Otodental Dysplasia, Otopalatodigital Syndrome, Otopalataldigital Syndrome Type II, Oudtshoorn Skin, Ovarian Dwarfism Turner Type, Ovary Aplasia Turner Type, OWR, Oxalosis, Oxidase deficiency, Oxycephaly, Oxycephaly, Oxycephaly-Acrocephaly, P-V, PA, PAC, Pachyonychia Ichtyosiforme, Pachyonychia Congenita with Natal Teeth, Pachyonychia Congenita, Pachyonychia Congenita Keratosis Disseminata Circumscripta (follicularis), Pachyonychia Congenita Jadassohn-Lewandowsky Type, PAF with MSA, Paget's Disease, Paget's Disease of Bone, Paget's Disease of the Breast, Paget's Disease of the Nipple, Paget's Disease of the Nipple and Areola, Pagon Syndrome, Painful Ophthalmoplegia, PAIS, Palatal Myoclonus, Palato-Oto-Digital Syndrome, Palatal-Oto-Digital Syndrome Type I, Palatal-Oto-Digital SyndromeType II, Pallister Syndrome, Pallister-Hall Syndrome, Pallister-Killian Mosaic Syndrome, Pallister Mosaic Aneuploidy, Pallister Mosaic Syndrome, Pallister Mosaic Syndrome Tetrasomy 12p, Pallister-W Syndrome, Palmoplantar Hyperkeratosis and Alopecia, Palsy, Pancreatic Fibrosis, Pancreatic Insufficiency and Bone Marrow Dysfiuction, Pancreatic Ulcerogenic Tumor Syndrome, Panmyelophthisis, Panmyelopathy, Pantothenate kinase associated neurodegeneration (PKAN), Papillon-Lefevre Syndrome, Papillotonic Psuedotabes, Paralysis Periodica Paramyotonica, Paralytic Beriberi, Paralytic Brachial Neuritis, Paramedian Lower Lip Pits-Popliteal Pyerygium Syndrome, Paramedian Diencephalic Syndrome, Paramyeloidosis, Paramyoclonus Multiple, Paramyotonia Congenita, Paramyotonia Congenita of Von Eulenburg, Parkinson's disease, Paroxysmal Atrial Tachycardia, Paroxysmal Cold Hemoglobinuria, Paroxysmal Dystonia, Paroxysmal Dystonia Choreathetosis, Paroxysmal Kinesigenic Dystonia, Paroxysmal Nocturnal Hemoglobinuria, Paroxysmal Normal Hemoglobinuria, Paroxysmal Sleep, Parrot Syndrome, Parry Disease, Parry-Romberg Syndrome, Parsonage-Turner Syndrome, Partial Androgen Insensitivity Syndrome, Partial Deletion of the Short Arm ofChromosome 4, Partial Deletion of the Short Arm of Chromosome 5, Partial Deletion of Short Arm of Chromosome 9, Partial Duplication 3q Syndrome, Partial Duplication 15q Syndrome, Partial Facial Palsy With Urinary Abnormalities, Partial Gigantism of Hands and Feet-Nevi-Hemihypertrophy-Macrocephaly, Partial Lipodystrophy, Partial Monosomy of Long Arm of Chromosome 11, Partial Monosomy of the Long Arm of Chromosome 13, Partial Spinal Sensory Syndrome, Partial Trisomy 11q, Partington Syndrome, PAT, Patent Ductus Arteriosus, Pathological Myoclonus, Pauciarticular-Onset Juvenile Arthritis, Pauciarticular-Onset Juvenile Arthritis, Paulitis, PBC, PBS, PC Deficiency, PC Deficiency Group A, PC Deficiency Group B, PC, Eulenburg Disease, PCC Deficiency, PCH, PCLD, PCT, PD, PDA, PDH Deficiency, PDH Deficiency, Pearson Syndrome Pyruvate Carboxylase Deficiency, Pediatric Obstructive Sleep Apnea, Peeling Skin Syndrome, Pelizaeus-Merzbacher Disease, Pelizaeus-Merzbacher Brain Sclerosis, Pelizaeus-Merzbacher Brain Sclerosis, Pellagra-Cerebellar Ataxia-Renal Aminoaciduria Syndrome, Pelvic Pain Syndrome, Pemphigus Vulgaris, Pena Shokeir II Syndrome, Pena Shokeir Syndrome Type II, Penile Fibromatosis, Penile Fibrosis, Penile Induration, Penta X Syndrome, Pentalogy of Cantrell, Pentalogy Syndrome, Pentasomy X, PEPCK Deficiency, Pepper Syndrome, Perheentupa Syndrome, Periarticular Fibrositis, Pericardial Constriction with Growth Failure, Pericollagen Amyloidosis, Perinatal Polycystic Kidney Diseases, Perineal Anus, Periodic Amyloid Syndrome, Periodic Peritonitis Syndrome, Periodic Somnolence and Morbid Hunger, Periodic Syndrome, Peripheral Cystoid Degeneration of the Retina, Peripheral Dysostosis-Nasal Hypoplasia-Mental Retardation, Peripheral Neuritis, Peripheral Neuropathy, Peritoneopericardial Diaphragmatic Hernia, Pernicious Anemia, Pernicious Anemia, Pernicious Anemia, Peromelia with Micrognathia, Peroneal Muscular Atrophy, Peroneal Nerve Palsy, Peroutka Sneeze, Peroxisomal Acyl-CoA Oxidase, Peroxisomal Beta-Oxidation Disorders, Peroxisomal Bifunctional Enzyme, Peroxisomal Thiolase, Peroxisomal Thiolase Deficiency, Persistent Truncus Arteriosus, Perthes Disease, Petit Mal Epilepsy, Petit Mal Variant, Peutz-Jeghers Syndrome, Peutz-Jeghers Syndrome, Peutz-Touraine Syndrome, Peutz-Touraine Syndrome, Peyronie Disease, Pfeiffer, Pfeiffer Syndrome Type I, PGA I, PGA II, PGA II, PGA III, PGK, PH Type I, PH Type I, Pharyngeal Pouch Syndrome, PHD Short-Chain Acyl-CoA Dehydrogenase Deficiency, Phenylalanine Hydroxylase Deficiency, Phenylalaninemia, Phenylketonuria, Phenylketonuria, Phenylpyruvic Oligophrenia, Phocomelia, Phocomelia Syndrome, Phosphoenolpyruvate Carboxykinase Deficiency, Phosphofructokinase Deficiency, Phosphoglycerate Kinase Deficiency, Phosphoglycerokinase, Phosphorylase 6 Kinase Deficiency, Phosphorylase Deficiency Glycogen Storage Disease, Phosphorylase Kinase Deficiency of Liver, Photic Sneeze Reflex, Photic Sneezing, Phototherapeutic keratectomy, PHS, Physicist John Dalton, Phytanic Acid Storage Disease, Pi Phenotype ZZ, PI, Pick Disease of the Brain, Pick's Disease, Pick's Disease, Pickwickian Syndrome, Pierre Robin Anomalad, Pierre Robin Complex, Pierre Robin Sequence, Pierre Robin Syndrome, Pierre Robin Syndrome with Hyperphalangy and Clinodactyly, Pierre-Marie's Disease, Pigmentary Degeneration of Globus Pallidus Substantia Nigra Red Nucleus, Pili Torti and Nerve Deafness, Pili Torti-Sensorineural Hearing Loss, Pituitary Dwarfism II, Pituitary Tumor after Adrenalectomy, Pityriasis Pilaris, Pityriasis Rubra Pilaris, PJS, PJS, PKAN, PKD, PKD, PKD1, PKD2, PKD3, PKU, PKU, PKU1, Plagiocephaly, Plagiocephaly, Plagiocephaly, Plasma Cell Myeloma, Plasma Cell Leukemia, Plasma Thromboplastin Component Deficiency, Plasma Transglutaminase Deficiency, Plastic Induration Corpora Cavernosa, Plastic Induration of the Penis, PLD, Plicated Tongue, PLS, PMD, Pneumorenal Syndrome, PNH, PNM, PNP Deficiency, POD, POH, Poikiloderma Atrophicans and Cataract, Poikiloderma Congenitale, Poland Anomaly, Poland Sequence, Poland Syndactyly, Poland Syndrome, Poliodystrophia Cerebri Progressiva, Polyarthritis Enterica, Polyarteritis Nodosa, Polyarticular-Onset Juvenile Arthritis Type I, Polyarticular-Onset Juvenile Arthritis Type II, Polyarticular-Onset Juvenile Arthritis Types I and II, Polychondritis, Polycystic Kidney Disease, Polycystic Kidney Disease Medullary Type, Polycystic Kidney Disease Medullary Type, Polycystic Liver Disease, Polycystic Ovary Disease, Polycystic Renal Diseases, Polydactyly-Joubert Syndrome, Polydysplastic Epidermolysis Bullosa, Polydystrophia Oligophrenia, Polydystrophic Dwarfism, Polyglandular Autoimmune Syndrome Type III, Polyglandular Autoimmune Syndrome Type II, Polyglandular Autoimmune Syndrome Type I, Polyglandular Autoimmune Syndrome Type II, Polyglandular Deficiency Syndrome Type II, Polyglandular Syndromes, Polymorphic Macula Lutea Degeneration, Polymorphic Macular Degeneration, Polymorphism of Platelet Glycoprotien lb, Polymorphous Corneal Dystrophy Hereditary, Polymyalgia Rheumatica, Polymyalgia Rheumatica, Polymyositis and Dermatomyositis,Primary Agammag-lobulinemi, Polyneuritis Peripheral, Polyneuropathy-Deafness-Optic Atrophy, Polyneuropathy Peripheral, Polyneuropathy and Polyradiculoneuropathy, Polyostotic Fibrous Dysplasia, Polyostotic Sclerosing Histiocytosis, Polyposis Familial, Polyposis Gardner Type, Polyposis Hamartomatous Intestinal, Polyposis Hamartomatous Intestinal, Polyposis-Osteomatosis-Epidermoid Cyst Syndrome, Polyposis Skin Pigmentation Alopecia and Fingernail Changes, Polyps and Spots Syndrome, Polyps and Spots Syndrome, Polyserositis Recurrent, Polysomy Y, Polysyndactyly with Peculiar Skull Shape, Polysyndactyly-Dysmorphic Craniofacies Greig Type, Pompe Disease, Pompe Disease, Popliteal Pterygium Syndrome, Porcupine Man, Porencephaly, Porencephaly, Porphobilinogen deaminase (PBG-D), Porphyria, Porphyria Acute Intermittant, Porphyria Acute Intermittent, Porphyria ALA-D, Porphyria Cutanea Tarda, Porphyria Cutanea Tarda, Porphyria Cutanea Tarda Hereditaria, Porphyria Cutanea Tarda Symptomatica, Porphyria Hepatica Variegate, Porphyria Swedish Type, Porphyria Variegate, Porphyriam Acute Intermittent, Porphyrins, Porrigo Decalvans, Port Wine Stains, Portuguese Type Amyloidosis, Post-Infective Polyneuritis, Postanoxic Intention Myoclonus, Postaxial Acrofacial Dysostosis, Postaxial Polydactyly, Postencephalitic Intention Myoclonus, Posterior Corneal Dystrophy Hereditary, Posterior Thalamic Syndrome, Postmyelographic Arachnoiditis, Postnatal Cerebral Palsy, Postoperative Cholestasis, Postpartum Galactorrhea-Amenorrhea Syndrome, Postpartum Hypopituitaeism, Postpartum Panhypopituitary Syndrome, Postpartum Panhypopituitarism, Postpartum Pituitary Necrosis, Postural Hypotension, Potassium-Losing Nephritis, Potassium Loss Syndrome, Potter Type I Infantile Polycystic Kidney Diseases, Potter Type III Polycystic Kidney Disease, PPH, PPS, Prader-Willi Syndrome, Prader-Labhart-Willi Fancone Syndrome, Prealbumin Tyr-77 Amyloidosis, Preexcitation Syndrome, Preexcitation Syndrome, Pregnenolone Deficiency, Premature Atrial Contractions, Premature Senility Syndrome, Premature Supraventricular Contractions, Premature Ventricular Complexes, Prenatal or Connatal Neuroaxonal Dystrophy, Presenile Dementia, Presenile Macula Lutea Retinae Degeneration, Primary Adrenal Insufficiency, Primary Agammaglobulinemias, Primary Aldosteronism, Primary Alveolar Hypoventilation, Primary Amyloidosis, Primary Anemia, Primary Anemia, Primary Beriberi, Primary Biliary, Primary Biliary Cirrhosis, Primary Brown Syndrome, Primary Carnitine Deficiency, Primary Central Hypoventilation Syndrome, Primary Ciliary Dyskinesia Kartagener Type, Primary Cutaneous Amyloidosis, Primary Dystonia, Primary Failure Adrenocortical Insufficiency, Primary Familial Hypoplasia of the Maxilla, Primary Hemochromatosis, Primary Hyperhidrosis, Primary Hyperoxaluria [Type I], Primary Hyperoxaluria Type 1 (PH1), Primary Hyperoxaluria Type 1, Primary Hyperoxaluria Type II, Primary Hyperoxaluria Type III, Primary Hypogonadism, Primary Intestinal Lymphangiectasia, Primary Lateral Sclerosis, Primary Nonhereditary Amyloidosis, Primary Obliterative Pulmonary Vascular Disease, Primary Progressive Multiple Sclerosis, Primary Pulmonary Hypertension, Primary Reading Disability, Primary Renal Glycosuria, Primary Sclerosing Cholangitis, Primary Thrombocythemia, Primary Thrombocythemia, Primary Tumors of Central Nervous System, Primary Visual Agnosia, Proctocolitis Idiopathic, Proctocolitis Idiopathic, Progeria of Adulthood, Progeria of Childhood, Progeroid Nanism, Progeriod Short Stature with Pigmented Nevi, Progeroid Syndrome of De Barsy, Progressive Autonomic Failure with Multiple System Atrophy, Progressive Bulbar Palsy, Progressive Bulbar Palsy Included, Progressive Cardiomyopathic Lentiginosis, Progressive Cerebellar Ataxia Familial, Progressive Cerebral Poliodystrophy, Progressive Choroidal Atrophy, Progressive Diaphyseal Dysplasia, Progressive Diaphyseal Dysplasia, Progressive Facial Hemiatrophy, Progressive Familial Myoclonic Epilepsy, Progressive Hemifacial Atrophy, Progressive Hypoerythemia, Progressive Infantile Poliodystrophy, Progressive Lenticular Degeneration, Progressive Lipodystrophy, Progressive Muscular Dystrophy of Childhood, Progressive Myoclonic Epilepsy, Progressive Osseous Heteroplasia, Progressive Pallid Degeneration Syndrome, Progressive Pallid Degeneration Syndrome, Progressive Spinobulbar Muscular Atrophy, Progressive Supranuclear Palsy, Progressive Systemic Sclerosis, Progressive Tapetochoroidal Dystrophy, Proline Oxidase Deficiency, Propionic Acidemia, Propionic Acidemia, Propionic Acidemia Type I (PCCA Deficiency), Propionic Acidemia Type II (PCCB Deficiency), Propionyl CoA Carboxylase Deficiency, Propionyl CoA Carboxylase Deficiency, Protanomaly, Protanopia, Protein-Losing Enteropathy Secondary to Congestive Heart Failure, Proteus Syndrome, Proximal Deletion of 4q Included, Proximal Deletion of 4q-Included, PRP, PRS, Prune Belly Syndrome, PS, Pseudo-Hurler Polydystrophy, Pseudo-Polydystrophy, Pseudoacanthosis Nigricans, Pseudoachondroplasia, Pseudocholinesterase Deficiency, Pseudogout Familial, Pseudohemophilia, Pseudohermaphroditism, Pseudohermaphroditism, Pseudohermaphroditism-Nephron Disorder-Wilm's Tumor, Pseudohypertrophic Muscular Dystrophy, Pseudohypoparathyroidism, Pseudohypoparathyroidism, Pseudohypophosphatasia, Pseudopolydystrophy, Pseudothalidomide Syndrome, Pseudoxanthoma Elasticum, Pseudoxanthoma Elasticum, Psoriasis, Psorospermosis Follicularis, PSP, PSS, Psychomotor Convulsion, Psychomotor Epilepsy, Psychomotor Equivalent Epilepsy, PTC Deficiency, Pterygium, Pterygium Colli Syndrome, Pterygium Universale, Pterygolymphangiectasia, Pulmonary Atresia, Pulmonary Lymphangiomyomatosis, Pulmonary Stenosis, Pulmonic Stenosis-Ventricular Septal Defect, Pulp Stones, Pulpal Dysplasia, Pulseless Disease, Pure Alymphocytosis, Pure Cutaneous Histiocytosis, Purine Nucleoside Phosphorylase Deficiency, Purpura Hemorrhagica, Purtilo Syndrome, PXE, PXE, PXE Dominant Type, PXE Recessive Type, Pycnodysostosis, Pyknodysostosis, Pyknoepilepsy, Pyroglutamic Aciduria, Pyroglutamicaciduria, Pyrroline Carboxylate Dehydrogenase Deficiency, Pyruvate Carboxylase Deficiency, Pyruvate Carboxylase Deficiency Group A, Pyruvate Carboxylase Deficiency Group B, Pyruvate Dehydrogenase Deficiency, Pyruvate Dehydrogenase Deficiency, Pyruvate Dehydrogenase Deficiency, Pyruvate Kinase Deficiency, q25-qter, q26 or q27-qter, q31 or 32-qter, QT Prolongation with Extracellular Hypohypocalcinemia, QT Prolongation without Congenital Deafness, QT Prolonged with Congenital Deafness, Quadriparesis of Cerebral Palsy, Quadriplegia of Cerebral Palsy, Quantal Squander, Quantal Squander, r4, r6, r14, r 18, r21, r22, Rachischisis Posterior, Radial Aplasia-Amegakaryocytic Thrombocytopenia, Radial Aplasia-Thrombocytopenia Syndrome, Radial Nerve Palsy, Radicular Neuropathy Sensory, Radicular Neuropathy Sensory, Radicular Neuropathy Sensory Recessive, Radicular Dentin Dysplasia, Rapid-onset Dystonia-parkinsonism, Rapp-Hodgkin Syndrome, Rapp-Hodgkin (hypohidrotic) Ectodermal Dysplasia syndrome, Rapp-Hodgkin Hypohidrotic Ectodermal Dysplasias, Rare hereditary ataxia with polyneuritic changes and deafness caused by a defect in the enzyme phytanic acid hydroxylase, Rautenstrauch-Wiedemann Syndrome, Rautenstrauch-Wiedemann Type Neonatal Progeria, Raynaud's Phenomenon, RDP, Reactive Functional Hypoglycemia, Reactive Hypoglycemia Secondary to Mild Diabetes, Recessive Type Kenny-Caffe Syndrome, Recklin Recessive Type Myotonia Congenita, Recklinghausen Disease, Rectoperineal Fistula, Recurrent Vomiting, Reflex Neurovascular Dystrophy, Reflex Sympathetic Dystrophy Syndrome, Refractive Errors, Refractory Anemia, Refrigeration Palsy, Refsum Disease, Refsum's Disease, Regional Enteritis, Reid-Barlow's syndrome, Reifenstein Syndrome, Reifenstein Syndrome, Reiger Anomaly-Growth Retardation, Reiger Syndrome, Reimann Periodic Disease, Reimann's Syndrome, Reis-Bucklers Corneal Dystrophy, Reiter's Syndrome, Reiter's Syndrome, Relapsing Guillain-Barre Syndrome, Relapsing-Remitting Multiple Sclerosis, Renal Agenesis, Renal Dysplasia-Blindness Hereditary, Renal Dysplasia-Retinal Aplasia Loken-Senior Type, Renal Glycosuria, Renal Glycosuria Type A, Renal Glycosuria Type B, Renal Glycosuria Type O, Renal-Oculocerebrodystrophy, Renal-Retinal Dysplasia with Medullary Cystic Disease, Renal-Retinal Dysplasia with Medullary Cystic Disease, Renal-Retinal Dystrophy Familial, Renal-Retinal Syndrome, Rendu-Osler-Weber. Syndrome, Respiratory Acidosis, Respiratory Chain Disorders, Respiratory Myoclonus, Restless Legs Syndrome, Restrictive Cardiomyopathy, Retention Hyperlipemia, Rethore Syndrome (obsolete), Reticular Dysgenesis, Retinal Aplastic-Cystic Kidneys-Joubert Syndrome, Retinal Cone Degeneration, Retinal Cone Dystrophy, Retinal Cone-Rod Dystrophy, Retinitis Pigmentosa, Retinitis Pigmentosa and Congenital Deafness, Retinoblastoma, Retinol Deficiency, Retinoschisis, Retinoschisis Juvenile, Retraction Syndrome, Retrobulbar Neuropathy, Retrolenticular Syndrome, Ret. Syndrome, Reverse Coaretion, Reye Syndrome, Reye's Syndrome, RGS, Rh Blood Factors, Rh Disease, Rh Factor Incompatibility, Rh Incompatibility, Rhesus Incompatibility, Rheumatic Fever, Rheumatoid Arthritis, Rheumatoid Myositis, Rhinosinusogenic Cerebral Arachnoiditis, Rhizomelic Chondrodysplasia Punctata (RCDP),Acatalasemia,Classical Refsum disease, RES, Rhythmical Myoclonus, Rib Gap Defects with Micrognathia, Ribbing Disease (obsolete), Ribbing Disease, Richner-Hanhart Syndrome, Rieger Syndrome, Rieter's Syndrome, Right Ventricular Fibrosis, Riley-Day Syndrome, Riley-Smith syndrome, Ring Chromosome 14, Ring Chromosome 18, Ring 4, Ring 4 Chromosome, Ring 6, Ring 6 Chromosome, Ring 9, Ring 9 Chromosome R9, Ring 14, Ring 15, Ring 15 Chromosome (mosaic pattern), Ring 18, Ring Chromosome 18, Ring 21, Ring 21 Chromosome, Ring 22, Ring 22 Chromosome, Ritter Disease, Ritter-Lyell Syndrome, RLS, RMSS, Roberts SC-Phocomelia Syndrome, Roberts Syndrome, Roberts Tetraphocomelia Syndrome, Robertson's Ectodermal Dysplasias, Robin Anomalad, Robin Sequence, Robin Syndrome, Robinow Dwarfism, Robinow Syndrome, Robinow Syndrome Dominant Form, Robinow Syndrome Recessive Form, Rod Myopathy, Roger Disease, Rokitansky's Disease, Romano-Ward Syndrome, Romberg Syndrome, Rootless Teeth, Rosenberg-Chutorian Syndrome, Rosewater Syndrome, Rosewater Syndrome, Rosselli-Gulienatti Syndrome, Rothmund-Thomson Syndrome, Roussy-Levy Syndrome, RP, RS X-Linked, RS, RS, RSDS, RSH Syndrome, RSS, RSTS, RTS, RTS, RTS, Rubella Congenital, Rubinstein Syndrome, Rubinstein-Taybi Syndrome, Rubinstein Taybi Broad Thumb-Hallux syndrome, Rufous Albinism, Ruhr's Syndrome, Russell's Diencephalic Cachexia, Russell's Syndrome, Russell Syndrome, Russell-Silver Dwarfism, Russell-Silver Syndrome, Russell-Silver Syndrome X-linked, Ruvalcaba-Myhre-Smith syndrome (RMSS), Ruvalcaba Syndrome, Ruvalcaba Type Osseous Dysplasia with Mental Retardation, Sacral Regression, Sacral Agenesis Congenital, SAE, Saethre-Chotzen Syndrome, Sakati, Sakati Syndrome, Sakati-Nyhan Syndrome, Salaam Spasms, Salivosudoriparous Syndrome, Salzman Nodular Corneal Dystrophy, Sandhoff Disease, Sanfilippo Syndrome, Sanfilippo Type A, Sanfilippo Type B, Santavuori Disease, Santavuori-Haltia Disease, Sarcoid of Boeck, Sarcoidosis, Sarcoidosis, Sathre-chotzen, Saturday Night Palsy, SBMA, SC Phocomelia Syndrome, SC Syndrome, SCA 3, SCAD Deficiency, SCAD Deficiency Adult-Onset Localized, SCAD Deficiency Congenital Generalized, SCAD, SCAD, SCAD, SCADH Deficiency, Scalded Skin Syndrome, Scalp Defect Congenital, Scaphocephaly, Scaphocephaly, Scaphocephaly, Scapula Elevata, Scapuloperoneal Myopathy, Scapuloperoneal Muscular Dystrophy, Scapuloperoneal Syndrome Myopathic Type, Scarring Bullosa, Scarring Bullosa, SCHAD, Schaumann's Disease, Scheie Syndrome, Schereshevkii-Tumer Syndrome, Schilder Disease, Schilder Encephalitis, Schilder's Disease, Schindler Disease Type I (Infantile Onset), Schindler Disease Infantile Onset, Schindler Disease, Schindler Disease Type II (Adult Onset), Schinzel Syndrome, Schinzel-Giedion Syndrome, Schinzel Acrocallosal Syndrome, Schinzel-Giedion Midface-Retraction Syndrome, Schizencephaly, Schmid Type Metaphyseal Chondrodysplasia, Schmid Metaphyseal Dysostosis, Schmid-Fraccaro Syndrome, Schmidt Syndrome, Schopf-Schultz-Passarge Syndrome, Schueller-Christian Disease, Schut-Haymaker Type, Schwartz-Jampel-Aberfeld Syndrome, Schwartz-Jampel Syndrome Types 1A and 1B, Schwartz-Jampel Syndrome, Schwartz-Jampel Syndrome Type 2, SCI, D SCID, Scleroderma, Scleroderma, Sclerosis Familial Progressive Systemic, Sclerosis Diffuse Familial Brain, Scott Craniodigital Syndrome With Mental Retardation, Scrotal Tongue, SCS, SCS, SD, SDS, SDYS, Seasonal Conjunctivitis, Sebaceous Nevus Syndrome, Sebaceous nevus, Seborrheic Keratosis, Seborrheic Warts, Seckel Syndrome, Seckel Type Dwarfism, Second Degree Congenital Heart Block, Secondary Amyloidosis, Secondary Blepharospasm, Secondary Non-tropical Sprue, Secondary Brown Syndrome, Secondary Beriberi, Secondary Generalized Amyloidosis, Secondary Dystonia, Secretory Component Deficiency, Secretory IgA Deficiency, SED Tarda, SED Congenital, SEDC, Segmental linear achromic nevus, Segmental Dystonia, Segmental Myoclonus, Seip Syndrome, Seitelberger Disease, Seitelberger Disease, Seizures, Selective Deficiency of IgG Subclasses, Selective Mutism, Selective Deficiency of IgG Subclass, Selective IgM Deficiency, Selective Mutism, Selective IgA Deficiency, Self-Healing Histiocytosis, Semilobar Holoprosencephaly, Seminiferous Tubule Dysgenesis, Senile Retinoschisis, Senile Warts, Senior-Loken Syndrome, Sensory Neuropathy Hereditary Type I, Sensory Neuropathy Hereditary Type II, Sensory Neuropathy Hereditary Type I, Sensory Radicular Neuropathy, Sensory Radicular Neuropathy, Sensory Radicular Neuropathy Recessive, Septic Progressive Granulomatosis, Septo-Optic Dysplasia, Serous Circumscribed Meningitis, Serum Protease Inhibitor Deficiency, Serum Camosinase Deficiency, Setleis Syndrome, Severe Combined Immunodeficiency, Severe Combined Immunodeficiency with Adenosine Deaminase Deficiency, Severe Combined Immunodeficiency (SCID), Sex Reversal, Sexual Infantilism, SGB Syndrome, Sheehan Syndrome, Shields Type Dentinogenesis Imperfecta, Shingles,varicella-zoster virus, Ship Beriberi, SHORT Syndrome, Short Arm 18 Deletion Syndrome, Short Chain Acyl CoA Dehydrogenase Deficiency, Short Chain Acyl-CoA Dehydrogenase (SCAD) Deficiency, Short Stature and Facial Telangiectasis, Short Stature Facial/Skeletal Anomalies-Retardation-Macrodontia, Short Stature-Hyperextensibility-Rieger Anomaly-Teething Delay, Short Stature-Onychodysplasia, Short Stature Telangiectatic Erythema of the Face, SHORT Syndrome, Shoshin Beriberi, Shoulder girdle syndrome, Shprintzen-Goldberg Syndrome, Shulman Syndrome, Shwachman-Bodian Syndrome, Shwachman-Diamond Syndrome, Shwachman Syndrome, Shwachman-Diamond-Oski Syndrome, Shwachmann Syndrome, Shy Drager Syndrome, Shy-Magee Syndrome, SI Deficiency, Sialidase Deficiency, Sialidosis Type I Juvenile, Sialidosis Type II Infantile, Sialidosis, Sialolipidosis, Sick Sinus Syndrome, Sickle Cell Anemia, Sickle Cell Disease, Sickle Cell-Hemoglobin C Disease, Sickle Cell-Hemoglobin D Disease, Sickle Cell-Thalassemia Disease, Sickle Cell Trait, Sideroblastic Anemias, Sideroblastic Anemia, Sideroblastosis, Sideroblastosis, SIDS, Siegel-Cattan-Mamou Syndrome, Siemens-Bloch type Pigmented Dermatosis, Siemens Syndrome, Siewerling-Creutzfeldt Disease, Siewert Syndrome, Silver Syndrome, Silver-Russell Dwarfism, Silver-Russell Syndrome, Simmond's Disease, Simons Syndrome, Simplex Epidermolysis Bullosa, Simpson Dysmorphia Syndrome, Simpson-Golabi-Behmel Syndrome, Sinding-Larsen-Johansson Disease, Singleton-Merten Syndrome, Sinus Arrhythmia, Sinus Venosus, Sinus tachycardia, Sirenomelia Sequence, Sirenomelus, Situs Inversus Bronchiectasis and Sinusitis, SJA Syndrome, Sjogren Larsson Syndrome Ichthyosis, Sjogren Syndrome, Sjogren Larsson Syndrome Ichthyosis, Sjogren's Syndrome, SJS, Skeletal dysplasia, Skeletal Dysplasia Weismann Netter Stuhl Type, Skin Peeling Syndrome, Skin Neoplasms, Skull Asymmetry and Mild Retardation, Skull Asymmetry and Mild Syndactyly, SLE, Sleep Epilepsy, Sleep Apnea, SLO, Sly Syndrome, SMA, SMA Infantile Acute Form, SMA I, SMA III, SMA type I, SMA type II, SMA type III, SMA3, SMAX1, SMCR, Smith Lemli Opitz Syndrome, Smith Magenis Syndrome, Smith-Magenis Chromosome Region, Smith-McCort Dwarfism, Smith-Opitz-Inborn Syndrome, Smith Disease, Smoldering Myeloma, SMS, SMS, SNE, Sneezing From Light Exposure, Sodium valproate, Solitary Plasmacytoma of Bone, Sorsby Disease, Sotos Syndrome, Souques-Charcot Syndrome, South African Genetic Porphyria, Spasmodic Dysphonia, Spasmodic Torticollis, Spasmodic Torticollis, Spasmodic Wryneck, Spastic Cerebral Palsy, Spastic Colon, Spastic Dysphonia, Spastic Paraplegia, SPD Calcinosis, Specific Antibody Deficiency with Normal Immunoglobulins, Specific Reading Disability, SPH2, Spherocytic Anemia, Spherocytosis, Spherophakia-Brachymorphia Syndrome, Sphingomyelin Lipidosis, Sphingomyelinase Deficiency, Spider fingers, Spielmeyer-Vogt Disease, Spielmeyer-Vogt-Batten Syndrome, Spina Bifida, Spina Bifida, Spina Bifida Aperta, Spinal Arachnoiditis, Spinal Arteriovenous Malformation, Spinal Ataxia Hereditofamilial, Spinal and Bulbar Muscular Atrophy, Spinal Diffuse Idiopathic Skeletal Hyperostosis, Spinal DISH, Spinal Muscular Atrophy, Spinal Muscular Atrophy, Spinal Muscular Atrophy All Types, Spinal Muscular Atrophy Type ALS, Spinal Muscular Atrophy-Hypertrophy of the Calves, Spinal Muscular Atrophy Type I, Spinal Muscular Atrophy Type III, Spinal Muscular Atrophy type 3, Spinal Muscular Atrophy-Hypertrophy of the Calves, Spinal Ossifying Arachnoiditis, Spinal Stenosis, Spino Cerebellar Ataxia, Spinocerebellar Atrophy Type I, Spinocerebellar Ataxia Type I (SCAl), Spinocerebellar Ataxia Type II (SCAII), Spinocerebellar Ataxia Type III (SCAIII), Spinocerebellar Ataxia Type III (SCA 3), Spinocerebellar Ataxia Type IV (SCAIV), Spinocerebellar Ataxia Type V (SCAV), Spinocerebellar Ataxia Type VI (SCA-VI), Spinocerebellar Ataxia Type VII (SCAVII), Spirochetal Jaundice, Splenic Agenesis Syndrome, Splenic Ptosis, Splenoptosis, Split Hand Deformity-Mandibulofacial Dysostosis, Split Hand Deformity-Mandibulofacial Dysostosis, Split Hand Deformity, Split-Hand Deformity, Spondyloarthritis, Spondylocostal Dysplasia—Type I, Spondyloepiphyseal Dysplasia Tarda, Spondylothoracic Dysplasia, Spondylotic Caudal Radiculopathy, Sponge Kidney, Spongioblastoma Multiforme, Spontaneous Hypoglycemia, Sprengel Deformity, Spring Ophthalmia, SRS, ST, Stale Fish Syndrome, Staphyloccal Scalded Skin Syndrome, Stargardt's Disease, Startle Disease, Status Epilepticus, Steele-Richardson-Olszewski Syndrome, Steely Hair Disease, Stein-Leventhal Syndrome, Steinert Disease, Stengel's Syndrome, Stengel-Batten-Mayou-Spielmeyer-Vogt-Stock Disease, Stenosing Cholangitis, Stenosis of the Lumbar Vertebral Canal, Stenosis, Steroid Sulfatase Deficiency, Stevanovic's Ectodermal Dysplasias, Stevens Johnson Syndrome, Stevens-Johnson Syndrome, STGD, Stickler Syndrome, Stickler Syndrome, Stiff-Man Syndrome, Stiff Man Syndrome, Stiff Person Syndrome, Still's Disease, Stilling-Turk-Duane Syndrome, Stillis Disease, Stimulus-Sensitive Myoclonus, Stone Man Syndrome, Stone Man, Streeter Anomaly, Striatonigral Degeneration Autosomal Dominant Type, Striopallidodentate Calcinosis, Stroma, Descemet's Membrane, Stromal Corneal Dystrophy, Struma Lymphomatosa, Sturge-Kalischer-Weber Syndrome, Sturge Weber Syndrome, Sturge-Weber Phakomatosis, Subacute Necrotizing Encephalomyelopathy, Subacute Necrotizing Encephalomyelopathy, Subacute Spongiform Encephalopathy, Subacute Necrotizing Encephalopathy, Subacute Sarcoidosis, Subacute Neuronopathic, Subaortic Stenosis, Subcortical Arteriosclerotic Encephalopathy, Subendocardial Sclerosis, Succinylcholine Sensitivity, Sucrase-Isomaltase Deficiency Congenital, Sucrose-Isomaltose Malabsorption Congenital, Sucrose Intolerance Congenital, Sudanophilic Leukodystrophy ADL, Sudanophilic Leukodystrophy Pelizaeus-Merzbacher Type, Sudanophilic Leukodystrophy Included, Sudden Infant Death Syndrome, Sudeck's Atrophy, Sugio-Kajii Syndrome, Summerskill Syndrome, Summit Acrocephalosyndactyly, Summitt's Acrocephalosyndactyly, Summitt Syndrome, Superior Oblique Tendon Sheath Syndrome, Suprarenal glands, Supravalvular Aortic Stenosis, Supraventricular tachycardia, Surdicardiac Syndrome, Surdocardiac Syndrome, SVT, Sweat Gland Abscess, Sweating Gustatory Syndrome, Sweet Syndrome, Swiss Cheese Cartilage Syndrome, Syndactylic Oxycephaly, Syndactyly Type I with Microcephaly and Mental Retardation, Syndromatic Hepatic Ductular Hypoplasia, Syringomyelia, Systemic Aleukemic Reticuloendotheliosis, Systemic Amyloidosis, Systemic Carnitine Deficiency, Systemic Elastorrhexis, Systemic Lupus Erythematosus, Systemic Mast Cell Disease, Systemic Mastocytosis, Systemic-Onset Juvenile Arthritis, Systemic-Onset Juvenile Arthritis, Systemic Sclerosis, Systopic Spleen, T-Lymphocyte Deficiency, Tachyalimentation Hypoglycemia, Tachycardia, Takahara syndrome, Takayasu Disease, Takayasu Arteritis, Takayasu Arteritis, Talipes Calcaneus, Talipes Equinovarus, Talipes Equinus, Talipes Varus, Talipes Valgus, Tandem Spinal Stenosis, Tangier Disease, Tapetoretinal Degeneration, TAR Syndrome, Tardive Dystonia, Tardive Muscular Dystrophy, Tardive Dyskinesia, Tardive Oral Dyskinesia, Tardivc Dyskinesia, Tardive Dystonia, Tardy Ulnar Palsy, Target Cell Anemia, Tarsomegaly, Tarui Disease, TAS Midline Defects Included, TAS Midline Defect, Tay Sachs Disease, Tay Sachs Sphingolipidosis, Tay Sachs Disease, Tay Syndrome Ichthyosis, Tay Sachs Sphingolipidosis, Tay Syndrome Ichthyosis, Taybi Syndrome Type I, Taybi Syndrome, TCD, TCOF1, TCS, TD, TDO Syndrome, TDO-I, TDO-II, TDO-III, Telangiectasis, Telecanthus with Associated Abnormalities, Telecanthus With Associated Abnormalities, Telecanthus-Hypospadias Syndrome, Temporal Lobe Epilepsy, Temporal Arteritis/Giant Cell Arteritis, Temporal Arteritis, TEN, Tendon Sheath Adherence Superior Obliqu, Tension Myalgia, Terminal Deletion of 4q Included, Terminal Deletion of 4q-Included, Terrian Corneal Dystrophy, Teschler-Nicola/Killian Syndrome, Tethered Spinal Cord Syndrome, Tethered Cord Malformation Sequence, Tethered Cord Syndrome, Tethered Cervical Spinal Cord Syndrome, Tetrahydrobiopterin Deficiencies, Tetrahydrobiopterin Deficiencies, Tetralogy of Fallot, Tetralogy of Fallot, Tetraphocomelia-Thrombocytopenia Syndrome, Tetrasomy Short Arm of Chromosome 9, Tetrasomy 9p, Tetrasomy Short Arm of Chromosome 18, Thalamic Syndrome, Thalamic Pain Syndrome, Thalamic Hyperesthetic Anesthesia, Thalassemia Intermedia, Thalassemia Minor, Thalassemia Major, Thiamine Deficiency, Thiamine-Responsive Maple Syrup Urine Disease, Thin-Basement-Membrane Nephropathy, Thiolase deficiency,RCDP,Acyl-CoA dihydroxyacetonephosphate acyltransferase, Third and Fourth Pharyngeal Pouch Syndrome, Third Degree Congenital (Complete) Heart Block, Thomsen Disease, Thoracic-Pelvic-Phalangeal Dystrophy, Thoracic Spinal Canal, Thoracoabdominal Syndrome, Thoracoabdominal Ectopia Cordis Syndrome, Three M Syndrome, Three-M Slender-Boned Nanism, Thrombasthenia of Glanzmann and Naegeli, Thrombocythemia Essential, Thrombocytopenia-Absent Radius Syndrome, Thrombocytopenia-Hemangioma Syndrome, Thrombocytopenia-Absent Radii Syndrome, Thrombophilia Hereditary Due to AT III, Thrombotic Thrombocytopenic Purpura, Thromboulcerative Colitis, Thromboulcerative Colitis, Thymic Dysplasia with Normal Immunoglobulins, Thymic Agenesis,Thymic Aplasia DiGeorge Type, Thymic Hypoplasia Agammaglobulinemias Primary Included, Thymic Hypoplasia DiGeorge Type, Thymus Congenital Aplasia, Tic Douloureux, Tics, Tinel's syndrome, Tolosa Hunt Syndrome, Tonic Spasmodic Torticollis, Tonic Pupil Syndrome, Tooth and Nail Syndrome, Tooth and Nail Syndrome, Torch Infection, TORCH Syndrome, Torsion Dystonia, Torticollis, Torticollis, Total Lipodystrophy, Total anomalous pulmonary venous connection, Touraine's Aphthosis, Tourette Syndrome, Tourette's disorder, Townes-Brocks Syndrome, Townes Syndrome, Toxic Paralytic Anemia, Toxic Epidermal Necrolysis, Toxopachyosteose Diaphysaire Tibio-Peroniere, Toxopachyosteose, Toxoplasmosis Other Agents Rubella Cytomegalovirus Herpes Simplex, Tracheoesophageal Fistula with or without Esophageal Atresia, Tracheoesophageal Fistula, Transient neonatal myasthenia gravis, Transitional Atrioventricular Septal Defect, Transposition of the great arteries, Transtelephonic Monitoring, Transthyretin Methionine-30 Amyloidosis (Type I), Trapezoidocephaly-Multiple Synostosis Syndrome, Treacher Collins Syndrome, Treacher Collins-Franceschetti Syndrome 1, Trevor Disease, Triatrial Heart, Tricho-Dento-Osseous Syndrome, Trichodento Osseous Syndrome, Trichopoliodystrophy, Trichorhinophalangeal Syndrome, Trichorhinophalangeal Syndrome, Tricuspid atresia, Trifunctional Protein Deficiency, Trigeminal Neuralgia, Triglyceride Storage Disease Impaired Long-Chain Fatty Acid Oxidation, Trigonitis, Trigonocephaly, Trigonocephaly, Trigonocephaly, Trigonocephaly Syndrome, Trigonocephaly “C” Syndrome, Trimethylaminuria, Triphalangeal Thumbs-Hypoplastic Distal Phalanges-Onychodystrophy, Triphalangeal Thumb Syndrome, Triple Symptom Complex of Behcet, Triple X Syndrome, Triplo X Syndrome, Triploid Syndrome, Triploidy, Triploidy Syndrome, Trismus-Pseudocamptodactyly Syndrome, Trisomy, Trisomy G Syndrome, Trisomy X, Trisomy 6q Partial, Trisomy 6q Syndrome Partial, Trisomy 9 Mosaic, Trisomy 9P Syndrome (Partial) Included, Trisomy llq Partial, Trisomy 14 Mosaic, Trisomy 14 Mosaicism Syndrome, Trisomy 21 Syndrome, Trisomy 22 Mosaic, Trisomy 22 Mosaicism Syndrome, TRPS, TRPS 1, TRPS2, TRPS3, True Hermaphroditism, True Hermaphroditism, Truncus arteriosus, Tryptophan Malabsorption, Tryptophan Pyrrolase Deficiency, TS, TTP, TTTS, Tuberous Sclerosis, Tubular Ectasia, Turcot Syndrome, Turner Syndrome, Turner-Kieser Syndrome, Turner Phenotype with Normal Chromosomes (Karyotype), Turner-Varny Syndrome, Turricephaly, Twin-Twin Transfusion Syndrome, Twin-to-Twin Transfusion Syndrome, Type A, Type B, Type AB, Type O, Type I Diabetes, Type I Familial Incomplete Male, Type I Familial Incomplete Male Pseudohermaphroditism, Type I Gaucher Disease, Type I (PCCA Deficiency), Type I Tyrosinemia, Type II Gaucher Disease, Type II Histiocytosis, Type II (PCCB Deficiency), Type II Tyrosinnemia, Type IIA Distal Arthrogryposis Multiplex Congenita, Type III Gaucher Disease, Type III Tyrosinemia, Type III Dentinogenesis Imperfecta, Typical Retinoschisis, Tyrosinase Negative Albinism (Type I), Tyrosinase Positive Albinism (Type II), Tyrosinemia type 1 acute form, Tyrosinemia type 1 chronic form, Tyrosinosis, UCE, Ulcerative Colitis, Ulcerative Colitis Chronic Non-Specific, Ulnar-Mammary Syndrome, Ulnar-Mammary Syndrome of Pallister, Ulnar Nerve Palsy, UMS, Unclassified FODs, Unconjugated Benign Bilirubinemiav, Underactivity of Parathyroid, Unilateral Ichthyosiform Erythroderma with Ipsilateral Malformations Limb, Unilateral Chondromatosis, Unilateral Defect of Pectoralis Muscle and Syndactyly of the Hand, Unilateral Hemidysplasia Type, Unilateral Megalencephaly, Unilateral Partial Lipodystrophy, Unilateral Renal Agenesis, Unstable Colon, Unverricht Disease, Unverricht-Lundborg Disease, Unverricht-Lundborg-Laf Disease, Unverricht Syndrome, Upper Limb—Cardiovascular Syndrome (Holt-Oram), Upper Motor Neuron Disease, Upper Airway Apnea, Upper Airway Apnea, Urea Cycle Defects or Disorders, Urea Cycle Disorder Arginase Type, Urea Cycle Disorder Arginino Succinase Type, Urea Cycle Disorders Carbamyl Phosphate Synthetase Type, Urea Cycle Disorder Citrullinemia Type, Urea Cycle Disorders N-Acrtyl Glutamate Synthetase Typ, Urea Cycle Disorder OTC Type, Urethral Syndrome, Urethro-Oculo-Articular Syndrome, Uridine Diphosphate Glucuronosyltransferase Severe Def. Type I, Urinary Tract Defects, Urofacial Syndrome, Uroporphyrinogen III cosynthase, Urticaria pigmentosa, Usher Syndrome, Usher Type I, Usher Type II, Usher Type III, Usher Type IV, Uterine Synechiae, Uoporphyrinogen I-synthase, Uveitis, Uveomeningitis Syndrome, V-CJD, VACTEL Association, VACTERL Association, VACTERL Syndrome, Valgus Calcaneus, Valine Transaminase Deficiency, Valinemia, Valproic Acid, Valproate acid exposure, Valproic acid exposure, Valproic acid, Van Buren's Disease, Van der Hoeve-Habertsma-Waardenburg-Gauldi Syndrome, Variable Onset Immunoglobulin Deficiency Dysgammaglobulinemia, Variant Creutzfeldt-Jakob Disease (V-CJD), Varicella Embryopathy, Variegate Porphyria, Variegate Porphyria, Variegate Porphyria, Vascular Birthmarks, Vascular Dementia Binswanger's Type, Vascular Erectile Tumor, Vascular Hemophilia, Vascular Malformations, Vascular Malformations of the Brain, Vasculitis, Vasomotor Ataxia, Vasopressin-Resistant Diabetes Insipidus, Vasopressin-Sensitive Diabetes Insipidus, VATER Association, Vcf syndrome, Vcfs, Velocardiofacial Syndrome, VeloCardioFacial Syndrome, Venereal Arthritis, Venous Malformations, Ventricular Fibrillation, Ventricular Septal Defects, Congenital Ventricular Defects, Ventricular Septal Defect, Ventricular Tachycardia, Venual Malformations, VEOHD, Vermis Aplasia, Vermis Cerebellar Agenesis, Vernal Keratoconjunctivitis, Verruca, Vertebral Anal Tracheoesophageal Esophageal Radial, Vertebral Ankylosing Hyperostosis, Very Early Onset Huntington's Disease, Very Long Chain Acyl-CoA Dehydrogenase (VLCAD) Deficiency, Vestibular Schwannoma, Vestibular Schwannoma Neurofibromatosis, Vestibulocerebellar, Virchow's Oxycephaly, Visceral Xanthogranulomatosis, Visceral Xantho-Granulomatosis, Visceral Myopathy-External Ophthalmoplegia, Visceromegaly-Umbilical Hernia-Macroglossia Syndrome, Visual Amnesia, Vitamin A Deficiency, Vitamin B-1 Deficiency, Vitelline Macular Dystrophy, Vitiligo, Vitiligo, Vitiligo Capitis, Vitreoretinal Dystrophy, VKC, VKH Syndrome, VLCAD, VLCAD, Vogt Syndrome, Vogt Cephalosyndactyly, Vogt Koyanagi Harada Syndrome, Vogt Koyanagi Harada Syndrome, Vogt Koyanagi Harada Syndrome, Von Bechterew-Strumpell Syndrome, Von Eulenburg Paramyotonia Congenita, Von Frey's Syndrome, Von Gierke Disease, Von Hippel-Lindau Syndrome, Von Mikulicz Syndrome, Von Recklinghausen Disease, Von Willebrandt Disease, VP, Vrolik Disease (Type II), VSD, VSD, Vulgaris Type Disorder of Comification, Vulgaris Type Ichthyosis, W Syndrome, Waardenburg Syndrome, Waardenburg-Klein Syndrome, Waardenburg Syndrome Type I (WS1), Waardenburg Syndrome Type II (WS2), Waardenburg Syndrome Type IIA (WS2A), Waardenburg Syndrome Type IIB (WS2B), Waardenburg Syndrome Type III (WS3), Waardenburg Syndrome Type IV (WS4), Waelsch's Syndrome, WAGR Complex, WAGR Syndrome, WAGR Syndrome, Waldenstroem's Macroglobulinemia, Waldenstrom's Purpura, Waldenstrom's Syndrome, Waldmann Disease, Walker-Warburg Syndrome, Wandering Spleen, Warburg Syndrome, Warm Antibody Hemolytic Anemia, Warm Reacting Antibody Disease, Wartenberg Syndrome, WAS, Water on the Brain, Watson Syndrome, Watson-Alagille Syndrome, Waterhouse-Friderichsen syndrome, Waxy Disease, WBS, Weaver Syndrome, Weaver-Smith Syndrome, Weber-Cockayne Disease, Wegener's Granulomatosis, Wegener's Granulomatosis, Weil Disease, Weil Syndrome, Weill-Marchesani, Weill-Marchesani Syndrome, Weill-Reyes Syndrome, Weismann-Netter-Stuhl Syndrome, Weissenbacher-Zweymuller Syndrome, Wells Syndrome, Wenckebach, Werdnig-Hoffman Disease, Werdnig-Hoffmann Disease, Werdnig-Hoffmann disease, Werdnig-Hoffinan Disease, Werdnig-Hoffinan Paralysis, Werlhofs Disease, Werner Syndrome, Wernicke's (C) I Syndrome, Wernicke's aphasia, Wernicke-Korsakoff Syndrome, West Syndrome, Wet Beriberi, WHCR, Whipple's Disease, Whipple Disease, Whistling face syndrome, Whistling Face-Windmill Vane Hand Syndrome, White-Darier Disease, Whitnall-Norman Syndrome, Whorled nevoid hypermelanosis, WHS, Wieacker Syndrome, Wieacher Syndrome, Wieacker-Wolff Syndrome, Wiedmann-Beckwith Syndrome, Wiedemann-Rautenstrauch Syndrome, Wildervanck Syndrome, Willebrand-Juergens Disease, Willi-Prader Syndrome, Williams Syndrome, Williams Syndrome, Williams-Beuren Syndrome, Wilms' Tumor, Wilms' Tumor-Aniridia-Gonadoblastoma-Mental Retardation Syndrome, Wilms Tumor Aniridia Gonadoblastoma Mental Retardation, Wilms' Tumor-Aniridia-Genitourinary Anomalies-Mental Retardation Syndrome, Wilms Tumor-Pseudohermaphroditism-Nephropathy, Wilms Tumor and Pseudohermaphroditism, Wilms Tumor-Pseuodohermaphroditism-Glomerulopathy, Wilson's Disease, Winchester Syndrome, Winchester-Grossman Syndrome, Wiskott-Aldrich Syndrome, Wiskott-Aldrich Type Immunodeficiency, Witkop Ectodermal Dysplasias, Witkop Tooth-Nail Syndrome, Wittmaack-Ekbom Syndrome, WM Syndrome, WMS, WMS, WNS, Wohlfart-Disease, Wohlfart-Kugelberg-Welander Disease, Wolf Syndrome, Wolf-Hirschhom Chromosome Region (WHCR), Wolf-Hirschhorn Syndrome, Wolff-Parkinson-White Syndrome, Wolff-Parkinson-White syndrome, Wolff Parkinson White Syndrome, Wolfram Syndrome, Wolman Disease (Lysomal Acid Lypase Deficiency), Woody Guthrie's Disease, WPW Syndrome, WPW Syndrome, Writer's Cramp, WS, WS, WS, WSS, WWS, Wyburn-Mason Syndrome, Wyburn-Mason Syndrome, X-Linked Addison's Disease, X-linked Adrenoleukodystrophy (X-ALD), X-linked Adult Onset Spinobulbar Muscular Atrophy, X-linked Adult Spinal Muscular Atrophy, X-Linked Agammaglobulinemia with Growth Hormone Deficiency, X-Linked Agammaglobulinemia, Lymphoproliferate X-Linked Syndrome, X-linked Cardiomyopathy and Neutropenia, X-Linked Centronuclear Myopathy, X-linked Copper Deficiency, X-linked Copper Malabsorption, X-Linked Dominant Conradi-Hunermann Syndrome, X-Linked Dominant Inheritance Agenesis of Corpus Callosum, X-Linked Dystonia-parkinsonism, X-Linked Ichthyosis, X Linked Ichthyosis, X-Linked Infantile Agammaglobulinemia, X-Linked Infantile Nectrotizing Encephalopathy, X-linked Juvenile Retinoschisis, X-linked Lissencephaly, X-linked Lymphoproliferative Syndrome, X-linked Mental Retardation-Clasped Thumb Syndrome, X-Linked Mental Retardation with Hypotonia, X-linked Mental Retardation and Macroorchidism, X-Linked Progressive Combined Variable Immunodeficiency, X-Linked Recessive Conradi-Hunermann Syndrome, X-Linked Recessive Severe Combined Immunodeficiency, X-Linked Recessive Severe Combined Immunodeficiency, X-Linked Retinoschisis, X-linked Spondyloepiphyseal Dysplasia, Xanthine Oxidase Deficiency (Xanthinuria Deficiency, Hereditary), Xanthinuria Deficiency, Hereditary (Xanthine Oxidase Deficiency), Xanthogranulomatosis Generalized, Xanthoma Tuberosum, Xeroderma Pigmentosum, Xeroderma Pigmentosum Dominant Type, Xeroderma Pigmentosum Type A I XPA Classical Form, Xeroderma Pigmentosum Type B II XPB, Xeroderma Pigmentosum Type E V XPE, Xeroderma Pigmentosum Type C III XPC, Xeroderma Pigmentosum Type D IV XPD, Xeroderma Pigmentosum Type F VI XPF, Xeroderma Pigmentosum Type G VII XPG, Xeroderma Pigmentosum Variant Type XP-V, Xeroderma-Talipes-and Enamel Defect, Xerodermic Idiocy, Xerophthalmia, Xerotic Keratitis, XLP, XO Syndrome, XP, XX Male Syndrome,Sex Reversal, XXXXX Syndrome, XXY Syndrome, XYY Syndrome, XYY Chromosome Pattern, Yellow Mutant Albinism, Yellow Nail Syndrome, YKL, Young Female Arteritis, Yunis-Varon Syndrome, YY Syndrome, Z-E Syndrome, Z- and -Protease Inhibitor Deficiency, Zellweger Syndrome, Zellweger syndrome, Zellweger cerebro-hepato-renal syndrome, ZES, Ziehen-Oppenheim Disease (Torsion Dystonia), Zimmermann-Laband Syndrome, Zinc Deficiency Congenital, Zinsser-Cole-Engman Syndrome, ZLS, Zollinger-Ellison Syndrome.

An agent includes proteinaceous or non-proteinaceous molecules such as antibodies, natural products, chemical entities or nucleic acid molecules (including antisense molecules, sense molecules, ribozymes, ds-RNA molecules or DNA-targeting molecules).

An “effective amount” means an amount necessary at least partly to attain the desired immune response (e.g. against AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725) or to delay the onset or inhibit progression or halt altogether the onset or progression of a particular condition.

In accordance with these methods, AGT-711, AGT-712, AGT-713, AGT-714, AGT-715, AGT-716, AGT-717, AGT-718, AGT-720, AGT-721, AGT-723, AGT-724, AGT-726, AGT-719 and/or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or agents capable of modulating the expression or activity of said molecules may be co-administered with one or more other compounds or other molecules. By “co-administered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect, the present invention relates to the use of an agent capable of modulating the expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or a derivative, homolog or analog thereof in the manufacture of a medicament for the treatment of a condition characterized inter alia a myopathy, obesity, anorexia, weight maintenance, diabetes, disorders associated with mitochondrial dysfunction, genetic disorders and/or metabolic energy levels.

In still yet another aspect, the present invention relates to the use of an agent capable of modulating the activity of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or a derivative, homolog, analog, chemical equivalent or mimetic thereof in the manufacture of a medicament for the treatment of a condition characterized by inter alia a myopathy, obesity, anorexia, weight maintenance, diabetes, disorders associated with mitochondrial dysfunction, genetic disorders and/or metabolic energy levels.

A further aspect of the present invention relates to the use AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or derivative, homolog or analog thereof or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or derivative, homolog, analog, chemical equivalent or mimetic thereof in the manufacture of a medicament for the treatment of a condition characterized by inter alia a myopathy, obesity, anorexia, weight maintenance, diabetes, disorders associated with mitochondrial dysfunction, genetic disorders and/or metabolic energy levels.

Still yet another aspect of the present invention relates to agents for use in modulating the expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or a derivative, homolog or analog thereof.

A further aspect relates to agents for use in AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 activity or a derivative, homolog, analog, chemical equivalent or mimetic thereof.

Still another aspect of the present invention relates to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and/or AGT-725 or derivative, homolog or analog thereof AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or derivative, homolog, analog, chemical equivalent or mimetic thereof for use in treating a condition characterized by one or more symptoms of inter alia a myopathy, obesity, anorexia, weight maintenance, diabetes, disorders associated with mitochondrial dysfunction, genetic disorders and/or metabolic energy levels.

In a related aspect of the present invention, the mammal undergoing treatment may be a human or an animal in need of therapeutic or prophylactic treatment.

The terms “treating” and “treatment” as used herein refer to a reduction in the severity and/or frequency of symptoms associated with inter alia a myopathy, obesity, anorexia, weight maintenance, diabetes, disorders associated with mitochondrial dysfunction, genetic disorders and/or metabolic energy levels, elimination of symptoms and/or the underlying cause, prevention of the occurrence of symptoms of disease and/or the underlying cause and improvement or remediation of damage.

“Treating” a subject may involve prevention of the disorder or disease condition or adverse physiological event in a susceptible individual as well as treatment of a clinically symptomatic individual by inhibiting a disease or disorder. Generally, such conditions involve, weakness (which may be intermittent), neuropathic pain, absent reflexes, gastrointestinal problem (gastroesophogeal reflux, delayed gastric emptying, constipation, pseudo-obstruction), fainting, absent or excessive sweating resulting in temperature regulation problems weakness, hypotonia, cramping, muscle pain, proximal renal tubular wasting resulting in loss of protein, magnesium, phosphorous, calcium and other electrolytes, cardiac conduction defects (heart blocks) and cardiomyopathy, hypoglycemia (low blood sugar) and liver failure, visual loss and blindness, hearing loss and deafness, diabetes and exocrine pancreatic failure (inability to make digestive enzymes), mitochondrial dysfunction, including failure to gain weight, short statue, fatigue and respiratory problems.

Accordingly, the present invention contemplates in one embodiment a composition comprising a modulator of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 expression or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 activity and one or more pharmaceutically acceptable carriers and/or diluents. In another embodiment, the composition comprises AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or a derivative, homolog, analog or mimetic thereof and one or more pharmaceutically acceptable carriers and/or diluents. The compositions may also comprise leptin or modulations of leptin activity or ob expression.

For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fingi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifingal agents, for example, parabens, chlorobutanol, phenol, sorbic acid, thirmerosal and the like. In many cases, it will be preferable to include isotonic agents, for example, sugars or. sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 and AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 including AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 themselves are suitably protected, they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavoring agent such as peppermint, oil of wintergreen, or cherry flavoring. When the dosage unit form is a capsule, it may contain, in addition to materials of the above type, a liquid carier, Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antiflngal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 will range from 0.01 ng/kg/body weight to above 10,000 mg/kg/body weight. Alternative amounts range from 0.1 ng/kg/body weight to above 1000 mg/kg/body weight. The active ingredients may be administered per minute, hour, day, week, month or year depending on the condition being treated, The route of administration may vary and includes intravenous, intraperitoneal, subcutaneous, intramuscular, intranasal, via suppository, via infusion, via drip, orally or via other convenient means.

The pharmaceutical composition may also comprise genetic molecules such as a vector capable of transfecting target cells where the vector carries a nucleic acid molecule capable of modulating AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 expression or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 activity. The vector may, for example, be a viral vector.

Still another aspect of the present invention is directed to antibodies to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 and their derivatives and homologs insofar as AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 are proteins. Such antibodies may be monoclonal or polyclonal and may be selected from naturally occurring antibodies to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or may be specifically raised AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or derivatives or homologs thereof. In the case of the latter, AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or their derivatives or homologs may first need to be associated with a carrier molecule. The antibodies and/or recombinant AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or their derivatives of the present invention are particularly useful as therapeutic or diagnostic agents.

For example, AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 and their derivatives can be used to screen for naturally occurring antibodies AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 which may occur in certain autoimmune diseases or where cell death is occurring. These may occur, for example, in some autoimmune diseases. Alternatively, specific antibodies can be used to screen for AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725. Techniques for such assays are well known in the art and include, for example, sandwich assays and ELISA.

Antibodies to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 of the present invention may be monoclonal or polyclonal and may be selected from naturally occurring antibodies to the AGT-711, AGT-712, AGT-713, AGT-714, AGT-715, AGT-716, AGT-717, AGT-718, AGT-720, AGT-721, AGT-723, AGT-724, AGT726, AGT-719, AGT-722 and AGT-725 or may be specifically raised to the AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or their derivatives. In the case of the latter, the AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 protein may need first to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725.

For example, specific antibodies can be used to screen AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 proteins. The latter would be important, for example, as a means for screening for levels of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 in a cell extract or other biological fluid or purifying AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies) directed to the first mentioned antibodies discussed above. Both the first and second antibodies may be used in detection assays or a first antibody may be used with a comm. ercially available anti-immunoglobulin antibody. An antibody as contemplated herein includes any antibody specific to any region of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725, or antigenic parts thereof, collecting serum from the animal, and isolating specific sera by any of the known immunoadsorbent techniques. Although antibodies produced by this method are utilizable in virtually any type of immunoassay, they are generally less favored because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Basic Facts about Hybridomas, in Compendium of Immunology Vol. II, ed. by Schwartz, 1981; Kohler and Milstein, Nature 256: 495-499, 1975; Kohler and Milstein, European Journal of Immunology 6: 511-519, 1976).

Another aspect of the present invention contemplates a method for detecting AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or a derivative or homolog thereof in a biological sample from a subject, said method comprising contacting said biological sample with an antibody specific for AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or their antigenic derivatives or homologs for a time and under conditions sufficient for a complex to form, and then detecting said complex.

The presence of the complex is indicative of the presence of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725. This assay may be quantitated or semi-quantitated to determine a propensity to develop obesity or other conditions or to monitor a therapeutic regimum.

The presence of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labeled antibody to a target.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 complex, a second antibody specific to the AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725, labeled with a reporter molecule capable of producing a detectable signal, is then added and incubated, allowing time sufficient for the formation of another complex of antibody-AGT-711, AGT-712, AGT-713, AGT-714₃ AGT-715, AGT-716, AGT-717, AGT-718, AGT-720, AGT-721, AGT-723, AGT-724, AGT-726, AGT-719, AGT-722 and AGT-725-labelled antibody. Any unreacted material is washed away, and the presence of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labeled antibody are added simultaneously to the bound antibody. These techniques are well known to those skilled in the art, including any minor variations as will be readily apparent. In accordance with the present invention, the sample is one which might contain AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

The solid surface is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking covalently binding or physically adsorbing, the polymer-antibody complex to the solid surface which is then washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to about 37° C.) to allow binding of any subunit present in the antibody. Following the incubation period, the antibody subunit solid phase is washed and dried and incubated with a second antibody specific for a portion of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725. The second antibody is liked to a repoter molecule which is used to indicate the binding of the second antibody to AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725.

An alternative method involves immobilizing the target molecules in the biological sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. A “reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody absorbs the light energy, inducing a state to excitability in the molecule, followed by emission of the light at a characteristic color visually detectable with a light microscope. As in the EIA, the fluorescent-labeled antibody is allowed to bind to the first antibody-hapten complex. After washing off the unbound reagent, the remaining tertiary complex is then exposed to the light of the appropriate wavelength. The fluorescence observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed.

The present invention also contemplates genetic assays such as involving PCR analysis to detect AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 or their derivatives.

The assays of the present invention may also extend to measuring AGT-711, AGT-712, AGT-713, AGT-714, AGT-715, AGT-716, AGT-717, AGT-718, AGT-720, AGT-721, AGT-723,AGT-724,AGT-726,AGT-719,AGT-722 andAGT-725 or AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 in association with ob or leptin.

The present invention is further described by the following non-limiting Examples.

### EXAMPLE 1

### Animals

A Psammomys obesus colony is maintained at Deakin University, with the breeding pairs fed ad libitum a diet of lucerne and chow. Experimental animals were weaned at four weeks of age and given a diet of standard laboratory chow from which 12% of energy was derived from fat, 63% from carbohydrate and 25% from protein (Barastoc, Pakenham, Australia). Animals were housed individually in a temperature controlled room (22 ∀1° C.) with a 12-12-hour light-dark cycle. At 18 weeks of age, animals were sacrificed and the tissues immediately removed, frozen in liquid N₂ and then stored at −80°.

For experimental purposes, Psammomys obesus can be classified into three groups according to their blood glucose and plasma insulin concentration, taken in the fed state at 16 weeks of age. Group A animals are normoglycemic (blood glucose <8.0 mmol/L) and normoinsulinemic (plasma insulin <150 μU/L), Group B animals are normoglycemic but hyperinsulinemic (plasma insulin >150 μU/L), and Group C animals are hyperglycemic (blood glucose >150 mU/I) and hyperinsulinemic.

### EXAMPLE 2

### Sequencing and Cloning of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725

AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 were all identified by differential display PCR using the RNAimage mRNA cDNA microarray analysis using an SDDC-2 arrayer (Biorad) and GenePix 4000 Scanner (Axon instruments). Hypothalamus, liver or muscle RNA from fed and fasted or energy restricted, lean and obese Psammomys obesus was compared. Sequencing reactions were carried out using ABI PRISM Big-Dye terminator cycle sequencing ready reaction kits and analyzed on an ABI 373 DNA sequencer. Gene database searches were performed at the National Center for Biotechnology Information using the BLAST network service. In order to obtain further mRNA sequence, 5′ and 3′ RACE (Rapid Amplification of cDNA Ends) was performed using the Marathon cDNA amplification kit (Clontech).

### EXAMPLE 3

### Analytical Methods

Whole blood glucose was measured using an enzymatic glucose analyzer (Model 27, Yellow Springs Instruments, Ohio). Plasma insulin concentrations were determined using a double antibody solid phase radioimmunoassay (Phadeseph, Kabi Pharmacia Diagnostics, Sweden).

### EXAMPLE 4

### Microarray Analysis

Microarray analysis was used in time course studies using the P. obesus animal model to identify changes in expression of genetic material.

cDNA microarray technology provides a powerful technical means to generate a gene expression database of both known genes and unknown transcripts. Using cDNA microarrays, comparative estimates can be obtained of the level of gene expression of large numbers of genes (up to 20,000 per microarray) in each sample. cDNA microarray generally involve a large number of DNA “spots” in an orderly array chemically coupled to the surface of a solid substrate, usually but not exclusively an optically flat glass microscope slide. Fluorescently labeled cDNAs are generated from experimental and reference RNA samples and then competitively hybridized to the gene chip. The experimental and reference cDNAs are labeled with a different fluorescent dye and the intensity of each fluor at each DNA spot gives an indication of the level of that particular RNA species n the experimental sample relative to the reference RNA. The ratio of fluorescence can be taken as a measure of the expression level of the gene corresponding to that spot in the experimental sample.

### EXAMPLE 5

### Clone Sequencing and Identification

Sequencing reactions were carried out using ABI PRISM Big-Dye terminator cycle sequencing ready reaction kits and analyzed on an ABI 373 DNA sequencer. Gene database searches were performed at the National Center for Biotechnology Information using the BLAST network service.

### EXAMPLE 6

### Quantitafion of Gene Expression

Animals were killed by lethal overdose of pentobarbitone (120 mg/kg) and the following tissues were removed: liver, spleen, kidney, heart, skeletal muscle (gastrocnemius), and adipose tissue from the suprascapular, perirenal, intramuscular and mesenteric fat depots. RNA was extracted from tissues using RNAzol B (Tel-Test). RNA was quantitated by spectrophotometry at 260 nm and electrophoresed through a glyoxal gel (Ambion) to check integrity. 1 μg of RNA was then reverse transcribed at 42° C. for 1 hr with AMV Reverse Transcriptase (Promega) according to the manufacturer's instructions. Oligonucleotide primers for the AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 gene PCR were chosen from the sequence previously determined. Primers were also designed to the Psammomys obesus β-actin gene to use as a housekeeping gene.

Gene expression of AGT-711, AGT-712, AGT-713, AGT-714, AGT-715, AGT-716, AGT-717, AGT-718, AGT-720, AGT-721, AGT-723, AGT-724, AGT-726, AGT-719, AGT-722 and AGT-725 in each cDNA sample was quantitated using Taqman PCR technology on an ABI Prism 7700 sequence detector. P-actin was used as an endogenous control to standardize the amount of cDNA added to a reaction. Primer sequences for the AGT-genes and β-Actin primers were as follows:

| | | |
|---|---|---|
| AGT-711: | | |
| Forward primer: | 5′-CACCCACTAGCATTTCTGTGATG-3′ | (SEQ ID NO:20) |
| Reverse primer: | 5′-CATTAGCACCAAGGAGTCAAGGT-3′ | (SEQ ID NO:21) |
| AGT-712: | | |
| Forward primer: | 5′-CTAACCATGCTTCCCCTCCAT-3′ | (SEQ ID NO:22) |
| Reverse primer: | 5′-CACCCTCATTCCACAAATGCTAT-3′ | (SEQ ID NO:23) |
| AGT-713: | | |
| Forward primer: | 5′-GTTGCTTTACAGTCTGTGGCAAA-3′ | (SEQ ID NO:24) |
| Reverse primer: | 5′-TGCATCCAGTGGTTGTGAGAA-3′ | (SEQ ID NO:25) |
| AGT-714: | | |
| Forward primer: | 5′-GAGGAACAGCCCTTTATGTAGGT-3′ | (SEQ ID NO:26) |
| Reverse primer: | 5′-GAAATGGATGACTTTGGGAAGAA-3′ | (SEQ ID NO:27) |
| AGT-715: | | |
| Forward primer: | 5′-AGGCTTACGGTCTGGACAACAG-3′ | (SEQ ID NO:28) |
| Reverse primer: | 5′-CGCTTTGCCGAATACCTCTAAA-3′ | (SEQ ID NO:29) |
| AGT-716: | | |
| Forward primer: | 5′-AAGTAATCTTCTGAAACCTAGAACCTCTTC-3′ | (SEQ ID NO:30) |
| Reverse primer: | 5′-CTGCCCAAAATAGGAGTGATCAC-3′ | (SEQ ID NO:31) |
| AGT-717: | | |
| Forward primer: | 5′-CCGGGTCAATGTGATTCATG-3′ | (SEQ ID NO:32) |
| Reverse primer: | 5′-GGTGCGAGCGATGTTTGTG-3′ | (SEQ ID NO:33) |
| AGT-718: | | |
| Forward primer: | 5′-TCTCGTTTATGTCTGCGTAATGACT-3′ | (SEQ ID NO:34) |
| Reverse primer: | 5′-CCCTGAAGAGCTGGGAGTACA-3′ | (SEQ ID NO:35) |
| AGT-719: | | |
| Forward primer: | 5′-AGGCTGGCTTTGACCTCACA-3′ | (SEQ ID NO:36) |
| Reverse primer: | 5′-GCGCACGCCTGTAATTCC-3′ | (SEQ ID NO:37) |
| AGT-720: | | |
| Forward primer: | 5′-GAGGCAGGAGGACAAGTTCAAA-3′ | (SEQ ID NO:38) |
| Reverse primer: | 5′-TCAGTGAGCATGTTTTTTCTTTCATT-3′ | (SEQ ID NO:39) |
| AGT-721: | | |
| Forward primer: | 5′-CCTCAAGCAAATGTTGAGAGAACA-3′ | (SEQ ID NO:40) |
| Reverse primer: | 5′-AACAGGAGGATCCAAGGTTTCAT-3′ | (SEQ ID NO:41) |
| AGT-722: | | |
| Forward primer: | 5′-GAGACCCTGTCTCAAGTTAGGAATG-3′ | (SEQ ID NO:42) |
| Reverse primer: | 5′-GTGTGCATGATCACGGATGTG-3′ | (SEQ ID NO:43) |
| AGT-723: | | |
| Forward primer: | 5′-AGCAGTATTTGTGCCCATCTCA-3′ | (SEQ ID NO:44) |
| Reverse primer: | 5′-AACTGACCAGCAGCCTGTTCA-3′ | (SEQ ID NO:45) |
| AGT-724: | | |
| Forward primer: | 5′-AGTTGACATGGGCCTTTCCA-3′ | (SEQ ID NO:46) |
| Reverse primer: | 5′-GGCTTGTGTTGAGTTAGCCTTCA-3′ | (SEQ ID NO:47) |
| AGT-725: | | |
| Forward primer: | 5′-CCTGAACTTCCCACTCACACTCT-3′ | (SEQ ID NO:48) |
| Reverse primer: | 5′-CAAAAGCCATAGAACAACCACATAGT-3′ | (SEQ ID NO:49) |
| AGT-726: | | |
| Forward primer: | 5′-GCTTCGGAGAGTTGGCTTTG-3′ | (SEQ ID NO:50) |
| Reverse primer: | 5′-GCCCCACAGTTTCACATTTGT-3′ | (SEQ ID NO:51) |
| β-actin forward: | 5′-GCAAAGACCTGTATGCCAACAC-3′; | (SEQ ID NO:17) |
| β-actin reverse: | 5′-GCCAGAGCAGTGATCTCTTTCTG-3′. | (SEQ ID NO:18) |
| β-actin gene: | 5′-TCCGGTCCACAATGCCTGGGTACAT-3′ | (SEQ ID NO: 19) |

All probes had the reporter dye FAM attached to the 5′ end and the quencher dye TAMRA attached to the 3′ end. PCR conditions were 50° C. for 2 min, 95° C. for 10 min followed by 40 cycles of 95° C. for 15 sec and 60° C. for 1 min.

Gene expression in each cDNA sample was quantitated using SYBR Green PCR on an ABI Prism 7700 sequence detector. β-actin was used as the endogenous control to standardize the amount of CDNA added to a reaction.

PCR conditions were 50° C. for 2 min, 95° C. for 10 min followed by 40 cycles of 95° C. for 15 sec and 60° C. for 1 min.

### EXAMPLE 7

### Statistical Analysis

All experimental data are expressed as means ±SEM. A one-way analysis of variance in combination with a LSD post-hoc test was used to compare means between and within groups, and a two-sample unpaired t-test was used where appropriate. In all instances probability values of <0.05 were considered significant.

### EXAMPLE 8

### Identification of Body Weight and Diabetes-Related Genes by Microarray Analysis

To identify novel genes that are associated with regulation of energy balance and/or glucose homeostasis, the inventors compared the hypothalamic or liver or muscle mRNA profile of lean, obese and diabetic Psammomys obesus in the fed and fasted state. Differentially expressed genes were highlighted by sophisticated data analysis.

### EXAMPLE 9

### AGT-711 Gene Expression in the Liver

Gene expression of AGT-711 was analysed in liver of P. obesus after dietary energy restriction. Expression in ad libitum fed Group C (obese, diabetic) P. obesus was significantly reduced compared to Group B (obese, IGT) P. obesus (p=0.024), as well as to dietary energy restricted Group C P. obesus (p=0.008) (see FIG. 2).

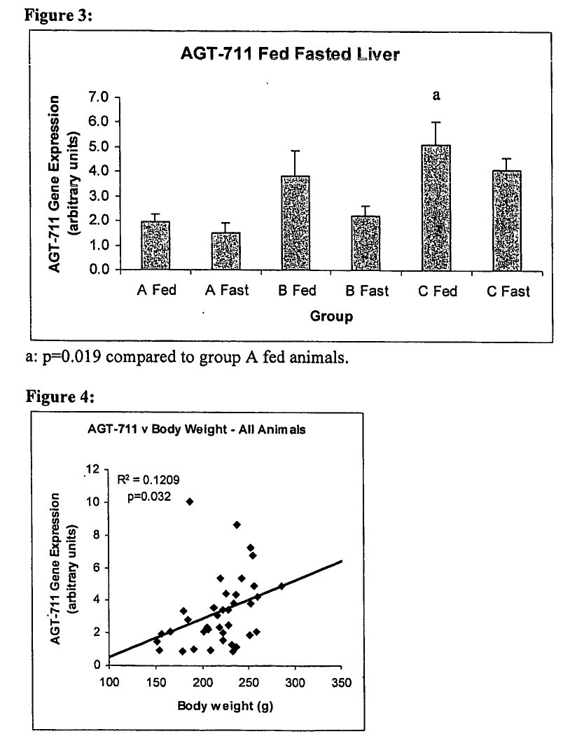

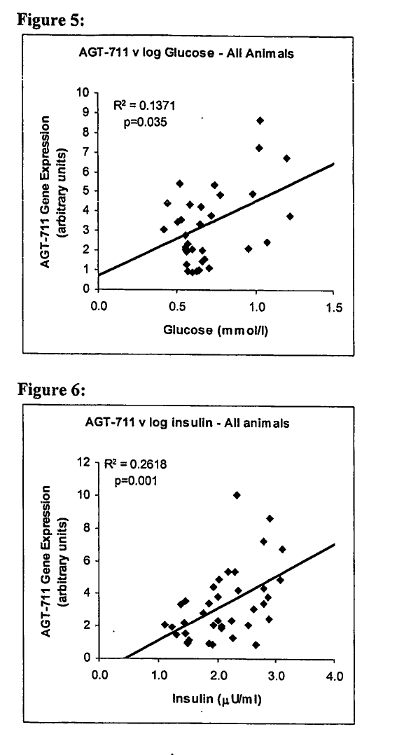

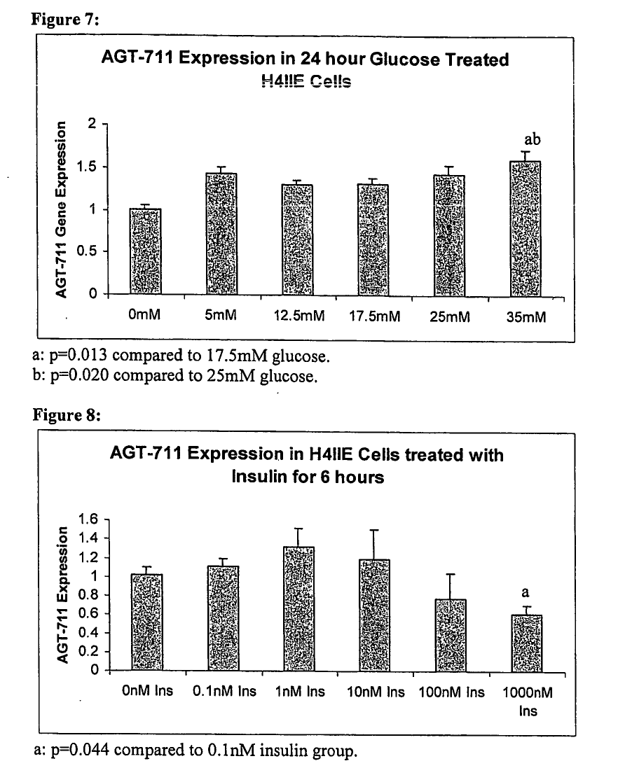

Due to the fact that AGT-711 was discovered in dietary energy restricted liver samples, AGT-711 gene expression was examined in the liver of ad libitum fed and fasted (24 h) P. obesus. No significant differences between the fed and fasted groups was seen, however there was a significant difference between Group C (obese, diabetic) and Group A (lean, nGT) P. obesus in the fed state (p=0.019) (see FIG. 4). There were significant positive correlations between hepatic AGT-711 gene expression and body weight (p=0.032) (see FIG. 5), blood glucose (p=0.035) (see FIG. 6) and plasma insulin concentration (p=0.001) (see FIG. 7).

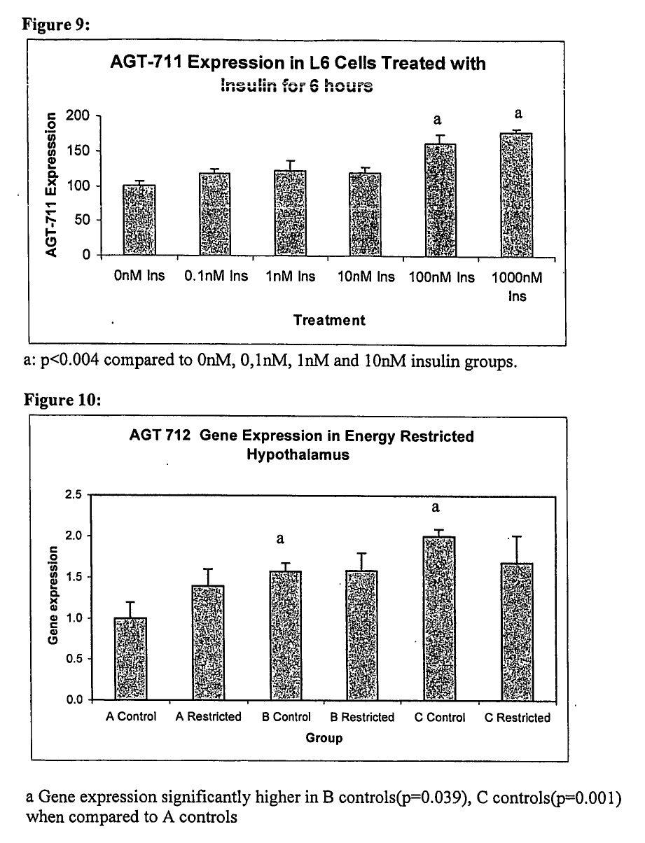

AGT-711 gene expression was also examined in the H4IIE rat liver cell line treated with various concentrations of glucose for 24 hr and insulin for 6 hr. AGT-711 gene expression after 35 mM glucose treatment was higher than some of the lower doses used (17.5 mM, p=0.013; and 25 mM, p=0.02) (see FIG. 8). In insulin stimulated H4IIE cells, there was a trend for higher insulin concentrations to decrease AGT-711 gene expression, however only the 1000 nM-treated group showed statistically significant suppression compared to the 0.1 nM-treated group (p=0.044) (see FIG. 9).

AGT-711 gene expression was also examined in rat L6 muscle cells treated with varying concentrations of glucose for 24 hr and insulin for 6 hr. There were no significant differences in AGT-711 gene expression after treatment with various concentrations of glucose (not shown). Insulin treatment for 6 hrs resulted in significant increases in AGT-711 gene expression at 100 nM and 1000 nM compared to 0 nM, 0.1 nM, 1 nM and 10 nM insulin (p<0.004) (see FIG. 10).

These expression data infer a role for AGT-711 in the regulation of glucose homeostasis in P. obesus, particularly in the liver where differences in AGT-711 gene expression were observed between lean, nGT and obese, diabetic animals, and AGT-711 gene expression was correlated with circulating glucose and insulin concentrations, as well as body weight.

### EXAMPLE 10

### Partial Sequence of Psammomys obesus AGT-711

The partial nucleotide sequence of Psammomys obesus AGT-17 cDNA is as follows:

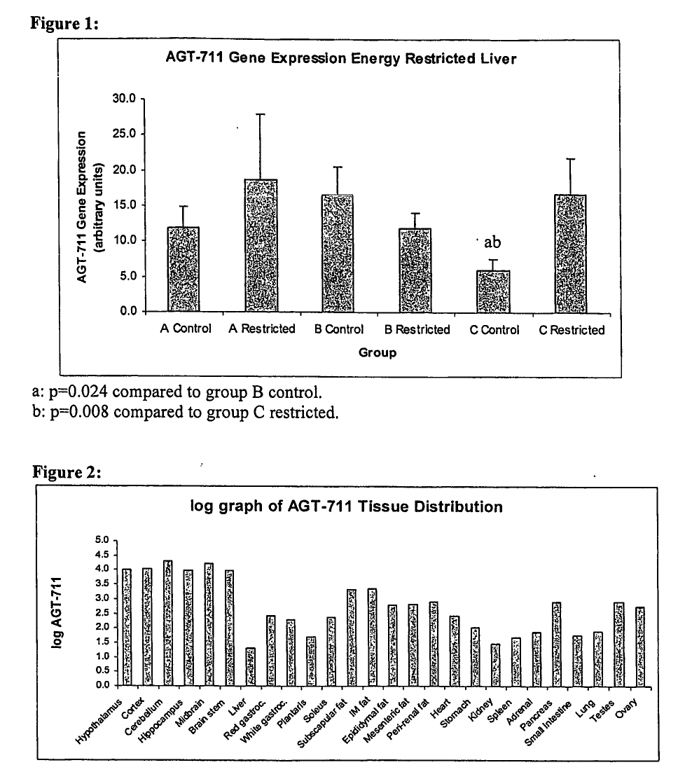

(see FIG. 1: SEQ ID NO:1)

TGAGAATTAAGATTATTGTATGATTGAAACATGAAACAGCTCATGTCTCT
GTTAGTAACATCAAAGGCAGTCACGTTTACACTGCAGTTAGAACTCTTAG
GGCACGTTGCTTTTCATCAGGCTTCCCCTGCTTTTGATTTGTGGCTGTTG
CTGATTTTTCGTATGTGGACATACACCTACCTCTTCTGTTGGAAAGAACA
TTTAAAATGAAACAAATTTTACTTAAAAAAAAATCAAGGAGTCCTTTAAT
GTAAATTTTAACTTTCAAATTACTTTCTTTATCTTACTTTTATTACAAAT
AGCACCCACTAGCATTTCTGTGATGTACCCACCTCTTTCNATGTGCTATT
TGACTGATGCTTGCTCCCTGGGATGACGTTGCAAAAAATCGACTAGTAGA
AATGAAGTGNAAATGCATTTTGGTGNATACCTTGACTCCTTGGTGCTAA
T.

The nucleotide sequence shows homology to Mus musculus elongation of very long chain fatty acids (FEN1/Elo2, SUR4/Elo3, yeast)-like 4 (Elovl4), mRNA.

### EXAMPLE 11

### AGT-711 Tissue Distribution

A SYBR Green PCR tissue distribution in Psammomys obesus was performed for AGT-711. Expression of AGT-711 is represented in FIG. 3 as a log graph due to the fact that expression in brain regions was 20-50 fold higher than in all other tissues including liver, adipose tissue and muscle tissue, although detectable levels of gene expression was seen in all tissues examined.

### EXAMPLE 12

### AGT-712 Gene Expression in the Hypothalamus

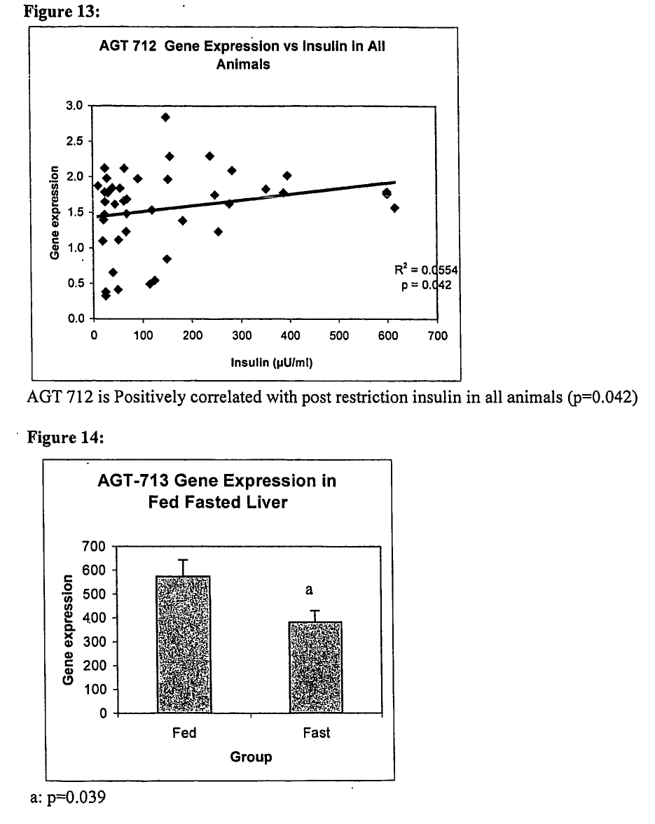

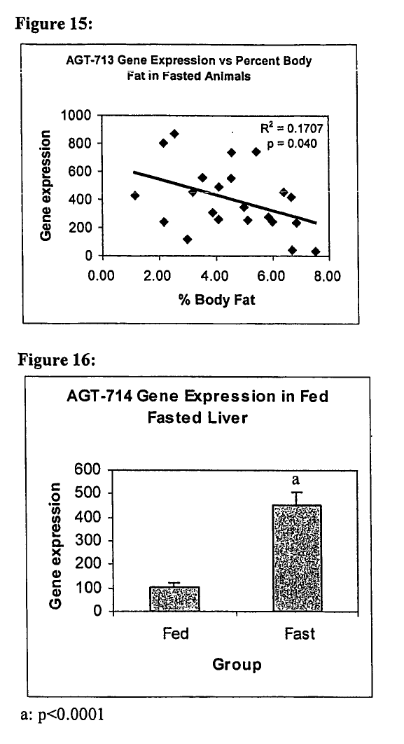

Hypothalamic AGT-712 gene expression was significantly higher in Group B (obese, IGT) (p=0.039) and Group C (obese, diabetic) (p=0.001) P. obesus compared with Group A (lean, nGT) P. obesus (see FIG. 12). Expression was positively correlated with blood glucose (p=0.014) (see FIG. 13) and insulin (p=0.016) (see FIG. 14) concentrations in P. obesus, and with plasma insulin concentrations after dietary energy restriction (p=0.042) (see FIG. 15).

### EXAMPLE 13

### Partial Sequence of Psammomys obesus AGT-712

The full AGT-712 cDNA sequence has not been elucidated as yet. The partial sequence obtained so far is as follows:

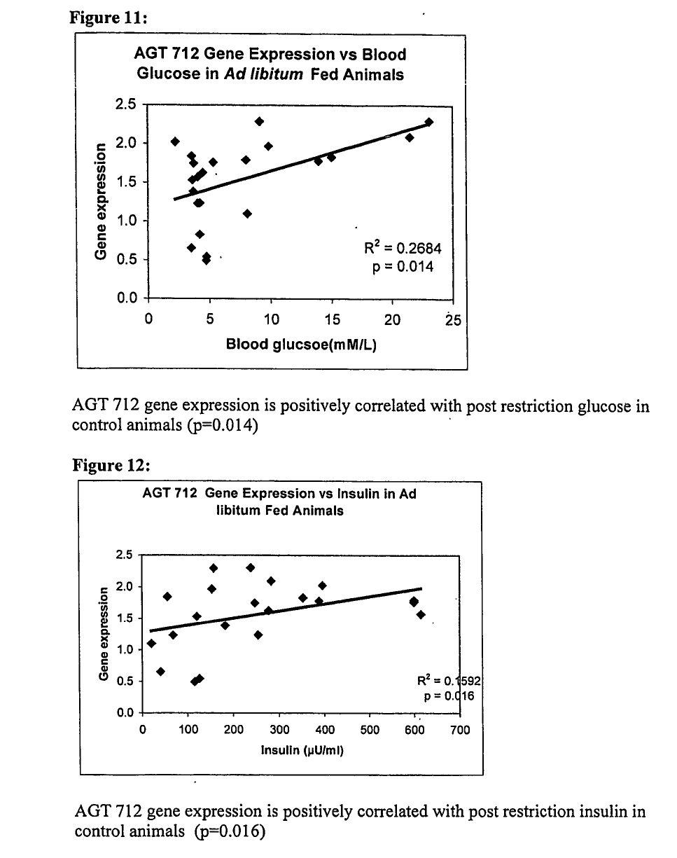

(see FIG. 11: SEQ ID NO:2)

CCCACGCGTCCGGTGAAGAATTAGTCTGCCTTGGCATTTATAAATGATTC
ACAACCAGTTTCCAGTTGGACCCCAAAAAACTATTTAATTATCAATGAGA
TAATATGTTTCCTTTCTGAATAATACATAAAAGAAATCAGAGGCTGGGAA
AAAAAGTTATGGATAAGAAGTTCCAGTCCAATAAAATTATATTTATCTGT
AGGAGTCATTTTTCCTAGATTTCTGTTTCTAACATCCTGGAAAAAAAATT
GAGTTTCTGGTAAATAGTCCAGAAATTATTTAGCTAATTTAATTTCTTTA
CATTTGAAGACTTTAGATTTATTTTCAGTATTTAGTTAATATAAAATTAA
AGGAAAATCAACTTAAACTTAGATCAACATTTAATATAACTTTATTTTTT
TGTGTACATAATAAATTTATCCAGAAGTTAAATGCATGGAAACATGATTT
TATAAAACAAAACTAACCATGCTTCCCCTCCATTGCCATATCTGCTCTTG
TAGGATGCTATTTTCTGTATGTTAAAATCGGGTGAATGAGATAGCATTTG
TGGAATGAGGGTGTGCAGTGCTTTATGTACAGGTAGTTAGAGTAAGGTGC
CTTGTTCTTCACTCGAATATATGAACATGCCAGTTAAACCTCTTTAAATT
TTTTTCTTTTACTATTCACCTCCTTAACTGAATCACACCAGCTTGCTTAG
AATCTCATGGAAGCAATGCTCAGGCAATTTCACTTTTTTGTTGNTGNTAT
TGNCTTCTTTTGAAAAGCTAATCATGGCACATGTAAGGGACNGGCGCGTT
CACTGCCCAGGCCATTGCATGGAATCACTGCAGTCTTGTCCTGCACATGG
GATCTACACTGGCTATCTATAGTAACTAGTGTAATTGAAGGCAGACAAGC
TTGACGCAGATAAGCTTTGGGCAGCTGAATAATCTACAGGGTTAGTTTTG
AGGATCAAATACTGGCTGATAATTATGATATTGCCACATGGTGAGTCTAT
GTGCTTGAATAAGTGCATCTTGTAGCATCTATGGGAAGTGTGGGGGGAAT
TAAGTGTTAGTGAAGAGAATATCTACCAAGTGGCTTTTTGTAAGATAGGA
AACAATAGAGTCCAAAGGGAACTTTTAAGAGATTGAAAAGGGGGAATAAT
ATTTCAGCATAAAATCCTAAATTTGAATAAAAGAAAGAAGAGGAGATTGG
NGCTGTTGCTGGTGTGTGCAGATTTTCTGGGTTCTTCCTTTTAATGTTCC
ACTGTTTTTCCTGTCAGTATTGTAGATGTGTGATGACTTGCCTGTCAAAT
TTATCAACTGTTGAGAAAGTAATAGTGTAAATCTTTGTGCCAAGGAAACC
TCTGCCAGTGTTGTGTCTCTTTACCTCATTCTTGTATTTGGTCCTGTCAA
GATTGTAGCTTGTCTGACAGAAGCCACAGCGNAGNCTGATGAAACTTGCC
AGGNCAGTTTTTTGAGCAGCTCCCAAAGCCGGAGGCCCAGCTTCTCAGCC
TTGGACAGCTTCNGCCTACCCGTAAACCTCAGTGGCATACTCTCTAACAG
TGTGTGTAGACTGGGATGCTGTTCACATGTGGCTTTGGATCATTTCTAAT
GTGACGGAATTTTATTTGTCTCTTGACAGCTTTGTAGAAATGCAATTTTT
ATAGTAAAGACATTGTTCATTTGGAAGTCAATACGTTATAAAATTGG.

This sequence demonstrates sequence homology to the hypothetical protein DJ971N18.2 {Homo sapiens}.

### EXAMPLE 14

### AGT-713 Gene Expression in Tire Liver

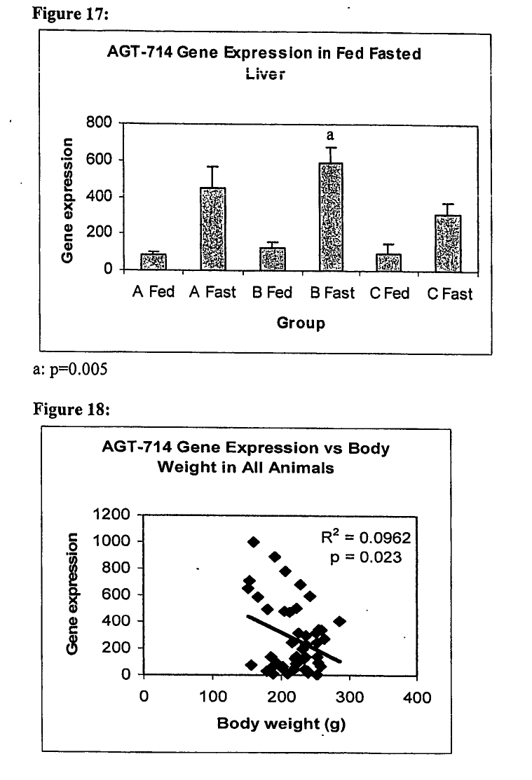

Hepatic AGT-713 gene expression was significantly decreased after fasting (24 h) (p=0.039) (see FIG. 17). AGT-713 expression was negatively correlated with percent body fat in fasted P. obesus (p=0.04) (see FIG. 18).

### EXAMPLE 15

### Partial Sequence of Psammomys obesus AGT-713

The partial nucleotide sequence of Psammomys obesus AGT-713 cDNA is as follows:

(see FIG. 16; SEQ ID NO:3)

AGCGTGGTCGCGGCCGAGGTACATAAAATGGCTAACTTATAGAGATCAAA
GGTTATTTTGGTTTGTTGTTTCAACTGTTTAACTGTTGCTTTACAGTCTG
TGGCAAAGCAGTGCATGGTTTAGAATGTGGCAGAACAAAGCAGTTCTCAC
AACCACTGGATGCAAAGAGAAGGGAAGAAAAAAAAAGAGAATAGGGTTGA
ATATCCCATTCAAGGGCATGCCTCCATGTATGGGTTCCTCTNATTAGCCA
CCCTCCCTCCCCGCCACAAGTAAATCCTAATAGCACCAGGGTCTGGGGTC
AATCGTTTACCATACAGCCATTTGTGAGAAACTTGTTAAGATAACATATA
TCACACTGCCTGTGTATGCTGAAATAAATTCATAAATTAATAACATTTCA
AAAAAAACCTGCCCGGGCGGCCGCTCGAA.

The nucleotide sequence does not match any known genes in the GenBank database.

### EXAMPLE 16

### AGT-714 Gene Expression in the Liver

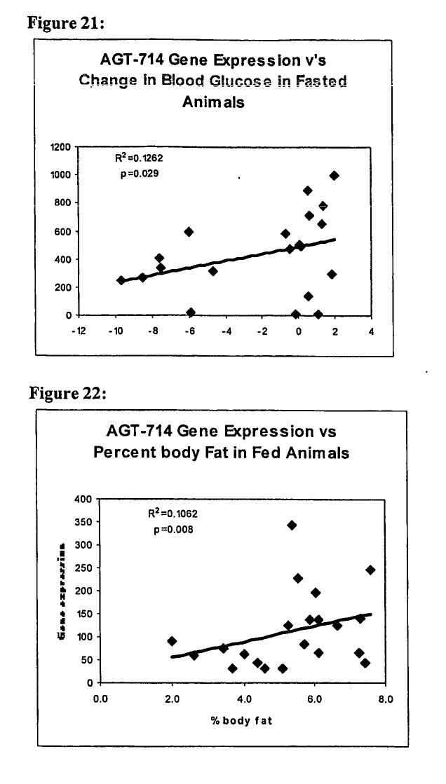

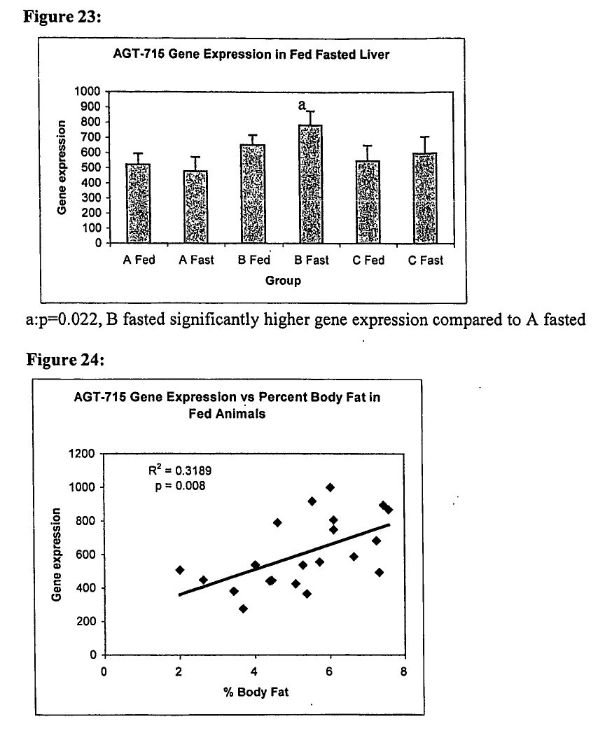

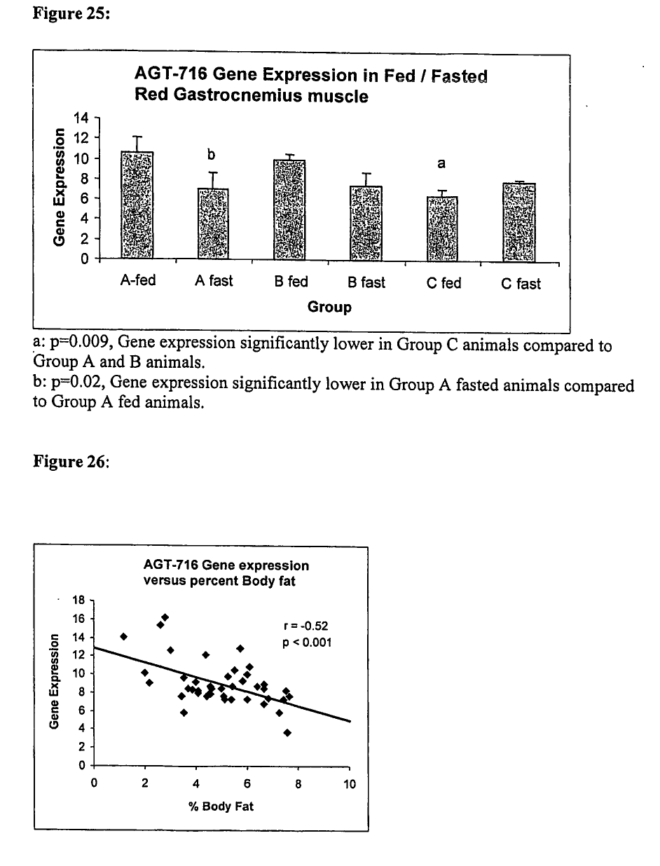

Hepatic AGT-714 gene expression was significantly increased after fasting (24 h) in P. obesus (p<0.001) (see FIG. 20). In addition, AGT-714 gene expression was significantly higher in Group B (obese, IGT) P. obesus after fasting (24 h) (p=0.005) (see FIG. 21). AGT-714 gene expression was negatively correlated with body weight (p=0.023) (see FIG. 22) and plasma insulin (p=0.017) (see FIG. 23) in P. obesus. In fasted (24 h) P. obesus, AGT-714 gene expression in the liver was negatively correlated with body weight (p=0.005) (see FIG. 24) and positively correlated with the change in blood glucose after fasting (p=0.029) (see FIG. 25). In addition, AGT-714 gene expression was positively correlated with percent body fat (p=0.008) (see FIG. 26).

### EXAMPLE 17

### Partial Sequence of Psammomys obesus AGT-714

The partial nucleotide sequence of Psammomys obesus AGT-714 cDNA is as follows:

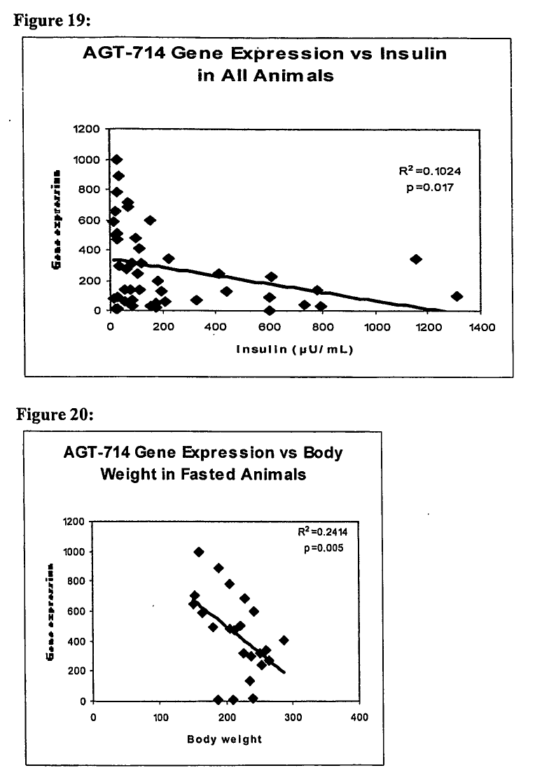

(see FIG. 19: SEQ ID NO:4)

TAGCGTGGTCGCGGCCGAGGTACTGACNTTCCATTCTGCTTCACATGAAA
TCAACGGAAGCTCTTTATTCAAGCTTTGGGCTCTCAGACACNGAAATGAG
ACCGTGACCATGGCAGTNGGACAGGAAAATTCTTGTTTGACCCTGAATGA
GAGCACANAGGCATGAATGTGGAGGTGCCAGCTTGCCTAATGAATGAGGG
AACAGCCCTTTATGTAGGTCAGCCGCTTTCCTTCCCCTGGGCTGCCGTGC
TTTCTTCCCAAAGTCATCCATTTCTGATAATGATGTGAATTGTCTGTTAG
CTATCAAGTACCTGCCCGGGCGGCCGCTCGAA.

The nucleotide sequence demonstrated sequence homology to Homo sapiens Chromosome 15q26.1 PAC clone pDJ105i19, complete sequence.

### EXAMPLE 18

### AGT-715 Gene Expression in the Liver

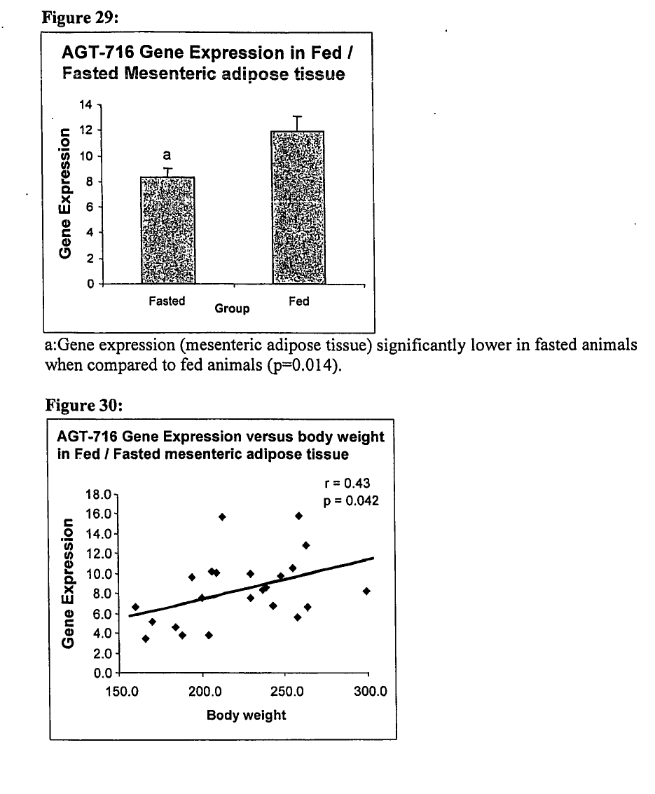

AGT-715 gene expression in the liver was increased after fasting (24 h) in Group B (obese, IGT) P. obesus compared with fasted Group A (lean, nGT) P. obesus (p=0.022) (see FIG. 28). Furthermore, hepatic AG-T-715 gene expression was positively associated with percent body fat in P. obesus (p=0.008) (see FIG. 29).

### EXAMPLE 19

### Partial Sequence of Psammomys obesus AGT-715

The partial nucleotide sequence of Psammomys obesus AGT-715 cDNA is as follows:

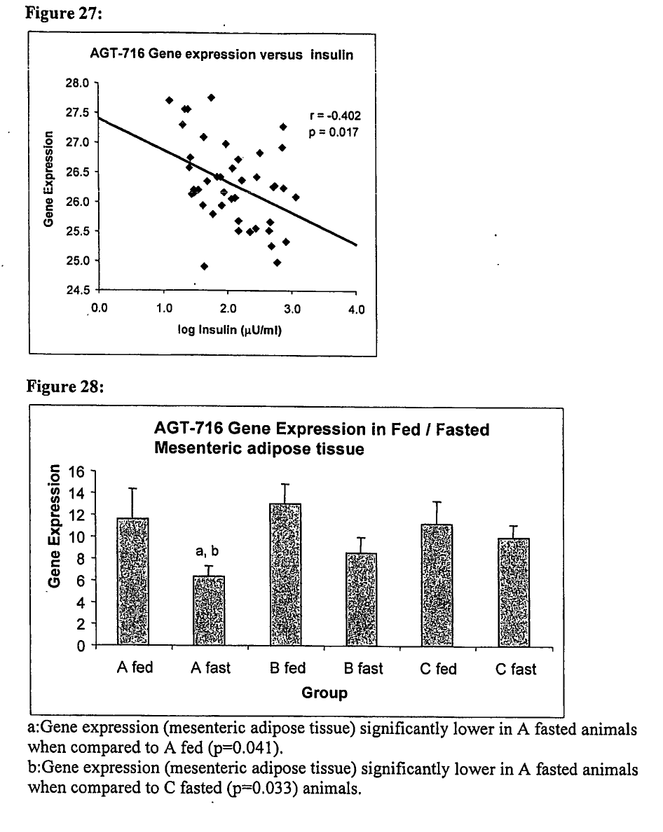

(see FIG. 27: SEQ ID NO:5)

AGCGTGGTCGCGGCCGAGGTCAGCTTTATACAGAAAACAAGGCTTAGCCT
CTGGACAACAGAGAAGCGCTCAGTCCCAAAGCCACAGGAAGGCCTTTTAC
CTGCTGCAGCCCTCGGCCATGGAGTACCTGCCCGGGCGGCCGCCTCGAAA
GCCGAA.

The nucleotide sequence does not match any known genes that are currently in the GenBank database.

### EXAMPLE 20

### AGT-716 Gene Expression in Adipose Tissue and Red Gastrocnemius Muscle

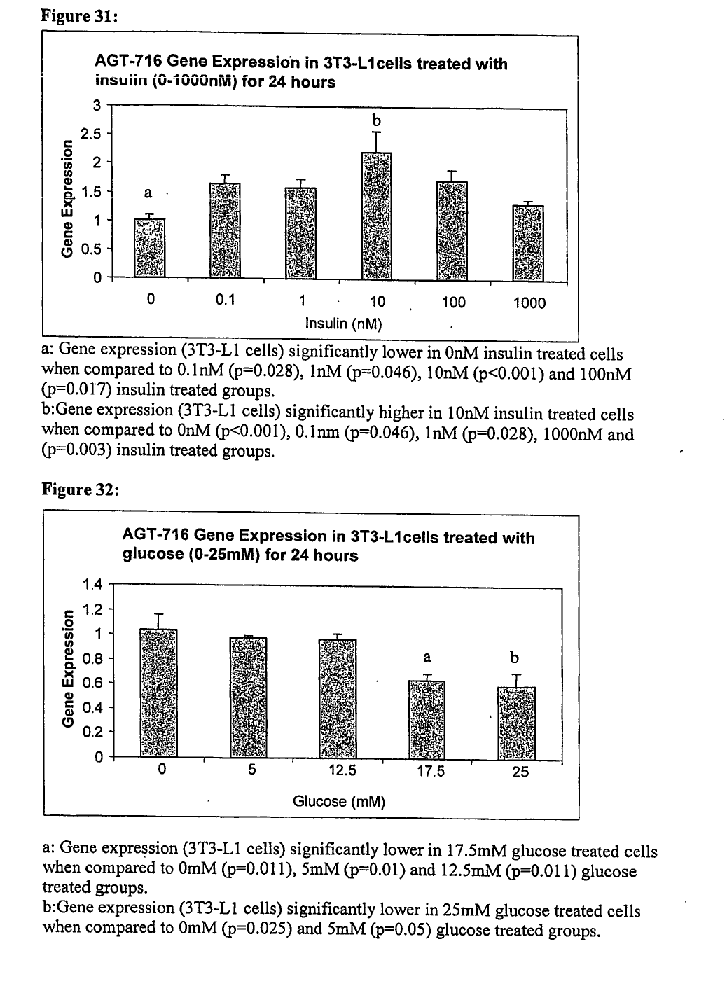

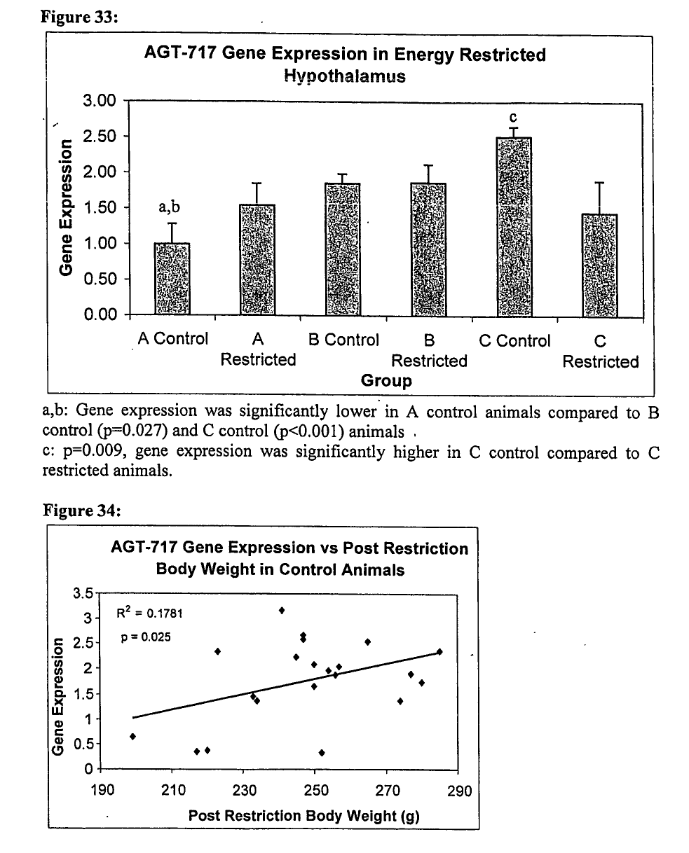

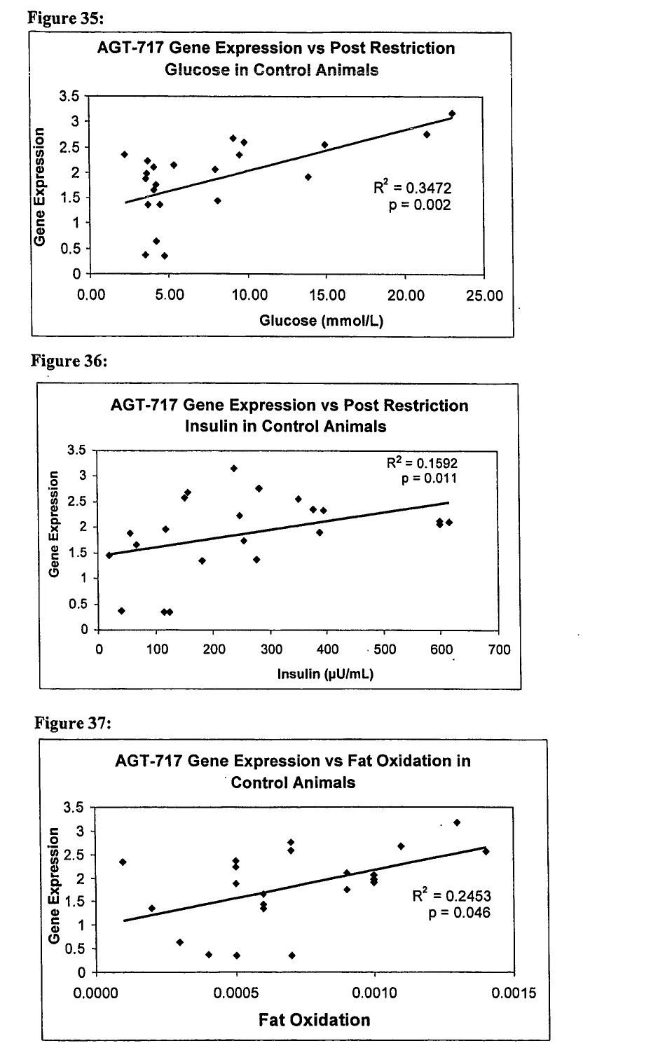

In P. obesus, AGT-716 gene expression was examined in red gastrocnemius muscle and mesenteric adipose tissue. In red gastrocnemius muscle, AGT-716 gene expression was significantly reduced in Group C (obese, diabetic) P. obesus (p=0.009) when compared to Group A (lean, nGT) and Group B (obese, IGT) P. obesus (see FIG. 31). AGT-716 gene expression in muscle was negatively correlated with percent body fat (p<0.001) (see FIG. 32) and plasma insulin concentration (p=0.017) (see FIG. 33) in P. obesus. AGT-716 expression in adipose tissue was reduced after fasting (24 h) in Group A (lean, nGT) P. obesus (p=0.041), but not Group B or Group C P. obesus. After a 24 h fast, AGT-716 gene expression in adipose tissue was significantly increased in Group C (obese, diabetic) compared with Group A (lean, nGT) P. obesus (p=0.033) (see FIG. 34). In all P. obesus, AGT-716 gene expression in adipose tissue was significantly reduced in the fasted state when compared to the fed state (p=0.014) (see FIG. 35). AGT-716 gene expression in adipose tissue was positively correlated with body weight in fasted (24 h) P. obesus (pw0.042) (see FIG. 36).

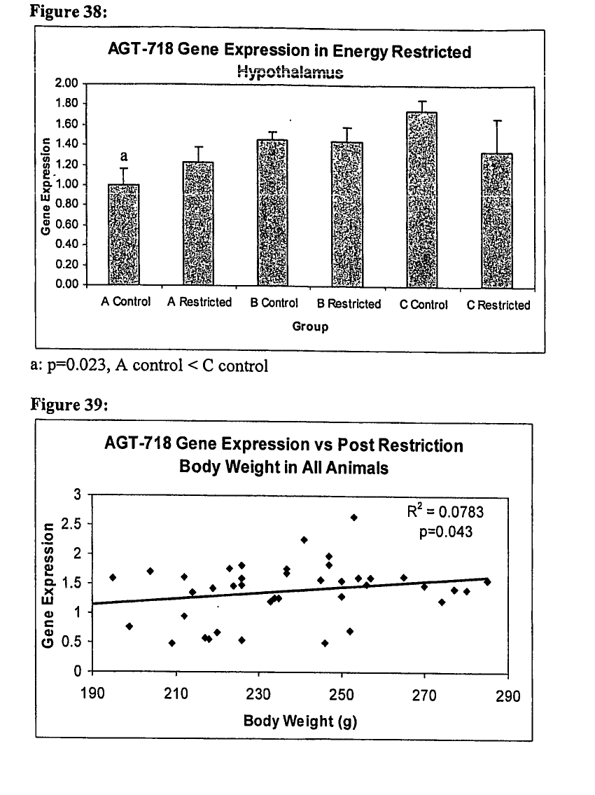

In vitro studies using differentiated 3T3-L1 adipocytes, showed that AGT-716 gene expression was significantly lower in cells without any insulin in comparison to the 0.1 nM (p=0.028), 1 nM (p=0.046), 1 nM (p<0.001), and 10 nM (p=0.017) insulin treated cells (see FIG. 37). High glucose (17.5 and 25 mM) treatment in 3T3-L1 cells led to a decrease in AGT-716 gene expression when compared to control cells (no glucose) and the 5 and 12.5 mM glucose treated cells (p<0.05) (see FIG. 38).

The finding that expression of AGT-716 was significantly reduced in the muscle of diabetic animals indicates that AGT-716 may play an important role in obesity/diabetes. Fasting in healthy P. obesus resulted in a decrease in AGT-716 expression whereas in obese/diabetic animals fasting had no effect. These results indicate that AGT-716 gene expression may be dysregulated in P. obesus in the fasted state. Furthermore, significant correlations between AGT-716 gene expression with plasma insulin, blood glucose, percent body fat and body weight support the role of AGT-716 in the development of obesity/diabetes and indicate that AGT-716 may be a target for therapy in human obesity/diabetes.

### EXAMPLE 21

### Partial Sequence of Psammomys obesus AGT-716

The partial nucleotide sequence of Psammomys obesus AGT-716 cDNA is as follows:

(see FIG. 30: SEQ ID NO:6)

AGACTGCCCGTGATCCATGAGCTTAGGATCAGGCATAAACTGTGTTTTAT
CTGAGGTGGATTCTGATTTCATCTTTGTCACAGAAGCAAATTCTTTTGGC
AAAGACACTGTTTTGGCATGTATTTCACCAGACCTTCCTGCCTTAGTTTC
TTCTTTTGGTTCAGGGAGTGATTGTTTTTCACTGCTTTTTGTCCACAATG
ACTTAATGGCTTGATAAATTTGTTGGCTTGTATCATATGTGTTTCTCCCA
GTTATCTTTGCAATTATTACTAACTGAGCTGGGTAGCAAACAGCTGCTCC
TAATGTGGCCATCCCTAGAGGATAAGTAATCTTCTGAAACCTAGAACCTC
TTCTTGCTGACAGCAAACCTGCCAGTCCTGAAGCTGTGATCACTCCTATT
TTGGGCAGAAAATCTTGAGGAGGGTTCTTCAGATAGATATATGTATC.

The nucleotide demonstrated sequence homology to a homo sapiens sequence similar to RIKEN cDNA 9430083G14 gene (LOC139322).

### EXAMPLE 22

### AGT-717 Gene Expression in the Hypothalamus

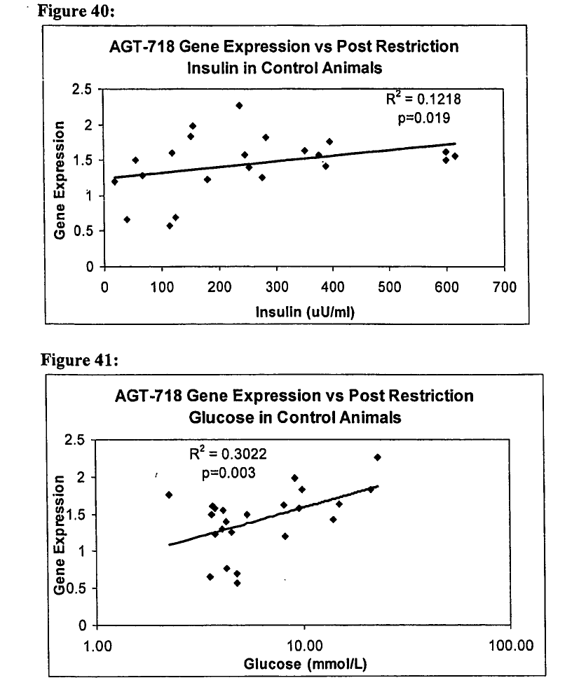

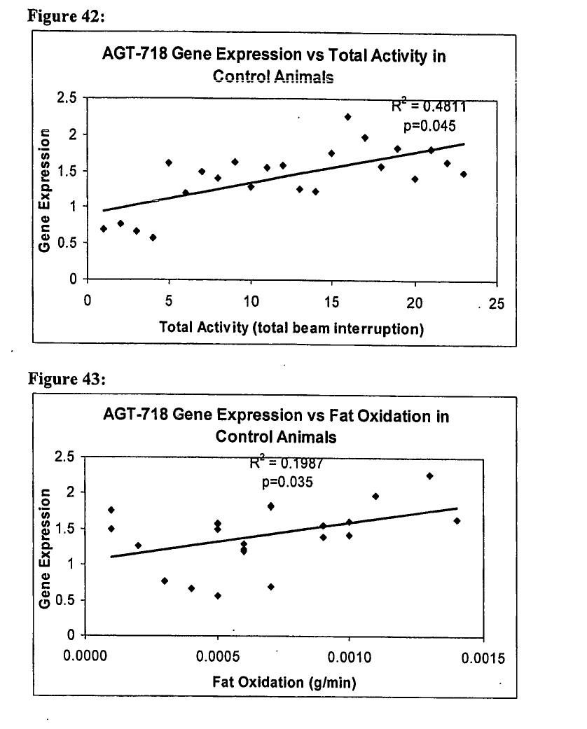

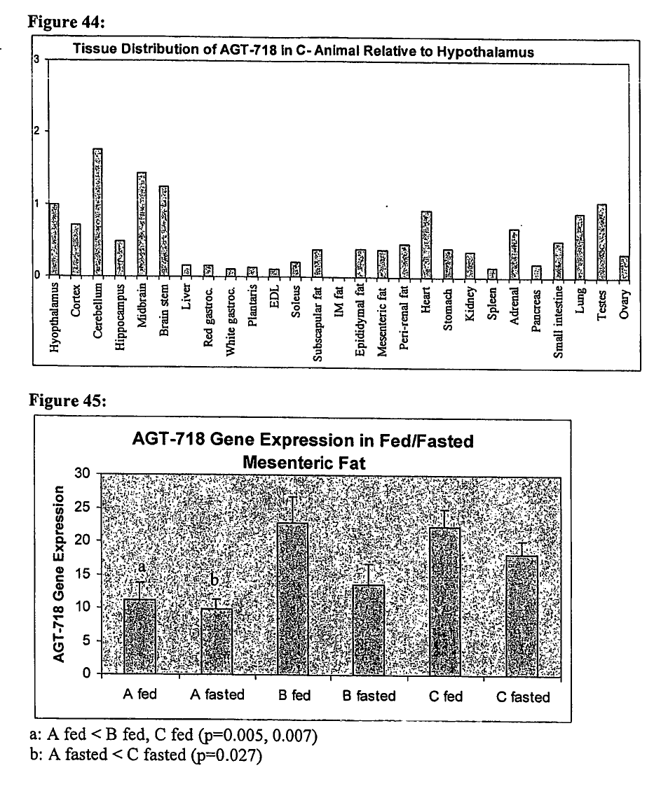

Hypothalamic AGT-717 gene expression, as measured by SYBR green real time PCR, was significantly elevated in Group B (obese, IGT) (p=0.027) and Group C (obese, diabetic) (p<0.001) P. obesus compared with Group A (lean, nGT) P. obesus (see FIG. 40). AGT-717 gene expression was significantly reduced after dietary energy restriction in Group C (obese, diabetic) P. obesus (p=0.009) (see FIG. 40), but not in Group A or Group B P. obesus. Hypothalamic AGT-717 gene expression was positively correlated with body weight (p=0.025) (see FIG. 41), blood glucose (p=0.002) (see FIG. 42), plasma insulin (p=0.011) (see FIG. 43) and whole-body fat oxidation rate (p=0.046) in P. obesus (see FIG. 44).

### EXAMPLE 23

### Partial Sequence of Psammomys obesus AGT-717

The partial nucleotide sequence of Psammomys obesus AGT-717 cDNA is as follows:

(see FIG. 39: SEQ ID NO:7)

CCGGAATTCCGGGATATCGTCGACCCACGCGTCCGGTGGCCAGGCCGGCC
TTGTTGGGGCTGCTGACTCCAGGTCTATTCTCAGCAATGGGTCACAGAGA
CTGAACTGCCACCATGATTGGAGGCTTATTCATCTATAATCACAAGGGGG
AGGTGCTCATCTCCCGAGTCTACAGAGATGACATCGGGAGGAACGCTGTG
GATGCCTTCCGGGTCAATGTGATTCATGCGCGGCAGCAGGTGCGAAGCCC
TGTCACAAACATCGCTCGCACCAGCTTCTTCCATGTTAAGCGGTCCAACA
TCTGGCTAGCAGCAGTCACCAAGCAGAATGTCAACGCCGCCATGGTCTTC
GAATTCCTCTATAAGATGTGTGATGTAATGGCTGCTTAC

The nucleotide demonstrated sequence homology to the Adaptor-related protein complex 2, mu 1 subunit (AP2M1).

### EXAMPLE 24

### AGT-718 Gene Expression it the Hypothalamus, Liver, Mesenteric Adipose Tissue and Red Gastrocnemius

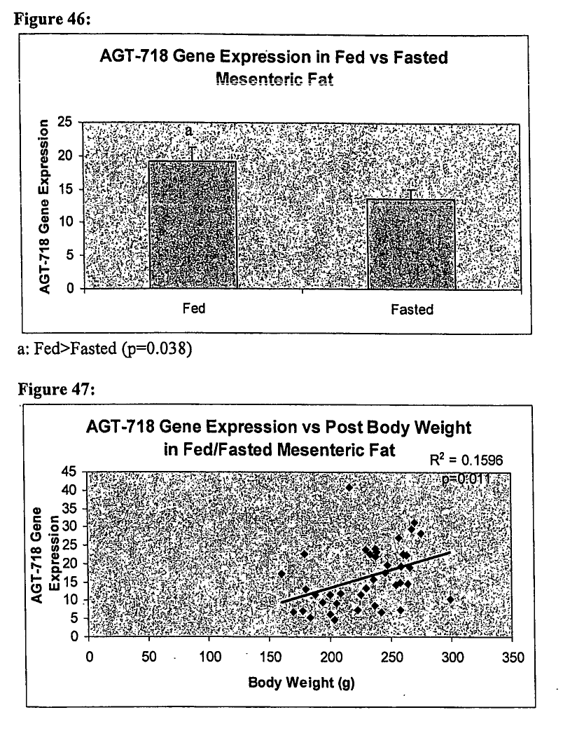

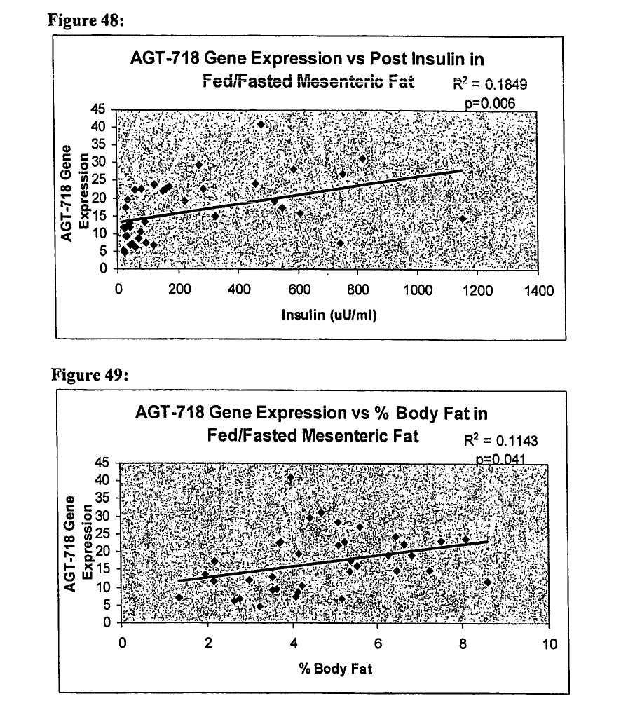

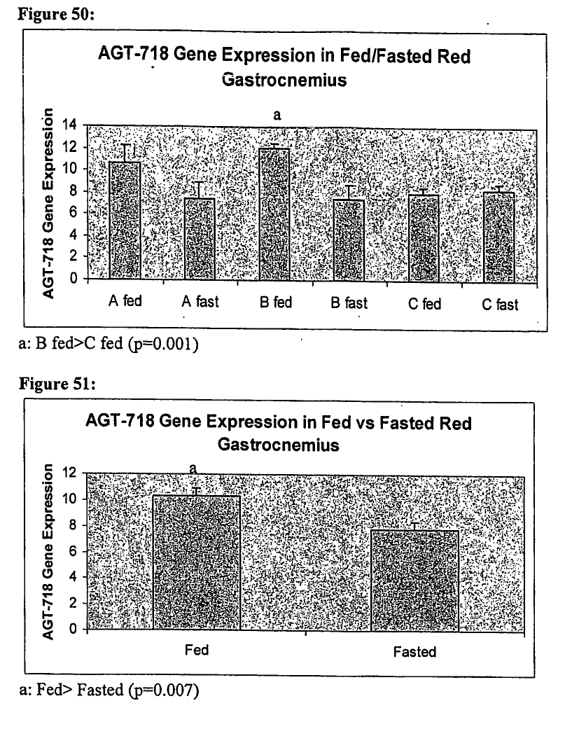

Hypothalamic AGT-718 gene expression was significantly increased in Group C (obese, diabetic) P. obesus compared with Group A (lean, nGT) P. obesus (p=0.023) (see FIG. 46). Hypothalamic AGT-718 gene expression was positively correlated with body weight (p=0.043) (see FIG. 47), plasma insulin (p=0.019) (see FIG. 48), blood glucose (p=0.003) (see FIG. 49), physical activity levels (p=0.045) (see FIG. 50) and whole-body fat oxidation rate (p=0.035) (see FIG. 51) in P. obesus.

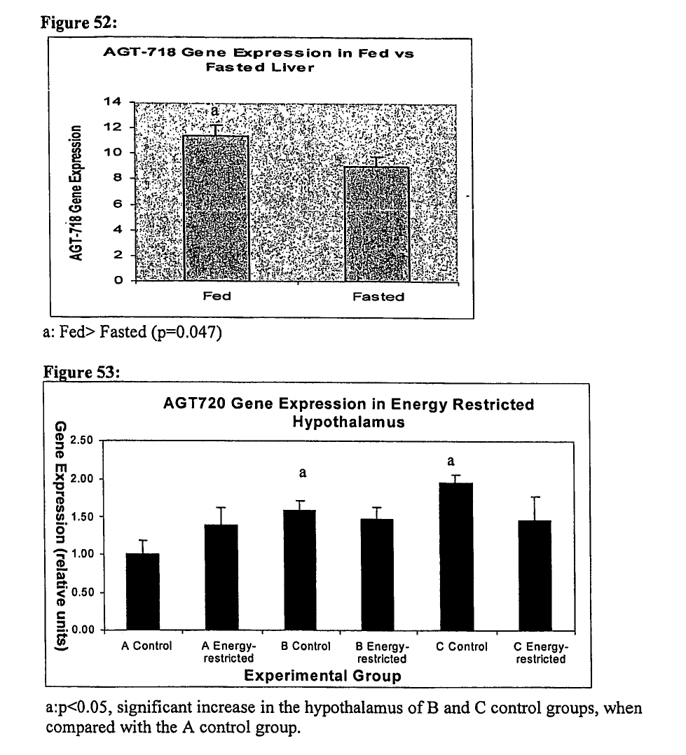

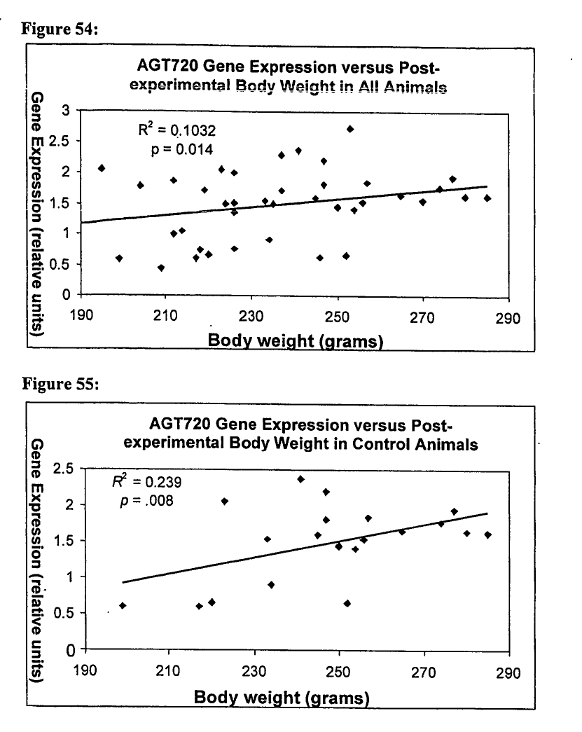

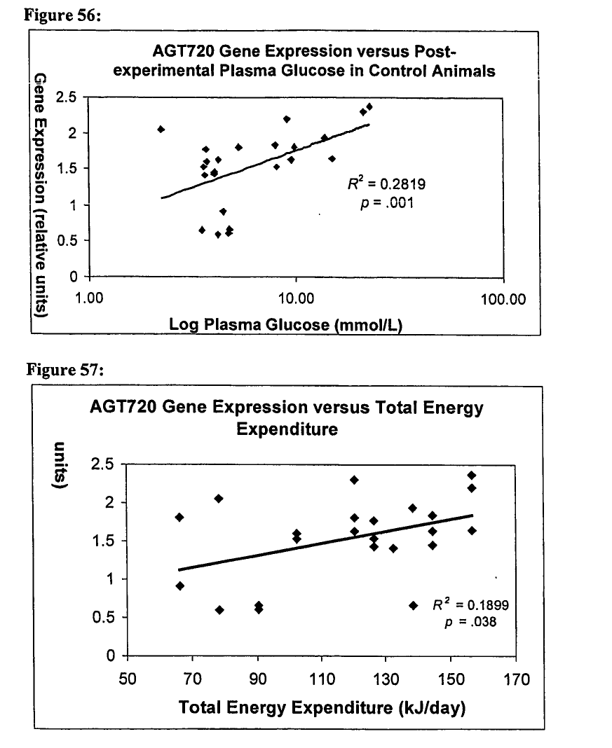

AGT-718 expression was also examined in mesenteric fat of P. obesus after fasting (24 h) or ad libitum feeding. In the fed state, expression of AGT-718 in mesenteric fat was elevated in Group B (obese, IGT) (p=0.005) and Group C (obese, diabetic) (p=0.007) P. obesus (see FIG. 53) compared with Group A (lean, nGT) P. obesus. After fasting for 24 h, mesenteric fat gene expression of AGT-718 was significantly elevated in Group C (obese, diabetic) P. obesus compared with Group A (lean, nGT) P. obesus (p=0.027) (see FIG. 53). Overall there was a significant difference in mesenteric fat AGT-718 gene expression in fed versus fasted (24 h) P. obesus (p=0.038) (see FIG. 54). Mesenteric fat AGT-718 gene expression was positively correlated with body weight (p=0.011) (see FIG. 55), plasma insulin concentration (p=0.006) (see FIG. 56) and percent body fat (p=0.041) (FIG. 57) in P. obesus.

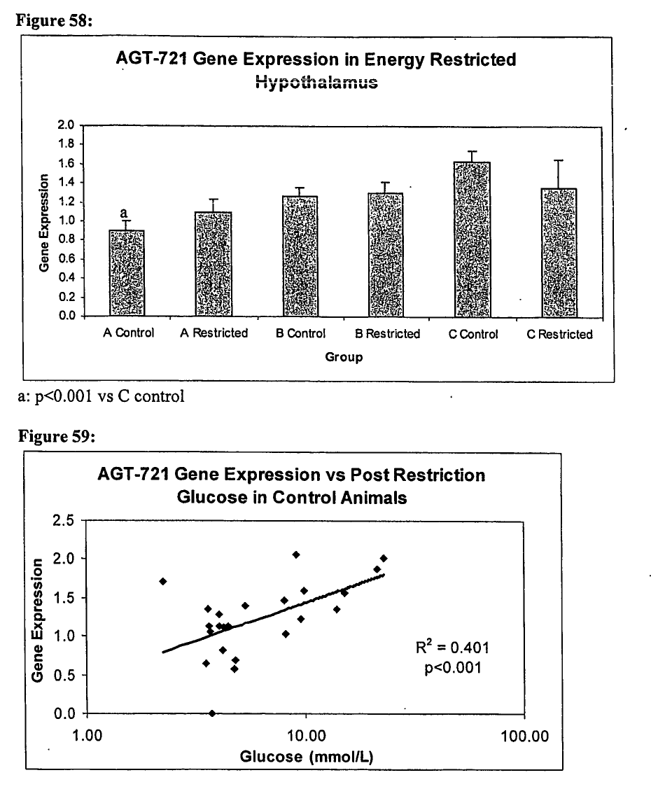

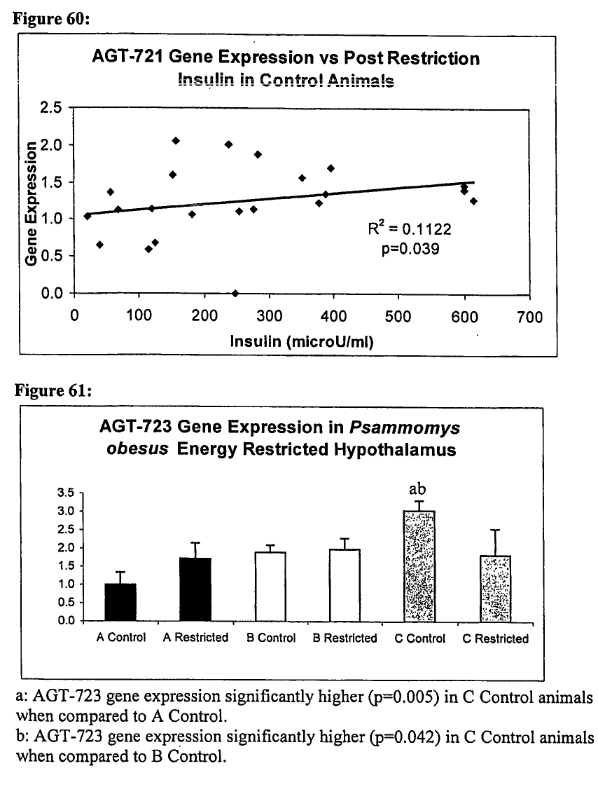

AGT-718 expression was also analysed in red gastrocnemius muscle of P. obesus after fasting (24 h) or ad libitum feeding. In the fed state, there was a significant decrease in Group C (obese, diabetic) P. obesus compared with Group B (obese, IGT) P. obesus (p=0.001) (see FIG. 58). Overall, there was a significant reduction in skeletal muscle AGT-718 gene expression after fasting (24 h) (p=0.007) (see FIG. 59). In the liver, a significant difference was observed when comparing fed to fasted (24 h) P. obesus (p=0.047) (see FIG. 60).

The significant differences in AGT-718 expression within this animal model of obesity and type 2 diabetes indicates that AGT-718 may be a functional target for the treatment of obesity and type 2 diabetes.

### EXAMPLE 25

### AGT-718 Tissue Distribution

A SYBR Green PCR tissue distribution in Psammomys obesus was performed for AGT-718. Expression of AGT-718 is represented in FIG. 52. Expression was ubiquitous, with highest levels in the brain, heart, adrenals, lung and testes.

### EXAMPLE 26

### Partial Sequence of Psammomys obesus AGT-718

The partial nucleotide sequence of Psammomys obesus AGT-717 cDNA is as follows:

(FIG. 45: SEQ ID NO:8)

GTCATCACACCTGTGGGAAATACCTTACACATTACTGTTCGTGAGTAGCT
GTGAGACATCACAGTGGCCTGGTCTGCAGTTGTTGCTCTAAATAATTCGT
TTAGAGTGTTTTTCTCGTTTATGTCTGCGTAATGACTAAAATAGTGAGCT
CAGGCCCAGTGGCACAGCTGTACTCCCAGCTCTTCAGGGGGCTGTGGCCG
GAGGACTGCTAGTTTGAGGCCAGCCCTGCAGTAAAGTAGTTACCAACAGT
GAGACTAAATAAAAAAAGTTCAGAGCACAAACGAATACTTGAAGCAAAAG
AGTCTTTCATTTTCTTTCTCTGACTGAAAAAAACAAGCACTTTCCGGAAG
CCAAGCACTTTTCATTGTAGCAAGGATGAGTTATTTACGTTTTAGGAAAT
TTTATCATGGATTAGTCTGATAATTTAAATTCTGGCCAGTCTGTTTTCAT
CTTTTTCTAGGTTTTACAGTATTTCCATCATACCTATGTAAAAGGAGAAT
TATATACTTGTATCATATTGGTGTTGATAATGTATATTCAGATAATAATA
TTTTGTTAGTGTCTTTATTATCTCAAAAGGAGTTCTAAAAGTAAATACTG
GAAGCATATACTTTTTAGACATTAATTATTTTATAATGAAACAACTGGCA
GTAAGACATTAGTGGTTAATATAGTCTATTTGTTCCAGTGACTGTAAAGC
TTGTCTCATAGTTCTGTCTGCTCATTAGAGAACGTATTGTGAAATTACCT
TCTGTGCATGATATATCTGTGCAATTGCAGT

The nucleotide sequence demonstrated homology to GenBank entry KIAA0033.

### EXAMPLE 27

### AGT-720 Gene Expression in the Hypothalamus

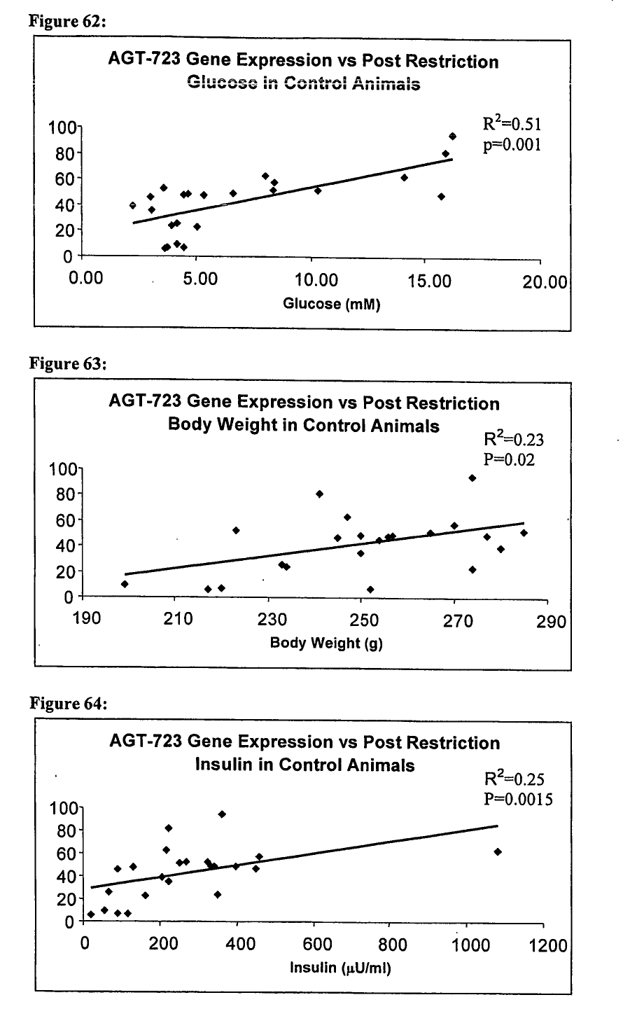

Hypothalamic AGT-720 expression was significantly increased in Group B (obese, IGT) and Group C (obese, diabetic) (p=0.05) (see FIG. 62) P. obesus compared with Group A (lean, nGT) P. obesus.

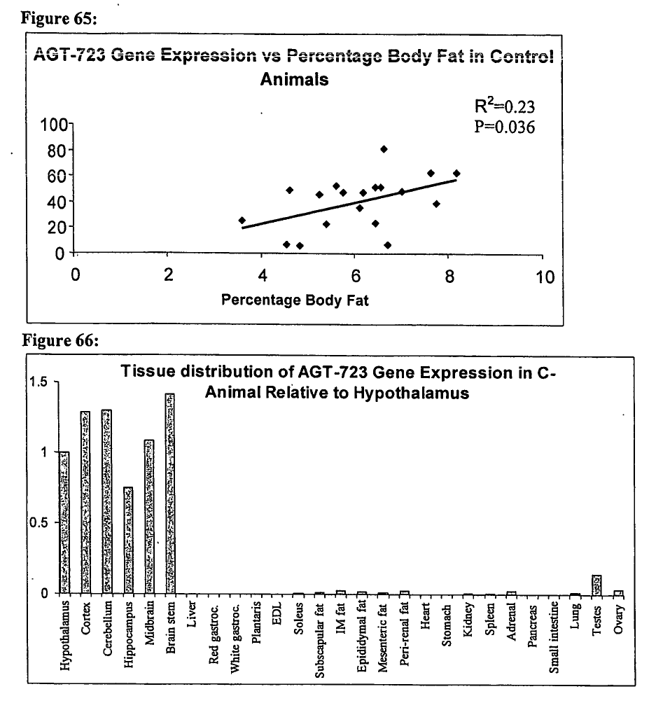

Hypothalamic AGT-720 gene expression was positively correlated with body weight in dietary energy restricted (p=0.014) (see FIG. 63) and ad libitum fed P. obesus (p=0.008) (see FIG. 64). In addition, hypothalamic AGT-720 gene expression was positively correlated with blood glucose (p=0.001) (see FIG. 65) and energy expenditure (p=0.038) (see FIG. 66) in P. obesus.

The significant elevation of AGT-720 gene expression in both obese and diabetic Psammomys obesus indicates that AGT-720 may be a good target in the treatment of human obesity and/or diabetes.

### EXAMPLE 28

### Partial Sequence of Psammomys obesus AGT-720

The partial nucleotide sequence of Psammomys obesus AGT-720 cDNA is as follows:

(see FIG. 61: SEQ ID NO:9)

TTTTTTTTTTTTTTTGTTAGAAATGGGAAAATTTTTCATCATCTCCAGAA
CTGTTTATTAATGATGCTAATAACTTTCTTATAGACCATTTAGACCATAC
AGTTCCAGACCACAACTTCCTGTTTCCAGAATTTGAAATTGTAACAGAAA
CAGAAGATGAGATTTTGCCTGAGGCTGTGGAAATGGAAGATTATTCTGAC
ATTGTAGGAAGCATGGGTAAGGAGTCCGTCCATCAATCTGACAGTTCAGG
GAAGACAGGCACCATCAGTATGTGTCTGTGGTGAGGTAGAGAATATCATG
GTAGAAGACTGCAGATCAAAACAGTTAACTGCTGAATATATGACAAGGGT
CCTTTGTAATTTAGTCTTTTAGTTTTACTTTTAGATACATGCTAGCTTAT
TTTCTCGGGAGCTCATGCTTTAGGGCTGTGTCTGTGGTGACCAACACGGG
TGCTGATCATAAATCCAGCATTCACCCTGCCATAGGTTGCTTATGCATGT
GCTCACTCAAACACTCTTTGCATCGTGCTTCCAGTAAAGAAACTGAAGTT
CTAGGTATCCTGTGTTTGAACAAAGAACTTAGGTTCAGATCTGACACTAC
AGTGTGAACTATAATCAGGTTTGTAAAATCGTAATCAGATTCTCAAGTTT
AGTCGTTTGTTTGCCACAAGCATTAAACAACGCGTGTGAGAATTGTTTCG
TGGCTCAGCTCATGCTTGCTAACAGAAATACACAACAGGAAGAATGGTAC
CACATAGTCAGGGGTCTGTTCGAAGGAGGGAGTGCAGAGAGCGCTCAGCT
ATGTAAAGTGTCTTGTGACTGGTAAAAGTTAAGGAGAAAGGCTGGAGAGA
TGGTCAGAAGTCACAAGCACTTGCTGTTCTTGTAGACGACCCAGGTTTGG
TCCCTAAAACCTGCACGGCAATTTGTAACTCTAGTTCCAGGTGATCCAGC
GCCCTCCTCTGGCCTTGAAGTACACATACATATAAACAAAACATTCAAAA
ACAAACAAAGAAACAAACAAATAGGAATACATACAGCGTAGGAGCTACCT
GGATAGAACTTAAATGTGTCTATTCATGTGTGTACTGTAAAAAAAACTGA
AAAAATCAGTGAGCATGTTTTTTCTTTCATTTTGAGAGAGGTTCTAATGA
AGGCCAAGCTGGCTTTGAACTTGTCCTCCTGCCTCCACTTCCTGAATACA
GATATTACATCATTTTACATCTGTCTGCTGTTATCTTAATGAGTAAACTT
ATATATAGGTCTAGGAAAATAGTACAGAGTTACAAAACAATATAGGTCTG
AACTTTTATTGGAAGATGCCAGCAGCTCATGAGTACATTTATCTACAACA
AAGATTT

The nucleotide sequence demonstrated sequence homology to PDCD2, a gene associated with programmed cell death 2.

### EXAMPLE 29

### AGT-721 Gene Expression in the Hypothalamus

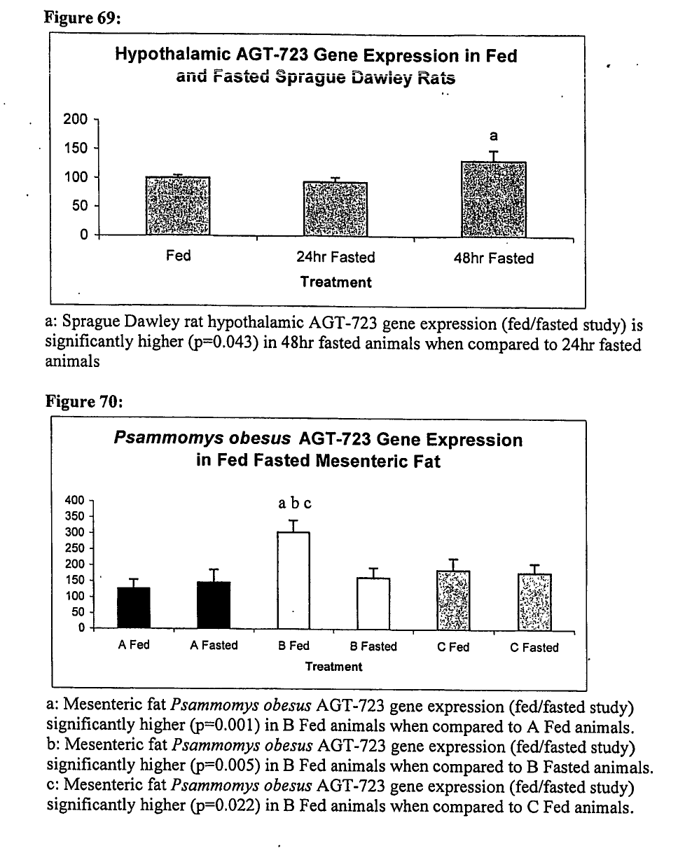

Hypothalamic AGT-721 gene expression was significantly increased in Group C (obese, diabetic) P. obesus compared with Group A (lean, nGT) P. obesus (p<0.001) (see FIG. 68). Hypothalamic AGT-721 gene expression was positively correlated with blood glucose (p<0.001) (see FIG. 69) and plasma insulin concentration (p=0.039) (see FIG. 70) in P. obesus.

### EXAMPLE 30

### Partial Sequence of Psammomys obesus AGT-721

The partial nucleotide sequence of Psammomys obesus AGT-720 CDNA is as follows:

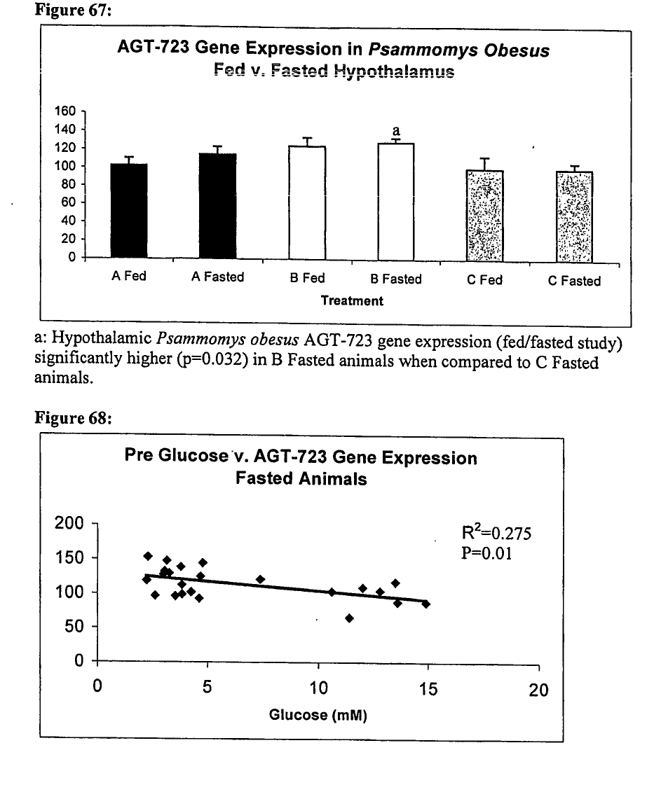

(see FIG. 67: SEQ ID NO:10)

GGCTAGAAAACTACAAGAGAAAAATTCACATGAAAAACCAAAATGAAGAA
AAGGCTGCTGAGCAGTTTCGGATGCGACTGAAAAACAAGCAAGATGAAAT
GAGGCTTGAAGGAGACCTGAGAAGAAGCCAGCGTGCTTGCCAGCAGCTTG
ATGCCCAGAAGAATATTCAGGTTCCCACAGAGGTATGGTACTGGATAAGG
CCTGAAGAAGACACTGAAGAAGAGGAAGTGGAGGAGGAACAGGATGAAGA
TGAATACACGAGTGAAGATTTGAGTGTGCTGGAAAAACTGCAGATCCTCA
CAGGCTACTTACGAGAAGAACATCTGTATTGCATTTGGTGTGGGACAGCC
TATGAAGATAAAGAAGACTTATCTTCCAATTGCCCAGGACCAACCTCTGC
AGATCATGACTGAGATTATTCCCAATAAAATAAAACCATGTATTCAGAAC
TACTACAGTGCTCAGTTAGAATGAAGCCAGAACTGGGGTAGCTACAGCCG
TCTAACTATGAGGACATCAGGAAGCACTTCCTGAGTGGAGGACGTTCTAC
AGAAGGATGACTAGTTCCTCAAGCAAATGTTGAGAGAACAGAAAACACAG
GGAGTATGCCACAGAGTAAAAGTCAACCAAATGAAACCTTGGATCCTCCT
GTTTATAAACAGCTATATACAAAAGACAGTTTTGAGAATATAAAATTGAA
TCCAGACTAATTATATGATAATTATATTTCTGTTTGTCTGAGTAGCAGTG
ATGTGTGATTTTGTTAATATTTCAAAATACTTGTCCATATAAGTCAACTT
TTAAGTATTTGCAGCTGAAACACTACAATGCTAAGAGTAACTTTAAAATT
TTCCAGCCAAAGAAACAAAAAGGAAGGGGTAGGTGTCACAGCAAAGGTAA
AATATTCTCTGTTAAAGCTGCACAATGGCACATACAATCTCATTATTCTT
GTATACGTTTGAAGACAAAAAAATATATGTAATAATTAATCTTGAATGAA
AATATTTTGTCTTAAAAAAATGTTAATGTGCCAGAGAACCTCAAGTTATG
GTGAACATTCAAGTGGGGATGCATGTTGTAAGTCTTTGCCTATTGAGCCT
GCAGATGTGTTTTTAATAGAAATGATATATTTGGTCTGTGATAAAAATTT
CTTAGGTCTGTTTCTATTGATTCAACTAGTTGTTTTGTTTGTTTGTTTGT
TTGTTTGTTTGTTTCAAAACTGTACCTGAAATTAGCAATTGAGGTCCTGC
TCTGCCCCACTGTTACTCTGATTAAGAAGATACTTGTTATTTCCTGGTTT
TTGGAGACAGACGATATATCCTCAGAAGTAAATAAAAGATGCTAAGAGAT
AAAAAAAAAAAAAAA.

The sequence does not match any genes or sequences currently in the public databases.

### EXAMPLE 31

### AGT-723 Gene Expression in thre Hypothalamus and Mesenteric Adipose Tissue

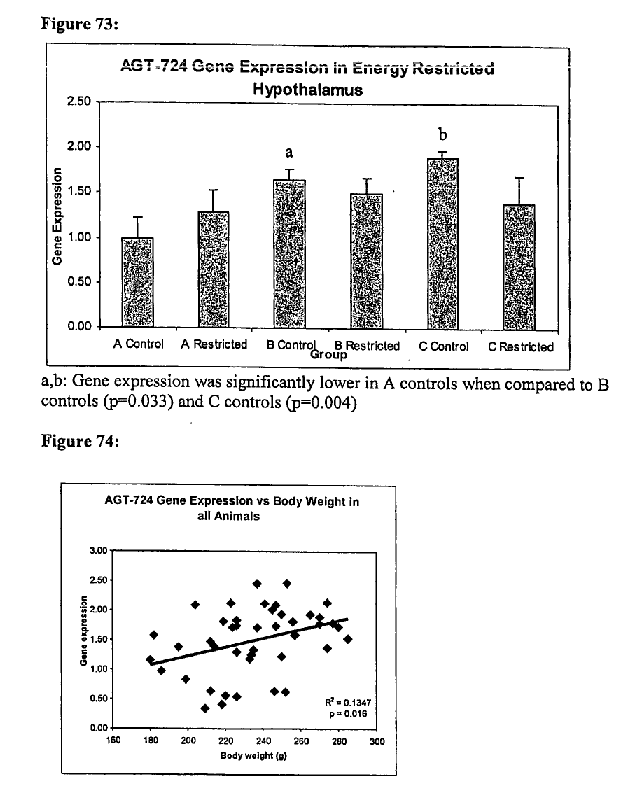

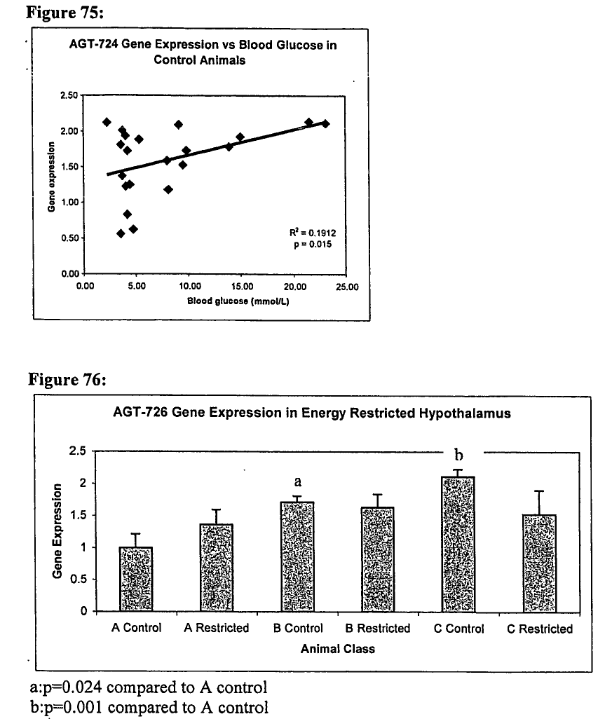

Hypothalamic AGT-723 gene expression was significantly elevated in Group C (obese, diabetic) P. obesus compared with Group B (obese, IGT) (p=0.042) (see FIG. 72) and Group A (lean, nGT) (p=0.005) (see FIG. 72) P. obesus. Hypothalamic AGT-723 gene expression was positively correlated with blood glucose (=0.001) (see FIG. 73), body weight (p=0.02) (see FIG. 74), plasma insulin (p=0.002) (see FIG. 75), and percentage body fat (p=0.036) (see FIG. 76) in P. obesus.

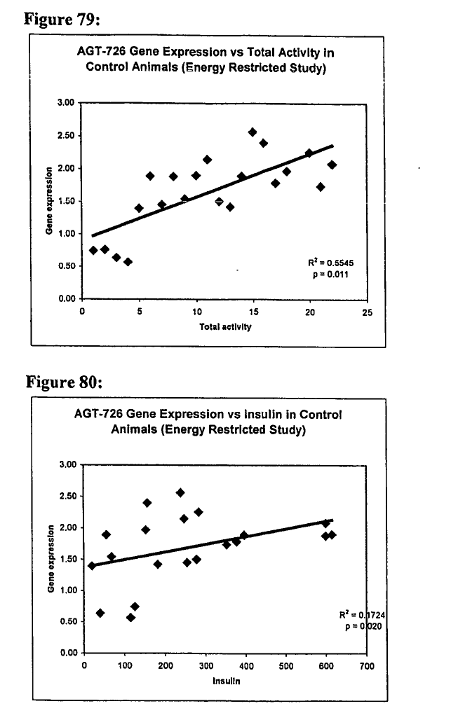

After fasting (24 h), hypothalamic AGT-723 gene expression was elevated in Group B (obese, IGT) P. obesus compared with Group C (obese, diabetic) P. obesus (p=0.032) (see FIG. 78). After fasting (24 h) hypothalamic AGT-723 gene expression was negatively correlated with blood glucose (p=0.01) (see FIG. 79). In Sprague-Dawley rats (Rattus norvegicus), there was a significant increase in hypothalamic AGT-723 gene expression after fasting (48 h) (p=0.043) (see FIG. 80).

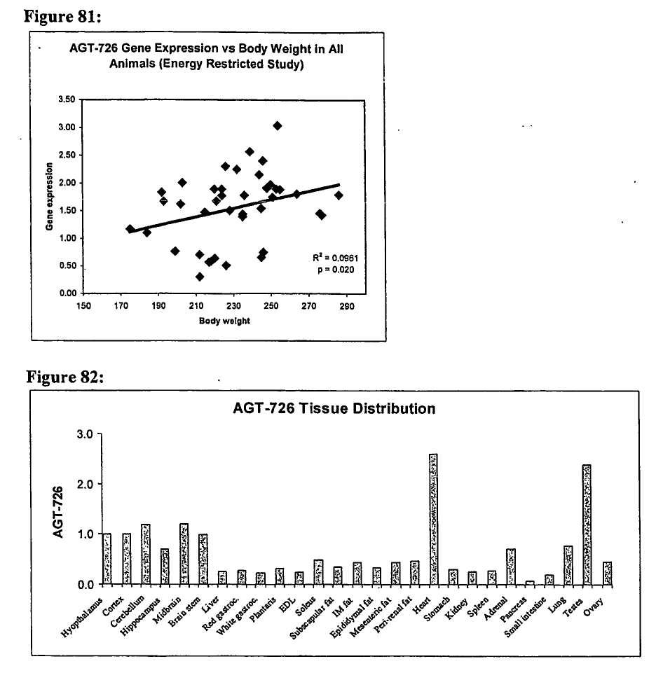

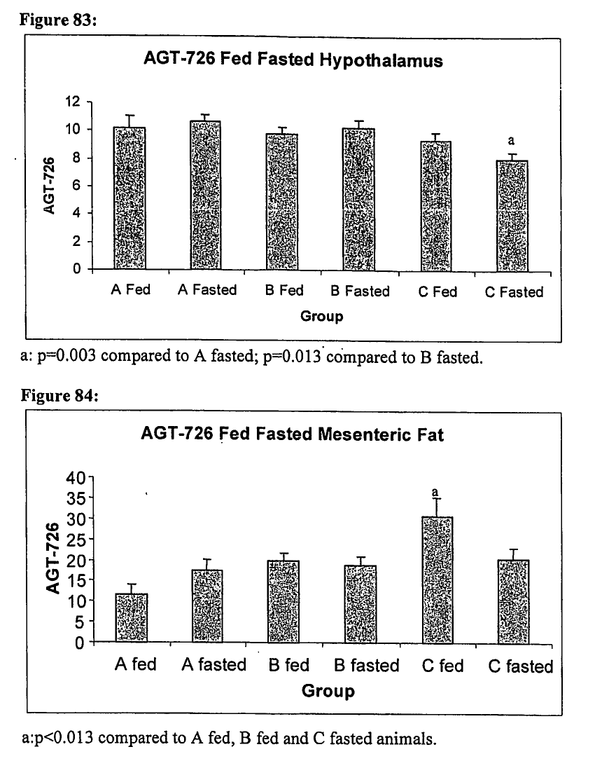

In mesenteric fat, AGT-723 gene expression was significantly elevated in Group B (obese, IGT) P. obesus compared with Group A (lean, nGT) P. obesus (p=0.001) (see FIG. 81), and was significantly reduced after fasting (24 h) in these animals (p=0.005) (see FIG. 81). Mesenteric fat gene expression of AGT-723 was positively correlated with body weight (p=0.03) (see FIG. 82) and plasma insulin concentration (p=0.003) (see FIG. 83) in P. obesus.

### EXAMPLE 32

### AGT-723 Tissue Distribution

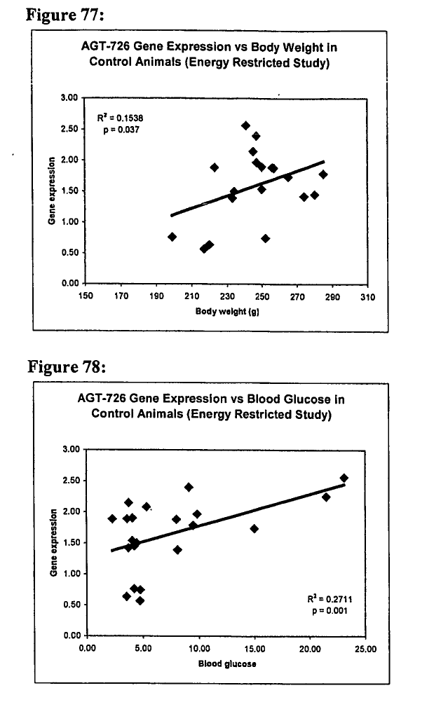

A SYBR Green PCR tissue distribution in Psammomys obesus was performed for AGT-723. Expression of AGT-723 is represented in FIG. 77. Expression was high in all areas of the brain, with minimal expression in adipose tissue and reproductive organs.

### EXAMPLE 33

### Partial Sequence of Psammomys obesus AGT-723

The partial nucleotide sequence of Psammomys obesus AGT-723 cDNA is as follows:

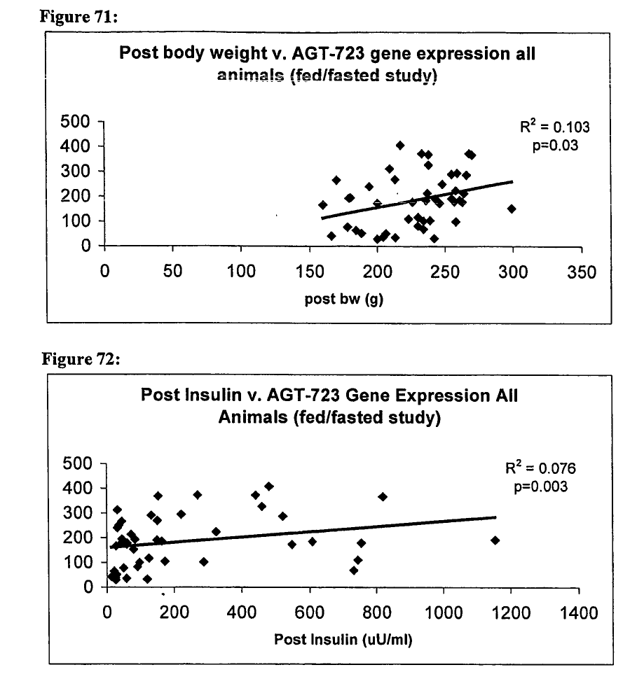

(see FIG. 71: SEQ ID NO:11)

CACCGCCCGCCGTCACGTCGGCGGAGGGCTGCGGGGCGCGTGCGATTGAG
CTGCTGGAGTTCTCAAGATGAAGTTTTCATTGGCGATCTCCTTTTTTATT
TTAATGTCCTTGTGGTTTGAAGAAGCTTGTTCTAAAGAAAAGTCTTCCAA
GAAAGGGAAGGGGAAAAAGAAGCAGTATTTGTGCCCATCTCAGCAGTCAC
CAGAGGACCTGGCGCGTGTGCCCCCCAACTCCACCAGTAATATCCTGAAC
AGGCTGCTGGTCAGTTATGACCCCAGGATCAGACCAAATTTCAAAGGTAT
TCCTGTCGATGTAGTAGTC.

The nucleotide demonstrated sequence homology to the Glycine receptor, beta subunit (Glrb).

### EXAMPLE 34

### AGT-724 Gene Expression in the Hypothalamus

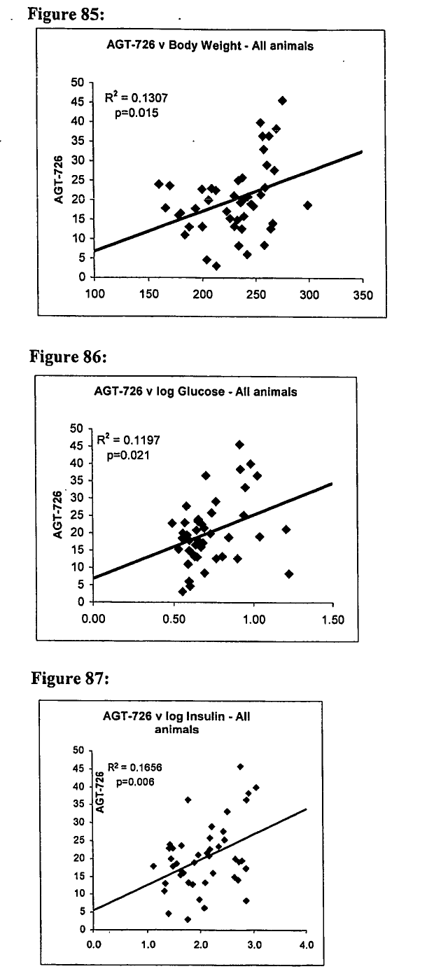

Hypothalamic AGT-724 gene expression was significantly elevated in Group B (obese, IGT) (p=0.033) and Group C (obese, diabetic) (p=0.004) P. obesus compared with Group A (lean, nGT) P. obesus (see FIG. 85). Hypothalamic AGT-724 gene expression was positively correlated with body weight (p=0.016) (see FIG. 86) and blood glucose (p=0.0 15) (see FIG. 87) in P. obesus. These observations suggest that AGT-724 may play a functional role in disorders of the metabolic syndrome, and may represent a new target for obesity/diabetes therapeutics.

### EXAMPLE 35

### Partial Sequence of Psammomys obesus AGT-724

The partial nucleotide sequence of Psammomys obesus AGT-724 cDNA is as follows:

(see FIG. 84: SEQ ID NO:12)

AAACTCTGTCTCAAAACAAAACAAAGTAACAAAGGGATAGGCCAAGTCTT
CTGAGAAGTTAGAGGCAAAGTGCTTGCTTGAGCCTAATGCTCTTCCCACA
GCCGTCCTCAGCCCAGGTCCTCTCCTCTCCTGCAATCAAGAGGATATTGC
TTTAGTTGACATGGGCCTTTCCACGCATCTGCTGAGGTGATTTTCCAGGT
AACTACAGGGGTGAAGGCTAACTCAACACAAGCCAAGAAATACAGAATCT
AGATATACCTGGTCTTACCTAGATGGGAGACCAGCACCGTCCAATAGCAG
GGCAGCCAGCCGCCCAGATCCTTATAGGTCTAGGAACACTGGATGACAGC
TGGTGAACATGGGCTGGAGTGACTGTGACCCCTTCAAGGGAAAGCCTCAG
CTCTTTTTACCTGTTAAGGAATGATGGAGATGCAGACAGTAGGAGAGTGG
TGTGGGAGAAATCCAAAGTGGCAATTTCCCAAACACTTTTTAAAATAAAT
CTTGTGTAGGATGACATAGGGCTTTGATTGTGGCTCAGTGGTACCATGCT
TGTCTTGGACACTTTAATGTGACAAGTCTGAGGGTCCGGTCTATCACTGG
AACTGCAAAAACCAAGAAACAACTGAAATGATAAAGCAGTTCAGAGCTGG
GAAGTTTATAATTTCTTTCTTCTTATACCTTTTCCCCCAAGTGGAAAATG
CTTAATTAGTTGAAGGGCCCCGGAACAGTCCTAAGAGCCTGGGAAACACT
TATTTGCAAGATGGGTGCATTCTGGTTTCCCTAAAACCACAACTCAAAAG
GAAGGTTGTGGGGACAGATGAGTCATGCTGCCTCAGACTAGTGGGTGAAA
GAGGAAGACAGTGTCCTGCCTGTGGCCTCATGCCAGCAGAACACCTGAGA
GTCTGAGGGAGGTTGTGTGACCTTGCCACCAAATGGTATACAGCCGAGCA
GGCGAAGANAGGGTGACAGAGTGGGCAGCCTTCTCTTCAGCAAGATGTAA
GGAACAATGCCAGTTGCTCATACTAACGAGAAGCAGCCCCTTTGCCAGAA
GGTTCTCAGTACGCCCTCCCTCCTTACATTCACTTTGTCCCTTTCAGAGA
GCCAGGTCACCACAAAGGCCACCTGGCCTGCCCACTCACTTCTGCCAAAA
TGTTGCATGCCAGCGTGGAAGACACTGCACAAATCCCAGTGTGTATTTAC
TTAGTGGACACAGATACCTTTAAAATAAAATAAATTCATTAATGAAAAAA
AAAAAAAAAAAA.

The nucleotide demonstrated sequence homology to the kinesin family member 5A.

### EXAMPLE 36

A GT-726 Gene Expression in the Hypothalamus and Red Gastrocnemius Tissue

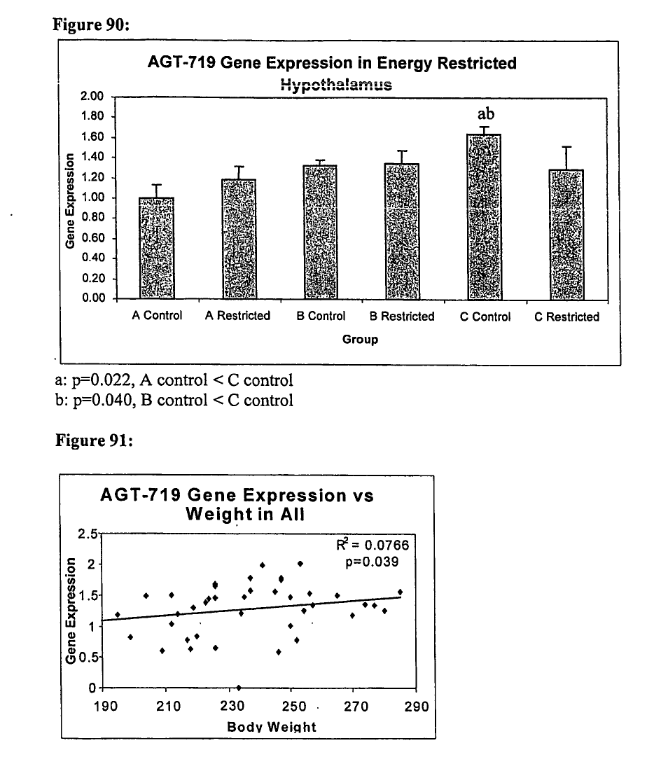

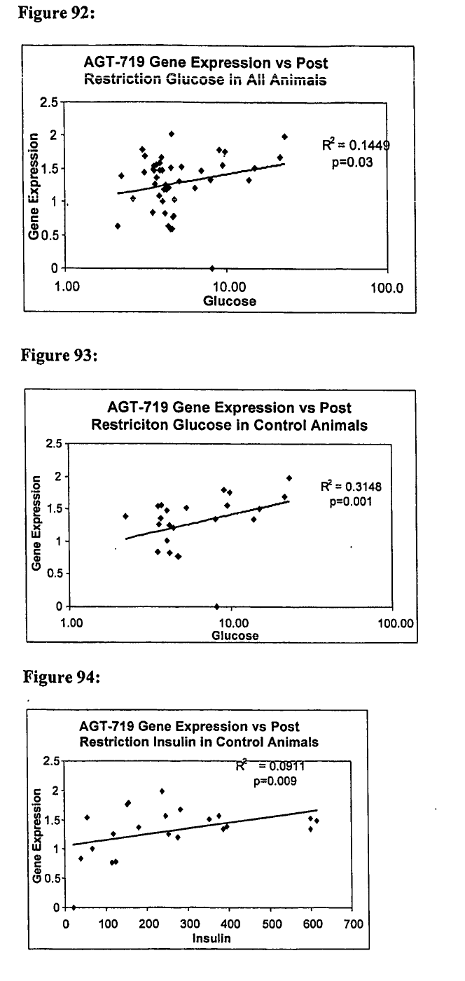

Hypothalamic AGT-726 gene expression was significantly elevated in Group B (obese, IGT) (p=0.024) and Group C (obese, diabetic) (p=0.001) P. obesus compared with Group A (lean, nGT) P. obesus (see FIG. 89). Hypothalamic AGT-726 gene expression was positively correlated with body weight (p=0.037) (see FIG. 90), blood glucose (p=0.001) (see FIG. 91), total physical activity (p=0.011) (see FIG. 92), and insulin (p=0.020) (see FIG. 93) in P. obesus, as well as being positively correlated with body weight (p=0.020) (see FIG. 94) in dietary energy restricted P. obesus.

Gene expression of AGT-726 was examined in the hypothalamus of P. obesus after fasting (24 h) or ad libitum feeding. After fasting (24 h), hypothalamic AGT-726 gene expression was reduced in Group C (obese, diabetic) P. obesus compared with Group B (obese, IGT) (p=0.013) and Group A (lean, nGT) (p=0.003) P. obesus (see FIG. 96).

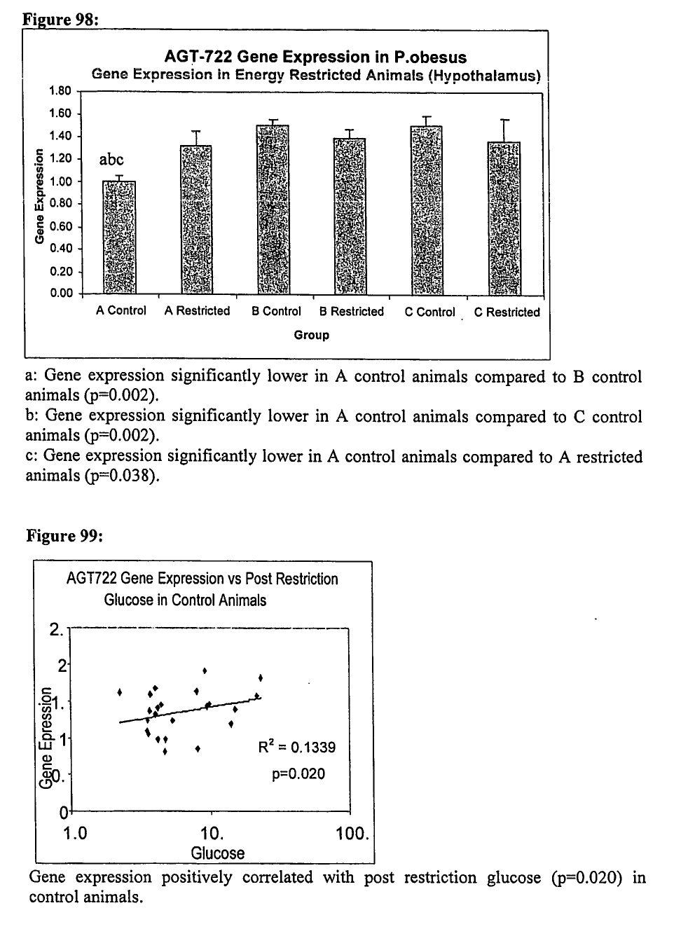

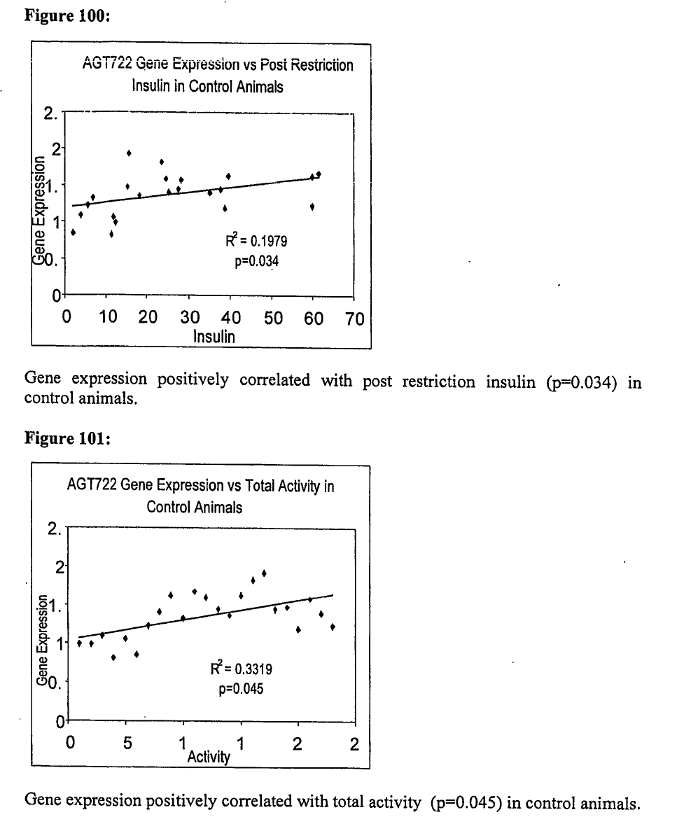

AGT-726 gene expression was examined in mesenteric fat of P. obesus after fasting (24 h) or ad libitum feeding. In the fed state, mesenteric fat AGT-726 gene expression was elevated in Group C (obese, diabetic) P. obesus compared with Group B (obese, IGT) (p=0.011) and Group A (lean, nGT) (p<0.001) P. obesus. Fasting (24 h) significantly reduced mesenteric fat gene expression of AGT-726 in Group C (obese, diabetic) P. obesus (p=0.012), but not in Group B or Group A P. obesus (see FIG. 97). Mesenteric fat AGT-726 gene expression was positively correlated with body weight (p=0.015) (see FIG. 98), blood glucose (p=0.021) (see FIG. 99) and plasma insulin concentration (p=0.006) in P. obesus (See FIG. 100).

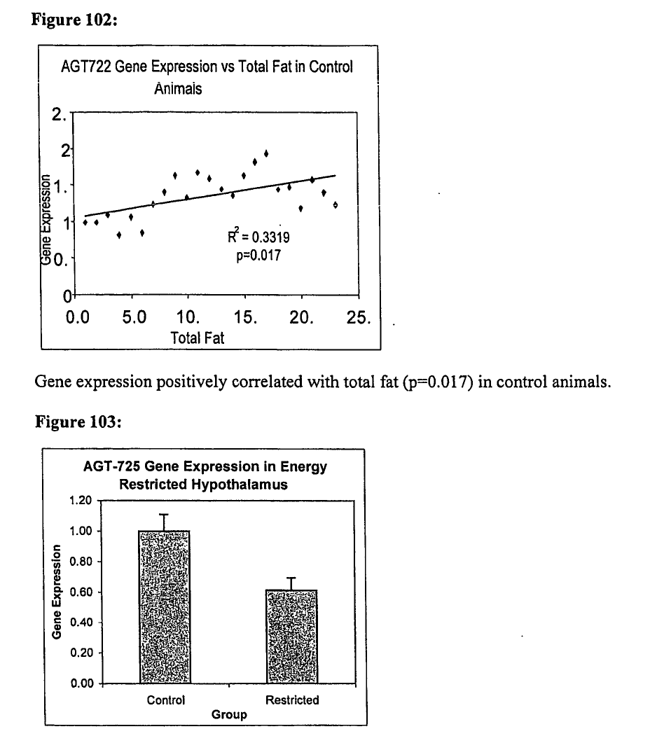

AGT-726 gene expression was also analysed in the red gastrocnemius muscle of P. obesus after fasting (24 h) or ad libitum feeding. In the fed state, AGT-726 gene expression in skeletal muscle was reduced in Group C (obese, diabetic) P. obesus compared with Group B (obese, IGT) (p=0.002) and Group A (lean, nGT) (p=0.018) P. obesus (See FIG. 101). Skeletal muscle AGT-726 gene expression was negatively correlated with blood glucose concentration (p=0.001) (See FIG. 102).

### EXAMPLE 37

### AGT-726 Tissue Distribution

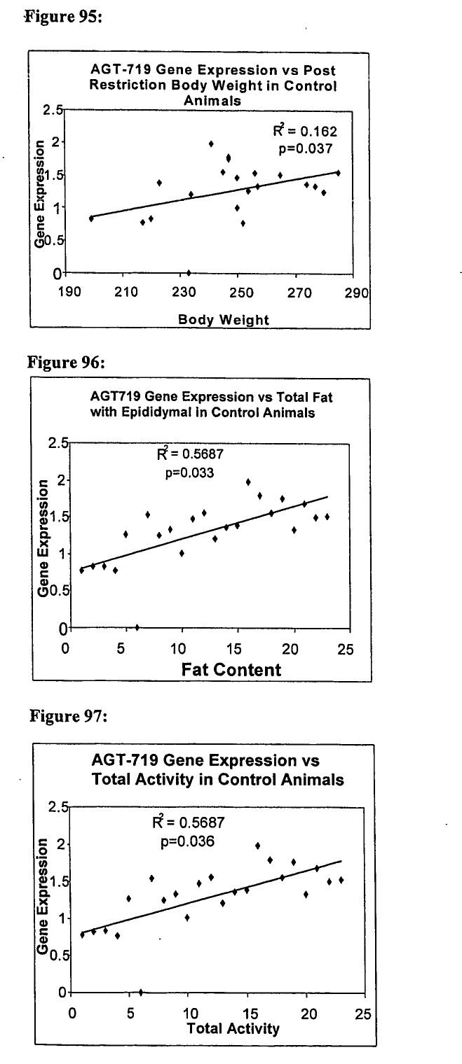

A gene expression profile for AGT 726 was generated from a tissue distribution set of Psammomys obesus cDNA. Expression was found to be highest in the heart and testes, relative to hypothalamic gene expression. Detectable levels of gene expression were found in all tissues examined (see FIG. 95).

### EXAMPLE 38

### Partial Sequence of Psammonys obesus AGT-726

The partial nucleotide sequence of Psammomys obesus AGT-726 cDNA is as follows:

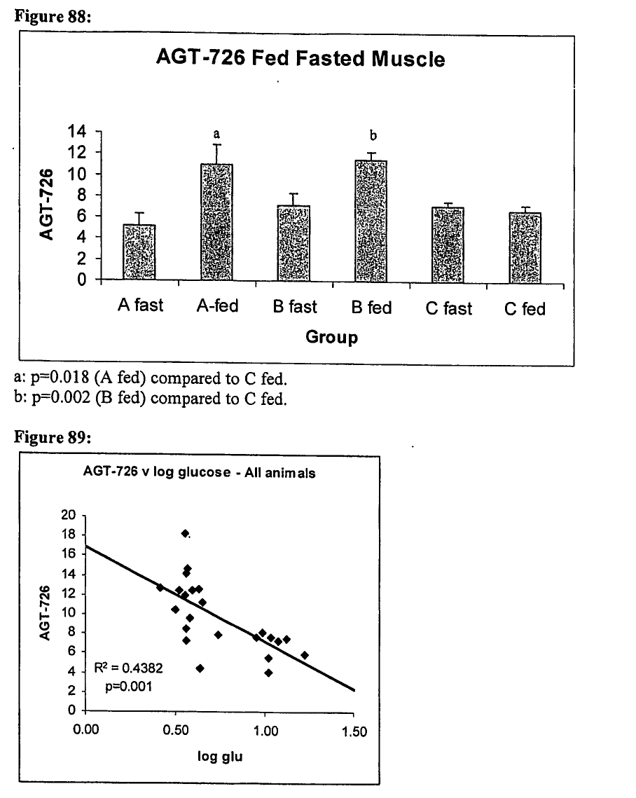

(FIG. 88 SEQ ID NO:13)

GCTGCTTTAGCCAAAGCCATCGAAAAGAACGTGTTATTTTCACACCTTGA
TGACAATGAGAGAAGTGACATTTTTGATGCTATGTTTCCAGTCTCCTTTA
TTGCTGGAGAGACAGTTATTCAGCAAGGTGATGAAGGGGATAACTTCTAT
GTGATTGATCAAGGAGAAATGGATGTCTATGTCAATAATGAATGGGCAAC
CAGCGTTGGGGAAGGAGGGAGCTTCGGAGAGTTGGCTTTGATTTATGGAA
CACCTAGAGCAGCCACTGTCAAAGCAAAGACAAATGTGAAACTGTGGGGC
ATCGACCGAGACAGCTACCGAAGAATCCTCATGGGAAGCACTCTGAGAAA
GAGGAAGATGTATG

The nucleotide sequence demonstrated sequence homology to Protein Kinase, cAMP-dependent, regulatory, type 1 alpha (PRKAR1A).

### EXAMPLE 39

### AGT-719 Gene Expression in the Hypothalamus

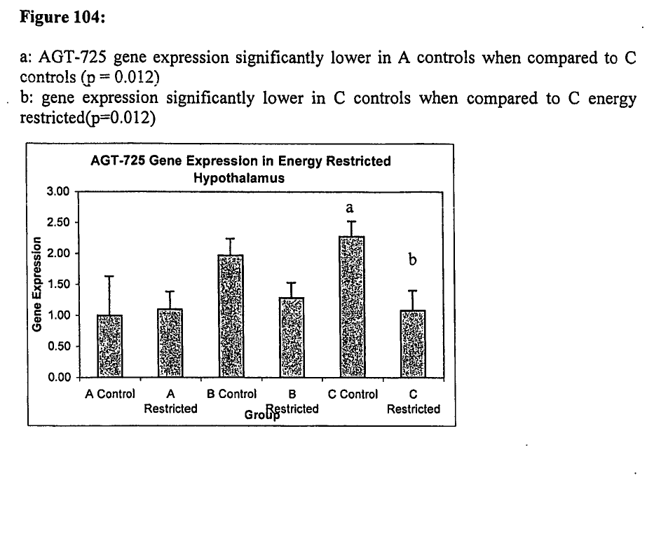

AGT-719 gene expression in the hypothalamus was found to be significantly elevated in Group C (obese, diabetic) P. obesus compared both Group A (lean, nGT) (p=0.022) and B (obese, IGT) (p=0.040) (see FIG. 104). When AGT-719 gene expression was examined in the hypothalamus, there was a positive correlation between body weight (p=0.039) (see

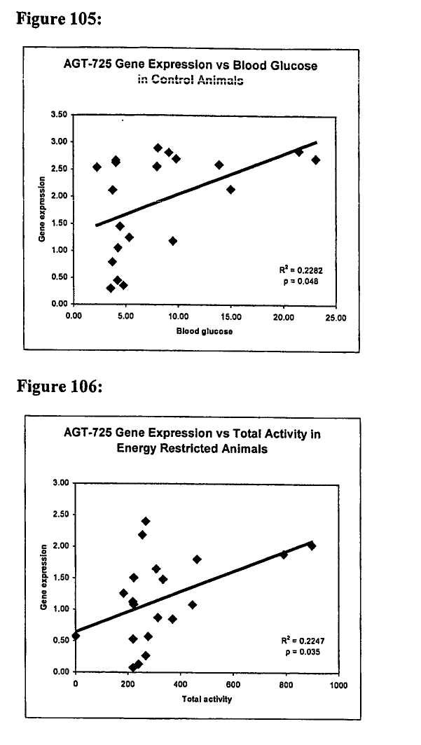

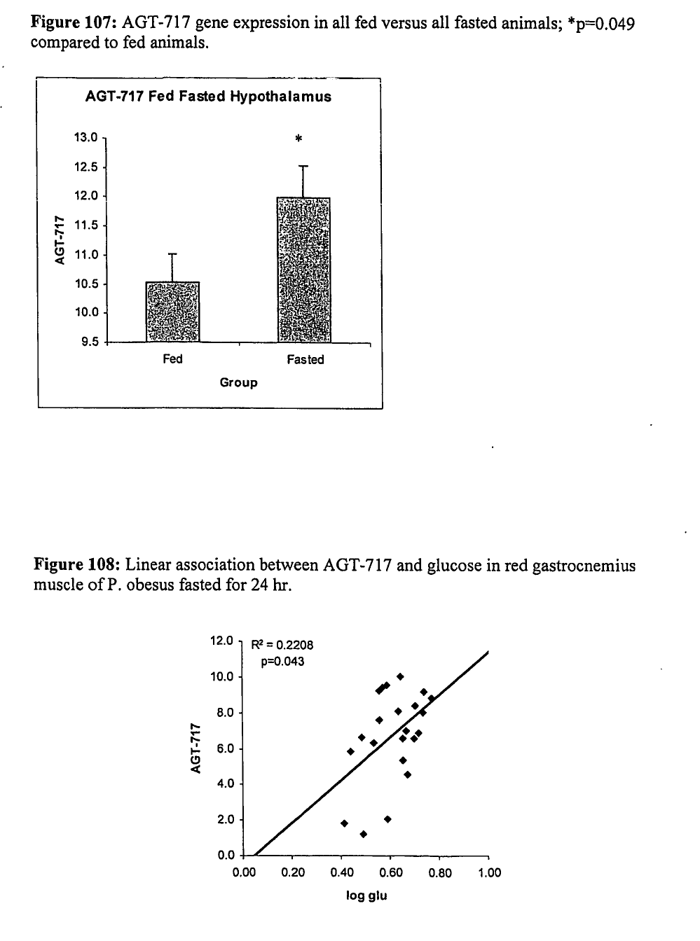

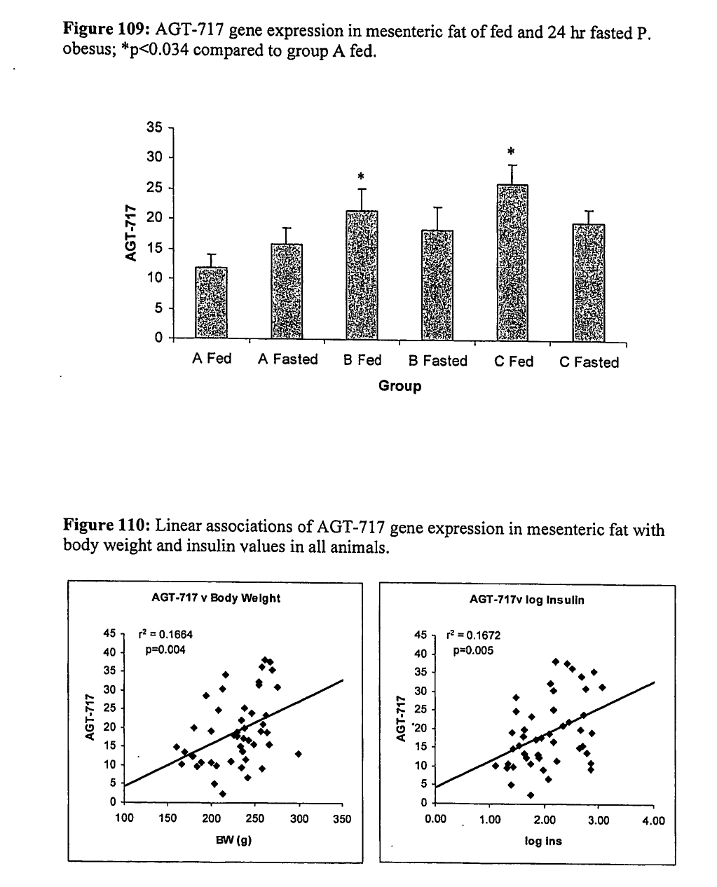

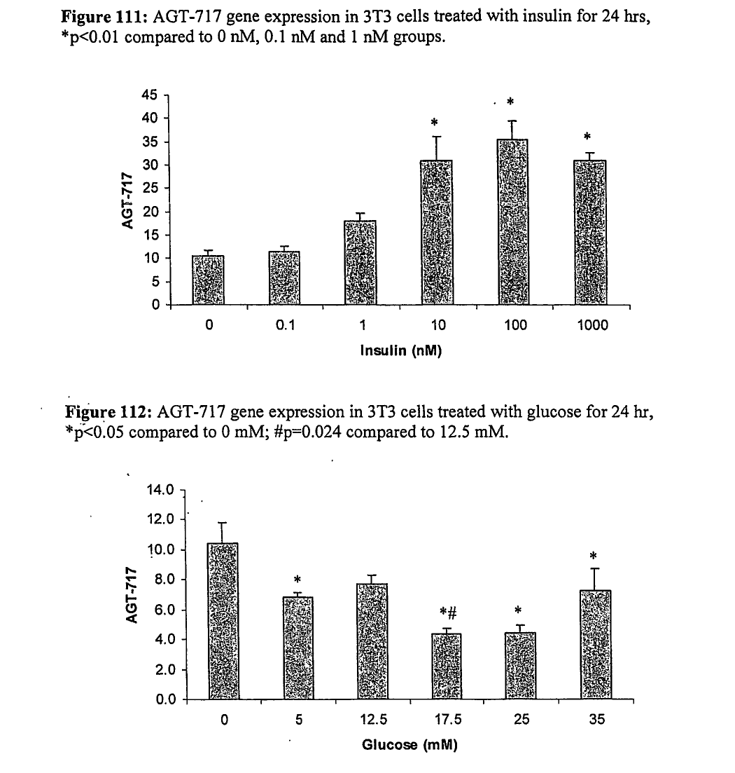

FIG. 105) and blood glucose levels (p=0.03) (see FIG. 106). Hypothalamic gene expression in control animals was shown to be positively correlated with blood glucose (p=0.001) (see FIG. 107), insulin levels (p=0.009) (see FIG. 108), body weight (p=0.037) (see FIG. 109) total body fat (p=0.033) (see FIG. 110) and total physical activity (p=0.036) (see FIG. 111).

### EXAMPLE 40

### Partial Sequence of Psammomys obesus AGT-719

The partial nucleotide sequence of Psammomys obesus AGT-726 cDNA is as follows:

(see FIG. 103: SEQ ID NO:14)

CCCACGCGTCCGCCCACGCGTCCGCGGACGCGTGGGCTGTCCTAGCCTTG
CTTTATAGACCAGGCTGGCTTTGACCTCACAGAGATCTGCTTGTTGACCC
TCCCCCAAGTGCTGGAATTACAGGCGTGCGCCACCATACCCAGCTCTGAT
ATCTCTTACATGACAAAAATCAAGTCACTATTAATGAAAATGATCTGCTC
ATTATAGATGGGAGGCTTAACAAATAAGTGATACATGCTAATTTCTGCCA
GTGTCTATTGCTTAATTGTTAATTGTGAGCAGATTACTGAATGCCTCGTC
TATTTTCTACATTTTATTTTACAATAACTCTTTGAGTAAGTTGAAGTTTA
ATTGTGTAGCAAATTTCTATCAGAGAACAATTTAAAGTG.

The nucleotide sequence demonstrated homology to FENS-1 (FYVE domain containing protein localised to endosomes-1), Phosphoinositide-binding protein SR1, WD repeat and FYVE domain containing 1 (WDFY1).

### EXAMPLE 41

### AGT-722 Gene Expression in the Hypothalamus

Hypothalamic AGT-722 gene expression was significantly increased in Group B (p=0.002) and Group C (p=0.002) ad libitum fed animals versus Group A ad libitum fed animals (see FIG. 113). Expression of AGT-722 was also significantly increased in group A energy restricted animals (p=0.038) as compared to group A ad libitum fed control animals (see FIG. 113). Significant correlations were observed between hypothalamic AGT-722 gene expression levels and blood glucose (p=0.020) (see FIG. 114), plasma insulin (p=0.034) (see FIG. 115), physical activity (p=0.045) (see FIG. 116) and total body fat (p=0.017) (see FIG. 117). These significant expression level differences and correlations in this animal model of type 2 diabetes and obesity suggest that AGT-722 may be a functional target in the treatment of human type 2 diabetes and obesity.

### EXAMPLE 42

### Partial Sequence of Psammomys obesus AGT-722

The partial nucleotide sequence of Psammornys obesus AGT-722 cDNA is as follows:

(see FIG. 112: SEQ ID NO:15)

CTATGACATGATGCCAGATTACAGGCAGATCCCATGTAACCAGAAGGACA
ATGTGTCTTCTAAAAAGGGAAGGTGTTACTGGAGCTTTTCTTTCCTCTGC
ACAGCATAGTATCAAAGTATGCCATTAATGAAGATTTATTCCATTACTGC
TATAAATTTTTAGTATAGGAAGAAAACTTTATCACGATCATGGGCCAGTT
AGCCTGATGTATTCAAGAACAAACAAGAGACCCTGTCTCAAGTTAGGAAT
GAGGACAAGTGCCTGAAGGTGCCCTCCAACCTCCACAGTTGTCCGAAGCA
CACATCCGTGATCATGCACACATCAGCACGCGTACAAAATTACACTTGAG
GCTTTAGTATCATGTGTTGATCATTTCAAACCATCAGAGGCAAACACTGA
AGTGGTATTTTCTGTCTCCTGCTTGCCAGTATCTACATTTTCCCTCACAT
TCTAGGTTAAAAAATGGTTCTTTTATAACATGAGCAATTTGTGATGTTTA
TTATAAGTAAATGTTGATGTCAGTGTTAGAATTAAAATTACTTGTAGTGA
TAAAAAAAAAAAAAAAAAAAAAAAAAAAAAAA.

The nucleotide sequence demonstrated sequence homology to the Eukaryotic translation initiation factor 1A (EIF 1A).

### EXAMPLE 43

### AGT-725 Gene Expression in Tire Hypothalamus

Hypothalamic AGT-725 gene expression is significantly lower in A controls when compared to C controls (p=0.012) (see FIG. 119). Gene expression is significantly lower in C controls when compared to C energy restricted (p=0.012) (see FIG. 120) and gene expression positively correlated with blood glucose (p=0.048) (see FIG. 121) in control animals, and positively correlated with total physical activity (p=0.035) (see FIG. 122) in energy restricted animals.

### EXAMPLE 44

### Partial Sequetice of Psammomys obesus AGT-725

The partial nucleotide sequence of Psammoinys obesus AGT-725 cDNA is as follows:

(see FIG. 118: SEQ ID NO:16)

AGAGAAATAAAGATGGAACTCGGATGTTTGAAAATCTTTACATTCAAATA
AGGCCAAATGTCACGGGGAGAAAAAAAAAAATGGGAAGGTGAGCTGCTAT
GACTGAGACCCCTGAACTTCCCACTCACACTCTCTGCAGAACCCCACAAA
ACGACACAGAACACTATGTGGTTGTTCTATGGCTTTTGTTTTTTCGAGGT
TCTTTCAAATATCCTTTGGCGATTGAAGCTTAAATTCCATTTGATTGGCT
TCACCGTCTGTAAATACTTAGCTATTAGCTGTAAGCACTACACGTAGATG
ACTTAACGACGGGCAGGTCCCAGCGATCATAGCTTTATGAC.

The nucleotide demonstrated sequence homology to Mus musculus 0 day neonate kidney cDNA, RIKEN full-length enriched library, clone:D630038G12 product and ENDOTHELIN B RECEPTOR PRECURSOR, full insert sequence.

### EXAMPLE 45

### AGT-711 Expression in Psammomys obesus Tissues

AGT-711 gene expression was assessed by semi-quantitative Real Time PCR in both red gastrocnemius muscle and mesenteric fat obtained from Psammomys obesus animals. The expression was compared between lean normoglycemic normoinsulinemic group A, obese normoglycemic hyperinsulinemic group B, and obese diabetic group C animals in both the fed and fasted states.

TABLE 4
AGT-711 gene expression (arbitrary units) ± SEM

| Group | Red Gastrocnemius muscle | Mesenteric fat |
|---|---|---|
| Fed group A | 10.52 ± 1.22 | 120.63 ± 26.00 |
| Fed group B | 24.97 ± 3.71^{a} | 287.88 ± 74.78^{c} |
| Fed group C | 14.51 ± 3.07 | 195.01 ± 50.43 |
| Fasted group A | 7.81 ± 7.81 | 74.92 ± 23.42 |
| Fasted group B | 20.05 ± 5.54^{b} | 176.29 ± 45.96 |
| Fasted group C | 14.48 ± 2.24 | 216.06 ± 55.90^{d} |

^{a}P = 0.001 and P = 0.010 to A fed and C fed animals, respectively
^{b}P = 0.015 to A fasted animals
^{d}P = 0.026 to A fed animals
^{d}P = 0.050 to A fasted animals

AGT-711 gene expression in the fasted state of all animals also positively correlated to % body fat (R²=0.240, P=0.033) and to post-insulin levels (R²=0.3224, P=0.006).

### EXAMPLE 46

### AGT-717 Gene Expression in P. obesus Tissues

AGT-717 gene expression was compared between hypothalamus cDNA from fed and fasted animals. A significant increase in the fasted state vs fed state (p=0.049) was observed when all animal groups were combined (FIG. 107).

A positive correlation was observed between AGT-717 gene expression and glucose levels, when gene expression was examined in red gastrocnemius muscle (FIG. 108).

Mesenteric fat expression of AGT-717 was seen to be increased in obese group B (p<0.034), and obese diabetic group C P. obesus (p<0.034) compared to lean group A animals in the fed state (FIG. 109). AGT-717 gene expression in mesenteric fat was also shown to be positively correlated with both body weight and insulin (FIG. 110).

In 3T3 cells treated with increasing concentrations of insulin for 24 hrs, AGT-717 gene expression significantly increased with higher insulin levels (FIG. 111). This was clearly seen in the 10 nM, 100 nM and 1000 nM groups. Conversely, in 3T3 cells treated with increasing concentrations of glucose, decreased expression of AGT-717 was seen (FIG. 112). AGT-717 gene expression is greatest at 0 mM glucose compared to all other groups (except 12.5 mM, p=0.05).

### BIBLIOGRAPHY

Altschul et al., Nucl. Acids Res. 25: 3389, 1997.
Australian Institute of Health and Welfare (AIHW), Heart, Stroke and Vascular diseases, Australian facts. AIHW Cat. No. CVD 7 Canberra: AIHW and the Heart Foundation of Australia, 1999.
Ausubel et al., “Current Protocols in Molecular Biology” John Wiley & Sons Inc, 1994-1998, Chapter 15.
Barnett et al., Diabetologia 37: 671-676, 1994a.
Barnett et al., Int. J. Obesity 18: 789-794, 1994b.
Barnett et al., Diabete Nutr Metab 8: 42-47, 1995.
Bonner and Laskey, Eur. J. Biochem. 46: 83, 1974.
Bouchard, C. The genetics of obesity. Boca Raton: CRC Press, 1994.
Collier et al., Ann New York Acad Sci 827: 50-63, 1997a.
Collier et al., Exp Clin Endocrinol Diabetes 105: 36-37, 1997b.
de Looper and Bhatia, Australia's Health Trends 2001. Australian Institute of Health and Welfare (AIHW) Cat. No. PHE 24. Canberra: AIHW, 2001.
Douillard and Hoffman, Basic Facts about Hybridomas, in Compendium of Immunology Vol. II, ed. by Schwartz, 1981.
Kohler and Milstein, Nature 256: 495-499, 1975.
Kohler and Milstein, European Journal of Immunology 6: 511-519, 1976.
Kopelman et al., Int J Obesity 18: 188-191, 1994.
Kopelman, Nature 404: 635-643, 2000.
Marmur and Doty, J. Mol. Biol. 5: 109, 1962.
Mokdad et al., JAMA. 282(16): 1519-22, 1999.
Mokdad, Diabetes Care 24(2): 412, 2001.
Must et al., JAMA. 282(16): 1523-1529, 1999.
National Health and Medical Research Council, Acting on Australia's weight: A strategy for the prevention of overweight and obesity. Canberra: National Health and Medical Research Council, 1996.
Risk Factor Prevalence Study Management Committee. Risk Factor Prevalence Study: Survey No. 3 1989. Canberra: National Heart Foundation of Australia and Australian Institute of Health, 1990.
Ravussin, E., Metabolism 44(Suppl 3): 12-14, 1995.
Shafrir, E. and Gutman, A., J. Basic Clin Physiol Pharm 4: 83-99, 1993.
Stellar, E., Psychol Rev 61: 5-22, 1954.
Walder et al., Obesity Res 5: 193-200, 1997a.
Waters, A-M. and Bennett, S. M. Risk Factors for cardiovascular disease: A summary of Australian data. Canberra: Australian Institute of Health and Welfare, 1995.
Wolf and Colditz, Obes Res. 6: 97-106, 1998.
Zimmet, P. Z., Diabetes Care 15(2): 232-247,1992.

1-46. (canceled)

47. An isolated nucleic acid molecule comprising a sequence of nucleotides encoding or complementary to a sequence encoding a molecule or derivative or homolog thereof associated with one or more of diabetes, obesity, a metabolic disorder, mitochondrial dysfunction, myopathy, genetic disorder or cancer wherein said nucleic acid molecule is selected from:
(i) a nucleic acid molecule comprises a nucleotide sequence as set forth in SEQ ID NO:7 or a nucleotide sequence having at least about 95% identity thereto or a nucleotide sequence capable of hybridizing to SEQ ID NO:7 or its complementary form under high stringency conditions; and
(ii) a nucleic acid molecule comprises a nucleotide sequence as set forth in SEQ ID NO:8 or a nucleotide sequence having at least about 95% identity thereto or a nucleotide sequence capable of hybridizing to SEQ ID NO:8 or its complementary form under high stringency conditions.

48. The isolated nucleic acid molecule of claim 47 wherein the nucleic acid molecule comprises the nucleotide sequence set forth in SEQ ID NO:7.

49. The isolated nucleic acid molecule of claim 47 wherein the nucleic acid molecule comprises the nucleotide sequence set forth in SEQ ID NO:8.

50. An isolated molecule encoded by a nucleic acid molecule comprising a sequence of nucleotides or amino acids encoded by a nucleic acid molecule which is expressed in a larger amount in hypothalamus tissue of obese animals compared to lean animals or in fasted animals compared to fed animals wherein the isolated molecule is encoded by a nucleic acid molecule selected from:
(i) a nucleic acid sequence as set forth in SEQ ID NO:7 or a nucleotide sequence having at least about 95% identity to SEQ ID NO:7 or a nucleotide sequence capable of hybridizing to SEQ ID NO:7 or its complementary form under high stringency conditions; and
(ii) a nucleic acid sequence as set forth in SEQ ID NO:8 or a nucleotide sequence having at least about 95% identity to SEQ ID NO:8 or a nucleotide sequence capable of hybridizing to SEQ ID NO:8 or its complementary form under high stringency conditions.

51. The isolated molecule of claim 50 wherein the molecule is a protein.

52. The isolated protein of claim 51 encoded by a nucleotide sequence set forth in SEQ ID NO:7.

53. The isolated protein of claim 51 encoded by a nucleotide sequence set forth in SEQ ID NO:8.

54. A method for modulating expression of one or more of a nucleic acid molecule comprising the nucleotide sequence as set forth in SEQ ID NO:7 and/or SEQ ID NO:8 in a mammal in order to treat one or more of diabetes, obesity, a metabolic disorder, a mitochondrial dysfiuction, a myopathy, a genetic disorder or cancer, said method comprising contacting said one or more nucleic acid molecules with an effective amount of a modulator of expression of said one or more nucleic acid molecules for a time and under conditions sufficient to up-regulate or down-regulate or otherwise modulate expression of said one or more nucleic acid molecules.

55. A method of modulating activity of a protein comprising an amino acid sequence encoded by the nucleotide sequence as set forth in SEQ ID NO:7 and/or SEQ ID NO:8 in a mammal in order to treat one or more of diabetes, obesity, a metabolic disorder, a mitochondrial dysfunction, a myopathy, a genetic disorder or cancer, said method comprising administering to said mammal a modulating effective amount of said protein for a time and under conditions sufficient to increase or decrease activity of said protein.

56. A method of treating a mammal suffering from a condition characterized by one or more symptoms of diabetes, obesity, a metabolic disorder, mitochondrial dysfunction, myopathy, genetic disorder or cancer, said method comprising administering to said mammal an effective amount of an agent for a time and under conditions sufficient to modulate the expression of a nucleic acid molecule comprising the nucleotide sequence as set forth in SEQ ID NO:7 and/or SEQ ID NO:8 or sufficient to modulate the activity of a protein comprising an amino acid sequence encoded by the nucleotide sequence as set forth in SEQ ID NO:7 and/or SEQ ID NO:8.

57. A method of treating a mammal suffering from a disease condition characterized by one or more symptoms of diabetes, obesity, a metabolic disorder, mitochondrial dysfunction, myopathy, genetic disorder or cancer, said method comprising administering to said mammal an effective amount of a nucleic acid molecule comprising the nucleotide sequence as set forth in SEQ ID NO:7 and/or SEQ ID NO:8 or a protein comprising an amino acid sequence encoded by the nucleotide sequence as set forth in SEQ ID NO:7 and/or SEQ ID NO:8.

58. A method of treating a mammal having a condition characterized by diabetes, obesity, a metabolic disorder, mitochondrial dysfunction, myopathy, genetic disorder or cancer which comprises adminstering an effective amount of an agent capable of modulating the expression of a nucleic acid molecule comprising the nucleotide sequence as set forth in SEQ ID NO:7 and/or SEQ ID NO:8 or a derivative, homolog or analog thereof for a time and a protein comprising an amino acid sequence encoded by the nucleotide sequence as set forth in SEQ ID NO:7 and/or SEQ ID NO:8 under conditions sufficient to modulate the expression of said nucleic acid or a derivative, homolog or analog thereof.

59. A method of treating a mammal having a condition characterized by diabetes, obesity, a metabolic disorder, mitochondrial dysfunction, myopathy, genetic disorder or cancer which comprises administering an effective amount of an agent capable of modulating the activity of a protein comprising an amino acid sequence encoded by the nucleotide sequence as set forth in SEQ ID NO:7 and/or SEQ ID NO:8 or a derivative, homolog, analog, chemical equivalent or mimetic thereof for time and under conditions sufficient to modulate the activity of said protein or a derivative, homolog, analog, chemical equivalent or mimetic thereof.

60. A method of treating a mammal having a condition characterized by diabetes, obesity, a metabolic disorder, mitochondrial dysfunction, myopathy, genetic disorder or cancer which comprises administering an effective amount of a nucleic acid molecule comprising the nucleotide sequence as set forth in SEQ ID NO:7 and/or SEQ ID NO:8 or derivative, homolog or analog thereof or of a protein comprising an amino acid sequence encoded by the nucleotide sequence as set forth in SEQ ID NO:7 and/or SEQ ID NO:8 or derivative, homolog, analog, chemical equivalent or mimetic thereof for time and under conditions sufficient to treat said condition.

61. A composition comprising a modulator of expression of a nucleic acid molecule comprising the nucleotide sequence as set forth in SEQ ID NO:7 and/or SEQ ID NO:8 or activity of a protein comprising an amino acid sequence encoded by the nucleotide sequence as set forth in SEQ ID NO:7 and/or SEQ ID NO:8, and one or more pharmaceutically acceptable carriers and/or diluents for use in treating one or more of diabetes, obesity, a metabolic disorder, mitochondrial dysfunction, myopathy, genetic disorder or cancer.

62. A method for detecting a disease or condition or a propensity for development of a disease or condition in a mammal said method comprising determining the level of expression of one or more of a nucleic acid molecule comprising the nucleotide sequence as set forth in SEQ ID NO:7 and/or SEQ ID NO:8 or the level of activity of a protein comprising an amino acid sequence encoded by the nucleotide sequence as set forth in SEQ ID NO:7 and/or SEQ ID NO:8 and comparing the results to a mammal not suffering the disease or condition.

63. The method of claim 62, wherein the disease is selected from the group consisting of diabetes, obesity or a metabolic disorder.

64. A method for detecting a disease or condition or a propensity for development of a disease or condition in a mammal said method comprising determining the level of expression of one or more of a nucleic acid molecule comprising the nucleotide sequence as set forth in SEQ ID NO:7 and/or SEQ ID NO:8 or the level of activity of a protein comprising an amino acid sequence encoded by the nucleotide sequence as set forth in SEQ ID NO:7 and/or SEQ ID NO:8 and comparing the results in a mammal which does not have the disease or condition.

65. The method of claim 64, wherein the disease is selected from the group consisting of diabetes, obesity or a metabolic disorder.
